(19) United States
(12) Patent Application Publication
(10) Pub. No.: US 2025/0044688 A1
(43) Pub. Date: Feb. 6, 2025

(54) RESIST COMPOSITION AND PATTERN FORMING PROCESS

(71) Applicant: Shin-Etsu Chemical Co., Ltd., Tokyo (JP)

(72) Inventors: Jun Hatakeyama, Joetsu-shi (JP); Masahiro Fukushima, Joetsu-shi (JP)

(73) Assignee: Shin-Etsu Chemical Co., Ltd., Tokyo (JP)

(21) Appl. No.: 18/784,039

(22) Filed: Jul. 25, 2024

(30) Foreign Application Priority Data
Jul. 28, 2023 (JP) 2023-123397

(51) Int. Cl. G03F 7/004; C07C 311/48; C07C 311/51; C07C 381/12; C07D 333/54; C07D 333/76; G03F 7/038; G03F 7/039

(52) U.S. Cl. CPC G03F 7/0045; C07C 311/48; C07C 311/51; C07C 381/12; C07D 333/54; C07D 333/76; G03F 7/0382; G03F 7/0392

(57) ABSTRACT

The resist composition exhibits a high sensitivity, reduced LWR, and improved CDU independent of whether it is of positive or negative tone. The resist composition comprises a sulfonium salt of N-carbonyltrifluoromethoxybenzenesulfonamide, N-carbonyldifluoromethoxybenzenesulfonamide, trifluoromethoxybenzenesulfonimide, and difluoromethoxybenzenesulfonimide as the quencher is provided. The resist composition offers a high sensitivity, reduced LWR and improved CDU independent of whether it is of positive or negative tone.

### CROSS-REFERENCE TO RELATED APPLICATIONS

This non-provisional application claims priority under 35 U.S.C. § 119(a) on Patent Application No. 2023-123397 filed in Japan on Jul. 28, 2023, the entire contents of which are hereby incorporated by reference.

### TECHNICAL FIELD

This invention relates to a resist composition and a patterning process using the composition.

### BACKGROUND ART

To meet the demand for higher integration density and operating speed of LSIs, the effort to reduce the pattern rule is in rapid progress. As the use of 5G high-speed communications and artificial intelligence (AI) is widely spreading, high-performance devices are needed for their processing. As the advanced miniaturization technology, manufacturing of microelectronic devices at the 5-nm node by the lithography using EUV of wavelength 13.5 nm has been implemented in a mass scale. Studies are made on the application of EUV lithography to 3-nm node devices of the next generation and 2-nm node devices of the next-but-one generation.

As the feature size reduces, image blurs due to acid diffusion become a problem. To insure resolution for fine patterns with a size of 45 nm et seq., not only an improvement in dissolution contrast is important as previously reported, but the control of acid diffusion is also important as reported in Non-Patent Document 1. Since chemically amplified resist compositions are designed such that sensitivity and contrast are enhanced by acid diffusion, an attempt to minimize acid diffusion by reducing the temperature and/or time of post-exposure bake (PEB) fails, resulting in drastic reductions of sensitivity and contrast.

A triangular tradeoff relationship among sensitivity, resolution, and edge roughness (LWR) has been pointed out. Specifically, a resolution improvement requires to suppress acid diffusion whereas a short acid diffusion distance leads to a decline of sensitivity.

The addition of an acid generator capable of generating a bulky acid is an effective means for suppressing acid diffusion. It was then proposed to incorporate repeat units derived from an onium salt having a polymerizable unsaturated bond in a polymer. Since this polymer functions as an acid generator, it is referred to as polymer-bound acid generator. Patent Document 1 discloses a sulfonium or iodonium salt having a polymerizable unsaturated bond, capable of generating a specific sulfonic acid. Patent Document 2 discloses a sulfonium salt having a sulfonic acid directly attached to the backbone.

Resist compositions adapted for the ArF lithography are typically based on (meth)acrylate polymers having acid labile groups. These acid labile groups undergo deprotection reaction when a photoacid generator capable of generating a sulfonic acid which is substituted at α-position with fluorine (referred to as “α-fluorinated sulfonic acid,” hereinafter) is used, but not when a photoacid generator capable of generating a sulfonic acid which is not substituted at α-position with fluorine (referred to as “α-non-fluorinated sulfonic acid,” hereinafter) or carboxylic acid is used. When a sulfonium or iodonium salt capable of generating α-fluorinated sulfonic acid is mixed with a sulfonium or iodonium salt capable of generating α-non-fluorinated sulfonic acid, the sulfonium or iodonium salt capable of generating α-non-fluorinated sulfonic acid undergoes ion exchange with the α-fluorinated sulfonic acid. Through the ion exchange, the α-fluorinated sulfonic acid once generated upon light exposure is converted back to the sulfonium or iodonium salt. Then the sulfonium or iodonium salt of α-non-fluorinated sulfonic acid or carboxylic acid functions as a quencher. Patent Document 3 discloses a resist composition comprising a sulfonium or iodonium salt capable of generating carboxylic acid as the quencher.

Since sulfonamide has an acidity approximate to that of carboxylic acid, a sulfonium salt of sulfonamide functions as a quencher. Patent Documents 4 and 5 disclose quenchers in the form of sulfonium salts capable of generating sulfonamide.

It is pointed out that the agglomeration of a quencher causes to degrade the dimensional uniformity or CDU of resist patterns. It is thus expected that the CDU of resist patterns after development is improved by preventing the quencher from agglomerating in the resist film or achieving a uniform distribution of the quencher.

The potential hazard to health of perfluoroalkyl substances (PFASs) is pointed out. The REACH Regulation of EU aims to put restrictions on the manufacture and sales of PFASs. A number of substances including PFASs are currently used in the field of semiconductor lithography. For example, materials containing PFASs are applied to surfactants, acid generators, quenchers and the like.

### CITATION LIST

Patent Document 1: JP-A 2006-045311 (U.S. Pat. No. 7,482,108)
Patent Document 2: JP-A 2006-178317
Patent Document 3: JP-A 2007-114431
Patent Document 4: JP-A 2012-108447
Patent Document 5: JP-A 2013-145256
Non-Patent Document 1: SPIE Vol. 6520 65203L-1 (2007)

### SUMMARY OF THE INVENTION

It is desired to have a quencher capable of reducing the roughness (LWR) of line patterns, improving the dimensional uniformity (CDU) of hole patterns, and increasing the sensitivity of a resist composition. To this end, image blurs due to acid diffusion must be significantly reduced.

An object of the invention is to provide a resist composition which exhibits a high sensitivity, reduced LWR, and improved CDU independent of whether it is of positive or negative tone, and a pattern forming process using the same.

The inventors have found that a sulfonium salt of N-carbonyltrifluoromethoxybenzenesulfonamide, N-carbonyldifluoromethoxybenzenesulfonamide, trifluoromethoxybenzenesulfonimide or difluoromethoxybenzenesulfonimide is an effective quencher for suppressing acid diffusion. Because of the effect of suppressing acid diffusion and the effect of preventing the quencher from agglomerating by the electronic repulsion between bulky groups such as trifluoromethoxy or difluoromethoxy, a resist composition using the sulfonium salt as the quencher exhibits reduced LWR, improved CDU, high resolution, and wide process margin.

In one aspect, the invention provides a resist composition comprising a quencher containing a sulfonium salt of at least one compound selected from N-carbonyltrifluoromethoxybenzenesulfonamide, N-carbonyldifluoromethoxybenzenesulfonamide, trifluoromethoxybenzenesulfonimide, and difluoromethoxybenzenesulfonimide.

In a preferred embodiment, the sulfonium salt has the formula (1).

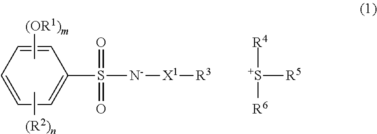

Herein m is an integer of 1 to 5, n is an integer of 0 to 4, m+n is from 1 to 5,
X¹ is —C(═O)— or —S(═O)₂—,
R¹ is trifluoromethyl or difluoromethyl,
R² is a C₁-C₆ saturated hydrocarbyl group, C₁-C₆ saturated hydrocarbyloxy group, hydroxy, carboxy, cyano, nitro or halogen,
R³ is a C₁-C₂₆ hydrocarbyl group which may contain at least one element selected from oxygen, sulfur, nitrogen, and halogen,
R⁴ to R⁶ are each independently halogen or a C₁-C₂₀ hydrocarbyl group which may contain a heteroatom, R⁴ and R⁵ may bond together to form a ring with the sulfur atom to which they are attached.

Often, the resist composition further comprises a base polymer.

In a preferred embodiment, the base polymer comprises repeat units having the formula (a1) or repeat units having the formula (a2).

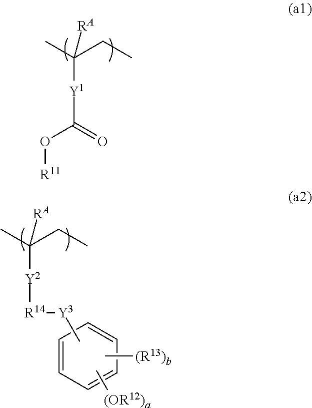

Herein R^{A} is each independently hydrogen or methyl,
Y¹ is a single bond, phenylene, naphthylene, or a C₁-C₁₂ linking group containing at least one moiety selected from ester bond, ether bond and lactone ring,
Y² is a single bond or ester bond,
Y³ is a single bond, ether bond or ester bond,
R¹¹ and R¹² are each independently an acid labile group,
R¹³ is fluorine, trifluoromethyl, cyano or a C₁-C₆ saturated hydrocarbyl group,
R¹⁴ is a single bond or a C₁-C₆ alkanediyl group in which some —CH₂— may be replaced by an ether bond or ester bond,
a is 1 or 2, b is an integer of 0 to 4, and a+b is from 1 to 5.

In a preferred embodiment, the resist composition is a chemically amplified positive resist composition.

In another embodiment, the base polymer is free of an acid labile group. The resist composition is then a chemically amplified negative resist composition.

In a preferred embodiment, the base polymer comprises repeat units of at least one type selected from repeat units having the formula (f1), repeat units having the formula (f2), and repeat units having the formula (f3).

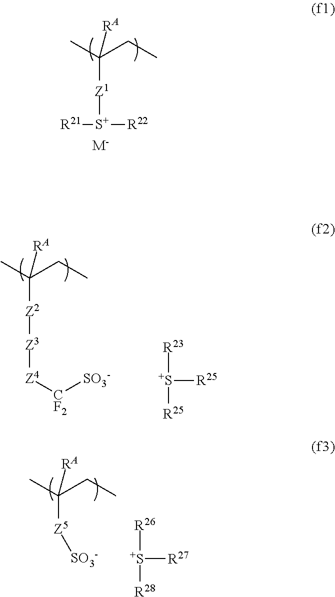

Herein R^{A} is each independently hydrogen or methyl,
Z¹ is a single bond, a C₁-C₆ aliphatic hydrocarbylene group, phenylene group, naphthylene group, or C₇-C₁₈ group obtained by combining the foregoing, or —O—Z¹¹—, —C(═O)—O—Z¹¹— or —C(═O)—NH—Z¹¹—, Z¹¹ is a C₁-C₆ aliphatic hydrocarbylene group, phenylene group, naphthylene group, or C₇-C₁₈ group obtained by combining the foregoing, which may contain a carbonyl moiety, ester bond, ether bond or hydroxy moiety,
Z² is a single bond or ester bond,
Z³ is a single bond, —Z³¹—C(═O)—O—, —Z³¹—O— or —Z³¹—O—C(═O)—, Z³¹ is a C₁-C₁₂ hydrocarbylene group, phenylene group, or C₇-C₁₈ group obtained by combining the foregoing, which may contain a carbonyl moiety, ester bond, ether bond, iodine or bromine,
Z⁴ is methylene, 2,2,2-trifluoro-1,1-ethanediyl or carbonyl,
Z⁵ is a single bond, methylene, ethylene, phenylene, fluorinated phenylene, trifluoromethyl-substituted phenylene group, —O—Z⁵¹—, —C(═O)—O—Z⁵¹—, or —C(═O)—NH—Z⁵¹—,
Z⁵¹ is a C₁-C₆ aliphatic hydrocarbylene group, phenylene group, fluorinated phenylene group, or trifluoromethyl-substituted phenylene group, which may contain a carbonyl moiety, ester bond, ether bond, halogen or hydroxy moiety,
R²¹ to R²⁸ are each independently halogen or a C₁-C₂₀ hydrocarbyl group which may contain a heteroatom, a pair of R²³ and R²⁴ or R²⁶ and R²⁷ may bond together to form a ring with the sulfur atom to which they are attached, and

M⁻ is a non-nucleophilic counter ion.

The resist composition may further comprise an acid generator capable of generating a strong acid. The preferred acid generator generates a sulfonic acid, imide acid or methide acid.

The resist composition may further comprise an organic solvent and/or a surfactant.

In another aspect, the invention provides a pattern forming process comprising the steps of applying the resist composition defined herein onto a substrate to form a resist film thereon, exposing the resist film to high-energy radiation, and developing the exposed resist film in a developer.

The high-energy radiation is typically KrF excimer laser, ArF excimer laser, EB or EUV of wavelength 3 to 15 nm.

### Advantageous Effects of Invention

The sulfonium salt of N-carbonyltrifluoromethoxybenzenesulfonamide, N-carbonyldifluoromethoxybenzenesulfonamide, trifluoromethoxybenzenesulfonimide or difluoromethoxybenzenesulfonimide serves as a quencher capable of suppressing acid diffusion. The salt is successful in achieving low acid diffusion performance and improving LWR and CDU. A resist composition having reduced LWR and improved CDU can be designed.

### DETAILED DESCRIPTION OF THE INVENTION

As used herein, the singular forms “a,” “an” and “the” include plural referents unless the context clearly dictates otherwise. “Optional” or “optionally” means that the subsequently described event or circumstances may or may not occur, and that description includes instances where the event or circumstance occurs and instances where it does not. The notation (Cn-Cm) means a group containing from n to m carbon atoms per group. In chemical formulae, the broken line (- - -) designates a point of attachment or valence bond. As used herein, the term “fluorinated” refers to a fluorine-substituted or fluorine-containing compound or group. The terms “group” and “moiety” are interchangeable.

The abbreviations and acronyms have the following meaning.
EB: electron beam
EUV: extreme ultraviolet
Mw: weight average molecular weight
Mn: number average molecular weight
Mw/Mn: molecular weight distribution or dispersity
GPC: gel permeation chromatography
PEB: post-exposure bake
PAG: photoacid generator
LWR: line width roughness
CDU: critical dimension uniformity

### [Resist Composition]

One embodiment of the invention is a resist composition comprising a quencher containing a sulfonium salt of at least one compound selected from N-carbonyltrifluoromethoxybenzenesulfonamide, N-carbonyldifluoromethoxybenzenesulfonamide, trifluoromethoxybenzenesulfonimide, and difluoromethoxybenzenesulfonimide. The sulfonium salt is referred to as Sulfonium Salt A, hereinafter.

### [Quencher]

Sulfonium Salt A preferably has the formula (1).

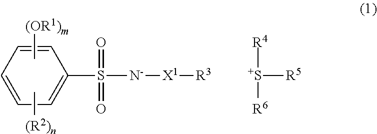

In formula (1), m is an integer of 1 to 5, n is an integer of 0 to 4, and m+n is from 1 to 5.

In formula (1), X¹ is —C(═O)— or —S(═O)₂—.

In formula (1), R¹ is trifluoromethyl or difluoromethyl.

In formula (1), R² is a C₁-C₆ saturated hydrocarbyl group, C₁-C₆ saturated hydrocarbyloxy group, hydroxy, carboxy, cyano, nitro or halogen.

The C₁-C₆ saturated hydrocarbyl group and saturated hydrocarbyl moiety in the C₁-C₆ saturated hydrocarbyloxy group represented by R² may be straight, branched or cyclic. Examples thereof include C₁-C₆ alkyl groups such as methyl, ethyl, n-propyl, isopropyl, n-butyl, isobutyl, sec-butyl, tert-butyl, n-pentyl, tert-pentyl, neopentyl, and n-hexyl; C₃-C₆ cyclic saturated hydrocarbon groups such as cyclopropyl, cyclobutyl, cyclopentyl, and cyclohexyl; and combinations thereof. Suitable halogen atoms represented by R² include fluorine, chlorine, bromine and iodine.

In formula (1), R³ is a C₁-C₂₆ hydrocarbyl group. The hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, hexadecyl, heptadecyl, octadecyl, nonadecyl and icosyl; C₃-C₂₀ cyclic saturated hydrocarbyl groups such as cyclopropyl, cyclopentyl, cyclohexyl, cycloheptyl, cyclopropylmethyl, cyclopropylethyl, cyclopropylpropyl, 4-methylcyclohexyl, cyclohexylmethyl, cyclohexylethyl, cyclohexylpropyl, norbornyl, norbornylmethyl, norbornylethyl, norbornylpropyl, adamantyl, adamantylmethyl, adamantylethyl, adamantylpropyl, and noradamantyl; C₂-C₂₀ alkenyl groups such as vinyl, propenyl, butenyl, and hexenyl; C₂-C₂₀ alkynyl groups such as ethynyl, propynyl, and butynyl; C₃-C₂₀ cyclic unsaturated aliphatic hydrocarbyl groups such as cyclohexenyl and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, anthryl, phenanthryl, phenalenyl, pyrenyl, indanyl, indenyl, tetralinyl, acenaphthenyl, acenaphthylenyl, and fluorenyl; C₇-C₂₀ aralkyl groups such as benzyl, phenethyl, phenylpropyl, naphthylmethyl, naphthylethyl and naphthylpropyl; and combinations thereof.

The hydrocarbyl group R³ may contain at least one element selected from oxygen, sulfur, nitrogen, and halogen. That is, in the hydrocarbyl group, some or all of the hydrogen atoms may be substituted by hydroxy, fluorine, chlorine, bromine, iodine, cyano, nitro, amino or carboxy, and some —CH₂— may be replaced by carbonyl, ether bond, ester bond, sulfonate ester bond, carbonate bond, amide bond, urethane bond, or urea bond.

Examples of the anion in Sulfonium Salt A are shown below, but not limited thereto.

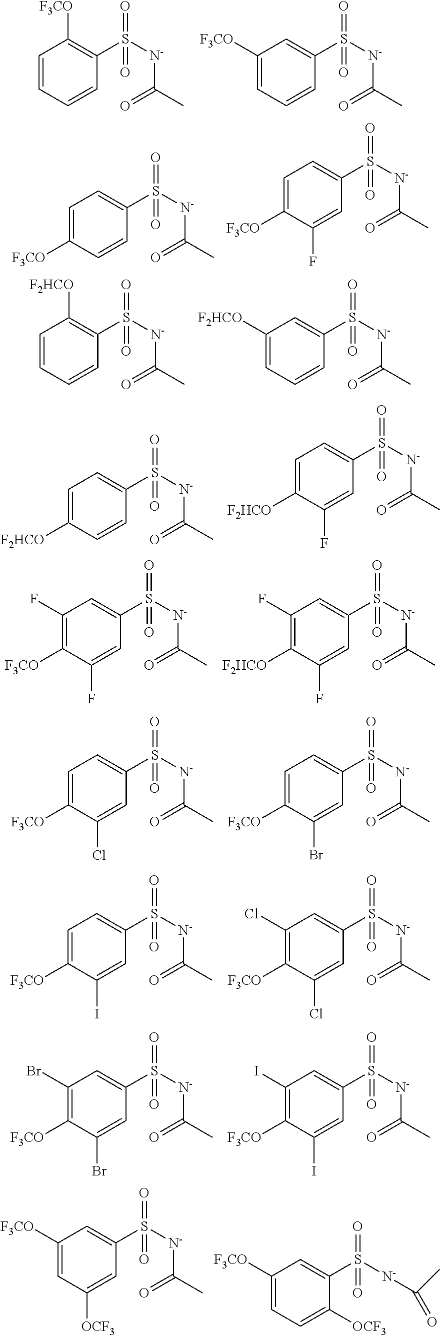

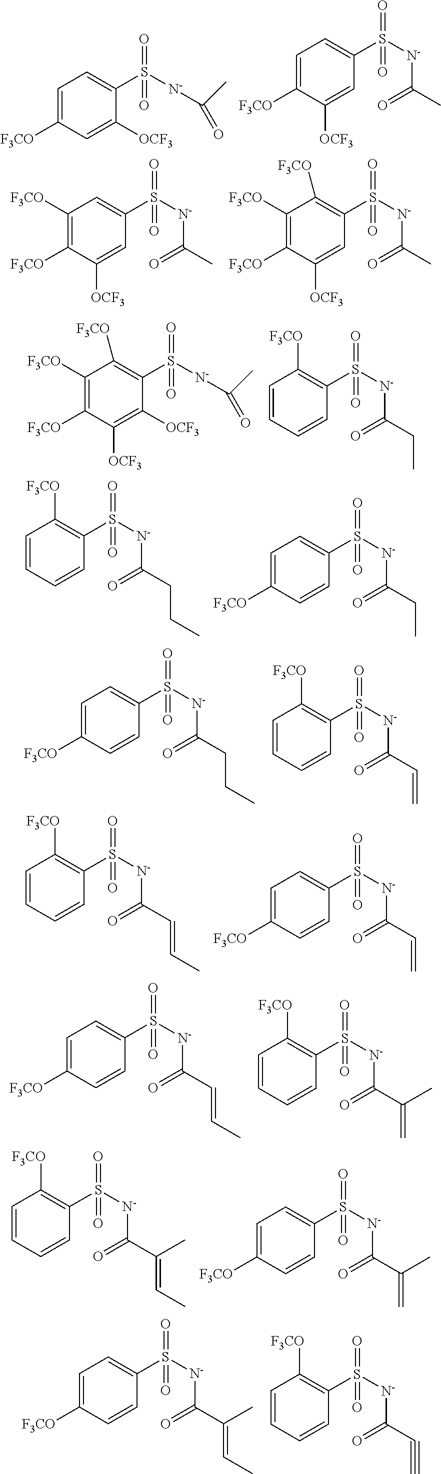

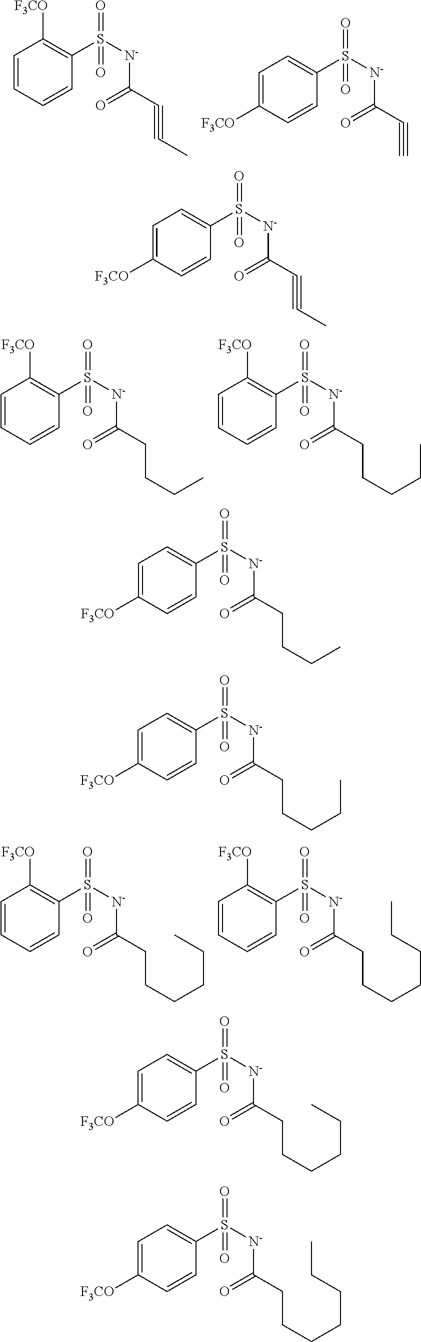

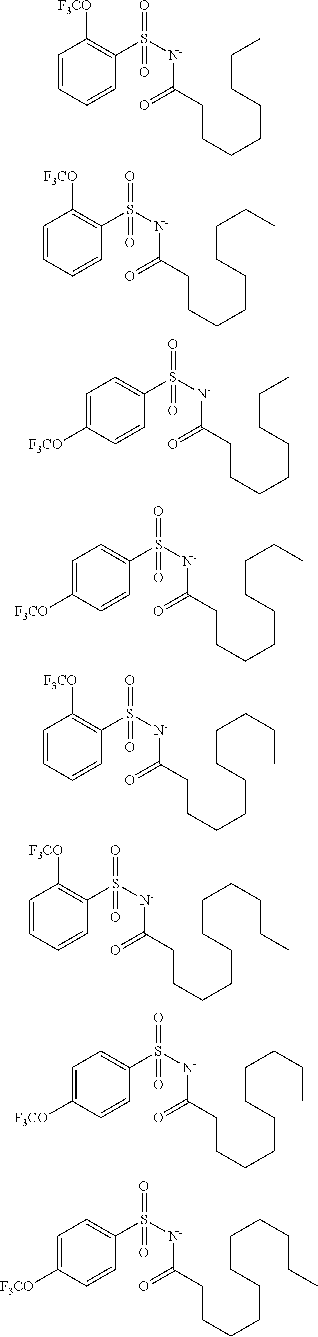

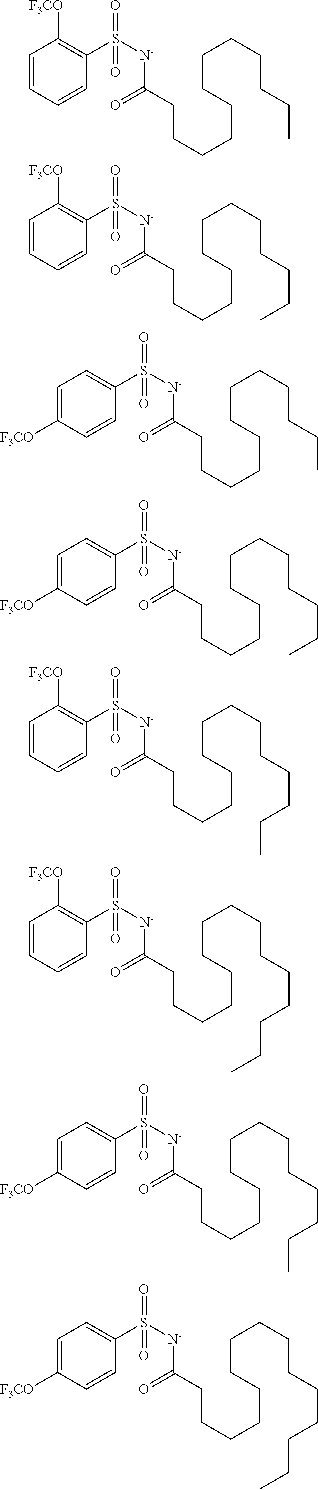

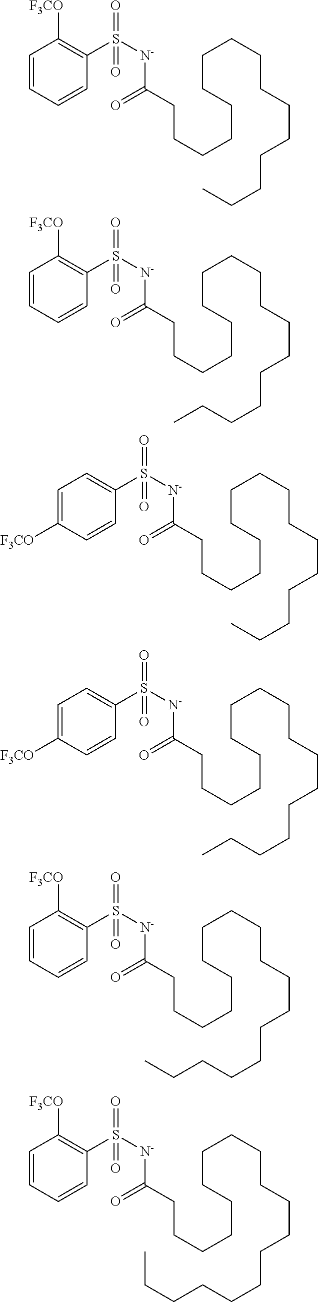 [SMILES: CCCCCCCCCCCCCCCCC(=O)[N-]S(=O)(=O)c1ccccc1OC(F)(F)F ; CCCCCCCCCCCCCCCCCC(=O)[N-]S(=O)(=O)c1ccccc1OC(F)(F)F ; CCCCCCCCCCCCCCCCC(=O)[N-]S(=O)(=O)c1ccc(OC(F)(F)F)cc1 ; CCCCCCCCCCCCCCCCCC(=O)[N-]S(=O)(=O)c1ccc(OC(F)(F)F)cc1 ; CCCCCCCCCCCCCCCCCCC(=O)[N-]S(=O)(=O)c1ccccc1OC(F)(F)F ; CCCCCCCCCCCCCCCCCCCC(=O)[N-]S(=O)(=O)c1ccccc1OC(F)(F)F]

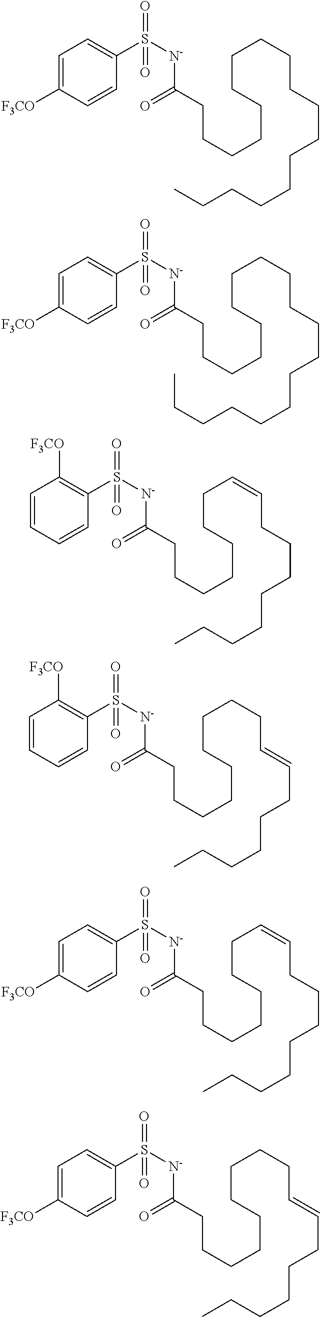 [SMILES: CCCCCCCCCCCCCCCCCCC(=O)[N-]S(=O)(=O)c1ccc(OC(F)(F)F)cc1 ; CCCCCCCCCCCCCCCCCCCC(=O)[N-]S(=O)(=O)c1ccc(OC(F)(F)F)cc1 ; CCCCCCCC/C=C\CCCCCCCC(=O)[N-]S(=O)(=O)c1ccccc1OC(F)(F)F ; CCCCCC/C=C/CCCCCCCCCC(=O)[N-]S(=O)(=O)c1ccccc1OC(F)(F)F ; CCCCCCCC/C=C\CCCCCCCC(=O)[N-]S(=O)(=O)c1ccc(OC(F)(F)F)cc1 ; CCCCCC/C=C/CCCCCCCCCC(=O)[N-]S(=O)(=O)c1ccc(OC(F)(F)F)cc1]

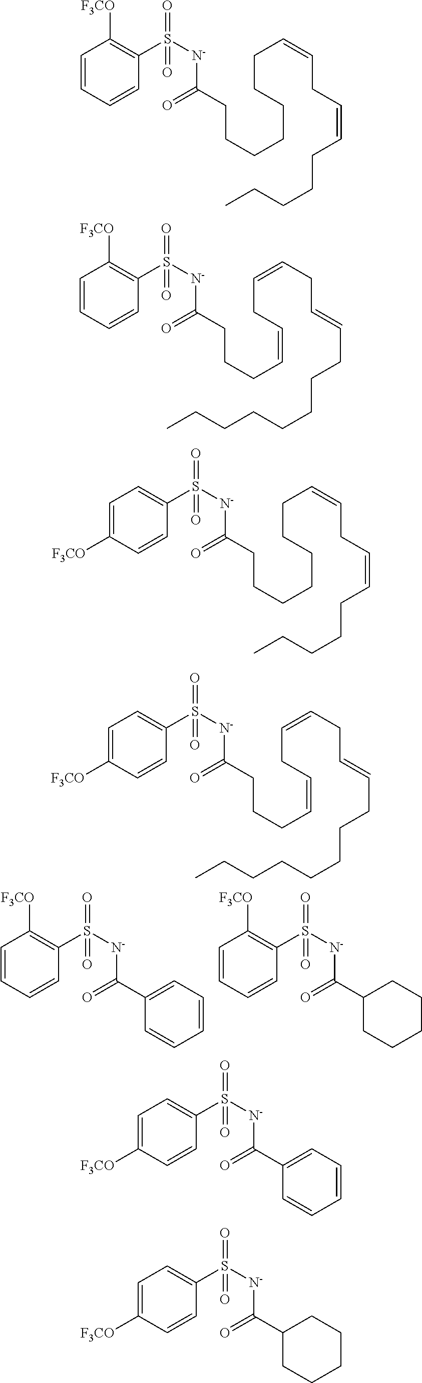

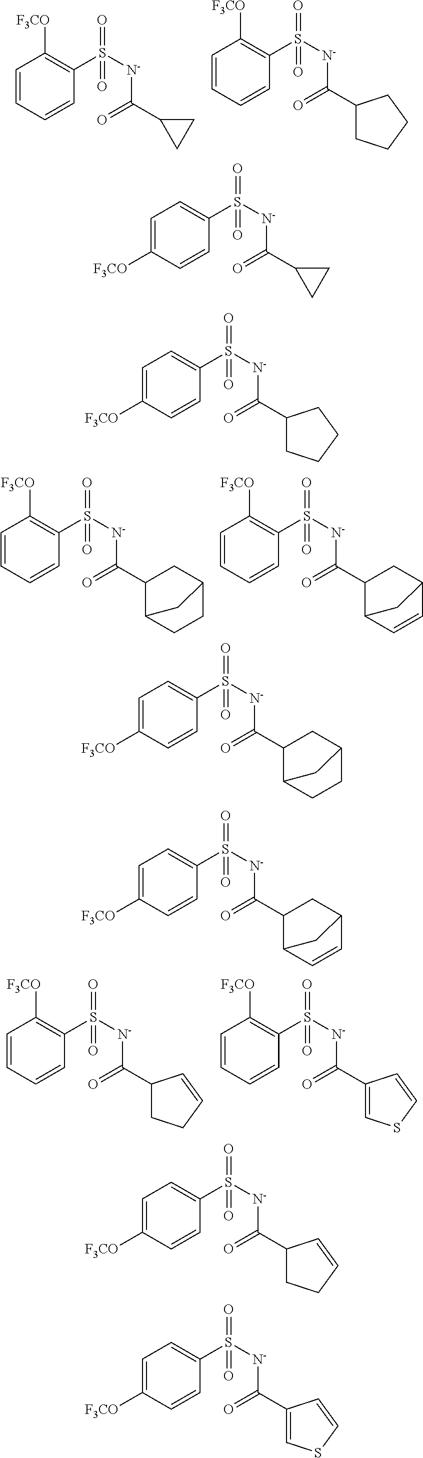

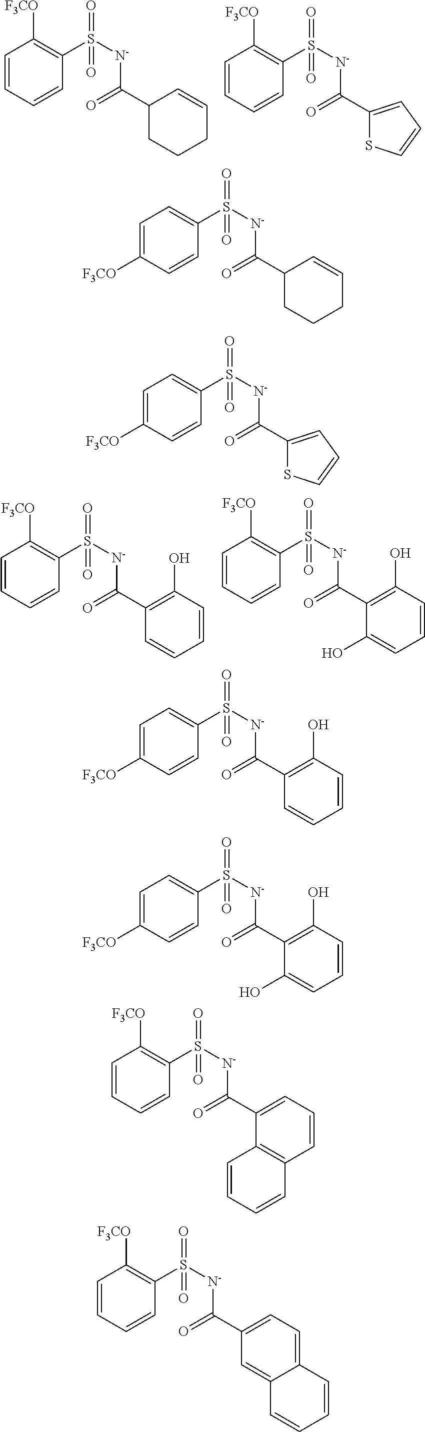

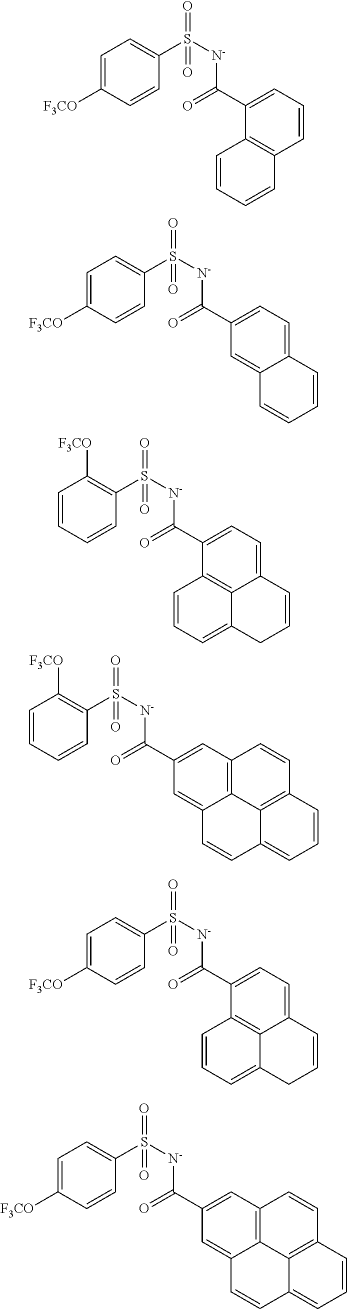 [SMILES: O=C([N-]S(=O)(=O)c1ccc(OC(F)(F)F)cc1)c1cccc2ccccc12 ; O=C([N-]S(=O)(=O)c1ccc(OC(F)(F)F)cc1)c1ccc2ccccc2c1 ; O=C([N-]S(=O)(=O)c1ccccc1OC(F)(F)F)c1ccc2c3c(cccc13)CC=C2 ; O=C([N-]S(=O)(=O)c1ccccc1OC(F)(F)F)c1cc2ccc3cccc4ccc(c1)c2c34 ; O=C([N-]S(=O)(=O)c1ccc(OC(F)(F)F)cc1)c1ccc2c3c(cccc13)CC=C2 ; O=C([N-]S(=O)(=O)c1ccc(OC(F)(F)F)cc1)c1cc2ccc3cccc4ccc(c1)c2c34]

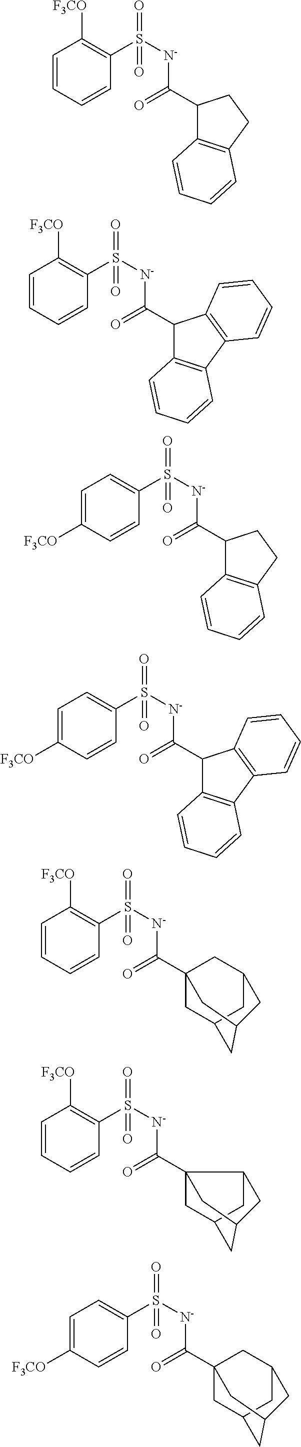

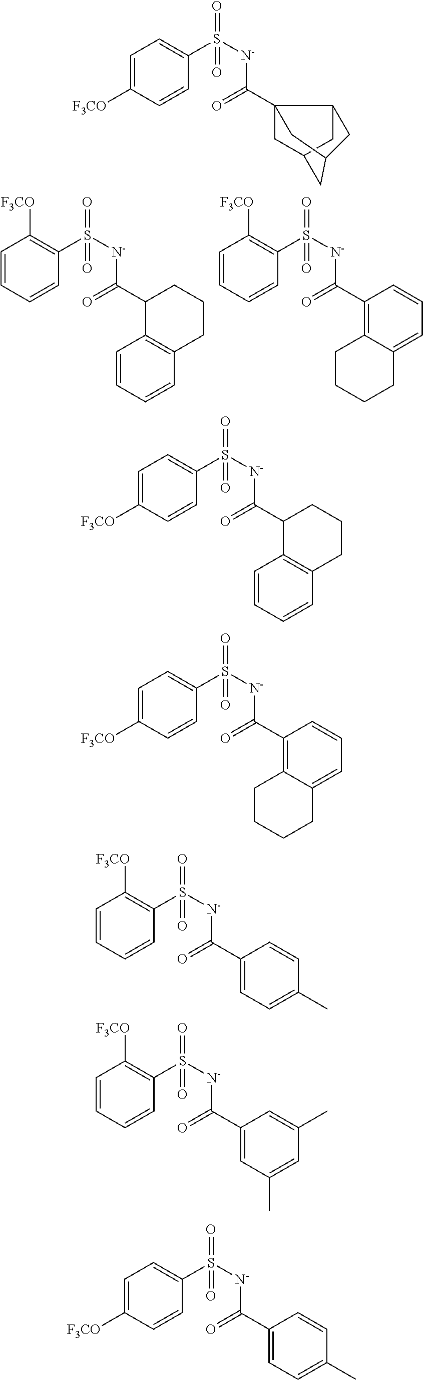

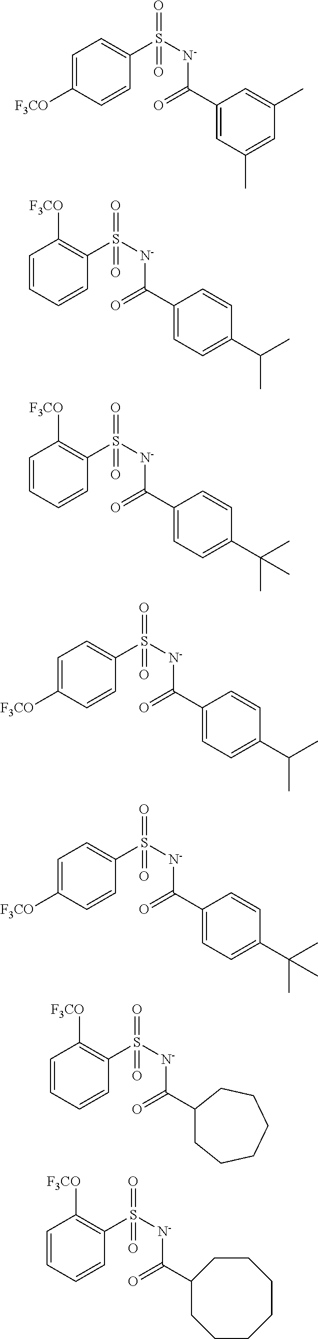

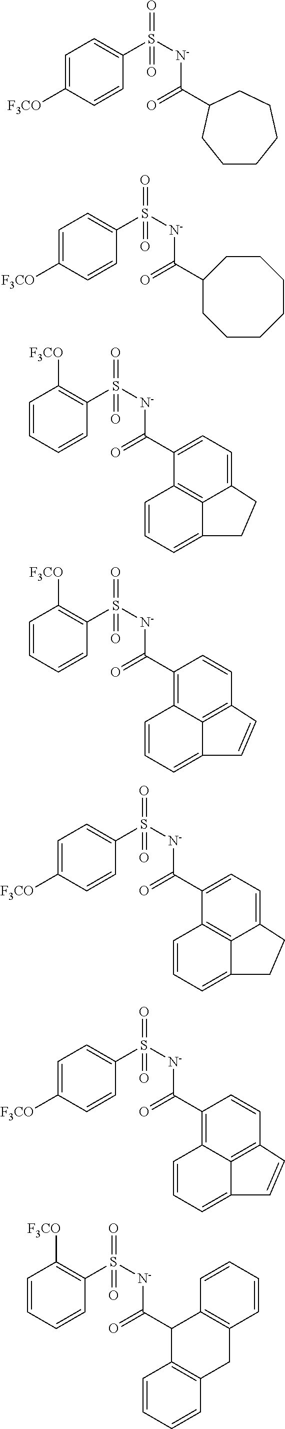

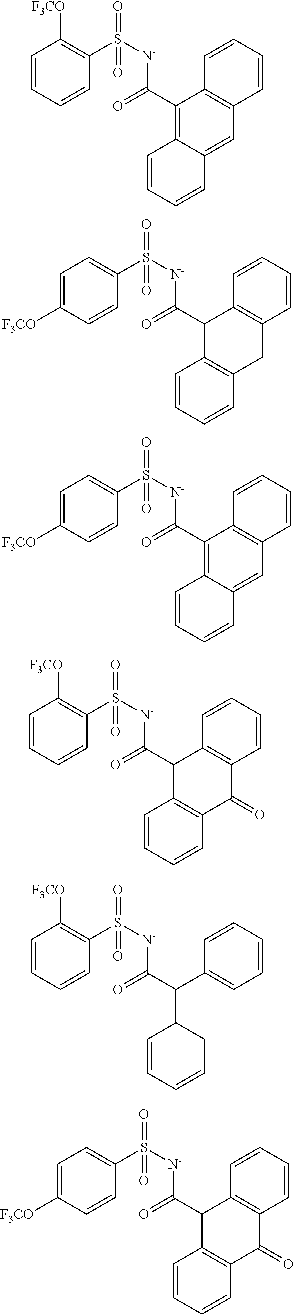 [SMILES: O=C([N-]S(=O)(=O)c1ccccc1OC(F)(F)F)c1c2ccccc2cc2ccccc12 ; O=C([N-]S(=O)(=O)c1ccc(OC(F)(F)F)cc1)C1c2ccccc2Cc2ccccc21 ; O=C([N-]S(=O)(=O)c1ccc(OC(F)(F)F)cc1)c1c2ccccc2cc2ccccc12 ; O=C1c2ccccc2C(C(=O)[N-]S(=O)(=O)c2ccccc2OC(F)(F)F)c2ccccc21 ; O=C([N-]S(=O)(=O)c1ccccc1OC(F)(F)F)C(c1ccccc1)C1C=CC=CC1 ; O=C1c2ccccc2C(C(=O)[N-]S(=O)(=O)c2ccc(OC(F)(F)F)cc2)c2ccccc21]

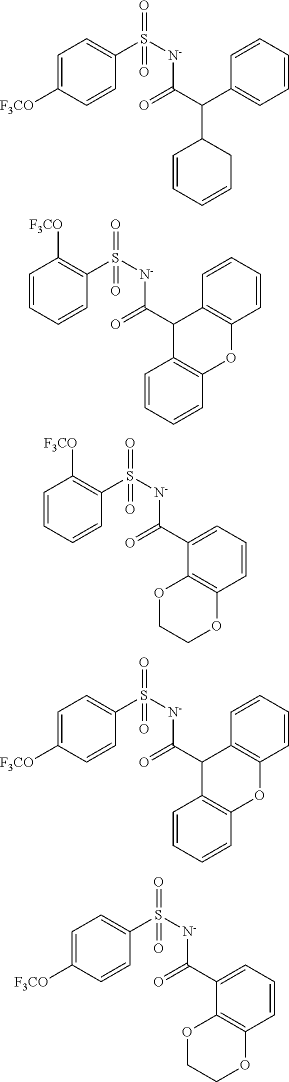 [SMILES: O=C([N-]S(=O)(=O)c1ccc(OC(F)(F)F)cc1)C(c1ccccc1)C1C=CC=CC1 ; O=C([N-]S(=O)(=O)c1ccccc1OC(F)(F)F)C1c2ccccc2Oc2ccccc21 ; O=C([N-]S(=O)(=O)c1ccccc1OC(F)(F)F)c1cccc2c1OCCO2 ; O=C([N-]S(=O)(=O)c1ccc(OC(F)(F)F)cc1)C1c2ccccc2Oc2ccccc21 ; O=C([N-]S(=O)(=O)c1ccc(OC(F)(F)F)cc1)c1cccc2c1OCCO2]

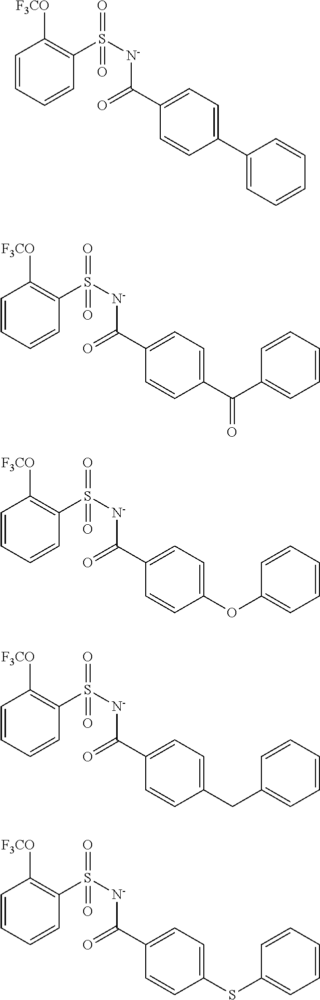 [SMILES: O=C([N-]S(=O)(=O)c1ccccc1OC(F)(F)F)c1ccc(-c2ccccc2)cc1 ; O=C([N-]S(=O)(=O)c1ccccc1OC(F)(F)F)c1ccc(C(=O)c2ccccc2)cc1 ; O=C([N-]S(=O)(=O)c1ccccc1OC(F)(F)F)c1ccc(Oc2ccccc2)cc1 ; O=C([N-]S(=O)(=O)c1ccccc1OC(F)(F)F)c1ccc(Cc2ccccc2)cc1 ; O=C([N-]S(=O)(=O)c1ccccc1OC(F)(F)F)c1ccc(Sc2ccccc2)cc1]

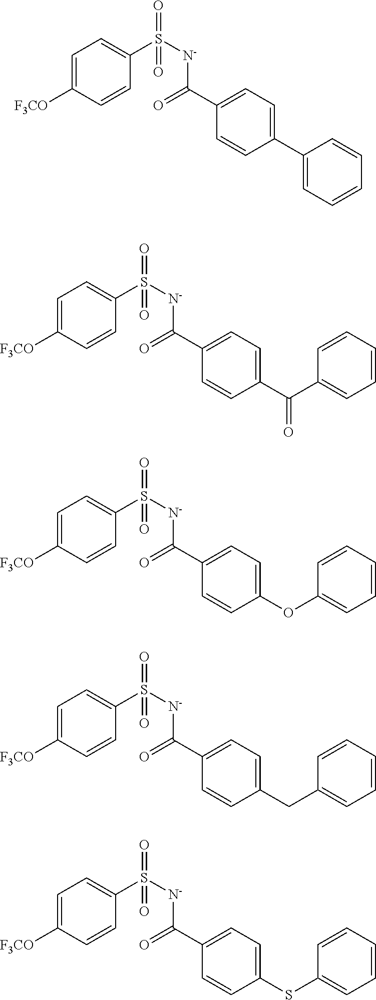 [SMILES: O=C([N-]S(=O)(=O)c1ccc(OC(F)(F)F)cc1)c1ccc(-c2ccccc2)cc1 ; O=C([N-]S(=O)(=O)c1ccc(OC(F)(F)F)cc1)c1ccc(C(=O)c2ccccc2)cc1 ; O=C([N-]S(=O)(=O)c1ccc(OC(F)(F)F)cc1)c1ccc(Oc2ccccc2)cc1 ; O=C([N-]S(=O)(=O)c1ccc(OC(F)(F)F)cc1)c1ccc(Cc2ccccc2)cc1 ; O=C([N-]S(=O)(=O)c1ccc(OC(F)(F)F)cc1)c1ccc(Sc2ccccc2)cc1]

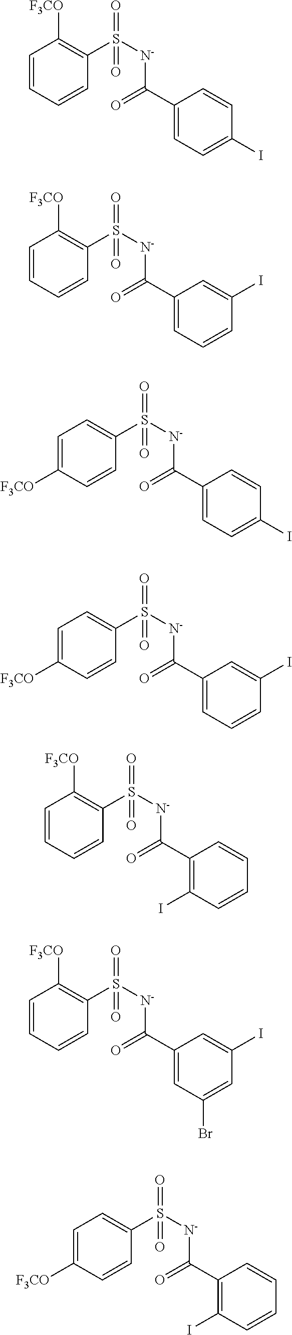

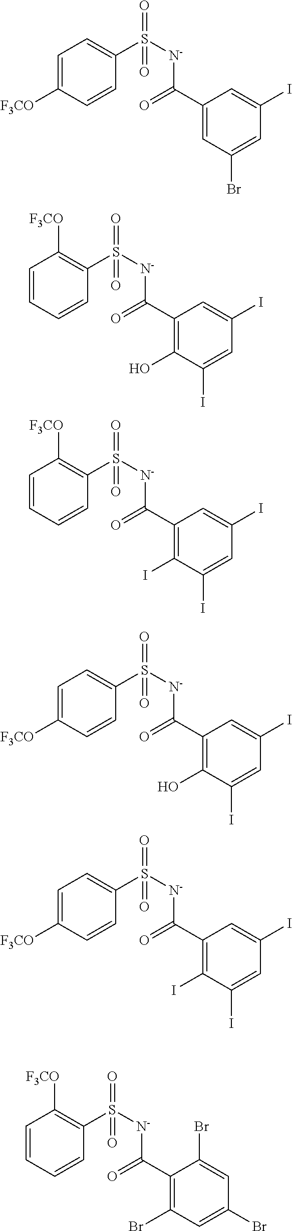 [SMILES: O=C([N-]S(=O)(=O)c1ccc(OC(F)(F)F)cc1)c1cc(Br)cc(I)c1 ; O=C([N-]S(=O)(=O)c1ccccc1OC(F)(F)F)c1cc(I)cc(I)c1O ; O=C([N-]S(=O)(=O)c1ccccc1OC(F)(F)F)c1cc(I)cc(I)c1I ; O=C([N-]S(=O)(=O)c1ccc(OC(F)(F)F)cc1)c1cc(I)cc(I)c1O ; O=C([N-]S(=O)(=O)c1ccc(OC(F)(F)F)cc1)c1cc(I)cc(I)c1I ; O=C([N-]S(=O)(=O)c1ccccc1OC(F)(F)F)c1c(Br)cc(Br)cc1Br]

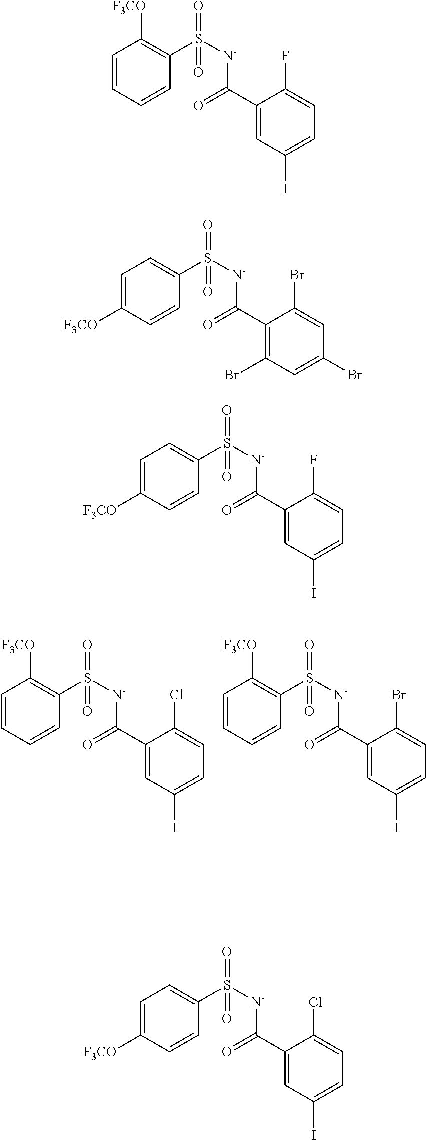 [SMILES: O=C([N-]S(=O)(=O)c1ccccc1OC(F)(F)F)c1cc(I)ccc1F ; O=C([N-]S(=O)(=O)c1ccc(OC(F)(F)F)cc1)c1c(Br)cc(Br)cc1Br ; O=C([N-]S(=O)(=O)c1ccc(OC(F)(F)F)cc1)c1cc(I)ccc1F ; O=C([N-]S(=O)(=O)c1ccccc1OC(F)(F)F)c1cc(I)ccc1Cl ; O=C([N-]S(=O)(=O)c1ccccc1OC(F)(F)F)c1cc(I)ccc1Br ; O=C([N-]S(=O)(=O)c1ccc(OC(F)(F)F)cc1)c1cc(I)ccc1Cl]

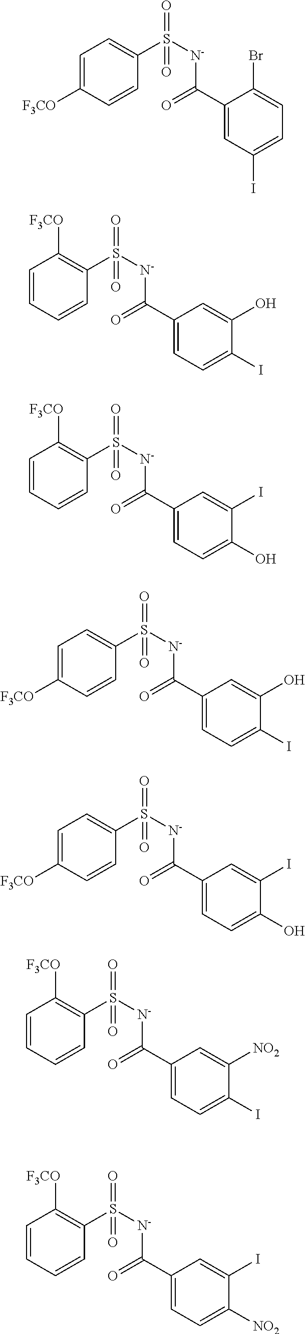

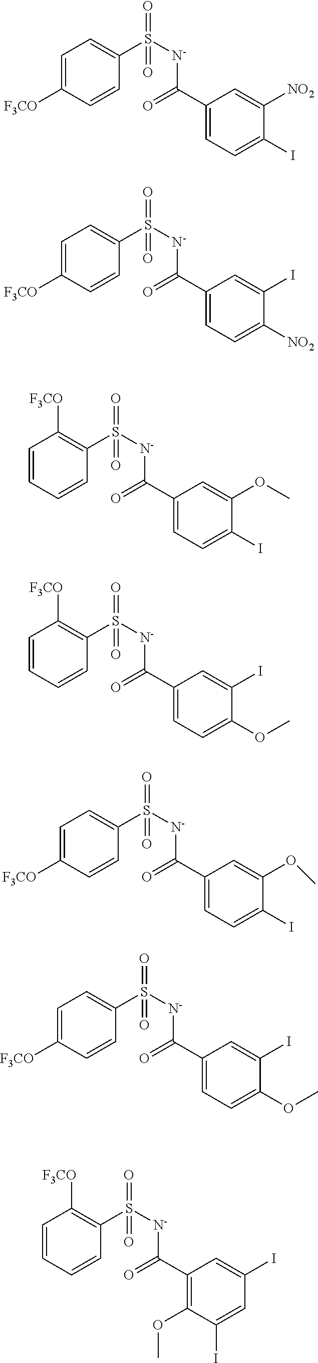

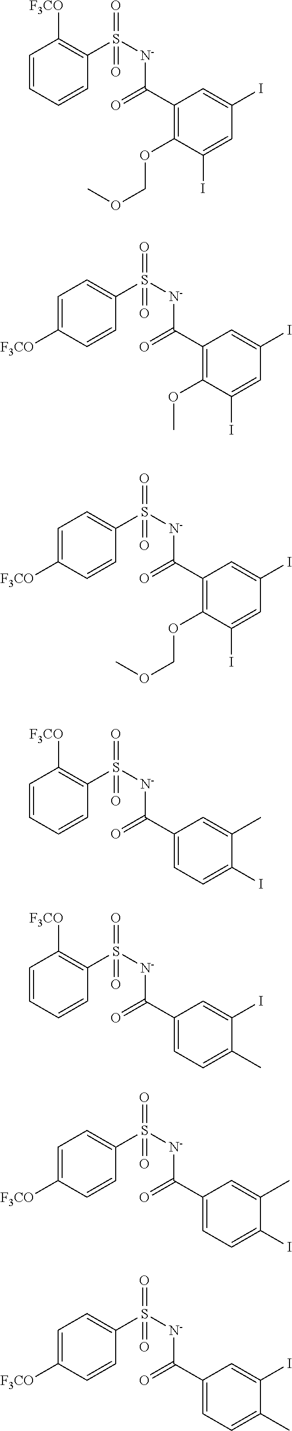

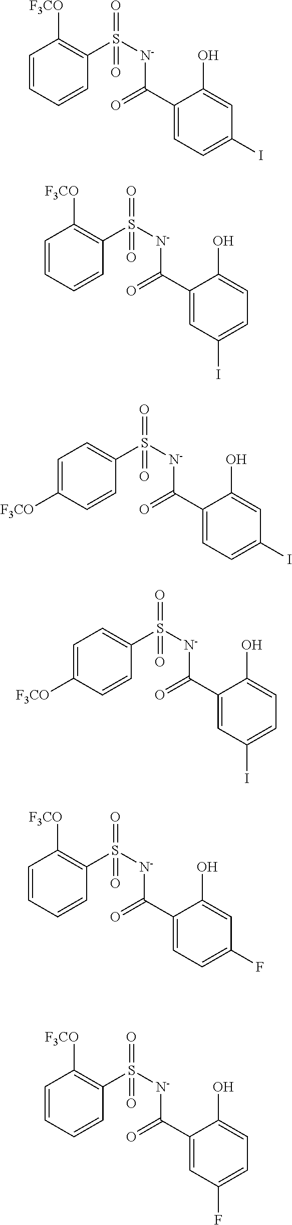 [SMILES: O=C([N-]S(=O)(=O)c1ccccc1OC(F)(F)F)c1ccc(I)cc1O ; O=C([N-]S(=O)(=O)c1ccccc1OC(F)(F)F)c1cc(I)ccc1O ; O=C([N-]S(=O)(=O)c1ccc(OC(F)(F)F)cc1)c1ccc(I)cc1O ; O=C([N-]S(=O)(=O)c1ccc(OC(F)(F)F)cc1)c1cc(I)ccc1O ; O=C([N-]S(=O)(=O)c1ccccc1OC(F)(F)F)c1ccc(F)cc1O ; O=C([N-]S(=O)(=O)c1ccccc1OC(F)(F)F)c1cc(F)ccc1O]

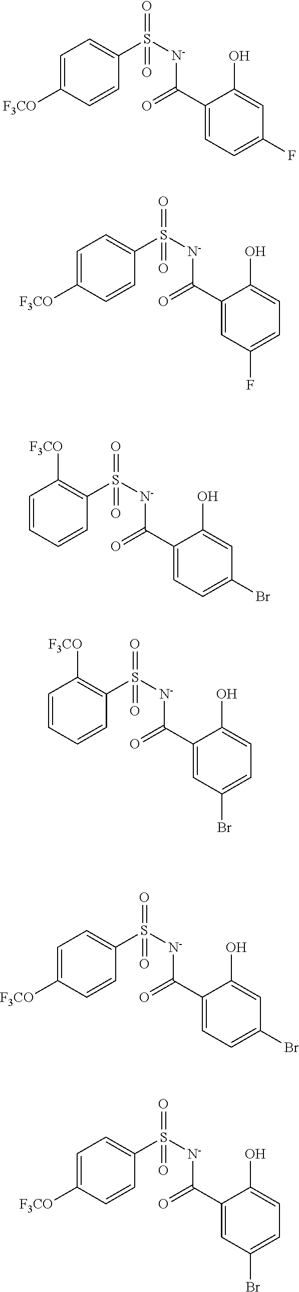 [SMILES: O=C([N-]S(=O)(=O)c1ccc(OC(F)(F)F)cc1)c1ccc(F)cc1O ; O=C([N-]S(=O)(=O)c1ccc(OC(F)(F)F)cc1)c1cc(F)ccc1O ; O=C([N-]S(=O)(=O)c1ccccc1OC(F)(F)F)c1ccc(Br)cc1O ; O=C([N-]S(=O)(=O)c1ccccc1OC(F)(F)F)c1cc(Br)ccc1O ; O=C([N-]S(=O)(=O)c1ccc(OC(F)(F)F)cc1)c1ccc(Br)cc1O ; O=C([N-]S(=O)(=O)c1ccc(OC(F)(F)F)cc1)c1cc(Br)ccc1O]

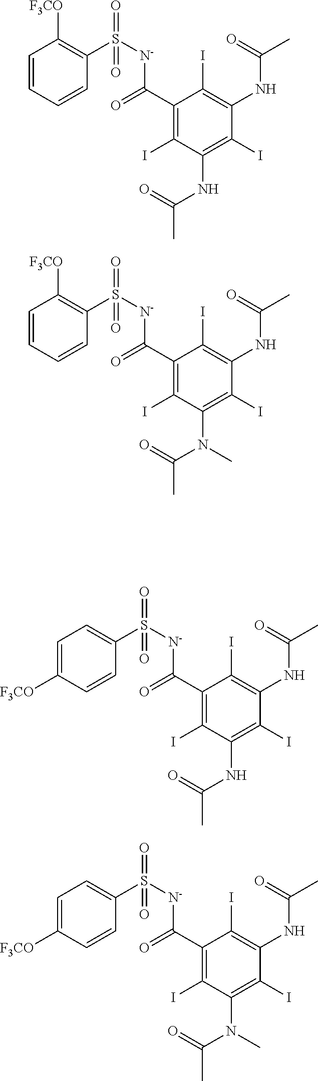 [SMILES: CC(=O)Nc1c(I)c(NC(C)=O)c(I)c(C(=O)[N-]S(=O)(=O)c2ccccc2OC(F)(F)F)c1I ; CC(=O)Nc1c(I)c(C(=O)[N-]S(=O)(=O)c2ccccc2OC(F)(F)F)c(I)c(N(C)C(C)=O)c1I ; CC(=O)Nc1c(I)c(NC(C)=O)c(I)c(C(=O)[N-]S(=O)(=O)c2ccc(OC(F)(F)F)cc2)c1I ; CC(=O)Nc1c(I)c(C(=O)[N-]S(=O)(=O)c2ccc(OC(F)(F)F)cc2)c(I)c(N(C)C(C)=O)c1I]

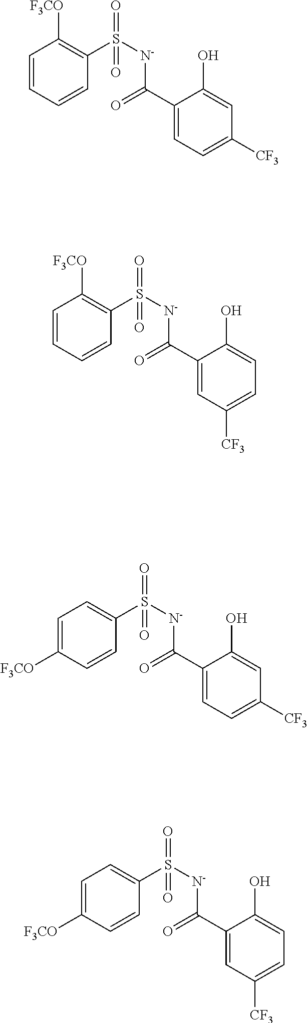 [SMILES: O=C([N-]S(=O)(=O)c1ccccc1OC(F)(F)F)c1ccc(C(F)(F)F)cc1O ; O=C([N-]S(=O)(=O)c1ccccc1OC(F)(F)F)c1cc(C(F)(F)F)ccc1O ; O=C([N-]S(=O)(=O)c1ccc(OC(F)(F)F)cc1)c1ccc(C(F)(F)F)cc1O ; O=C([N-]S(=O)(=O)c1ccc(OC(F)(F)F)cc1)c1cc(C(F)(F)F)ccc1O]

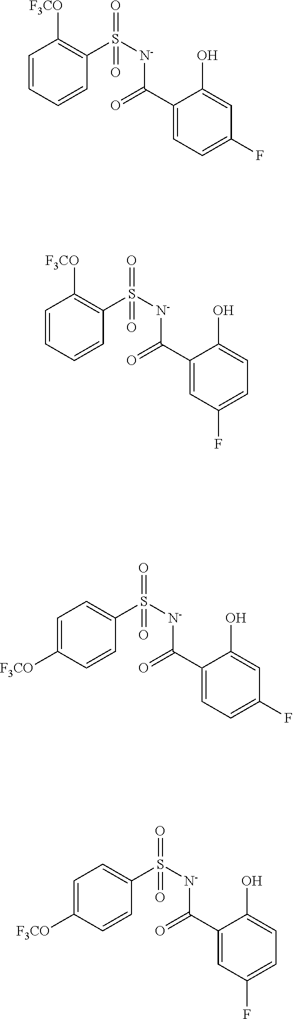 [SMILES: O=C([N-]S(=O)(=O)c1ccccc1OC(F)(F)F)c1ccc(F)cc1O ; O=C([N-]S(=O)(=O)c1ccccc1OC(F)(F)F)c1cc(F)ccc1O ; O=C([N-]S(=O)(=O)c1ccc(OC(F)(F)F)cc1)c1ccc(F)cc1O ; O=C([N-]S(=O)(=O)c1ccc(OC(F)(F)F)cc1)c1cc(F)ccc1O]

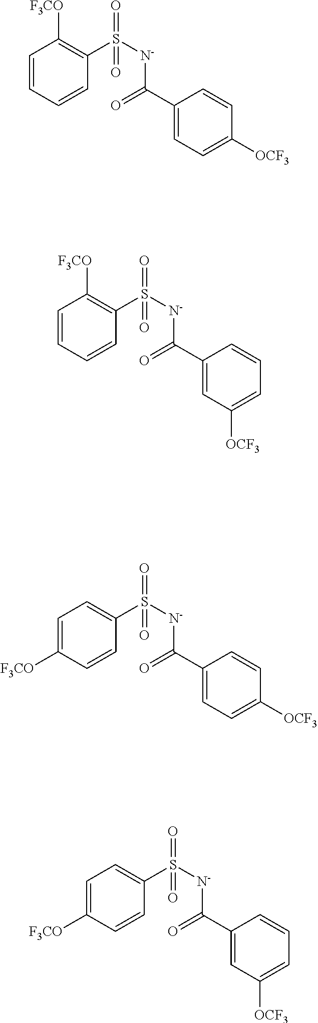 [SMILES: O=C([N-]S(=O)(=O)c1ccccc1OC(F)(F)F)c1ccc(OC(F)(F)F)cc1 ; O=C([N-]S(=O)(=O)c1ccccc1OC(F)(F)F)c1cccc(OC(F)(F)F)c1 ; O=C([N-]S(=O)(=O)c1ccc(OC(F)(F)F)cc1)c1ccc(OC(F)(F)F)cc1 ; O=C([N-]S(=O)(=O)c1ccc(OC(F)(F)F)cc1)c1cccc(OC(F)(F)F)c1]

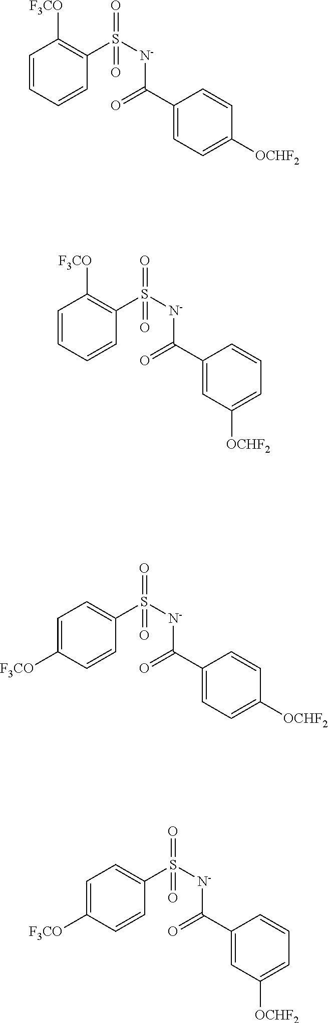 [SMILES: O=C([N-]S(=O)(=O)c1ccccc1OC(F)(F)F)c1ccc(OC(F)F)cc1 ; O=C([N-]S(=O)(=O)c1ccccc1OC(F)(F)F)c1cccc(OC(F)F)c1 ; O=C([N-]S(=O)(=O)c1ccc(OC(F)(F)F)cc1)c1ccc(OC(F)F)cc1 ; O=C([N-]S(=O)(=O)c1ccc(OC(F)(F)F)cc1)c1cccc(OC(F)F)c1]

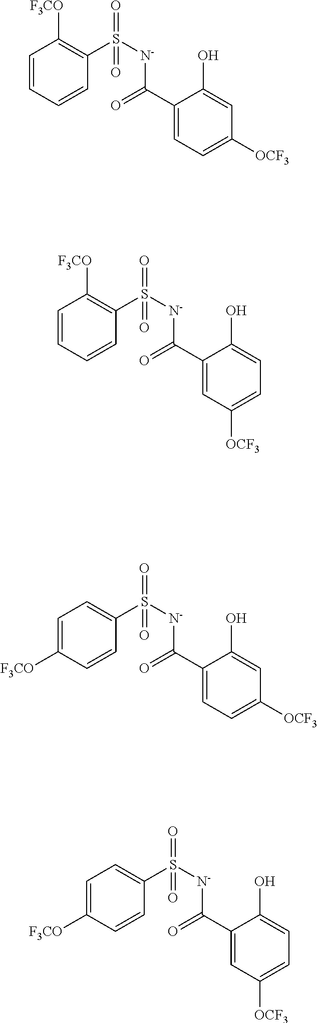 [SMILES: O=C([N-]S(=O)(=O)c1ccccc1OC(F)(F)F)c1ccc(OC(F)(F)F)cc1O ; O=C([N-]S(=O)(=O)c1ccccc1OC(F)(F)F)c1cc(OC(F)(F)F)ccc1O ; O=C([N-]S(=O)(=O)c1ccc(OC(F)(F)F)cc1)c1ccc(OC(F)(F)F)cc1O ; O=C([N-]S(=O)(=O)c1ccc(OC(F)(F)F)cc1)c1cc(OC(F)(F)F)ccc1O]

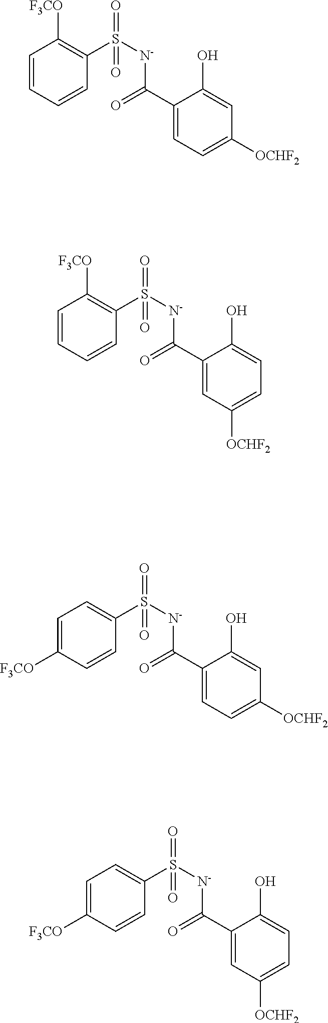 [SMILES: O=C([N-]S(=O)(=O)c1ccccc1OC(F)(F)F)c1ccc(OC(F)F)cc1O ; O=C([N-]S(=O)(=O)c1ccccc1OC(F)(F)F)c1cc(OC(F)F)ccc1O ; O=C([N-]S(=O)(=O)c1ccc(OC(F)(F)F)cc1)c1ccc(OC(F)F)cc1O ; O=C([N-]S(=O)(=O)c1ccc(OC(F)(F)F)cc1)c1cc(OC(F)F)ccc1O]

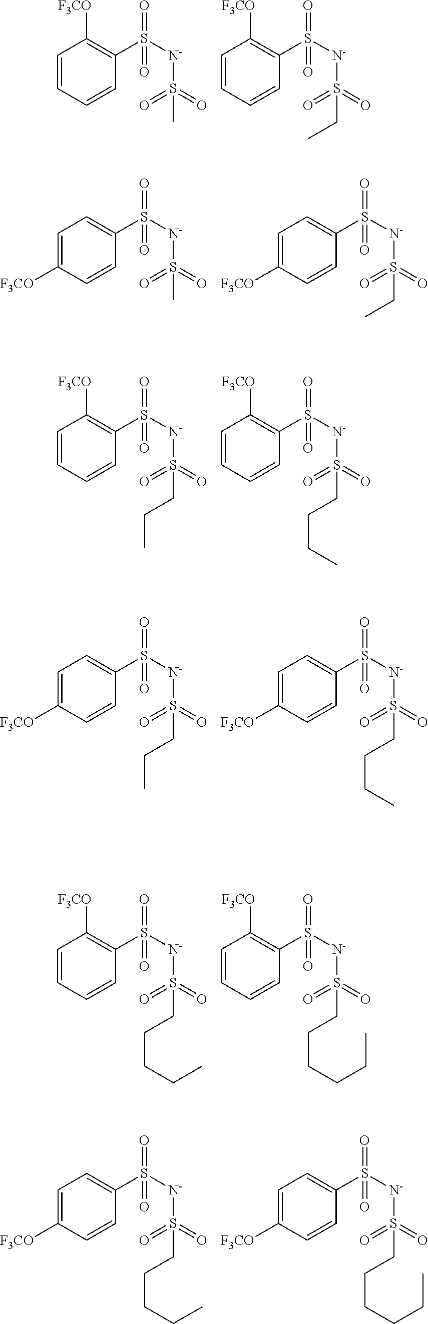

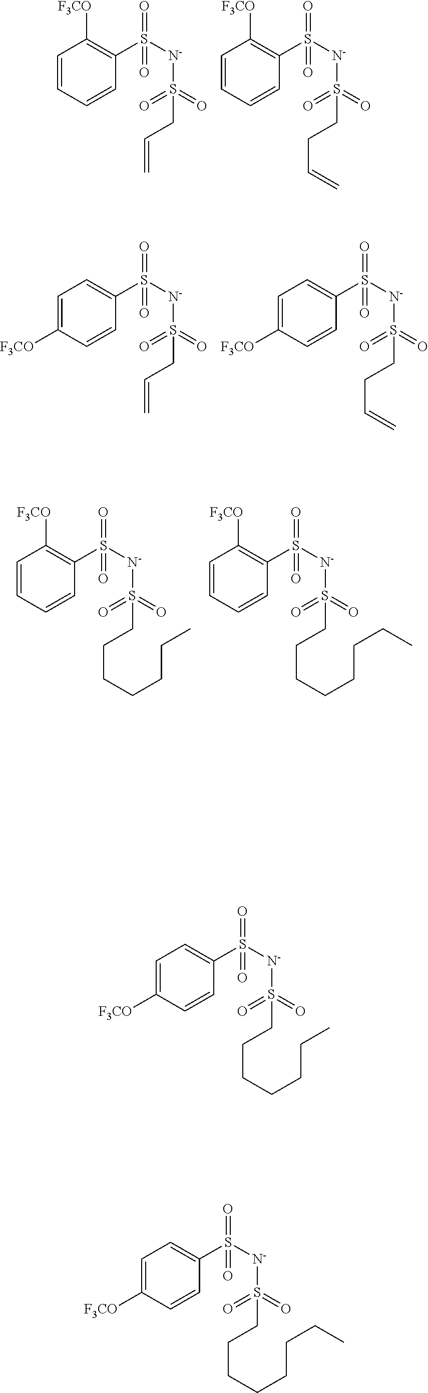

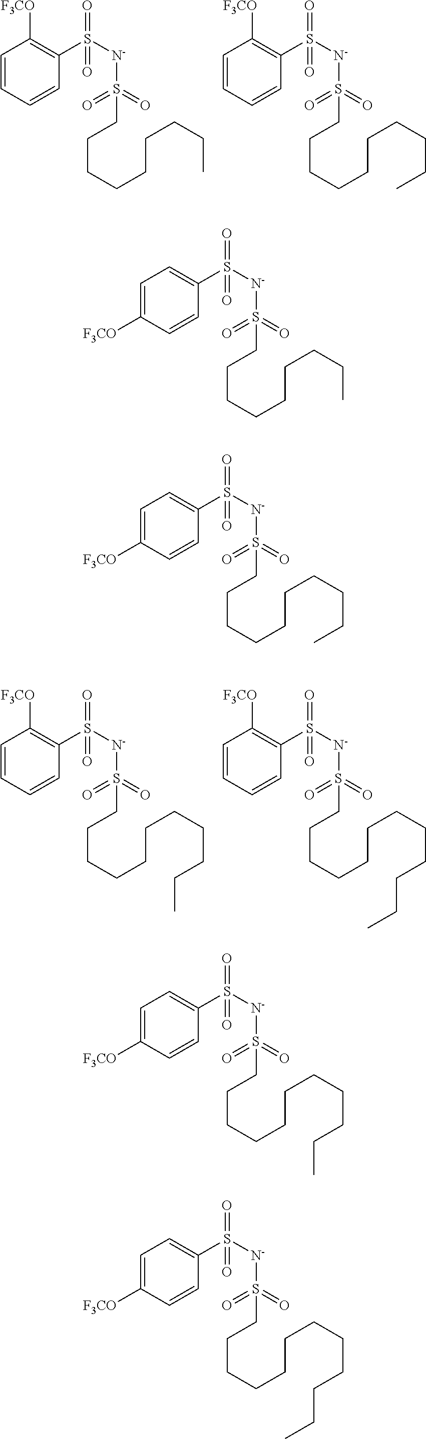

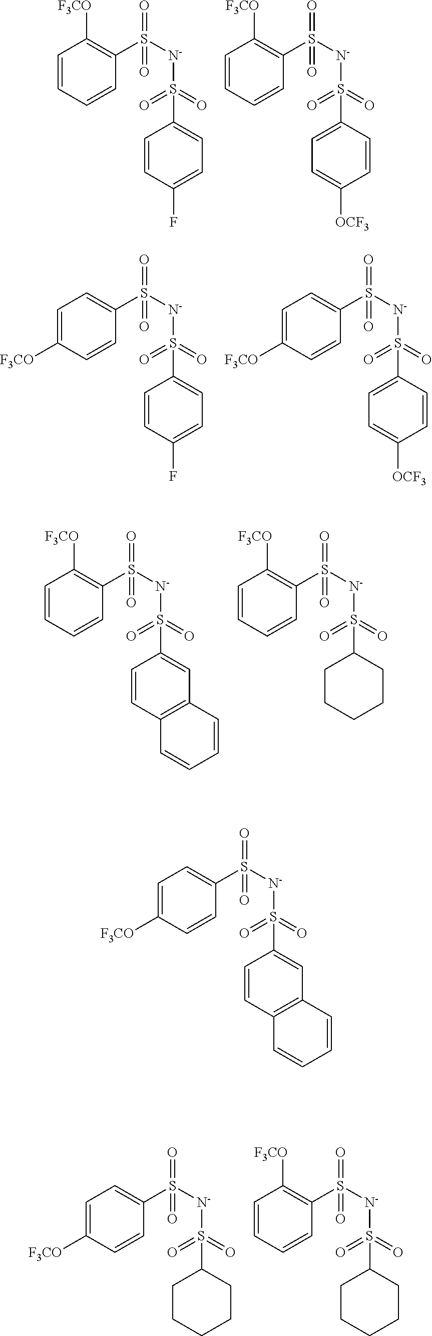

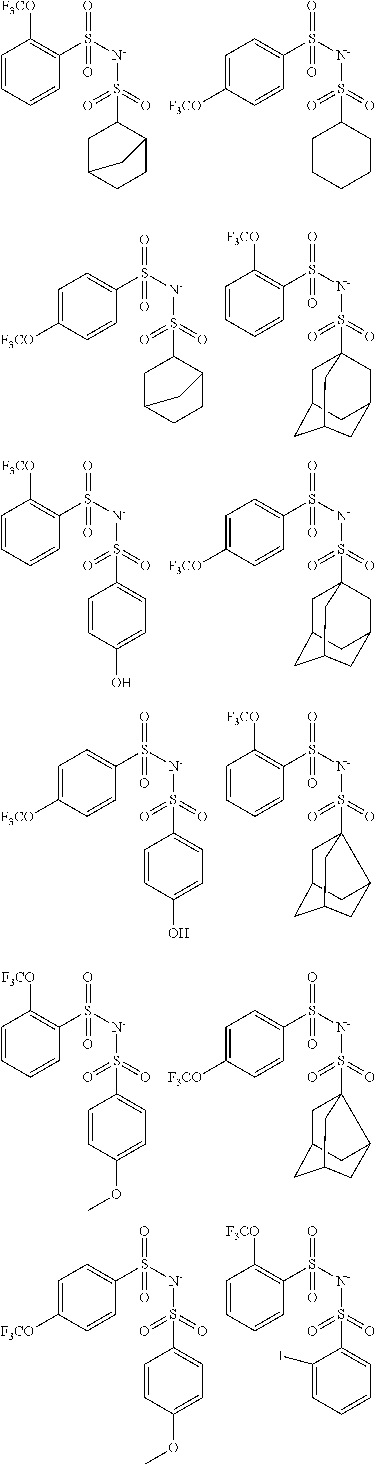

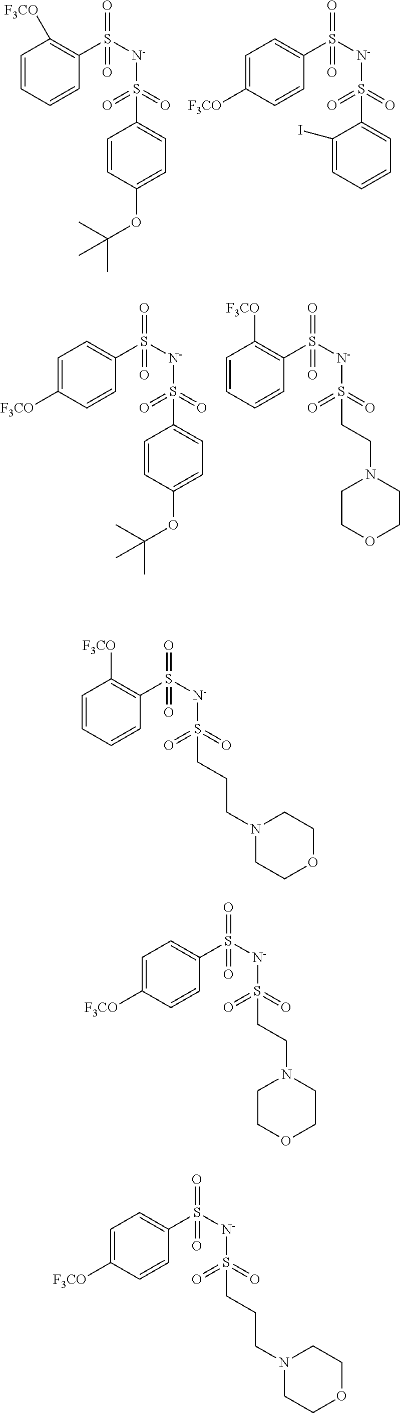

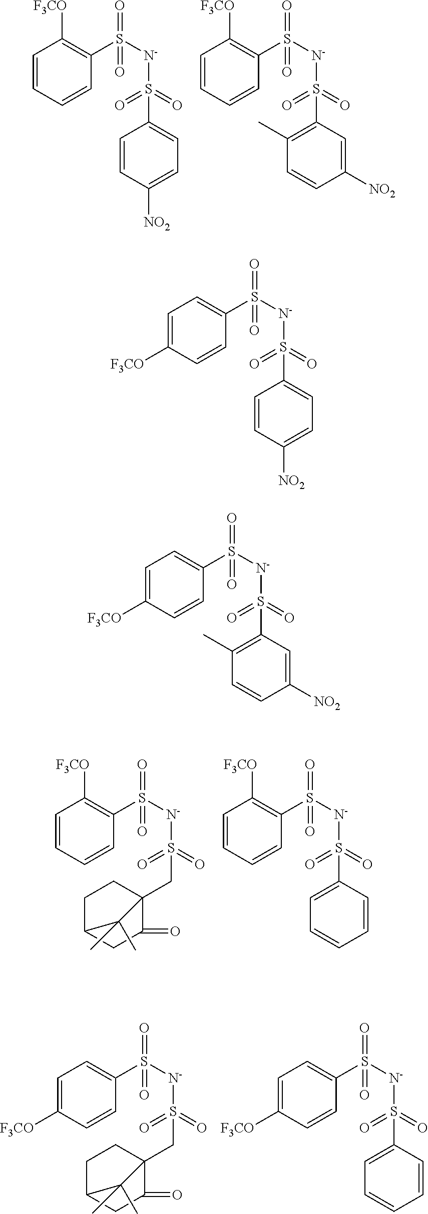

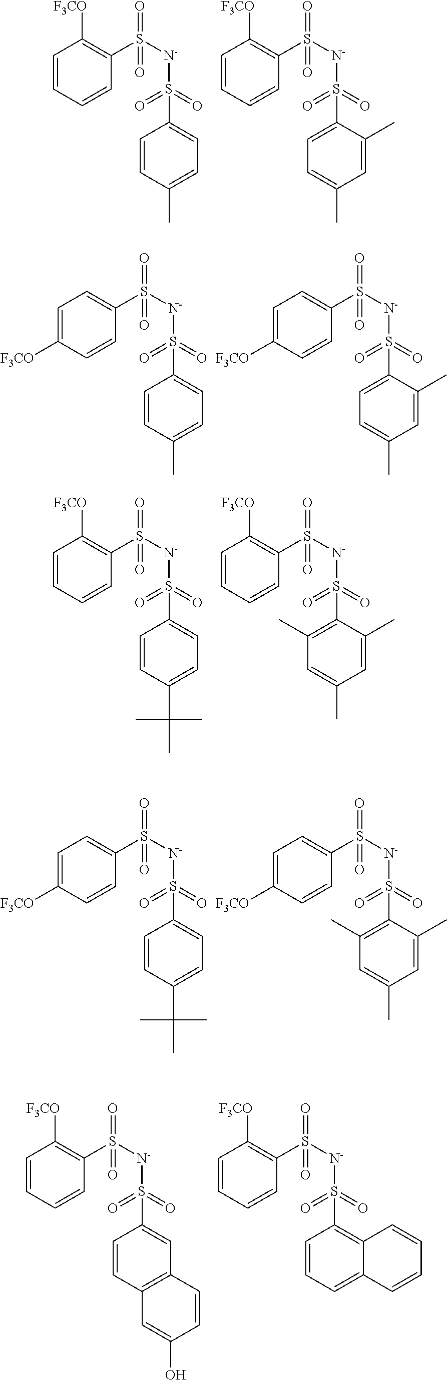

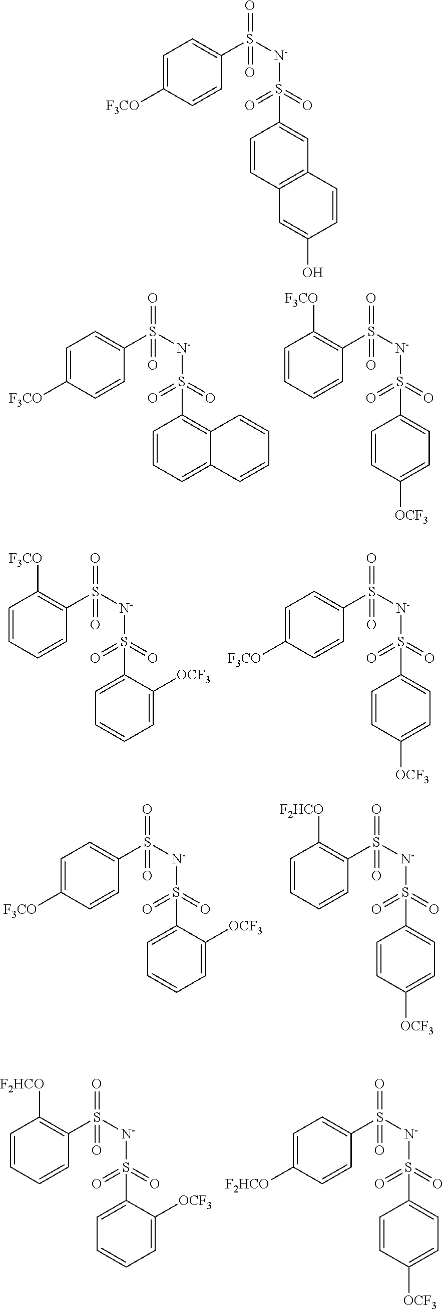

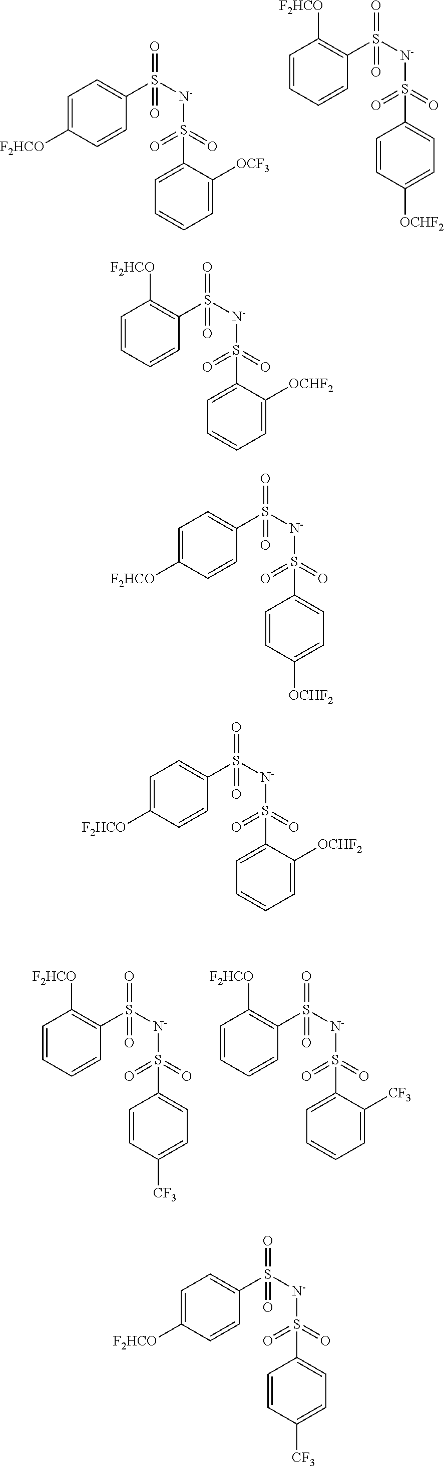

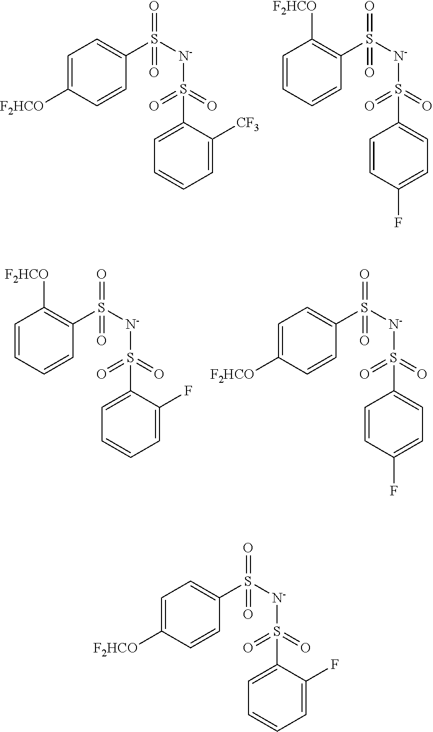 [SMILES: O=S(=O)([N-]S(=O)(=O)c1ccccc1C(F)(F)F)c1ccc(OC(F)F)cc1 ; O=S(=O)([N-]S(=O)(=O)c1ccccc1OC(F)F)c1ccc(F)cc1 ; O=S(=O)([N-]S(=O)(=O)c1ccccc1OC(F)F)c1ccccc1F ; O=S(=O)([N-]S(=O)(=O)c1ccc(OC(F)F)cc1)c1ccc(F)cc1 ; O=S(=O)([N-]S(=O)(=O)c1ccccc1F)c1ccc(OC(F)F)cc1]

For the synthesis of the anion in Sulfonium Salt A, the method described in JP-A 2018-130534, paragraph [0083] may be used. That is, a halide or ammonium salt is obtained by reacting trifluoromethoxybenzenesulfonamide or difluoromethoxybenzenesulfonamide with a carboxylic acid chloride. The halide or ammonium salt is also obtained from similar reaction using a sulfonic acid chloride instead of the carboxylic acid chloride.

In formula (1), R⁴ to R⁶ are each independently halogen or a C₁-C₂₀ hydrocarbyl group which may contain a heteroatom. Suitable halogen atoms include fluorine, chlorine, bromine, and iodine. The C₁-C₂₀ hydrocarbyl group may be saturated or unsaturated and straight, branched or cyclic. Examples thereof include C₁-C₂₀ alkyl groups such as methyl, ethyl, n-propyl, isopropyl, n-butyl, isobutyl, sec-butyl, tert-butyl, n-pentyl, n-hexyl, n-octyl, n-nonyl, n-decyl, undecyl, dodecyl, tridecyl, tetradecyl, pentadecyl, heptadecyl, octadecyl, nonadecyl, and icosyl; C₃-C₂₀ cyclic saturated hydrocarbyl groups such as cyclopropyl, cyclopentyl, cyclohexyl, cyclopropylmethyl, 4-methylcyclohexyl, cyclohexylmethyl, norbornyl, and adamantyl; C₂-C₂₀ alkenyl groups such as vinyl, propenyl, butenyl, and hexenyl; C₂-C₂₀ alkynyl groups such as ethynyl, propynyl, and butynyl; C₃-C₂₀ cyclic unsaturated aliphatic hydrocarbyl groups such as cyclohexenyl and norbornenyl; C₆-C₂₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, and tert-butylnaphthyl; C₇-C₂₀ aralkyl groups such as benzyl and phenethyl; and combinations thereof.

In the hydrocarbyl group, some or all of the hydrogen atoms may be substituted by a moiety containing a heteroatom such as oxygen, sulfur, nitrogen or halogen, and some constituent —CH₂— may be replaced by a moiety containing a heteroatom such as oxygen, sulfur or nitrogen, so that the group may contain a hydroxy, fluorine, chlorine, bromine, iodine, cyano, nitro, mercapto, carbonyl, ether bond, ester bond, sulfonate ester bond, carbonate bond, lactone ring, sultone ring, carboxylic anhydride (—C(═O)—O—C(═O)—), or haloalkyl moiety.

Also, R⁴ and R⁵ may bond together to form a ring with the sulfur atom to which they are attached. Rings of the structure shown below are preferred.

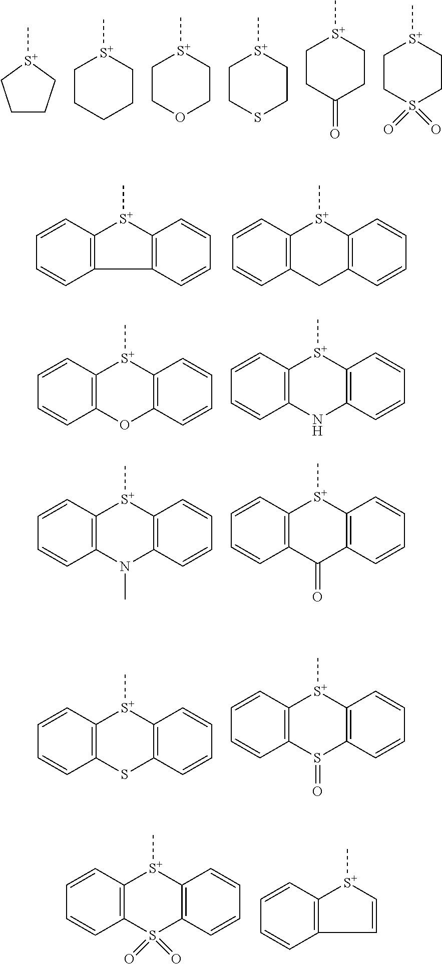

Herein, the broken line denotes a point of attachment to R⁶.

Examples of the cation in Sulfonium Salt A are shown below, but not limited thereto.

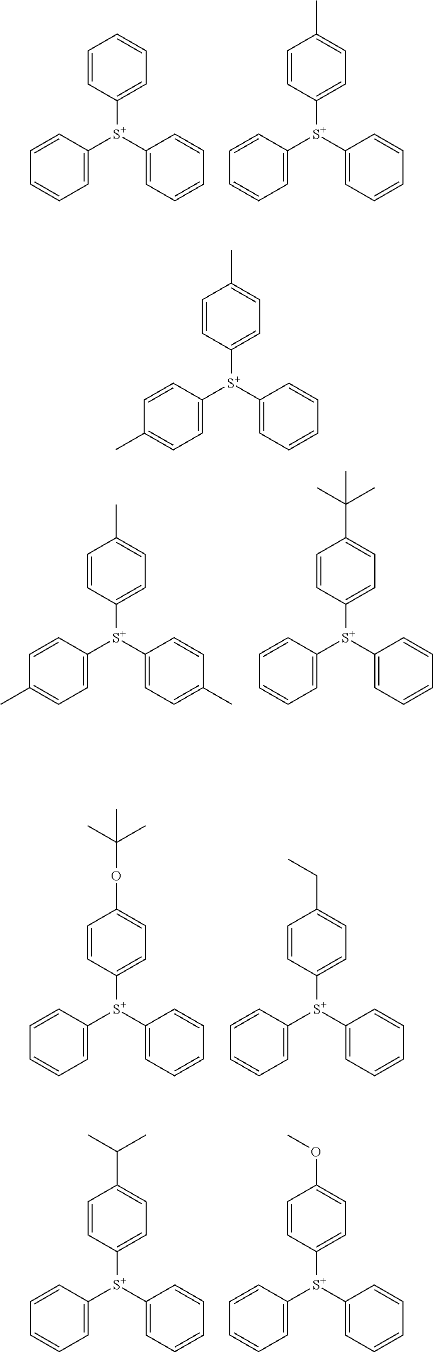

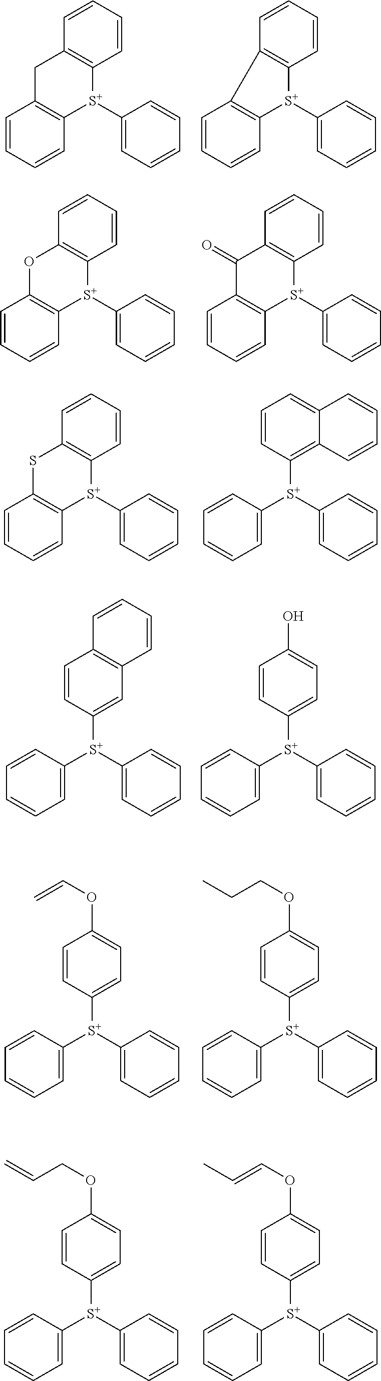

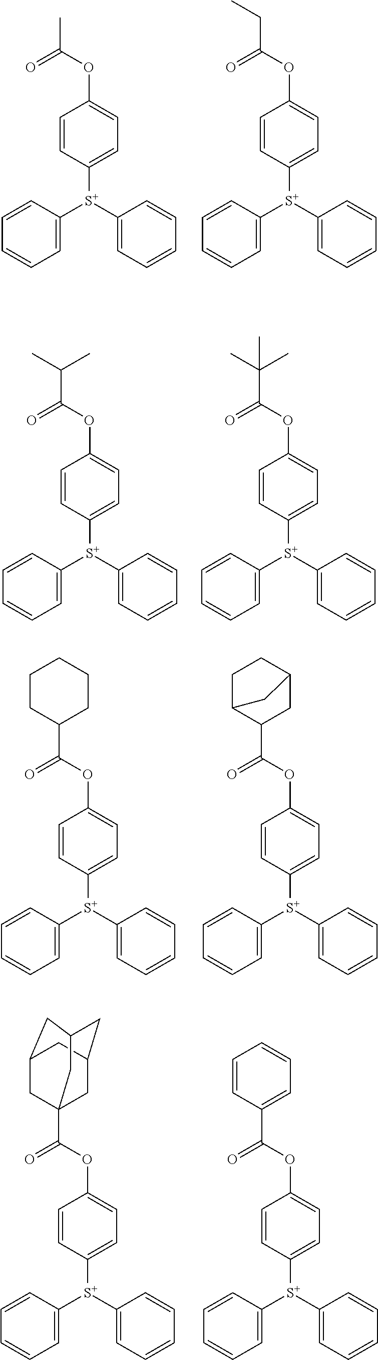

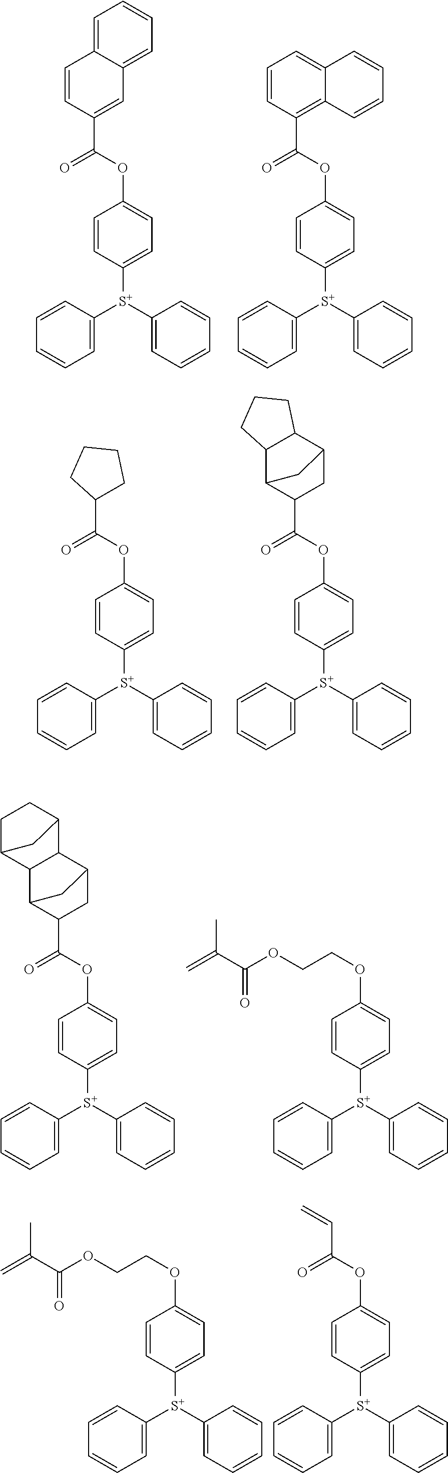

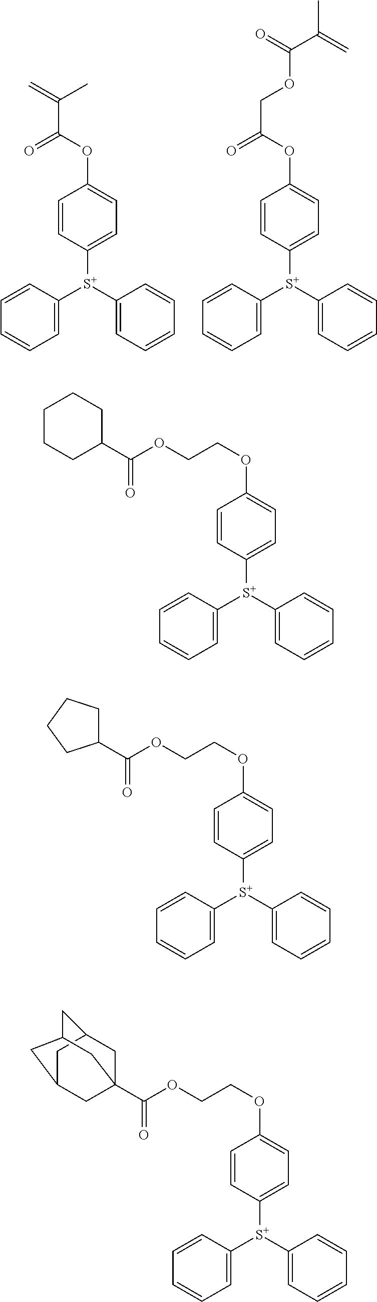 [SMILES: C=C(C)C(=O)Oc1ccc([S+](c2ccccc2)c2ccccc2)cc1 ; C=C(C)C(=O)OCC(=O)Oc1ccc([S+](c2ccccc2)c2ccccc2)cc1 ; O=C(OCCOc1ccc([S+](c2ccccc2)c2ccccc2)cc1)C1CCCCC1 ; O=C(OCCOc1ccc([S+](c2ccccc2)c2ccccc2)cc1)C1CCCC1 ; O=C(OCCOc1ccc([S+](c2ccccc2)c2ccccc2)cc1)C12CC3CC(CC(C3)C1)C2]

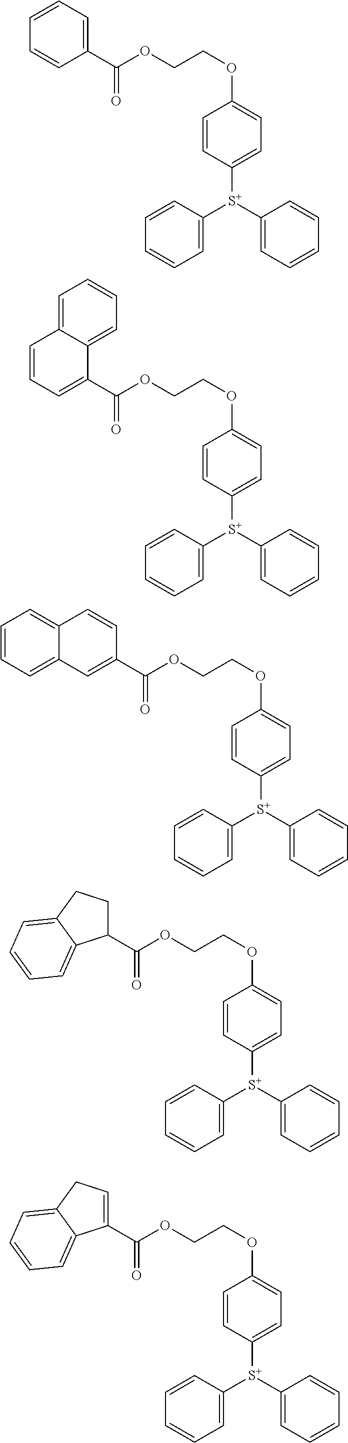 [SMILES: O=C(OCCOc1ccc([S+](c2ccccc2)c2ccccc2)cc1)c1ccccc1 ; O=C(OCCOc1ccc([S+](c2ccccc2)c2ccccc2)cc1)c1cccc2ccccc12 ; O=C(OCCOc1ccc([S+](c2ccccc2)c2ccccc2)cc1)c1ccc2ccccc2c1 ; O=C(OCCOc1ccc([S+](c2ccccc2)c2ccccc2)cc1)C1CCc2ccccc21 ; O=C(OCCOc1ccc([S+](c2ccccc2)c2ccccc2)cc1)C1=CCc2ccccc21]

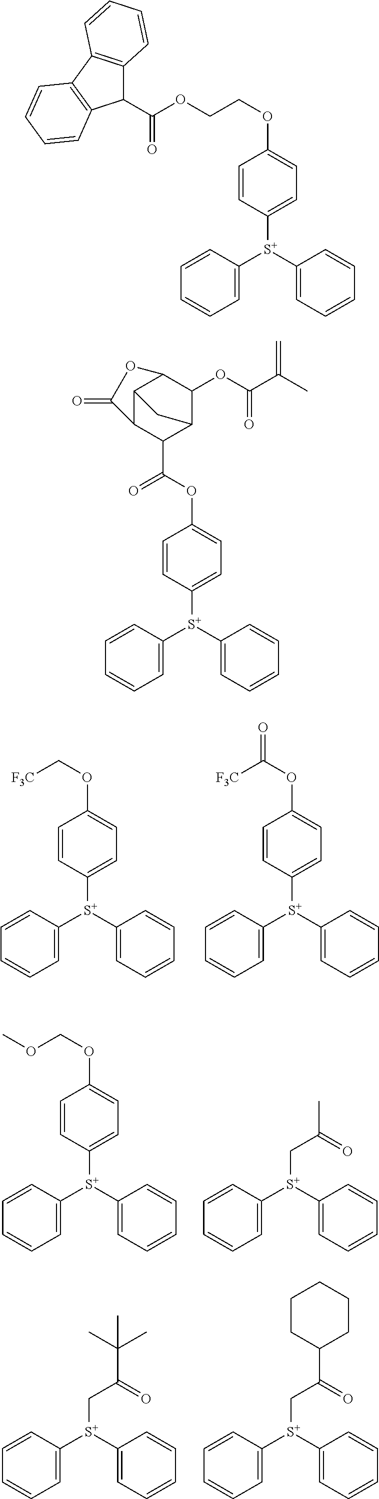

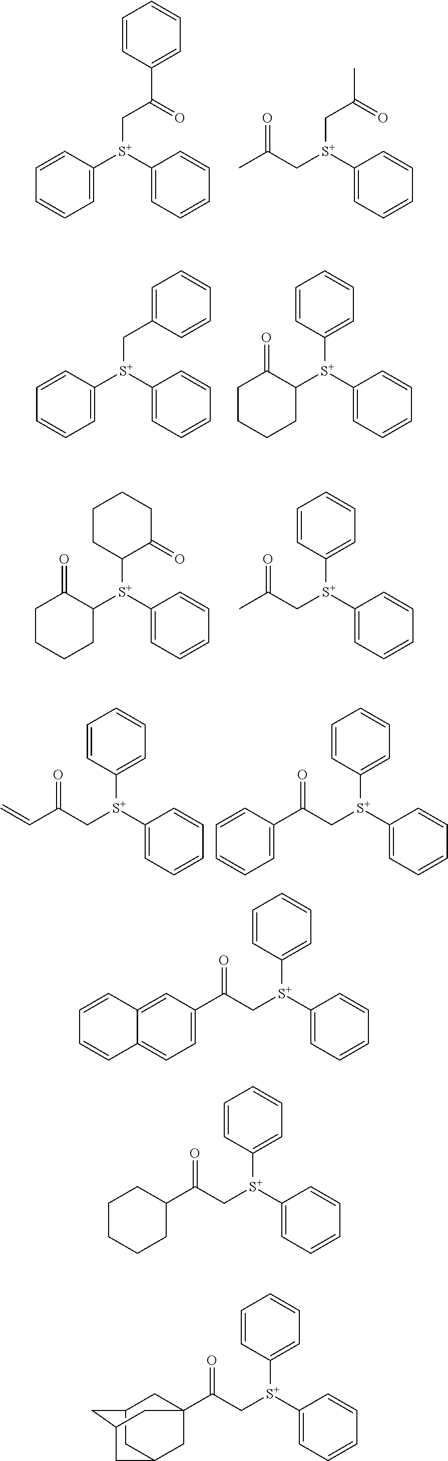

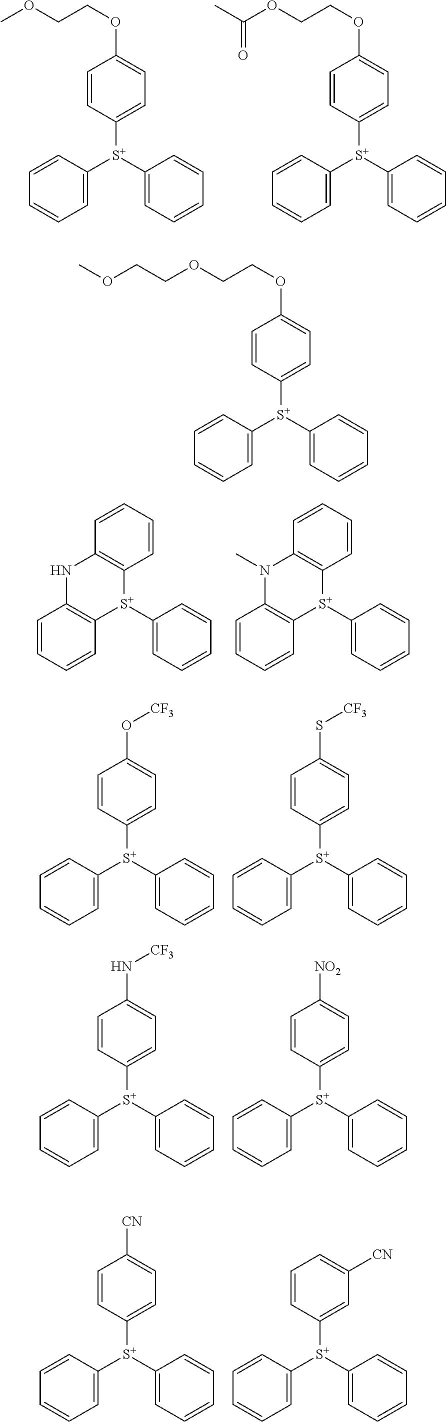

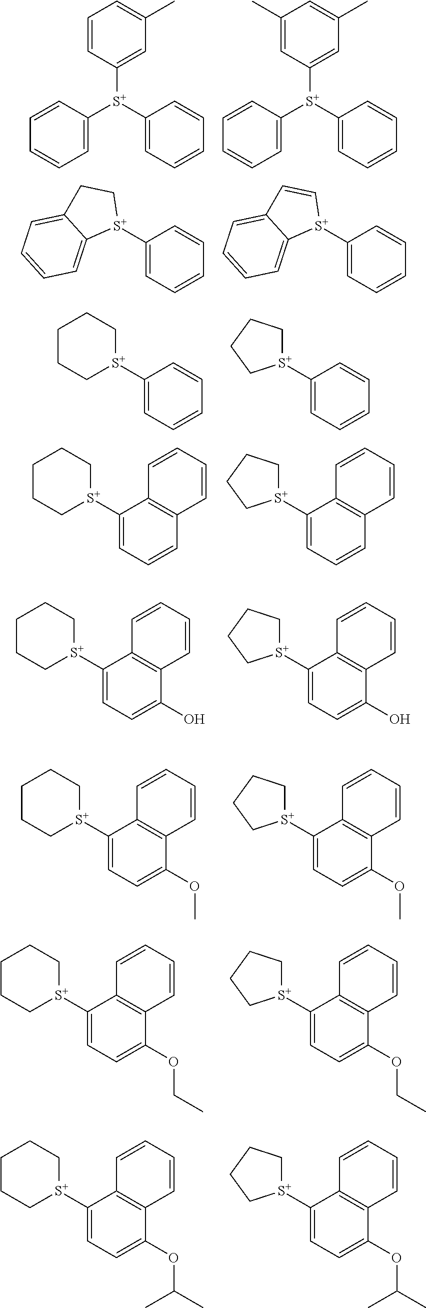

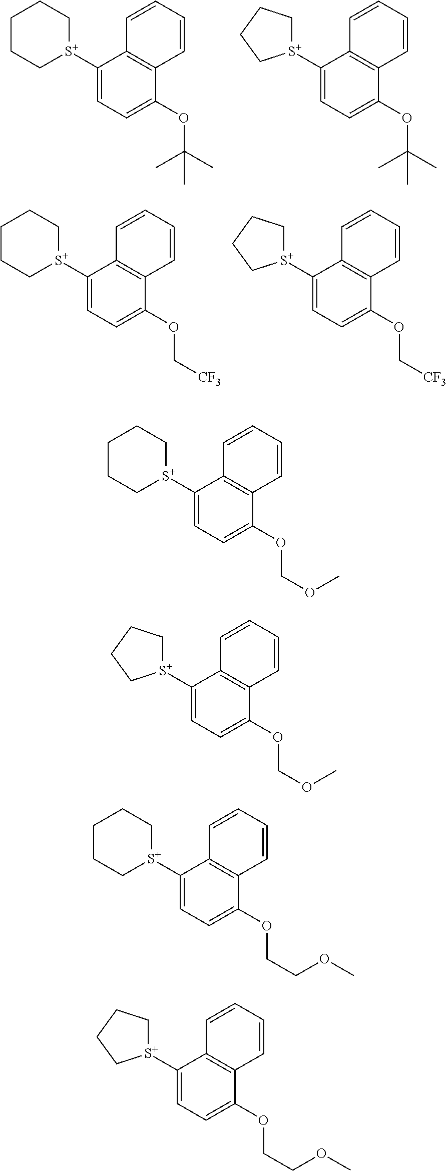

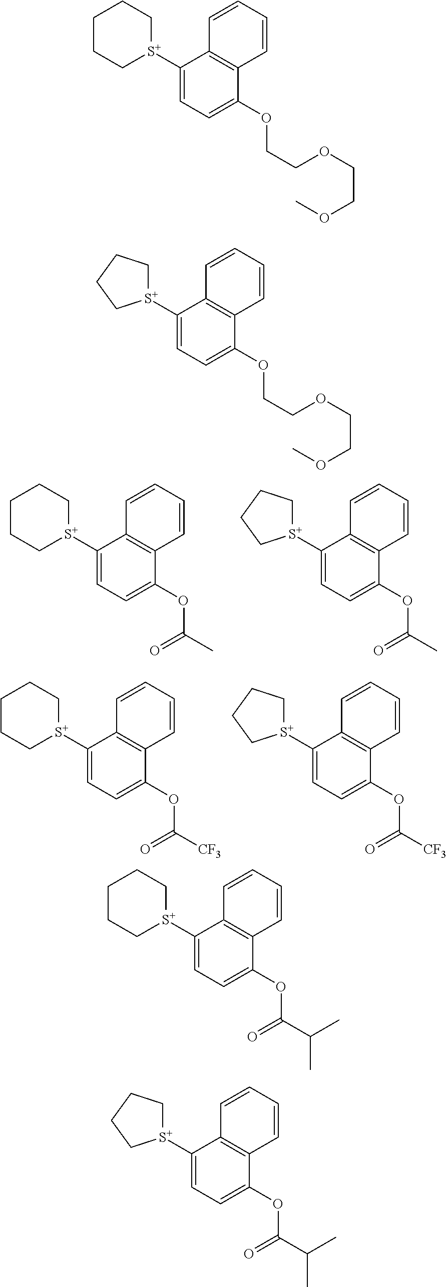

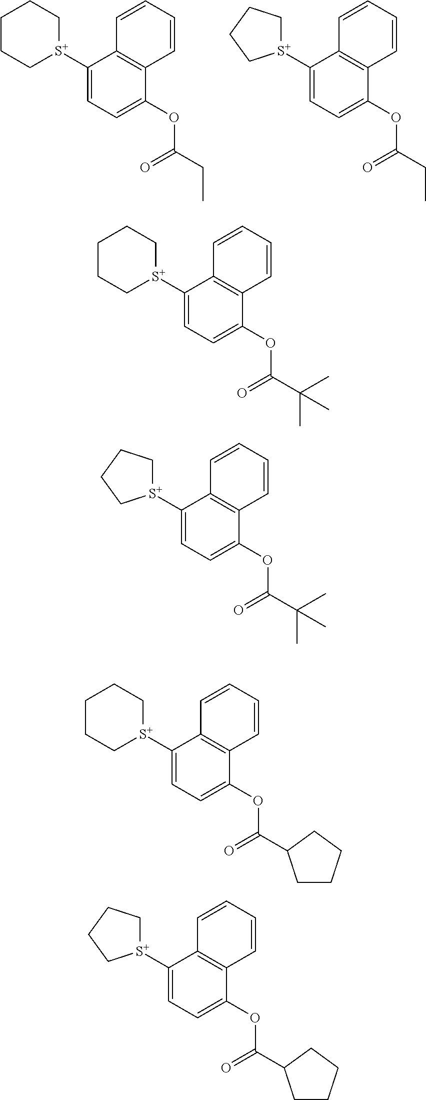 [SMILES: CCC(=O)Oc1ccc([S+]2CCCCC2)c2ccccc12 ; CCC(=O)Oc1ccc([S+]2CCCC2)c2ccccc12 ; CC(C)(C)C(=O)Oc1ccc([S+]2CCCCC2)c2ccccc12 ; CC(C)(C)C(=O)Oc1ccc([S+]2CCCC2)c2ccccc12 ; O=C(Oc1ccc([S+]2CCCCC2)c2ccccc12)C1CCCC1 ; O=C(Oc1ccc([S+]2CCCC2)c2ccccc12)C1CCCC1]

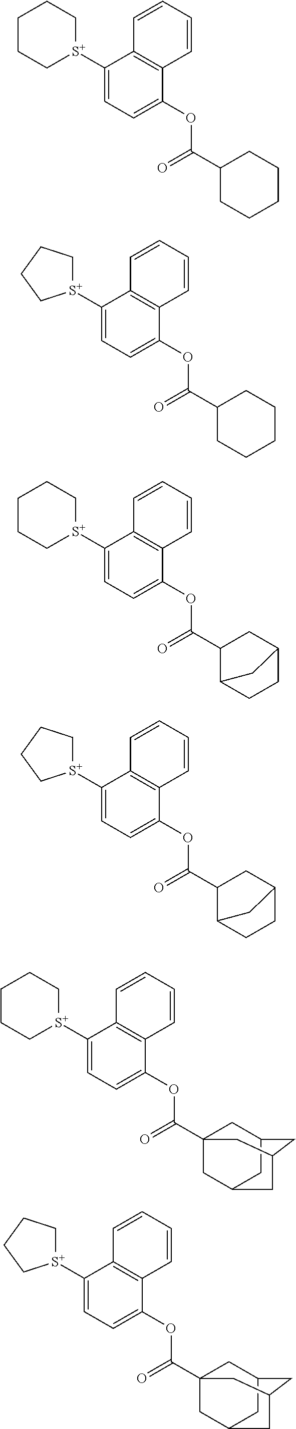 [SMILES: O=C(Oc1ccc([S+]2CCCCC2)c2ccccc12)C1CCCCC1 ; O=C(Oc1ccc([S+]2CCCC2)c2ccccc12)C1CCCCC1 ; O=C(Oc1ccc([S+]2CCCCC2)c2ccccc12)C1CC2CCC1C2 ; O=C(Oc1ccc([S+]2CCCC2)c2ccccc12)C1CC2CCC1C2 ; O=C(Oc1ccc([S+]2CCCCC2)c2ccccc12)C12CC3CC(CC(C3)C1)C2 ; O=C(Oc1ccc([S+]2CCCC2)c2ccccc12)C12CC3CC(CC(C3)C1)C2]

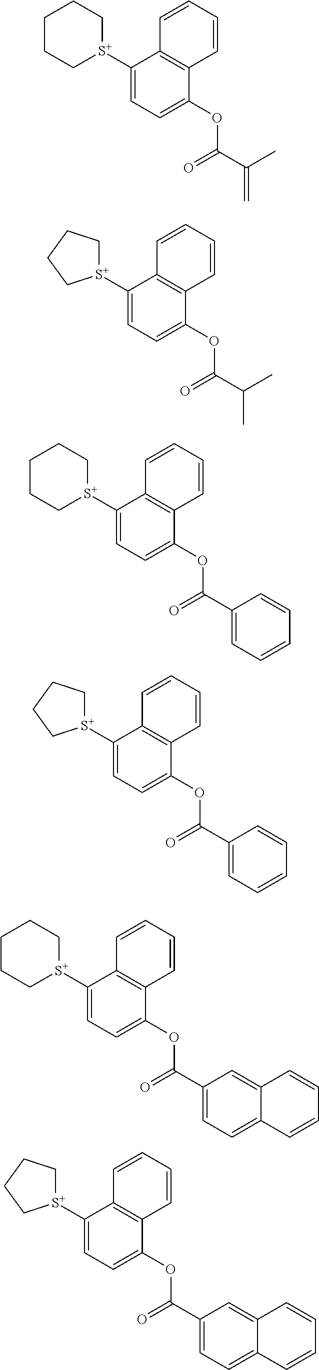 [SMILES: C=C(C)C(=O)Oc1ccc([S+]2CCCCC2)c2ccccc12 ; CC(C)C(=O)Oc1ccc([S+]2CCCC2)c2ccccc12 ; O=C(Oc1ccc([S+]2CCCCC2)c2ccccc12)c1ccccc1 ; O=C(Oc1ccc([S+]2CCCC2)c2ccccc12)c1ccccc1 ; O=C(Oc1ccc([S+]2CCCCC2)c2ccccc12)c1ccc2ccccc2c1 ; O=C(Oc1ccc([S+]2CCCC2)c2ccccc12)c1ccc2ccccc2c1]

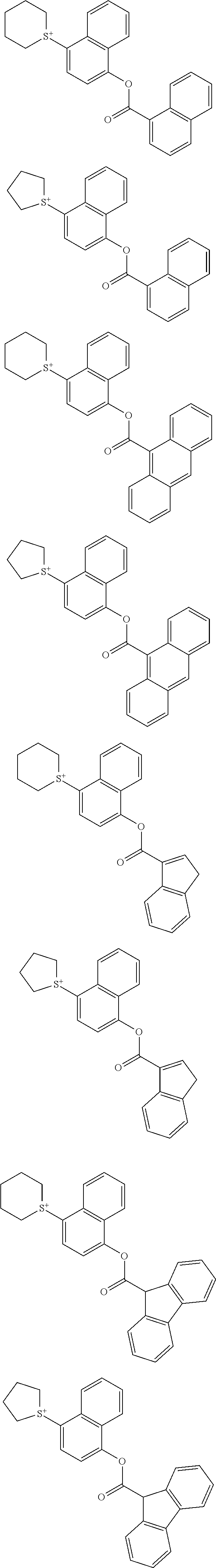

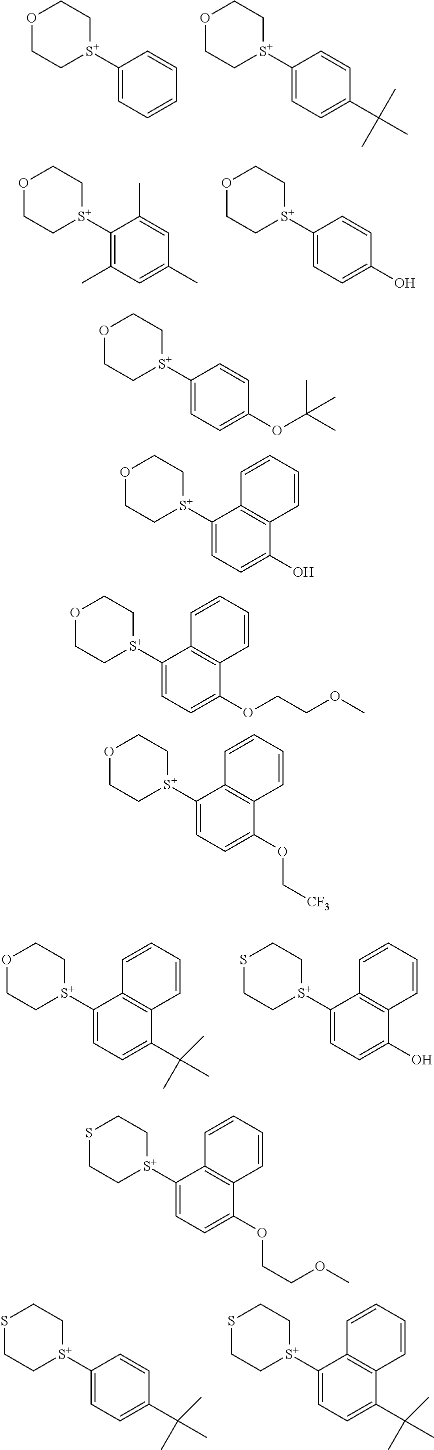

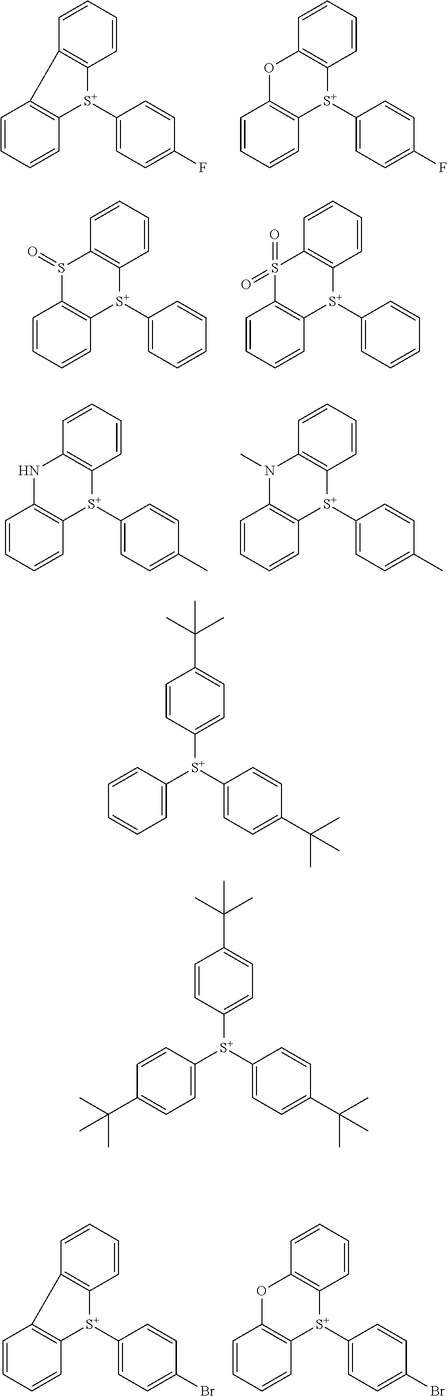

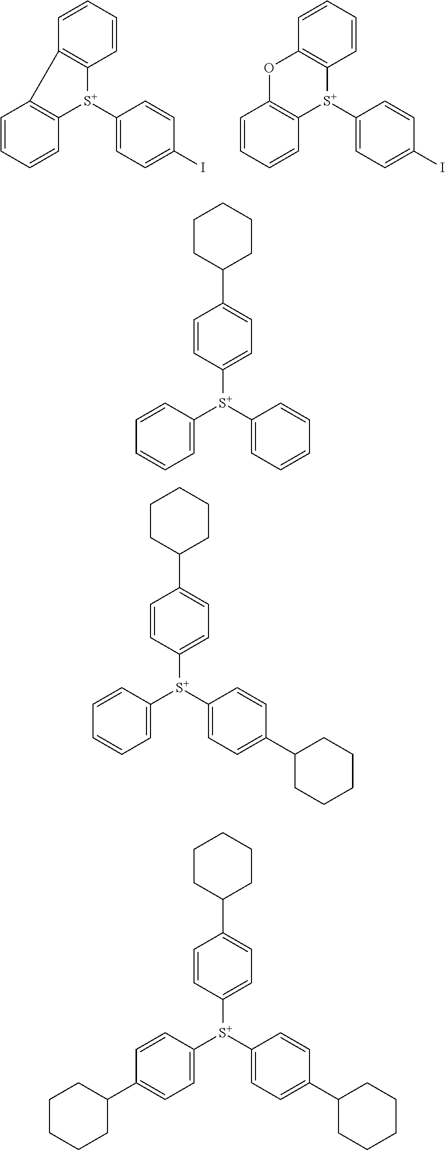 [SMILES: Ic1ccc(-[s+]2c3ccccc3c3ccccc32)cc1 ; Ic1ccc([S+]2c3ccccc3Oc3ccccc32)cc1 ; c1ccc([S+](c2ccccc2)c2ccc(C3CCCCC3)cc2)cc1 ; c1ccc([S+](c2ccc(C3CCCCC3)cc2)c2ccc(C3CCCCC3)cc2)cc1 ; c1cc([S+](c2ccc(C3CCCCC3)cc2)c2ccc(C3CCCCC3)cc2)ccc1C1CCCCC1]

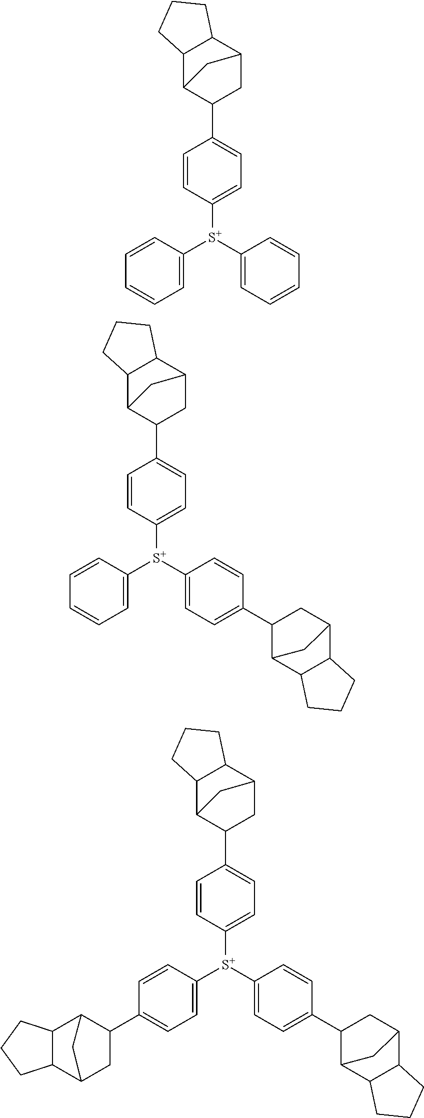 [SMILES: c1ccc([S+](c2ccccc2)c2ccc(C3CC4CC3C3CCCC43)cc2)cc1 ; c1ccc([S+](c2ccc(C3CC4CC3C3CCCC43)cc2)c2ccc(C3CC4CC3C3CCCC43)cc2)cc1 ; c1cc([S+](c2ccc(C3CC4CC3C3CCCC43)cc2)c2ccc(C3CC4CC3C3CCCC43)cc2)ccc1C1CC2CC1C1CCCC21]

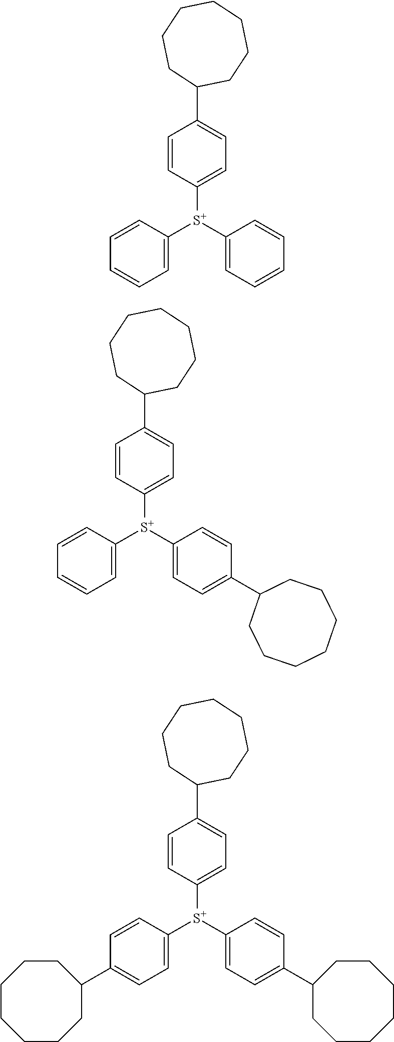 [SMILES: c1ccc([S+](c2ccccc2)c2ccc(C3CCCCCCC3)cc2)cc1 ; c1ccc([S+](c2ccc(C3CCCCCCC3)cc2)c2ccc(C3CCCCCCC3)cc2)cc1 ; c1cc([S+](c2ccc(C3CCCCCCC3)cc2)c2ccc(C3CCCCCCC3)cc2)ccc1C1CCCCCCC1]

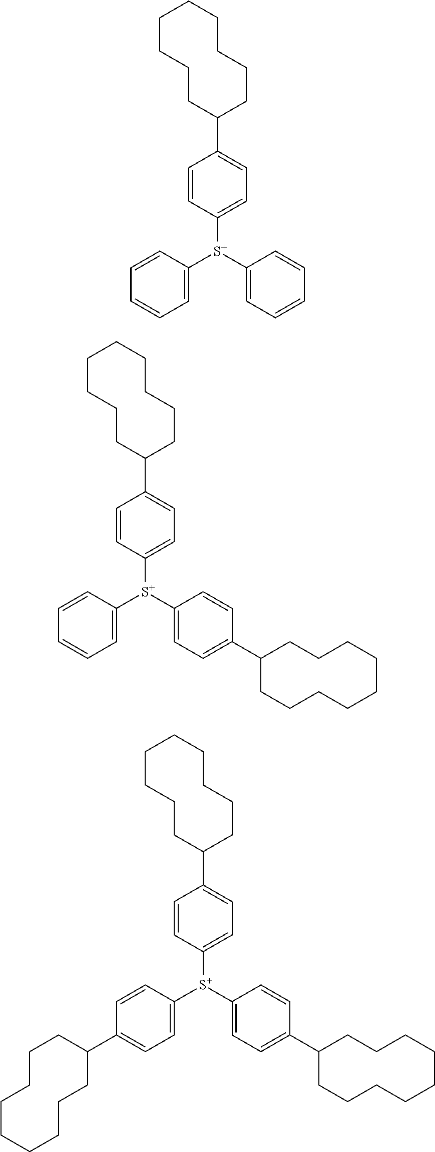 [SMILES: c1ccc([S+](c2ccccc2)c2ccc(C3CCCCCCCCC3)cc2)cc1 ; c1ccc([S+](c2ccc(C3CCCCCCCCC3)cc2)c2ccc(C3CCCCCCCCC3)cc2)cc1 ; c1cc([S+](c2ccc(C3CCCCCCCCC3)cc2)c2ccc(C3CCCCCCCCC3)cc2)ccc1C1CCCCCCCCC1]

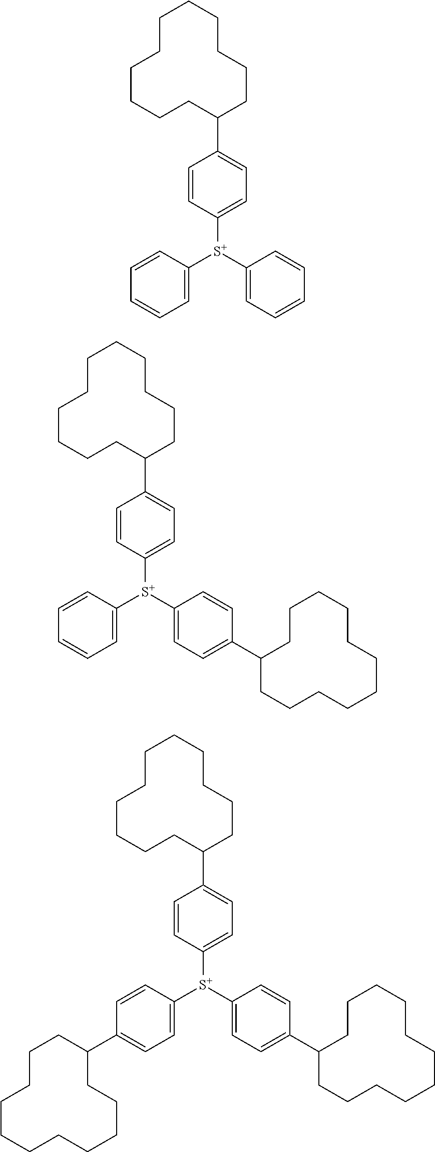 [SMILES: c1ccc([S+](c2ccccc2)c2ccc(C3CCCCCCCCCCC3)cc2)cc1 ; c1ccc([S+](c2ccc(C3CCCCCCCCCCC3)cc2)c2ccc(C3CCCCCCCCCCC3)cc2)cc1 ; c1cc([S+](c2ccc(C3CCCCCCCCCCC3)cc2)c2ccc(C3CCCCCCCCCCC3)cc2)ccc1C1CCCCCCCCCCC1]

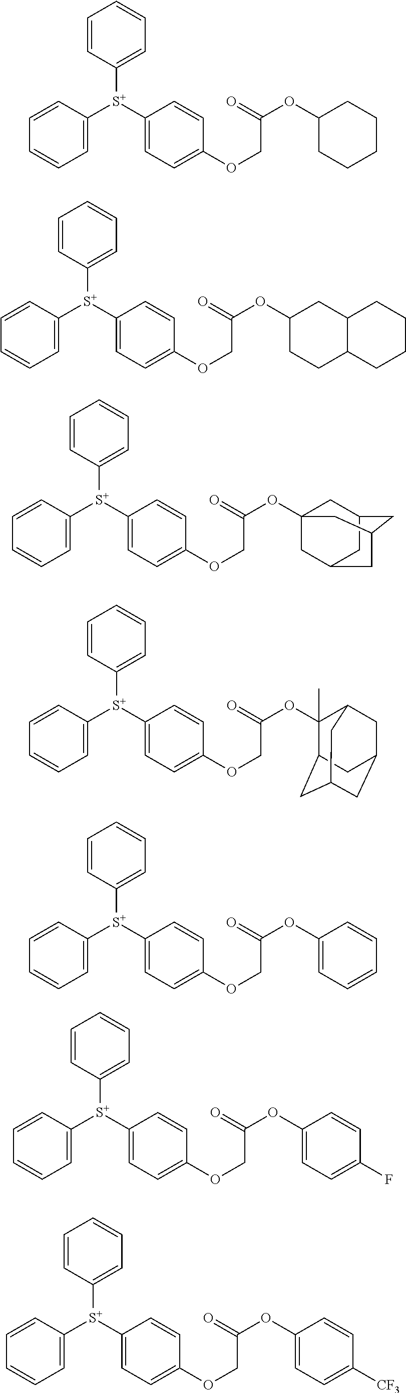

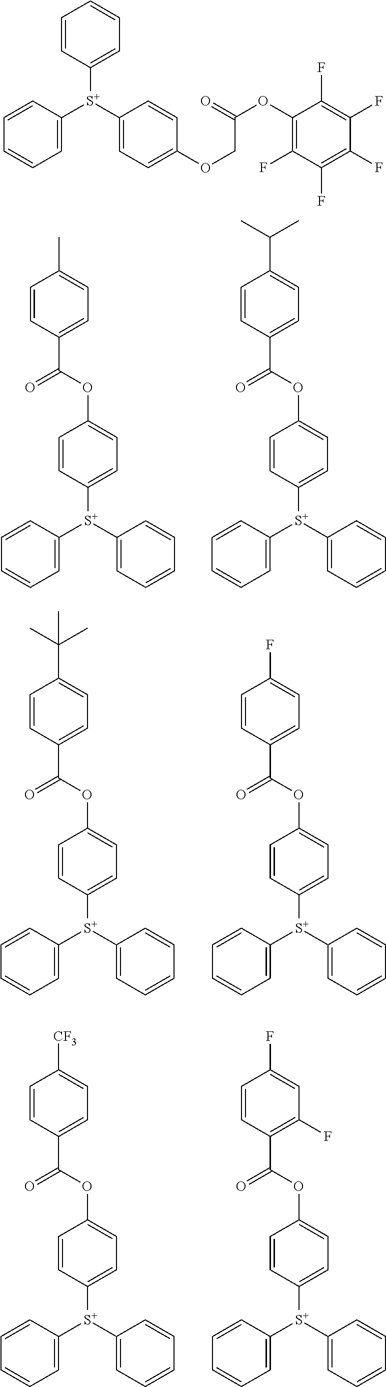

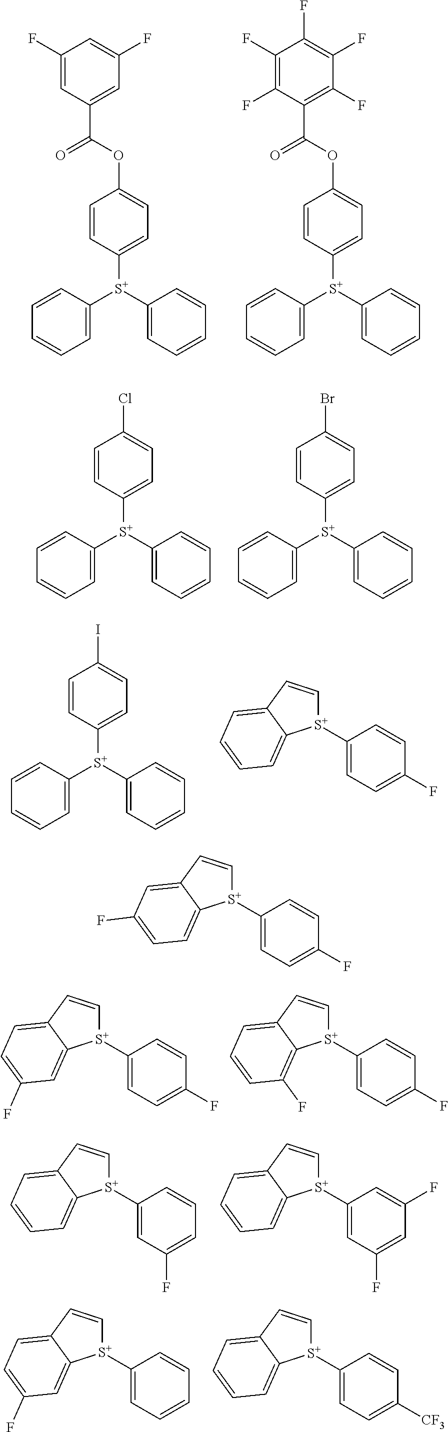

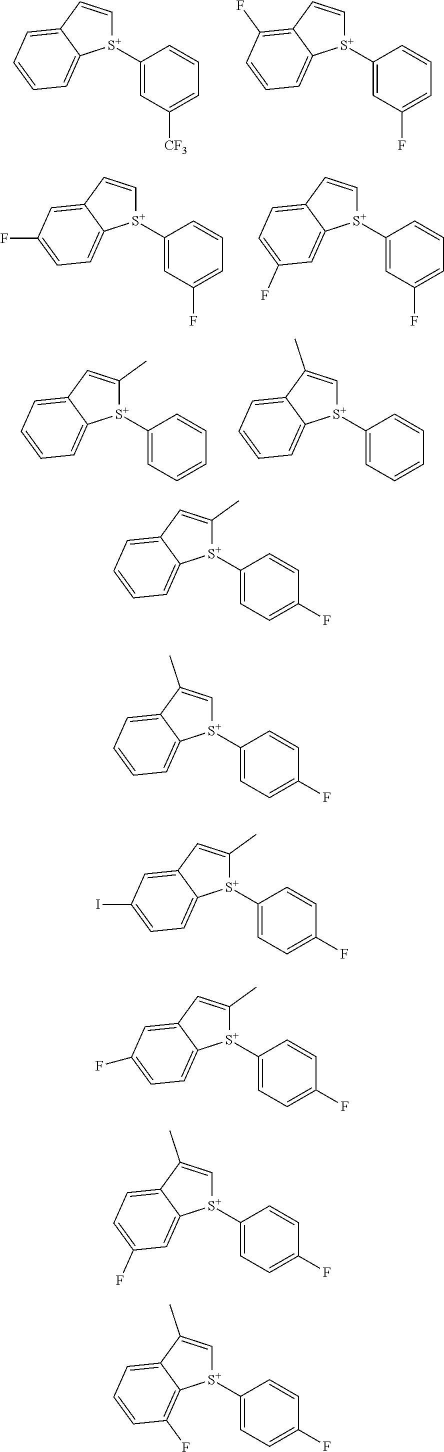

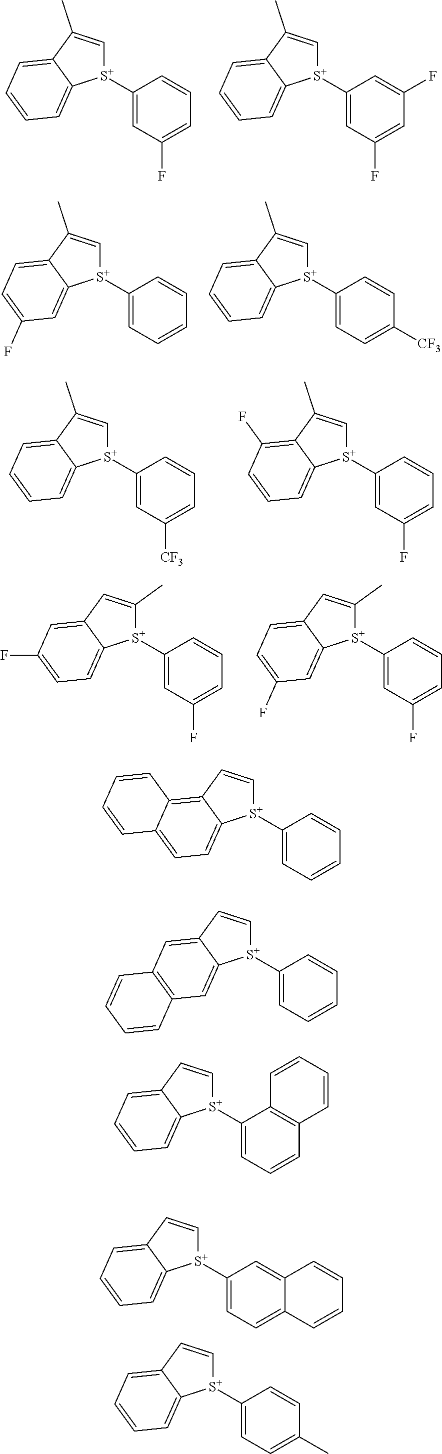

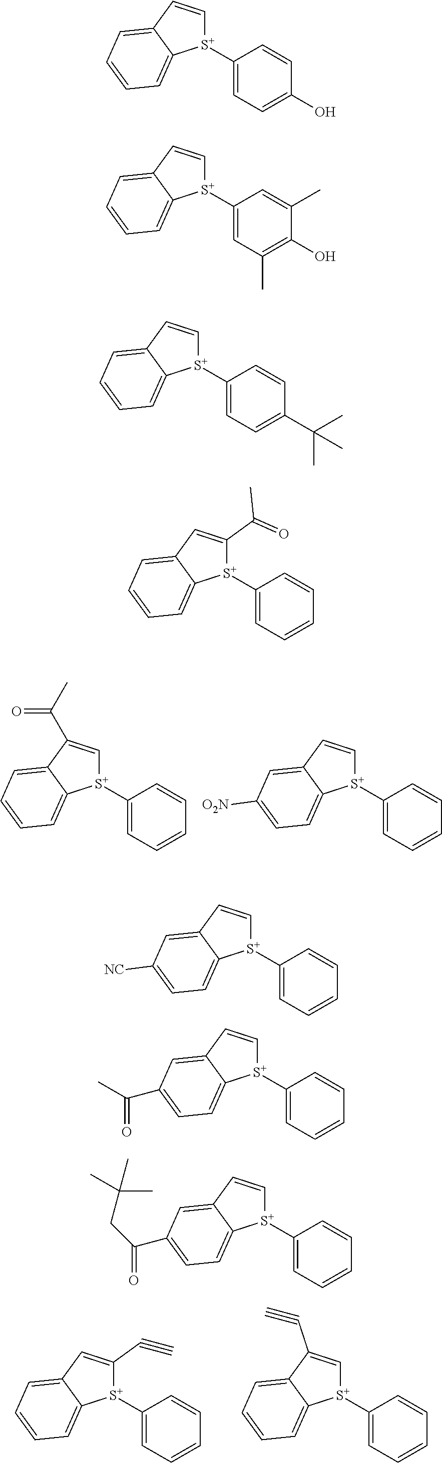

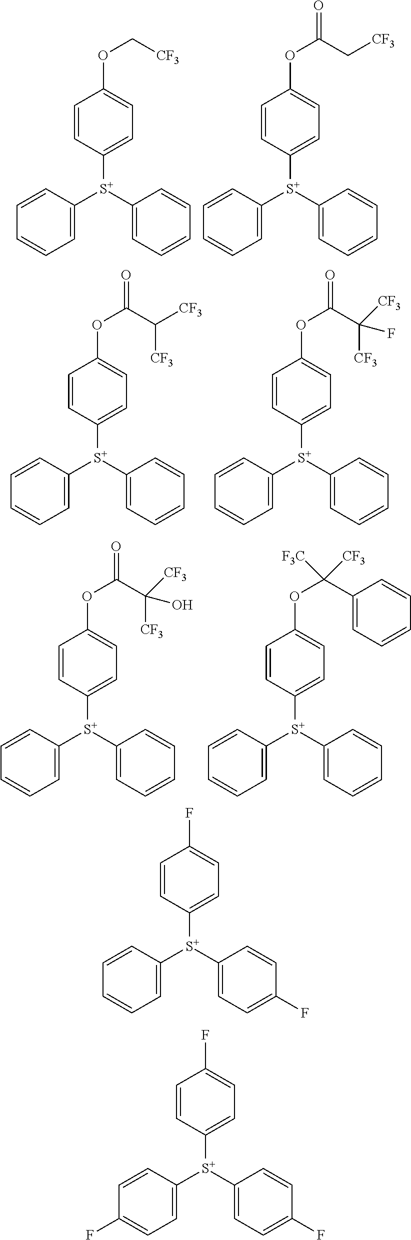

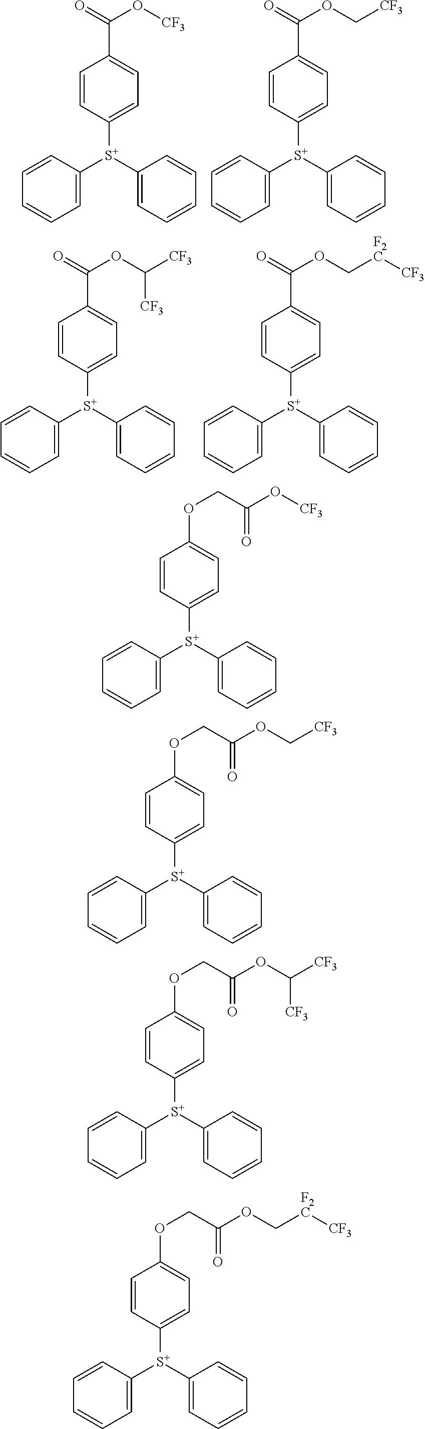

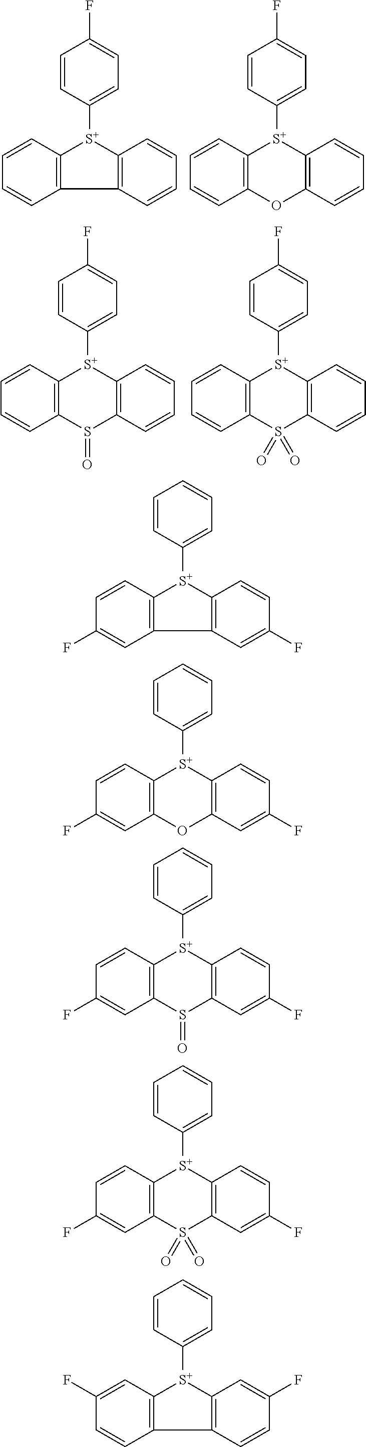

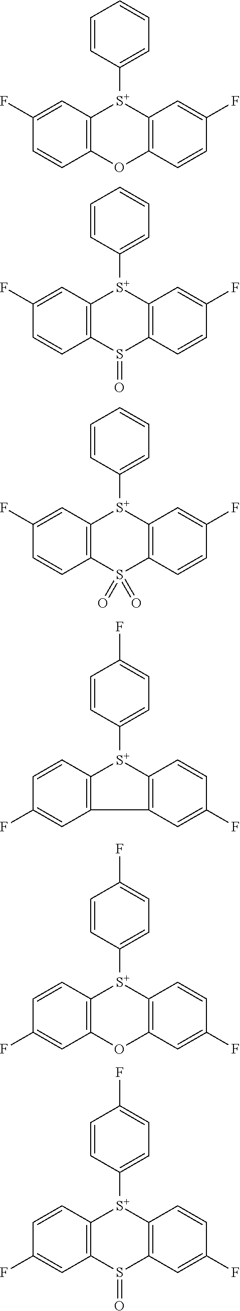 [SMILES: Fc1ccc2c(c1)[S+](c1ccccc1)c1cc(F)ccc1O2 ; O=S1c2ccc(F)cc2[S+](c2ccccc2)c2cc(F)ccc21 ; O=S1(=O)c2ccc(F)cc2[S+](c2ccccc2)c2cc(F)ccc21 ; Fc1ccc(-[s+]2c3ccc(F)cc3c3cc(F)ccc32)cc1 ; Fc1ccc([S+]2c3ccc(F)cc3Oc3cc(F)ccc32)cc1 ; O=S1c2cc(F)ccc2[S+](c2ccc(F)cc2)c2ccc(F)cc21]

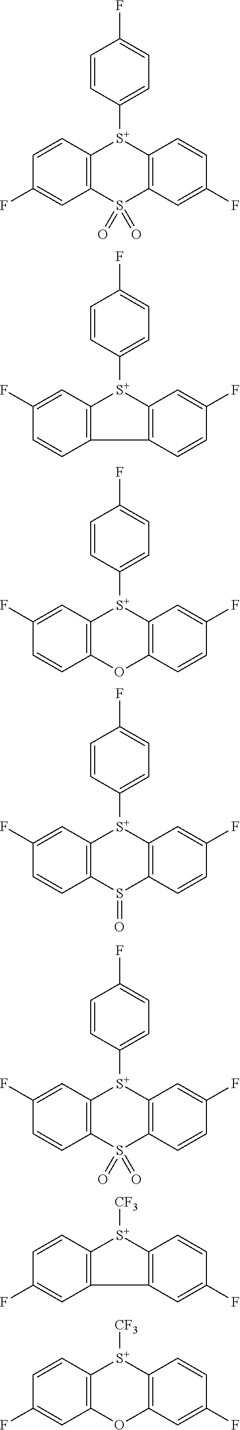

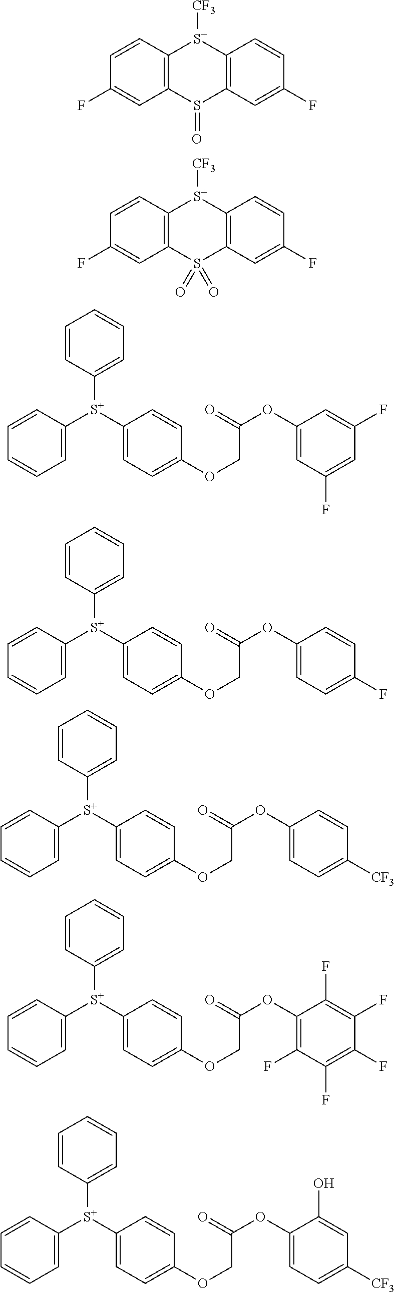

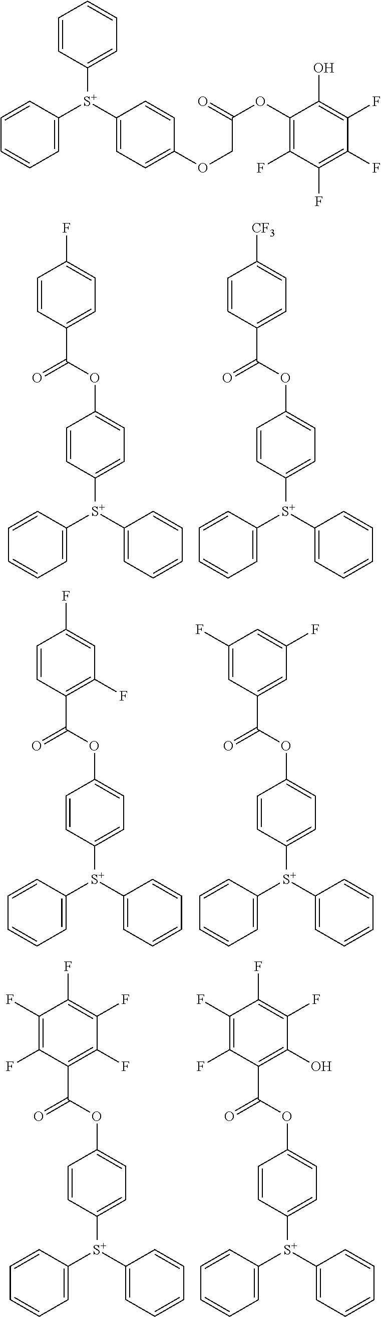

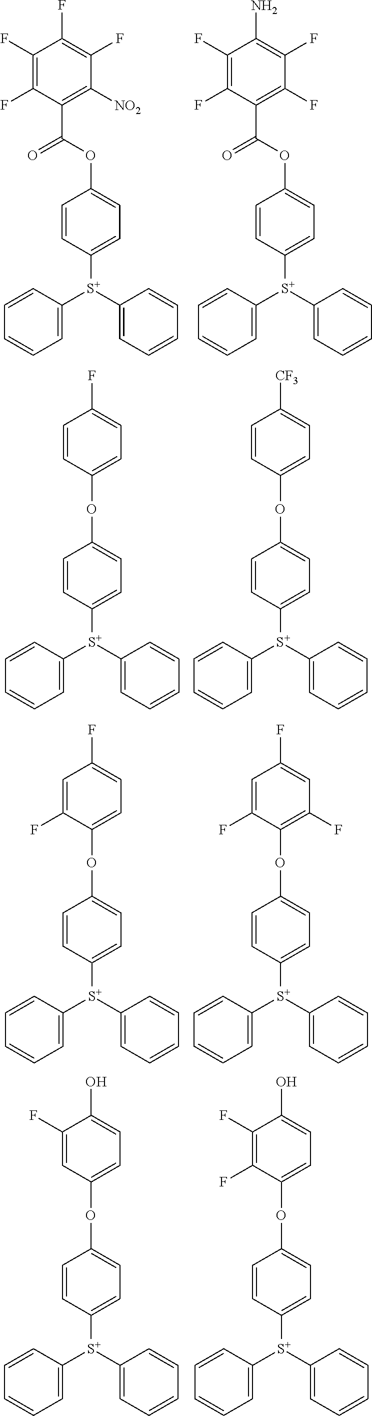

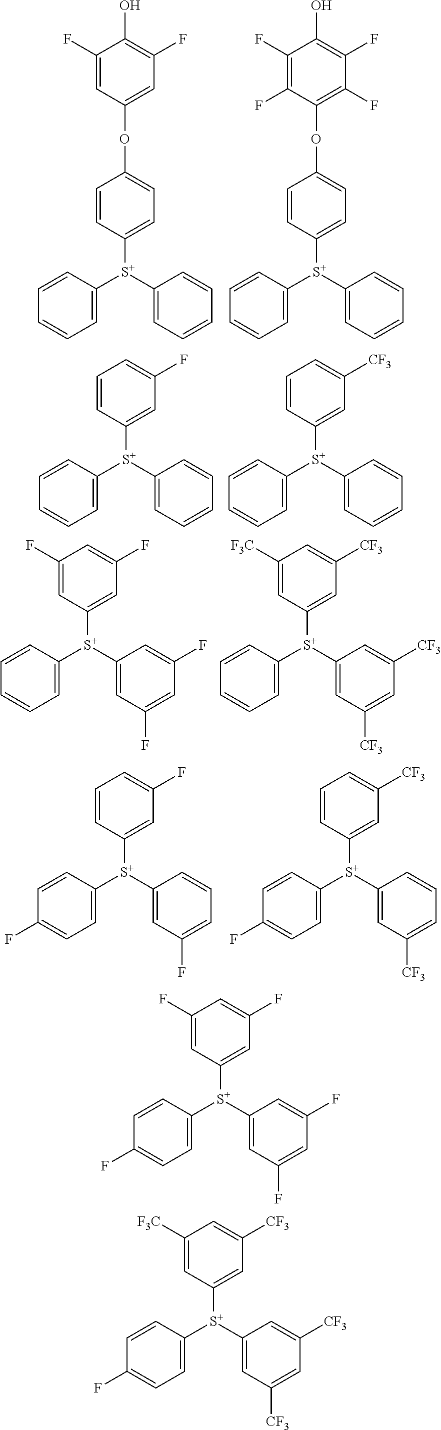

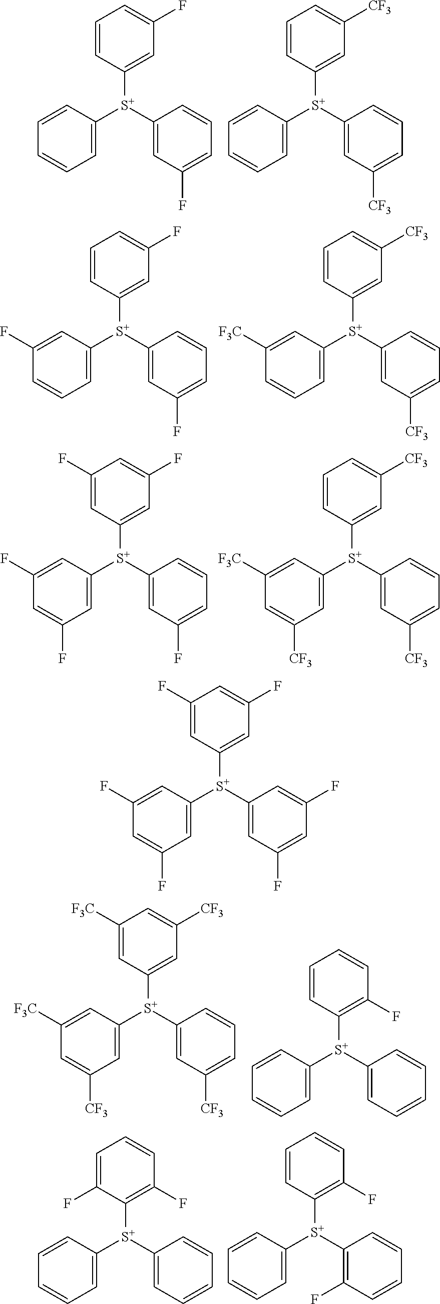

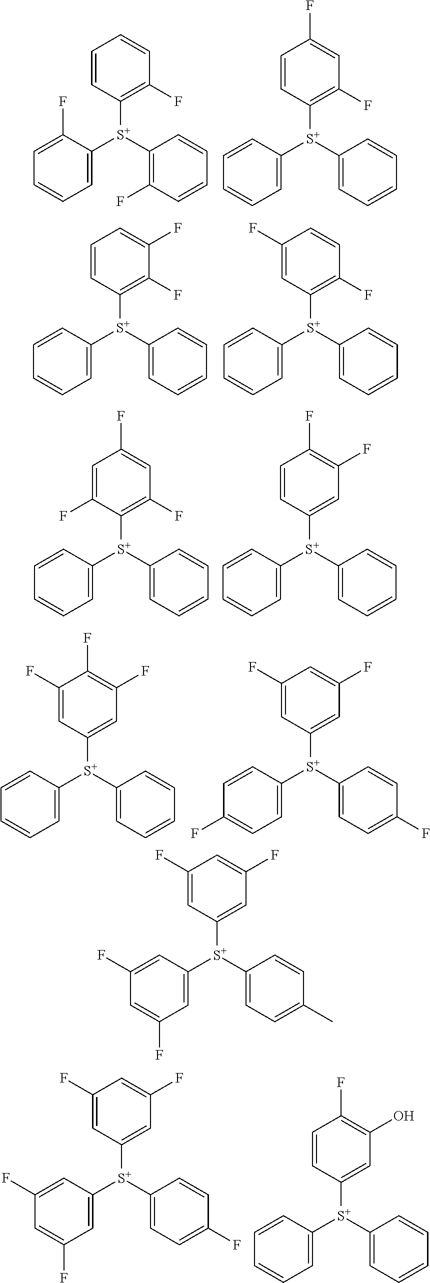

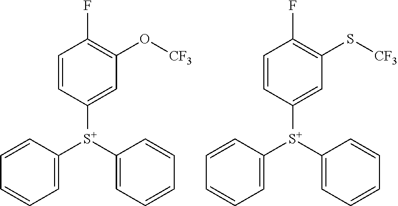 [SMILES: Fc1ccc([S+](c2ccccc2)c2ccccc2)cc1OC(F)(F)F ; Fc1ccc([S+](c2ccccc2)c2ccccc2)cc1SC(F)(F)F]

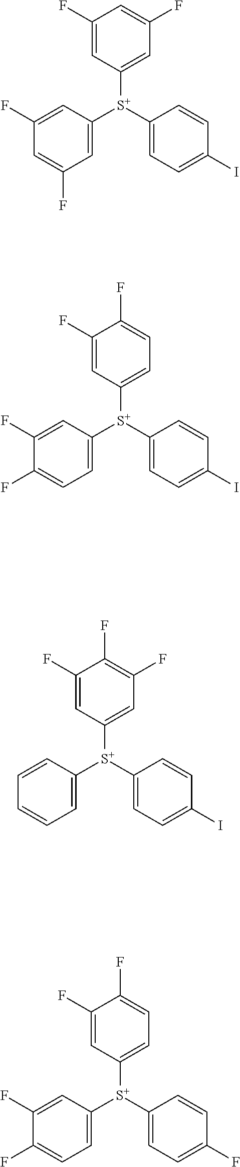 [SMILES: Fc1cc(F)cc([S+](c2ccc(I)cc2)c2cc(F)cc(F)c2)c1 ; Fc1ccc([S+](c2ccc(I)cc2)c2ccc(F)c(F)c2)cc1F ; Fc1cc([S+](c2ccccc2)c2ccc(I)cc2)cc(F)c1F ; Fc1ccc([S+](c2ccc(F)c(F)c2)c2ccc(F)c(F)c2)cc1]

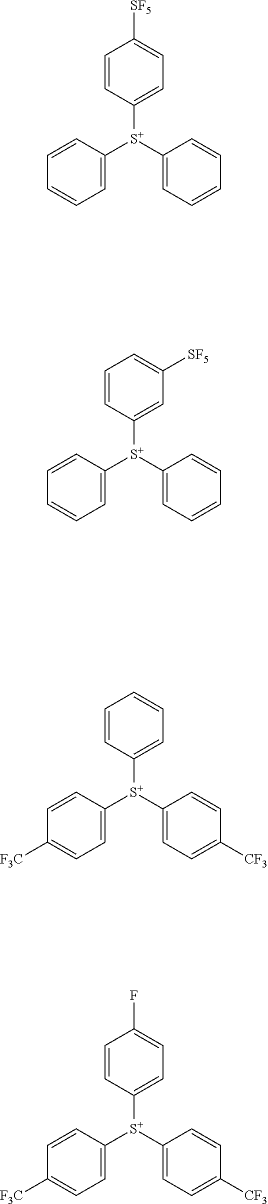

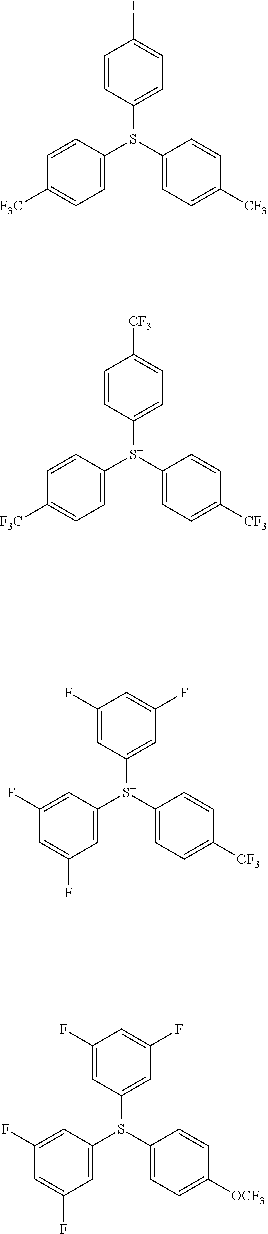 [SMILES: FC(F)(F)c1ccc([S+](c2ccc(I)cc2)c2ccc(C(F)(F)F)cc2)cc1 ; FC(F)(F)c1ccc([S+](c2ccc(C(F)(F)F)cc2)c2ccc(C(F)(F)F)cc2)cc1 ; Fc1cc(F)cc([S+](c2ccc(C(F)(F)F)cc2)c2cc(F)cc(F)c2)c1 ; Fc1cc(F)cc([S+](c2ccc(OC(F)(F)F)cc2)c2cc(F)cc(F)c2)c1]

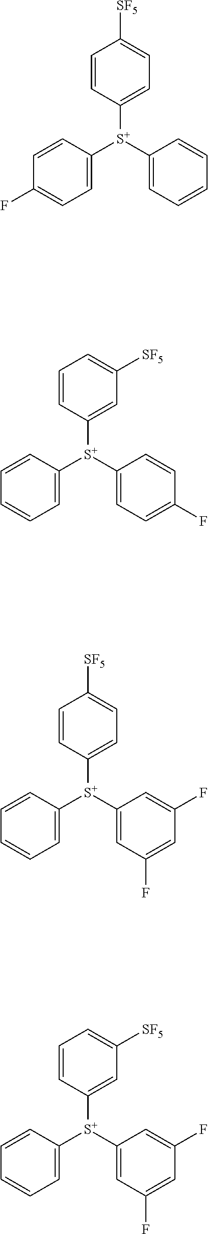

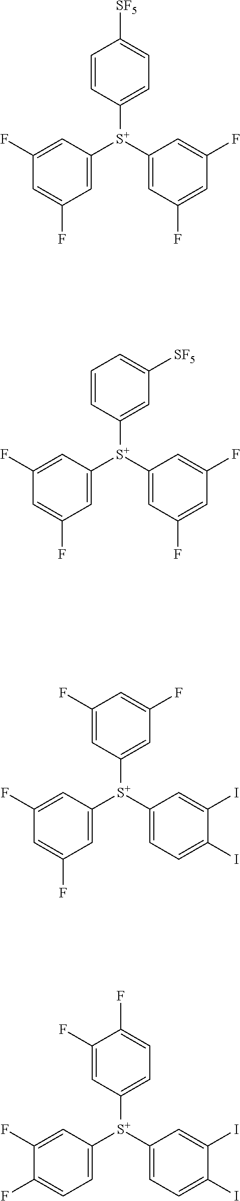

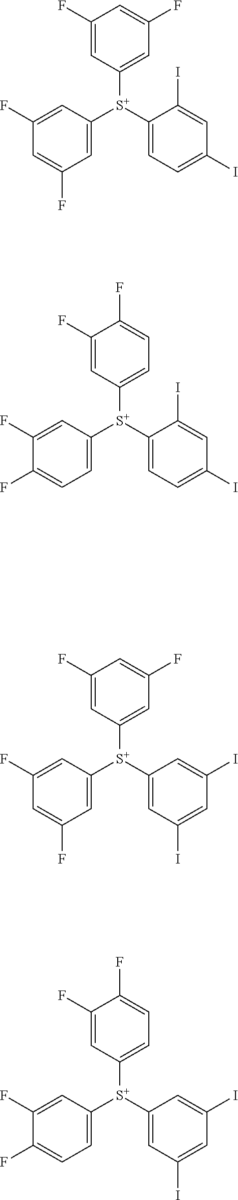 [SMILES: Fc1cc(F)cc([S+](c2cc(F)cc(F)c2)c2ccc(I)cc2I)c1 ; Fc1ccc([S+](c2ccc(F)c(F)c2)c2ccc(I)cc2I)cc1F ; Fc1cc(F)cc([S+](c2cc(F)cc(F)c2)c2cc(I)cc(I)c2)c1 ; Fc1ccc([S+](c2cc(I)cc(I)c2)c2ccc(F)c(F)c2)cc1F]

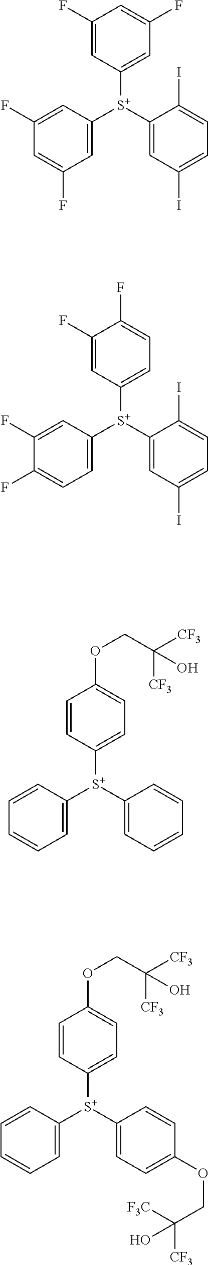 [SMILES: Fc1cc(F)cc([S+](c2cc(F)cc(F)c2)c2cc(I)ccc2I)c1 ; Fc1ccc([S+](c2ccc(F)c(F)c2)c2cc(I)ccc2I)cc1F ; OC(COc1ccc([S+](c2ccccc2)c2ccccc2)cc1)(C(F)(F)F)C(F)(F)F ; OC(COc1ccc([S+](c2ccccc2)c2ccc(OCC(O)(C(F)(F)F)C(F)(F)F)cc2)cc1)(C(F)(F)F)C(F)(F)F]

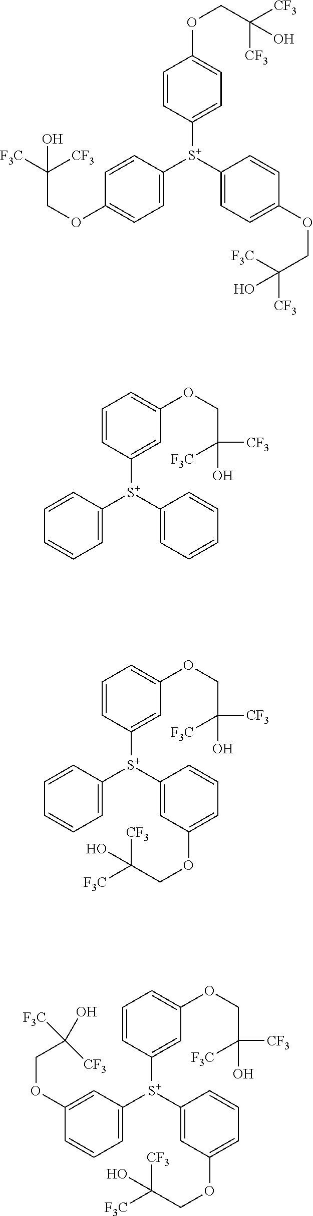 [SMILES: OC(COc1ccc([S+](c2ccc(OCC(O)(C(F)(F)F)C(F)(F)F)cc2)c2ccc(OCC(O)(C(F)(F)F)C(F)(F)F)cc2)cc1)(C(F)(F)F)C(F)(F)F ; OC(COc1cccc([S+](c2ccccc2)c2ccccc2)c1)(C(F)(F)F)C(F)(F)F ; OC(COc1cccc([S+](c2ccccc2)c2cccc(OCC(O)(C(F)(F)F)C(F)(F)F)c2)c1)(C(F)(F)F)C(F)(F)F ; OC(COc1cccc([S+](c2cccc(OCC(O)(C(F)(F)F)C(F)(F)F)c2)c2cccc(OCC(O)(C(F)(F)F)C(F)(F)F)c2)c1)(C(F)(F)F)C(F)(F)F]

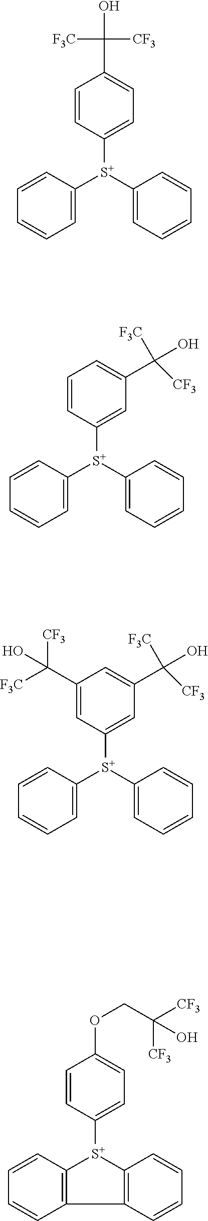 [SMILES: OC(c1ccc([S+](c2ccccc2)c2ccccc2)cc1)(C(F)(F)F)C(F)(F)F ; OC(c1cccc([S+](c2ccccc2)c2ccccc2)c1)(C(F)(F)F)C(F)(F)F ; OC(c1cc([S+](c2ccccc2)c2ccccc2)cc(C(O)(C(F)(F)F)C(F)(F)F)c1)(C(F)(F)F)C(F)(F)F ; OC(COc1ccc(-[s+]2c3ccccc3c3ccccc32)cc1)(C(F)(F)F)C(F)(F)F]

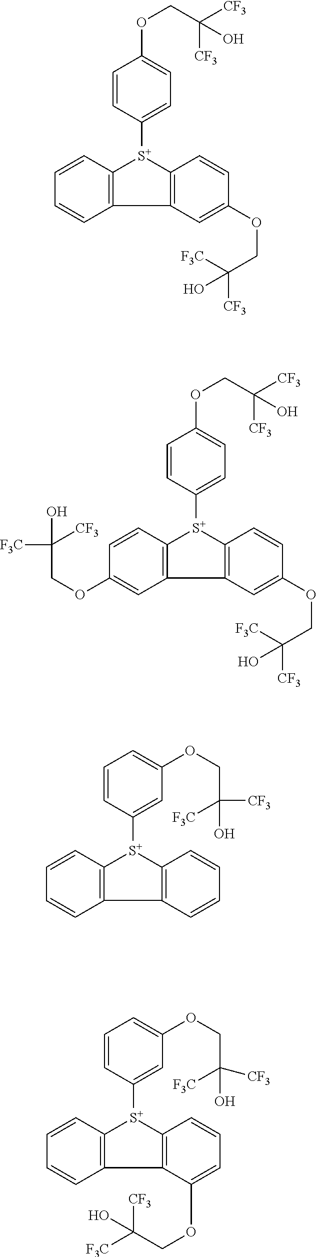 [SMILES: OC(COc1ccc(-[s+]2c3ccccc3c3cc(OCC(O)(C(F)(F)F)C(F)(F)F)ccc32)cc1)(C(F)(F)F)C(F)(F)F ; OC(COc1ccc(-[s+]2c3ccc(OCC(O)(C(F)(F)F)C(F)(F)F)cc3c3cc(OCC(O)(C(F)(F)F)C(F)(F)F)ccc32)cc1)(C(F)(F)F)C(F)(F)F ; OC(COc1cccc(-[s+]2c3ccccc3c3ccccc32)c1)(C(F)(F)F)C(F)(F)F ; OC(COc1cccc(-[s+]2c3ccccc3c3c(OCC(O)(C(F)(F)F)C(F)(F)F)cccc32)c1)(C(F)(F)F)C(F)(F)F]

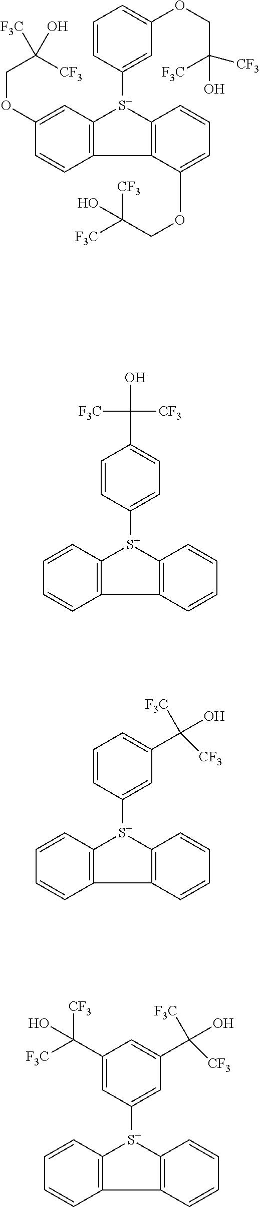 [SMILES: OC(COc1cccc(-[s+]2c3cc(OCC(O)(C(F)(F)F)C(F)(F)F)ccc3c3c(OCC(O)(C(F)(F)F)C(F)(F)F)cccc32)c1)(C(F)(F)F)C(F)(F)F ; OC(c1ccc(-[s+]2c3ccccc3c3ccccc32)cc1)(C(F)(F)F)C(F)(F)F ; OC(c1cccc(-[s+]2c3ccccc3c3ccccc32)c1)(C(F)(F)F)C(F)(F)F ; OC(c1cc(-[s+]2c3ccccc3c3ccccc32)cc(C(O)(C(F)(F)F)C(F)(F)F)c1)(C(F)(F)F)C(F)(F)F]

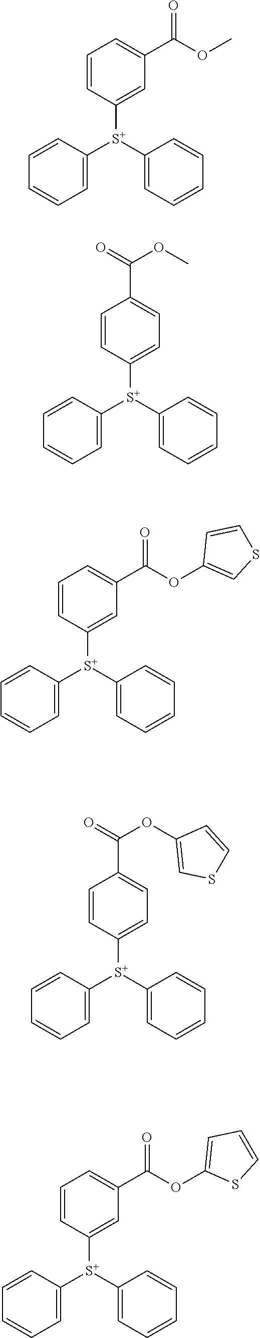 [SMILES: COC(=O)c1cccc([S+](c2ccccc2)c2ccccc2)c1 ; COC(=O)c1ccc([S+](c2ccccc2)c2ccccc2)cc1 ; O=C(Oc1ccsc1)c1cccc([S+](c2ccccc2)c2ccccc2)c1 ; O=C(Oc1ccsc1)c1ccc([S+](c2ccccc2)c2ccccc2)cc1 ; O=C(Oc1cccs1)c1cccc([S+](c2ccccc2)c2ccccc2)c1]

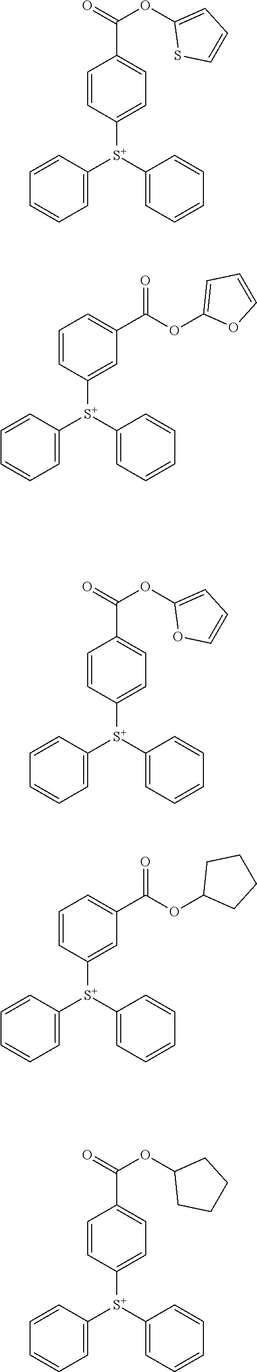 [SMILES: O=C(Oc1cccs1)c1ccc([S+](c2ccccc2)c2ccccc2)cc1 ; O=C(Oc1ccco1)c1cccc([S+](c2ccccc2)c2ccccc2)c1 ; O=C(Oc1ccco1)c1ccc([S+](c2ccccc2)c2ccccc2)cc1 ; O=C(OC1CCCC1)c1cccc([S+](c2ccccc2)c2ccccc2)c1 ; O=C(OC1CCCC1)c1ccc([S+](c2ccccc2)c2ccccc2)cc1]

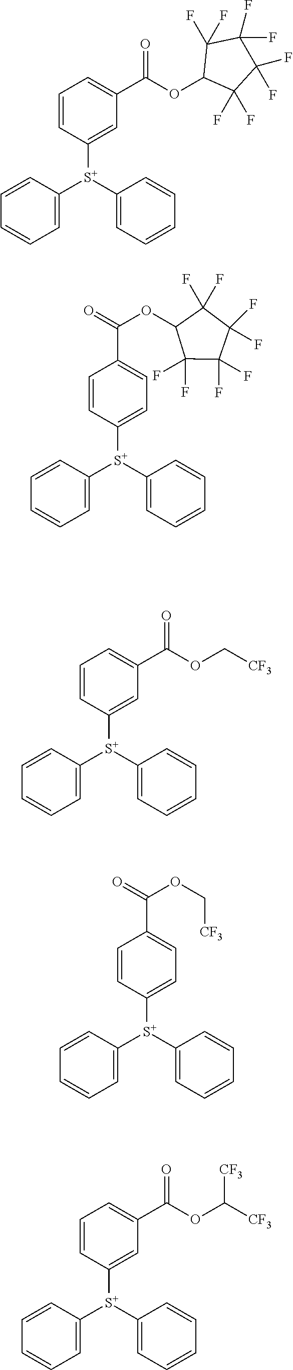 [SMILES: O=C(OC1C(F)(F)C(F)(F)C(F)(F)C1(F)F)c1cccc([S+](c2ccccc2)c2ccccc2)c1 ; O=C(OC1C(F)(F)C(F)(F)C(F)(F)C1(F)F)c1ccc([S+](c2ccccc2)c2ccccc2)cc1 ; O=C(OCC(F)(F)F)c1cccc([S+](c2ccccc2)c2ccccc2)c1 ; O=C(OCC(F)(F)F)c1ccc([S+](c2ccccc2)c2ccccc2)cc1 ; O=C(OC(C(F)(F)F)C(F)(F)F)c1cccc([S+](c2ccccc2)c2ccccc2)c1]

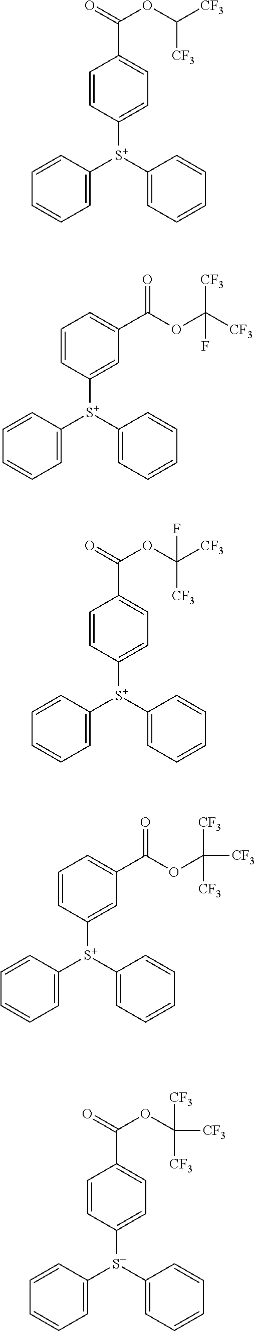 [SMILES: O=C(OC(C(F)(F)F)C(F)(F)F)c1ccc([S+](c2ccccc2)c2ccccc2)cc1 ; O=C(OC(F)(C(F)(F)F)C(F)(F)F)c1cccc([S+](c2ccccc2)c2ccccc2)c1 ; O=C(OC(F)(C(F)(F)F)C(F)(F)F)c1ccc([S+](c2ccccc2)c2ccccc2)cc1 ; O=C(OC(C(F)(F)F)(C(F)(F)F)C(F)(F)F)c1cccc([S+](c2ccccc2)c2ccccc2)c1 ; O=C(OC(C(F)(F)F)(C(F)(F)F)C(F)(F)F)c1ccc([S+](c2ccccc2)c2ccccc2)cc1]

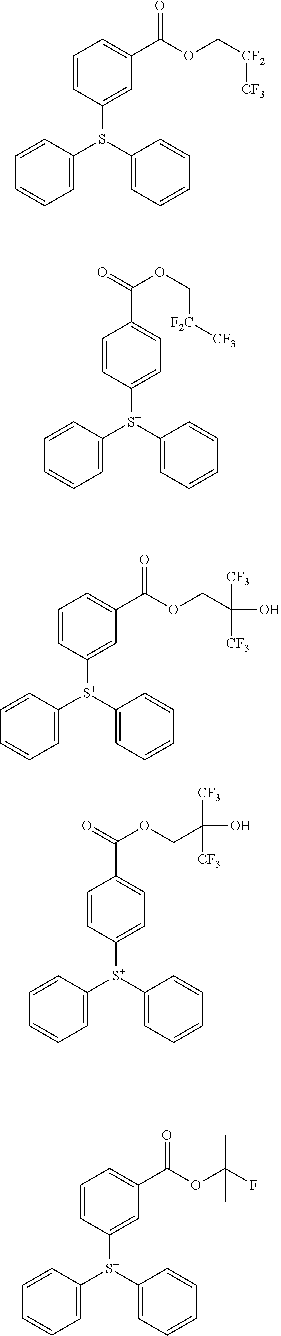 [SMILES: O=C(OCC(F)(F)C(F)(F)F)c1cccc([S+](c2ccccc2)c2ccccc2)c1 ; O=C(OCC(F)(F)C(F)(F)F)c1ccc([S+](c2ccccc2)c2ccccc2)cc1 ; O=C(OCC(O)(C(F)(F)F)C(F)(F)F)c1cccc([S+](c2ccccc2)c2ccccc2)c1 ; O=C(OCC(O)(C(F)(F)F)C(F)(F)F)c1ccc([S+](c2ccccc2)c2ccccc2)cc1 ; CC(C)(F)OC(=O)c1cccc([S+](c2ccccc2)c2ccccc2)c1]

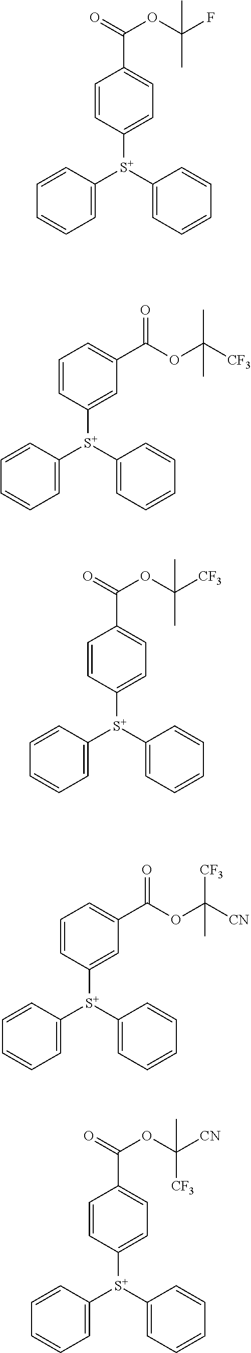 [SMILES: CC(C)(F)OC(=O)c1ccc([S+](c2ccccc2)c2ccccc2)cc1 ; CC(C)(OC(=O)c1cccc([S+](c2ccccc2)c2ccccc2)c1)C(F)(F)F ; CC(C)(OC(=O)c1ccc([S+](c2ccccc2)c2ccccc2)cc1)C(F)(F)F ; CC(C#N)(OC(=O)c1cccc([S+](c2ccccc2)c2ccccc2)c1)C(F)(F)F ; CC(C#N)(OC(=O)c1ccc([S+](c2ccccc2)c2ccccc2)cc1)C(F)(F)F]

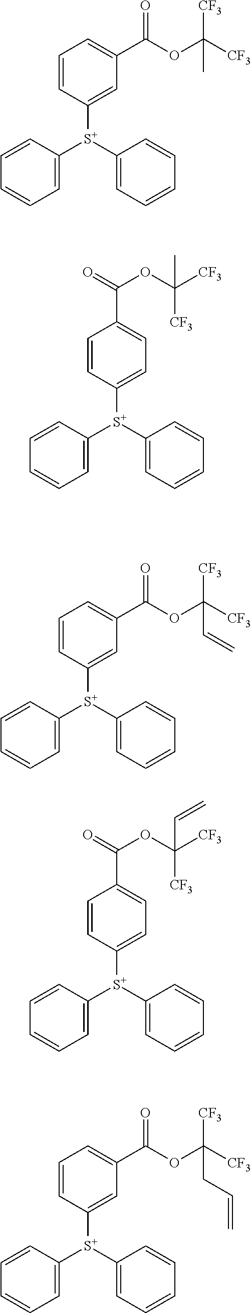 [SMILES: CC(OC(=O)c1cccc([S+](c2ccccc2)c2ccccc2)c1)(C(F)(F)F)C(F)(F)F ; CC(OC(=O)c1ccc([S+](c2ccccc2)c2ccccc2)cc1)(C(F)(F)F)C(F)(F)F ; C=CC(OC(=O)c1cccc([S+](c2ccccc2)c2ccccc2)c1)(C(F)(F)F)C(F)(F)F ; C=CC(OC(=O)c1ccc([S+](c2ccccc2)c2ccccc2)cc1)(C(F)(F)F)C(F)(F)F ; C=CCC(OC(=O)c1cccc([S+](c2ccccc2)c2ccccc2)c1)(C(F)(F)F)C(F)(F)F]

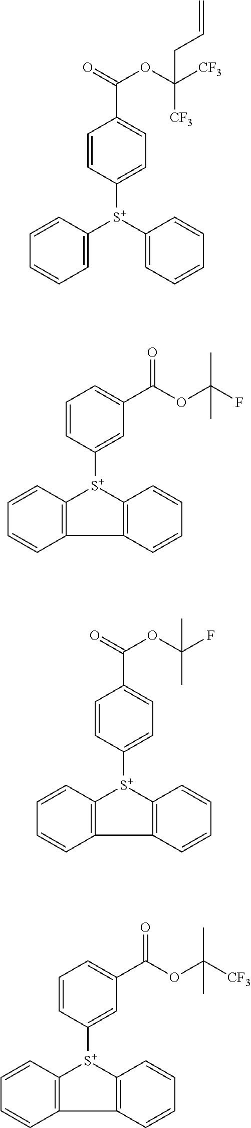 [SMILES: C=CCC(OC(=O)c1ccc([S+](c2ccccc2)c2ccccc2)cc1)(C(F)(F)F)C(F)(F)F ; CC(C)(F)OC(=O)c1cccc(-[s+]2c3ccccc3c3ccccc32)c1 ; CC(C)(F)OC(=O)c1ccc(-[s+]2c3ccccc3c3ccccc32)cc1 ; CC(C)(OC(=O)c1cccc(-[s+]2c3ccccc3c3ccccc32)c1)C(F)(F)F]

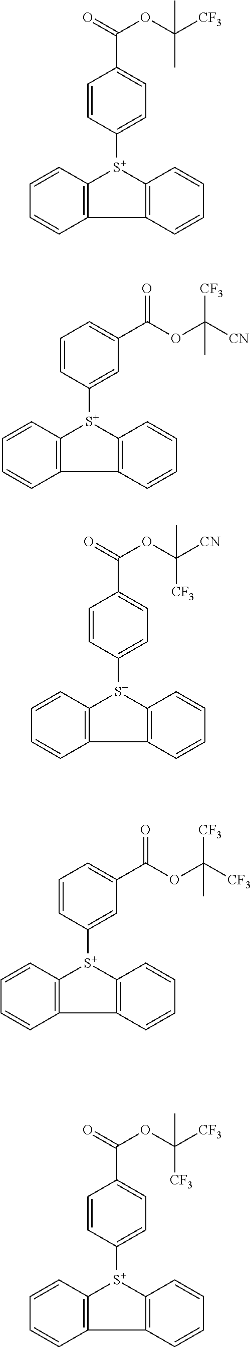 [SMILES: CC(C)(OC(=O)c1ccc(-[s+]2c3ccccc3c3ccccc32)cc1)C(F)(F)F ; CC(C#N)(OC(=O)c1cccc(-[s+]2c3ccccc3c3ccccc32)c1)C(F)(F)F ; CC(C#N)(OC(=O)c1ccc(-[s+]2c3ccccc3c3ccccc32)cc1)C(F)(F)F ; CC(OC(=O)c1cccc(-[s+]2c3ccccc3c3ccccc32)c1)(C(F)(F)F)C(F)(F)F ; CC(OC(=O)c1ccc(-[s+]2c3ccccc3c3ccccc32)cc1)(C(F)(F)F)C(F)(F)F]

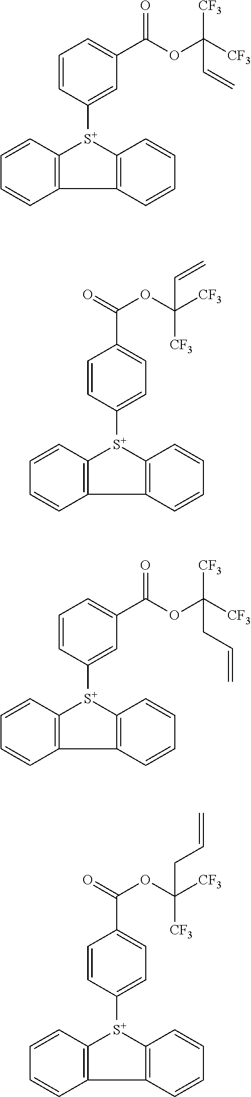 [SMILES: C=CC(OC(=O)c1cccc(-[s+]2c3ccccc3c3ccccc32)c1)(C(F)(F)F)C(F)(F)F ; C=CC(OC(=O)c1ccc(-[s+]2c3ccccc3c3ccccc32)cc1)(C(F)(F)F)C(F)(F)F ; C=CCC(OC(=O)c1cccc(-[s+]2c3ccccc3c3ccccc32)c1)(C(F)(F)F)C(F)(F)F ; C=CCC(OC(=O)c1ccc(-[s+]2c3ccccc3c3ccccc32)cc1)(C(F)(F)F)C(F)(F)F]

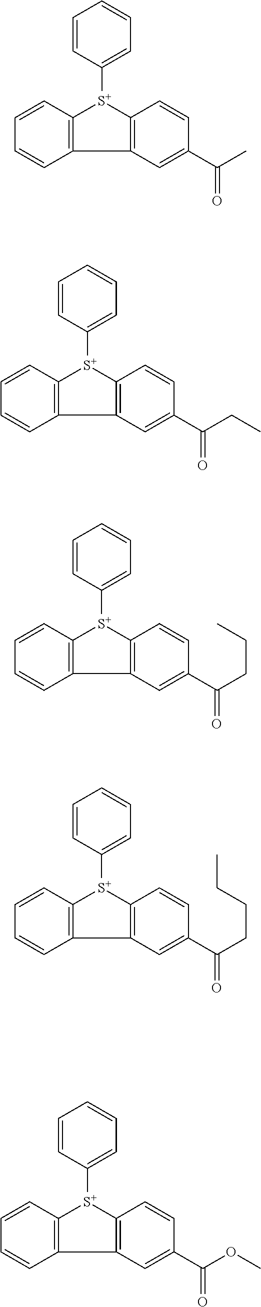 [SMILES: CC(=O)c1ccc2c(c1)c1ccccc1[s+]2-c1ccccc1 ; CCC(=O)c1ccc2c(c1)c1ccccc1[s+]2-c1ccccc1 ; CCCC(=O)c1ccc2c(c1)c1ccccc1[s+]2-c1ccccc1 ; CCCCC(=O)c1ccc2c(c1)c1ccccc1[s+]2-c1ccccc1 ; COC(=O)c1ccc2c(c1)c1ccccc1[s+]2-c1ccccc1]

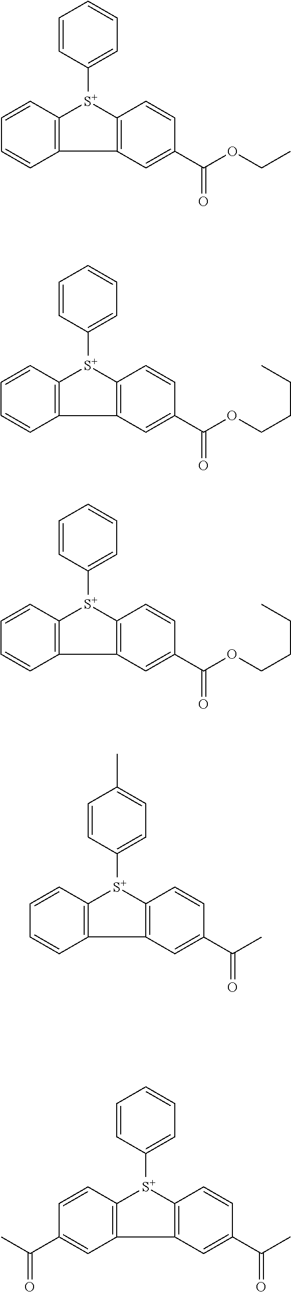

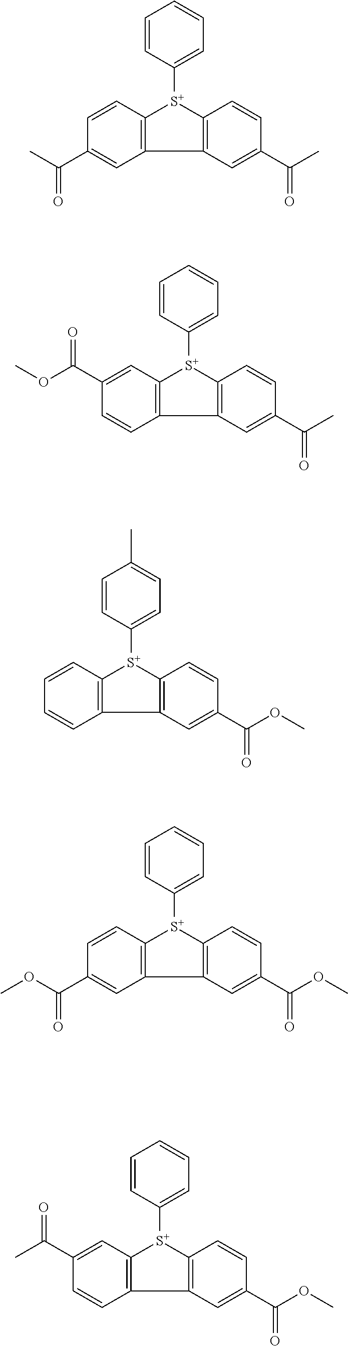

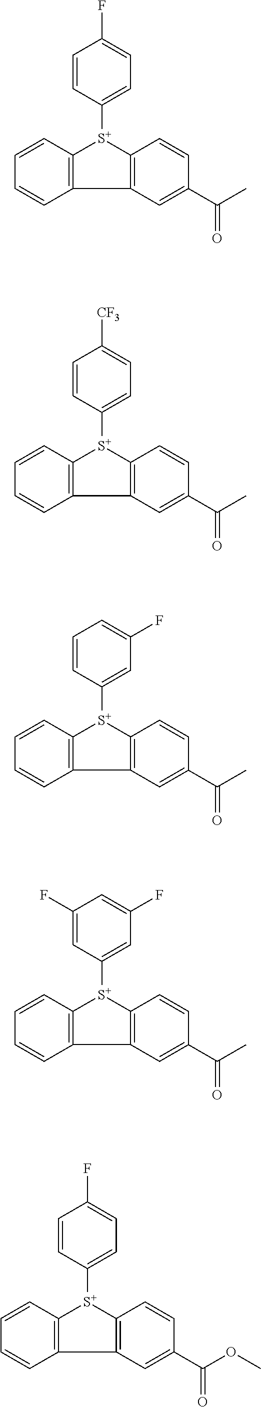 [SMILES: CC(=O)c1ccc2c(c1)c1ccccc1[s+]2-c1ccc(F)cc1 ; CC(=O)c1ccc2c(c1)c1ccccc1[s+]2-c1ccc(C(F)(F)F)cc1 ; CC(=O)c1ccc2c(c1)c1ccccc1[s+]2-c1cccc(F)c1 ; CC(=O)c1ccc2c(c1)c1ccccc1[s+]2-c1cc(F)cc(F)c1 ; COC(=O)c1ccc2c(c1)c1ccccc1[s+]2-c1ccc(F)cc1]

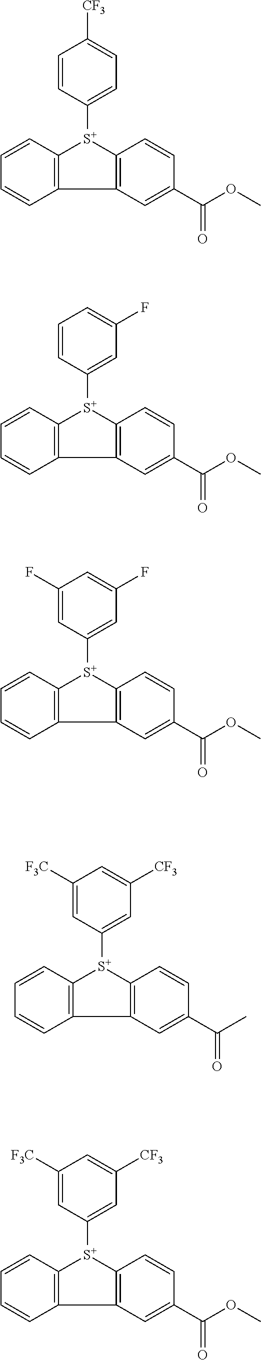 [SMILES: COC(=O)c1ccc2c(c1)c1ccccc1[s+]2-c1ccc(C(F)(F)F)cc1 ; COC(=O)c1ccc2c(c1)c1ccccc1[s+]2-c1cccc(F)c1 ; COC(=O)c1ccc2c(c1)c1ccccc1[s+]2-c1cc(F)cc(F)c1 ; CC(=O)c1ccc2c(c1)c1ccccc1[s+]2-c1cc(C(F)(F)F)cc(C(F)(F)F)c1 ; COC(=O)c1ccc2c(c1)c1ccccc1[s+]2-c1cc(C(F)(F)F)cc(C(F)(F)F)c1]

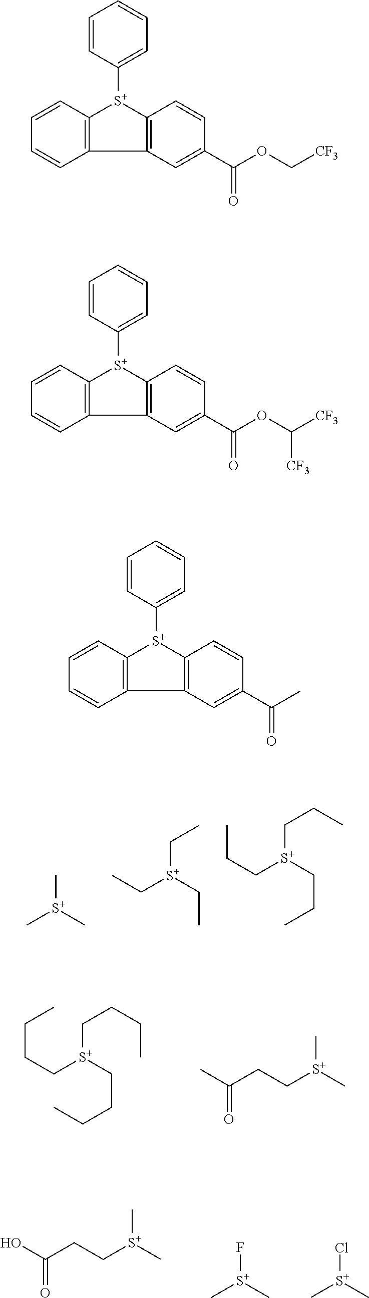

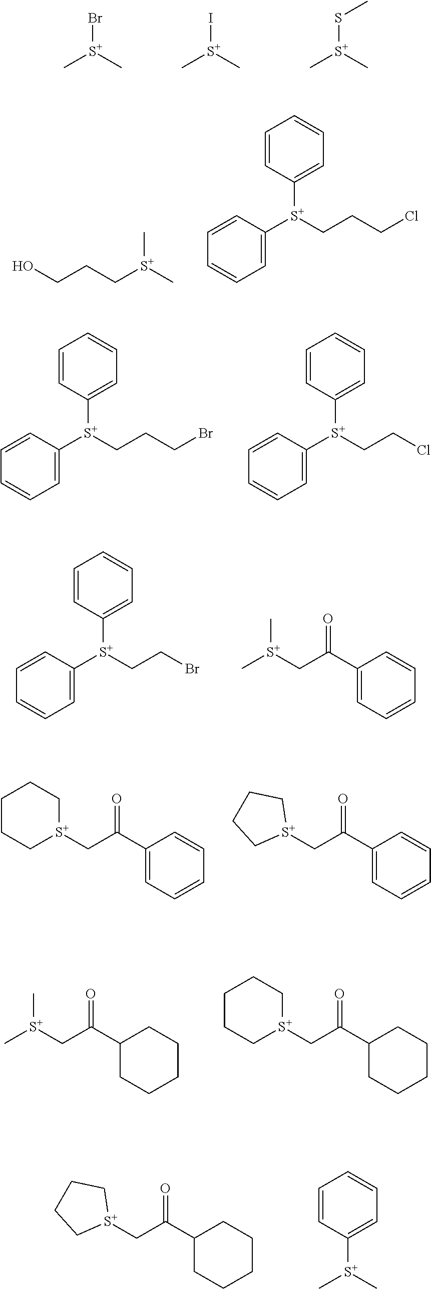

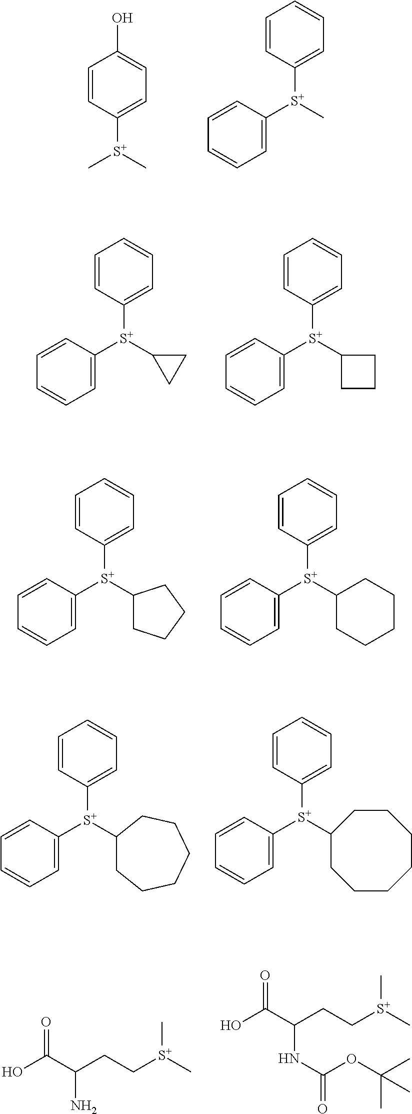

Sulfonium Salt A may be synthesized, for example, by ion exchange between a hydrochloride or carbonate salt having a sulfonium cation and a protonic acid or ammonium salt having an anion.

In the resist composition, Sulfonium Salt A is preferably used in an amount of 0.001 to 50 parts by weight, more preferably 0.01 to 40 parts by weight per 100 parts by weight of the base polymer to be described below. Sulfonium Salt A may be used alone or in admixture of two or more.

### [Base Polymer]

In one embodiment, the resist composition contains a base polymer. In the case of positive resist compositions, the base polymer comprises repeat units containing an acid labile group. The preferred repeat units containing an acid labile group are repeat units having the formula (a1) or repeat units having the formula (a2), which are also referred to as repeat units (a1) or (a2).

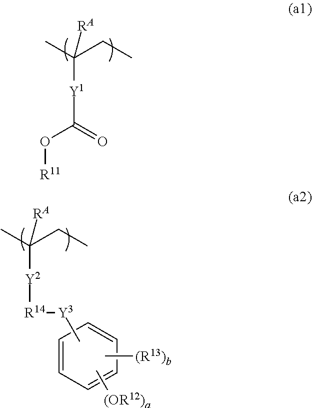

In formulae (a1) and (a2), R^{A} is each independently hydrogen or methyl. Y¹ is a single bond, phenylene group, naphthylene group, or a C₁-C₁₂ linking group containing at least one moiety selected from ester bond, ether bond and lactone ring. Y² is a single bond or ester bond. Y³ is a single bond, ether bond or ester bond. R¹¹ and R¹² are each independently an acid labile group. R¹³ is fluorine, trifluoromethyl, cyano or a C₁-C₆ saturated hydrocarbyl group. R¹⁴ is a single bond or a C₁-C₆ alkanediyl group in which some —CH₂— may be replaced by an ether bond or ester bond. The subscript “a” is 1 or 2, “b” is an integer of 0 to 4, and the sum of a+b is from 1 to 5.

Examples of the monomer from which repeat units (a1) are derived are shown below, but not limited thereto. Herein R^{A} and R¹¹ are as defined above.

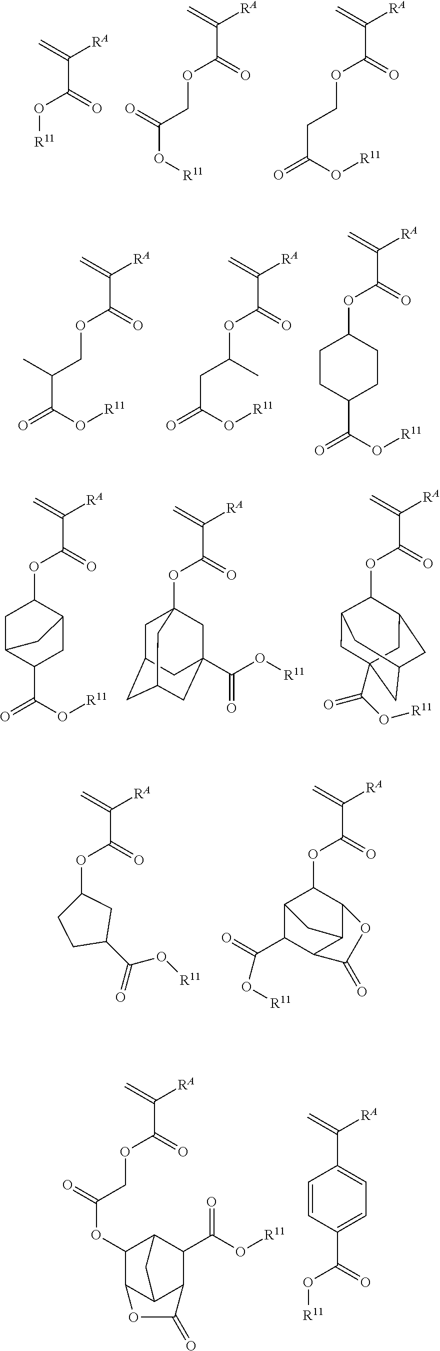

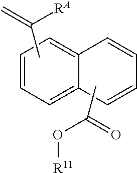

Examples of the monomer from which repeat units (a2) are derived are shown below, but not limited thereto. Herein R^{A} and R¹² are as defined above.

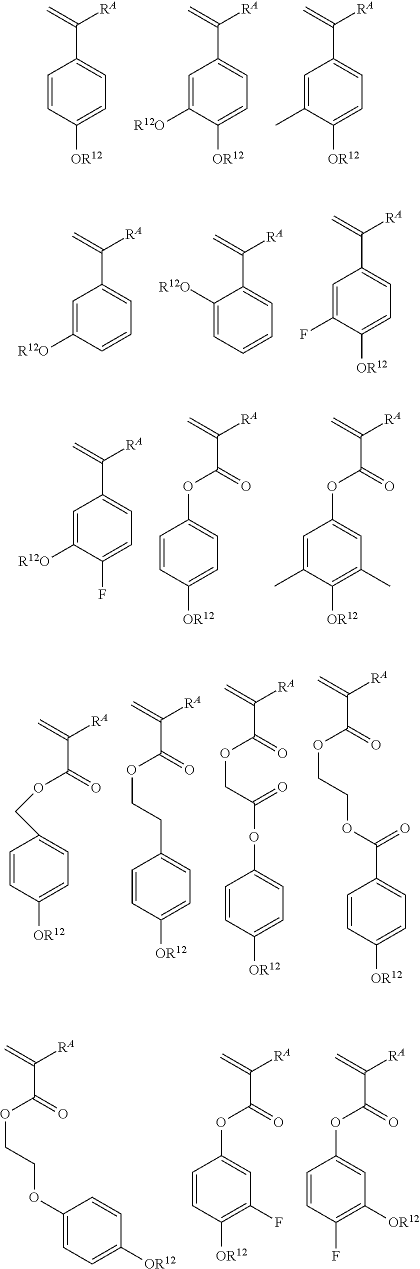

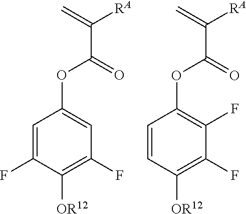

The acid labile groups represented by R¹¹ and R¹² may be selected from a variety of such groups, for example, those groups described in JP-A 2013-080033 (U.S. Pat. No. 8,574,817) and JP-A 2013-083821 (U.S. Pat. No. 8,846,303).

Typical of the acid labile group are groups of the following formulae (AL-1) to (AL-3).

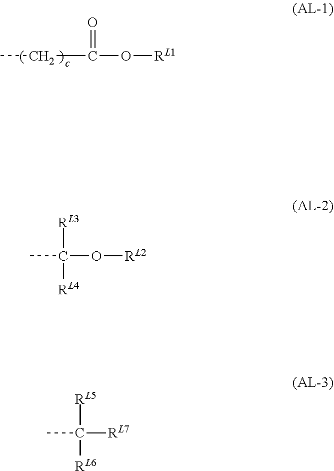

In formulae (AL-1) and (AL-2), R^{L1} and R^{L2} are each independently a C₁-C₄₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₄₀ saturated hydrocarbyl groups are preferred, and C₁-C₂₀ saturated hydrocarbyl groups are more preferred.

In formula (AL-1), c is an integer of 0 to 10, preferably 1 to 5.

In formula (AL-2), R^{L3} and R^{L4} are each independently hydrogen or a C₁-C₂₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ saturated hydrocarbyl groups are preferred. Any two of R^{L2}, R^{L3} and R^{L4} may bond together to form a C₃-C₂₀ ring with the carbon atom or carbon and oxygen atoms to which they are attached. The ring preferably contains 4 to 16 carbon atoms and is typically alicyclic.

In formula (AL-3), R^{L5}, R^{L6} and R^{L7} are each independently a C₁-C₂₀ hydrocarbyl group which may contain a heteroatom such as oxygen, sulfur, nitrogen or fluorine. The hydrocarbyl group may be saturated or unsaturated and straight, branched or cyclic. Inter alia, C₁-C₂₀ saturated hydrocarbyl groups are preferred. Any two of R^{L5}, R^{L6} and R^{L7} may bond together to form a C₃-C₂₀ ring with the carbon atom to which they are attached. The ring preferably contains 4 to 16 carbon atoms and is typically alicyclic.

The base polymer may further comprise repeat units (b) having a phenolic hydroxy group as an adhesive group. Examples of suitable monomers from which repeat units (b) are derived are given below, but not limited thereto. Herein R^{A} is as defined above.

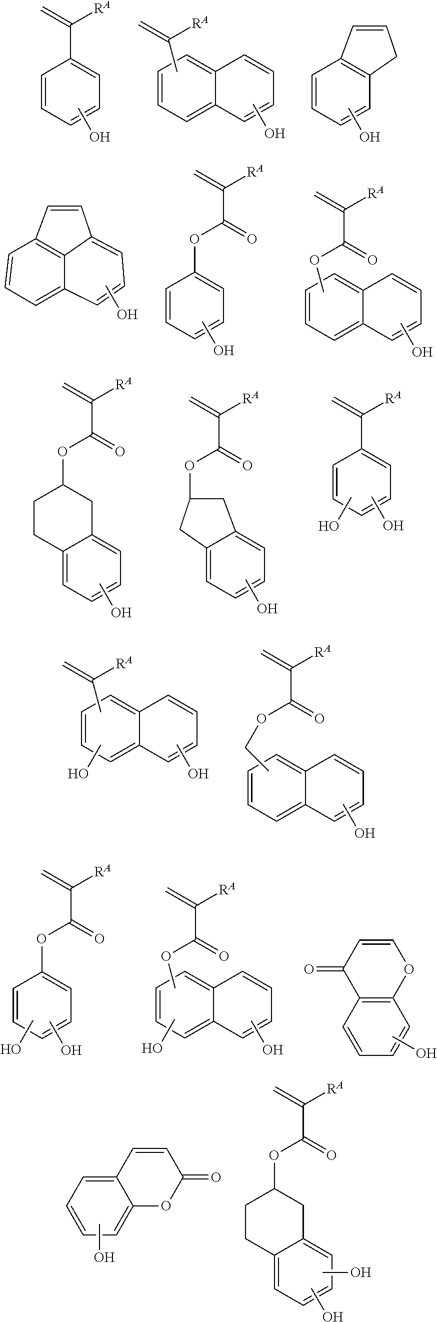

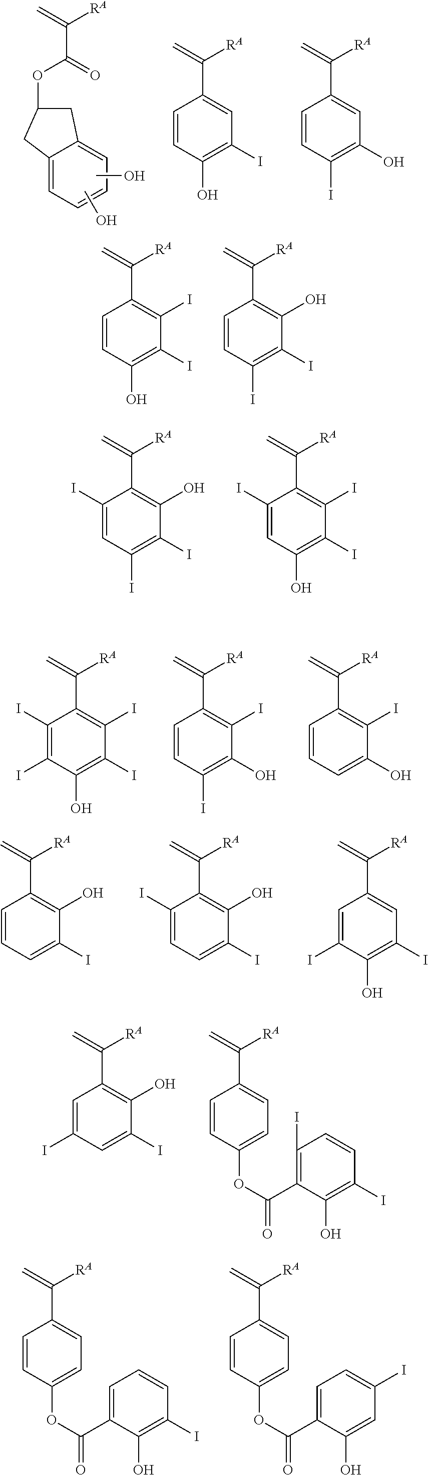

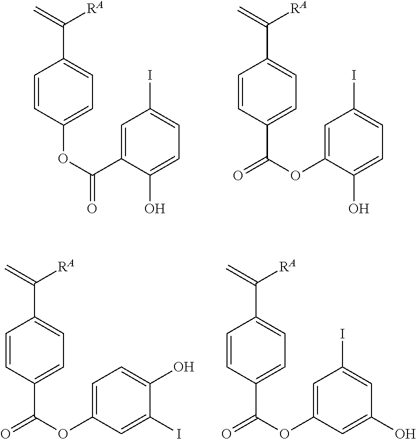

The base polymer may further comprise repeat units (c) having another adhesive group selected from hydroxy group (other than the foregoing phenolic hydroxy), lactone ring, sultone ring, ether bond, ester bond, sulfonate ester bond, carbonyl group, sulfonyl group, cyano group, and carboxy group. Examples of suitable monomers from which repeat units (c) are derived are given below, but not limited thereto. Herein R^{A} is as defined above.

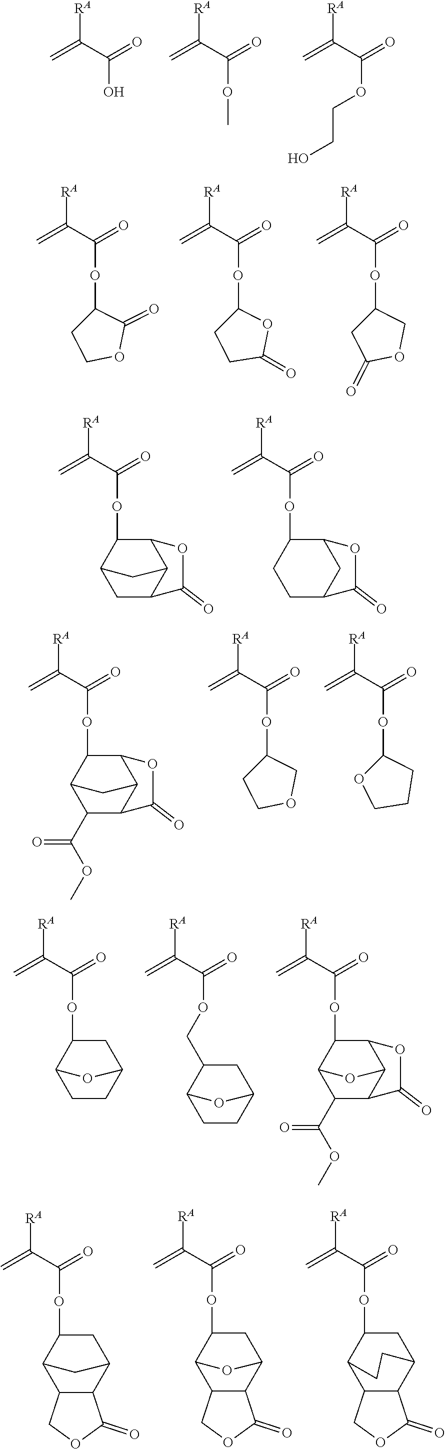

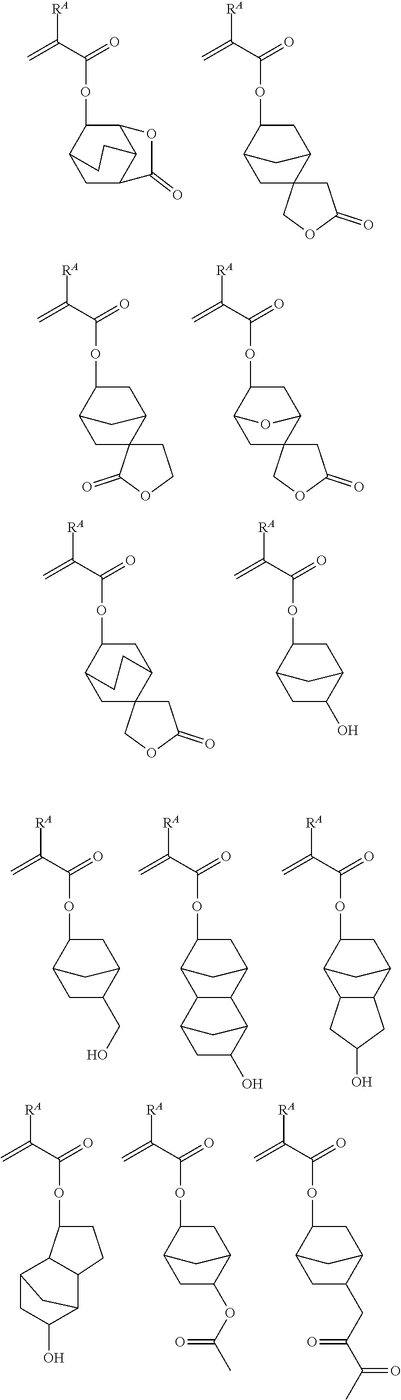

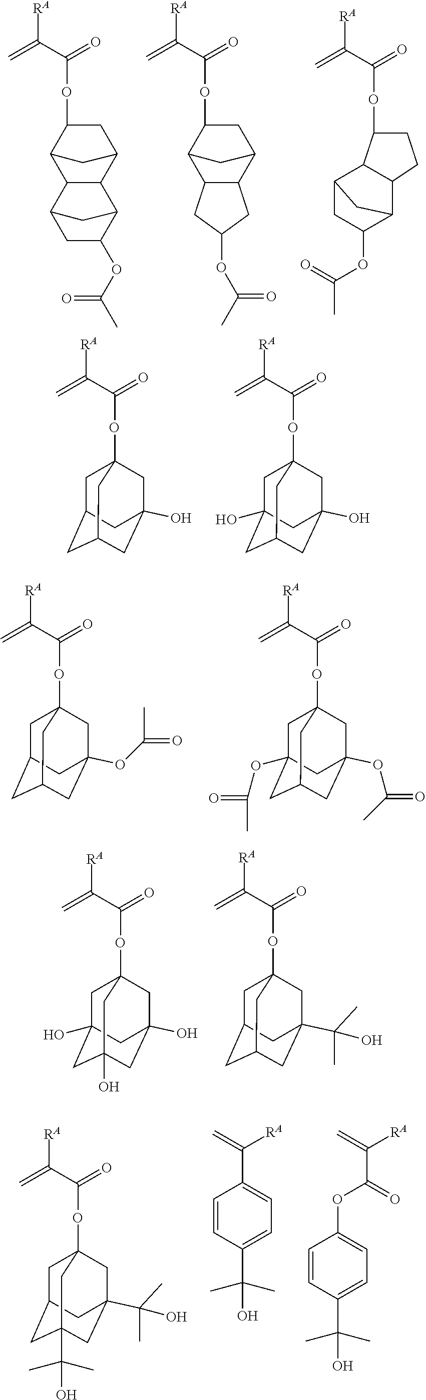

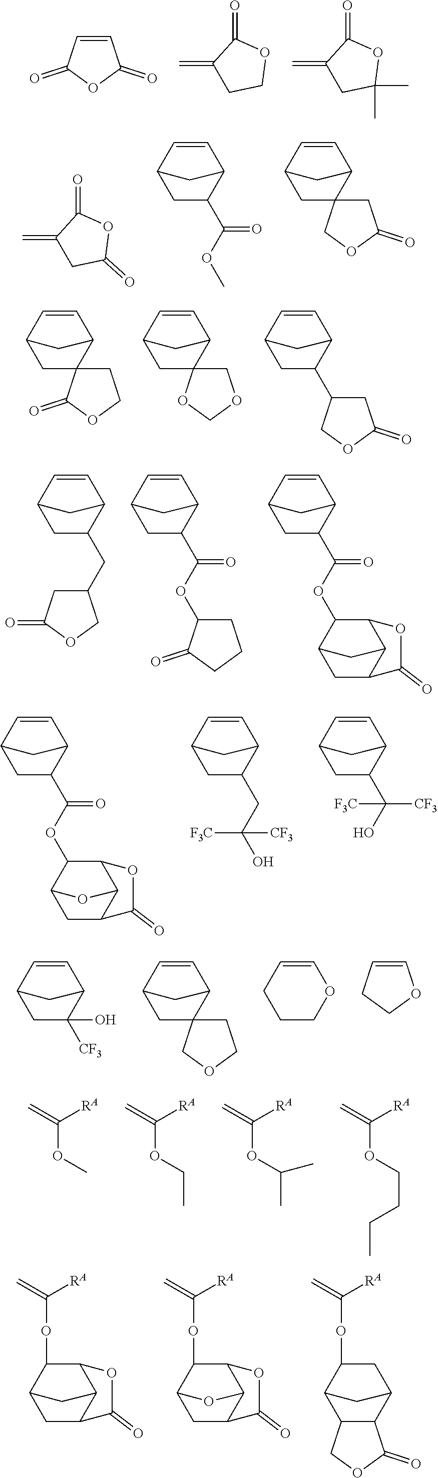

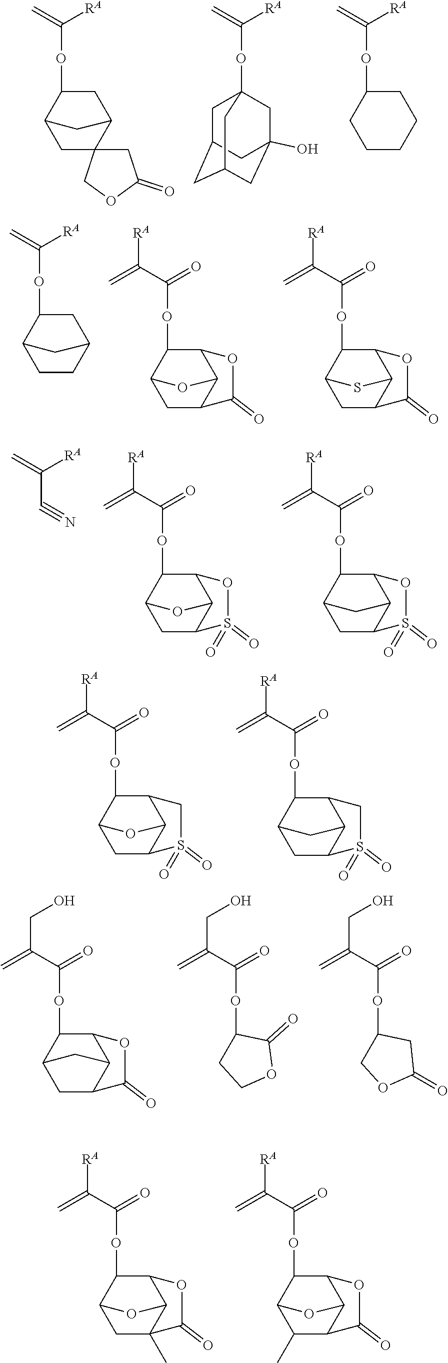

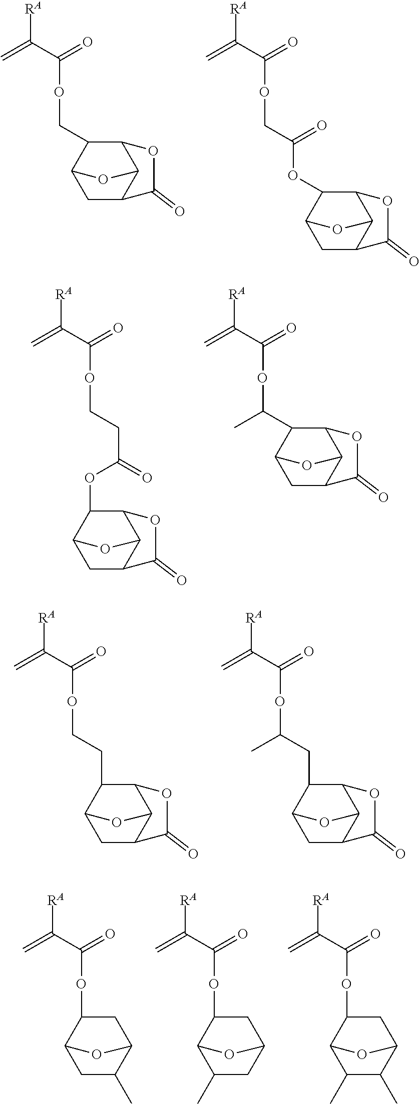

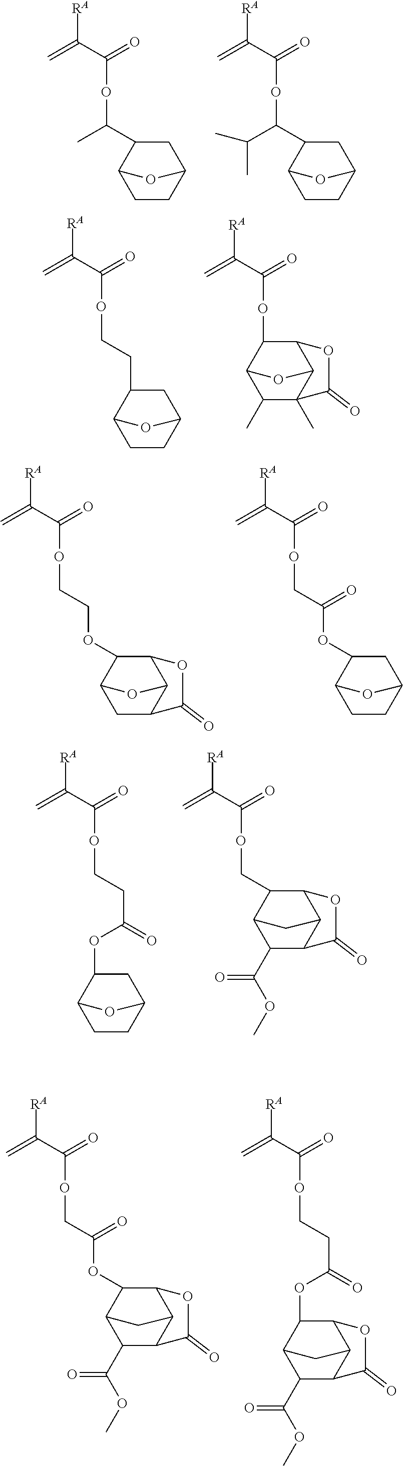

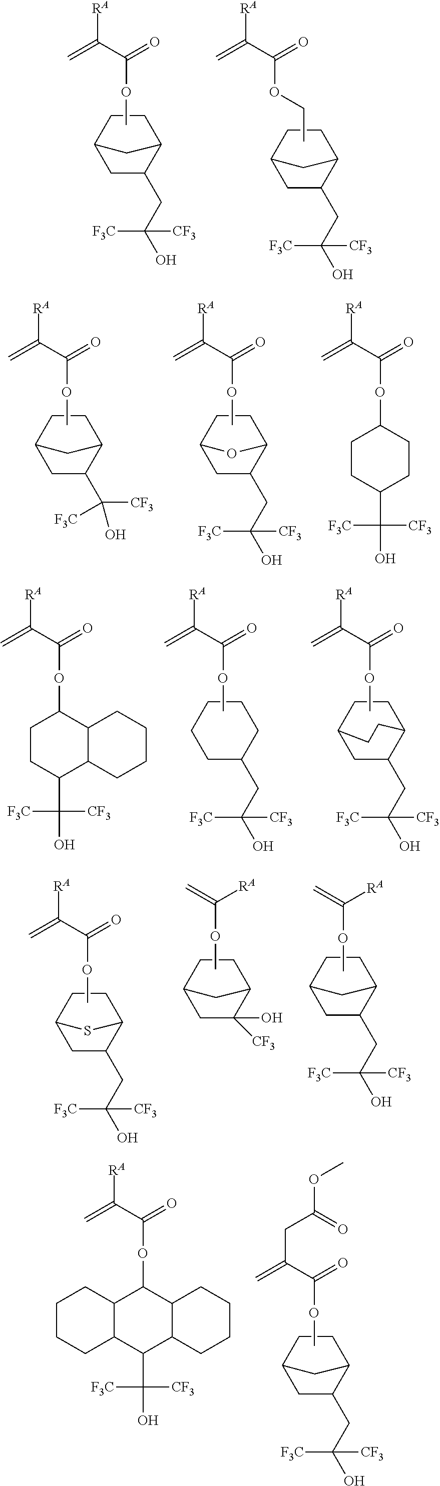

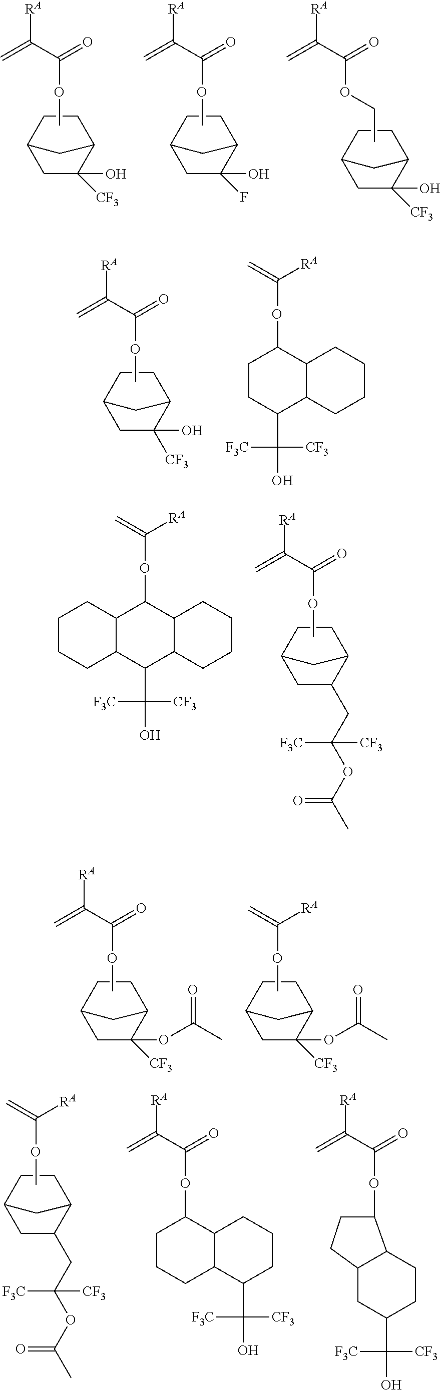

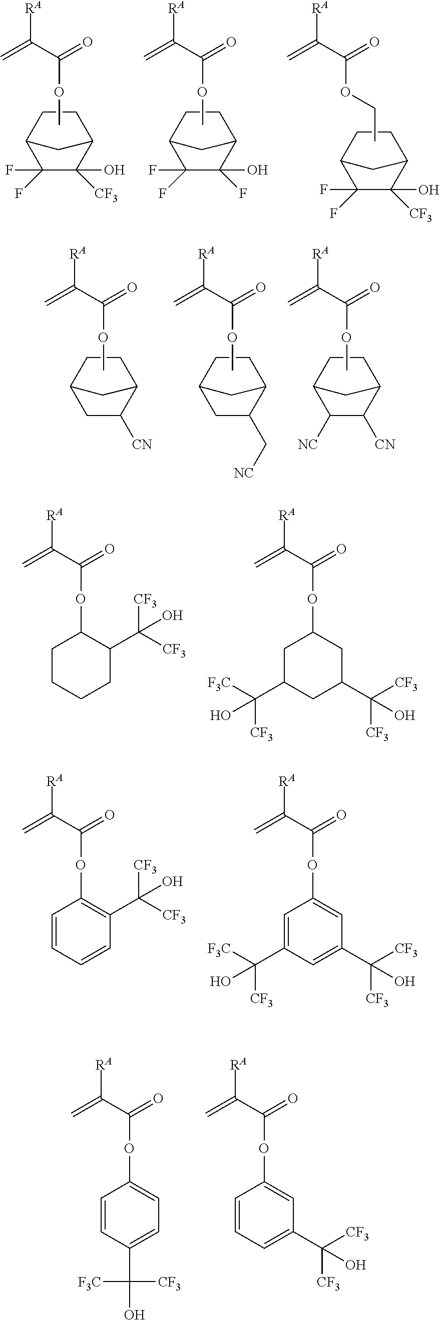

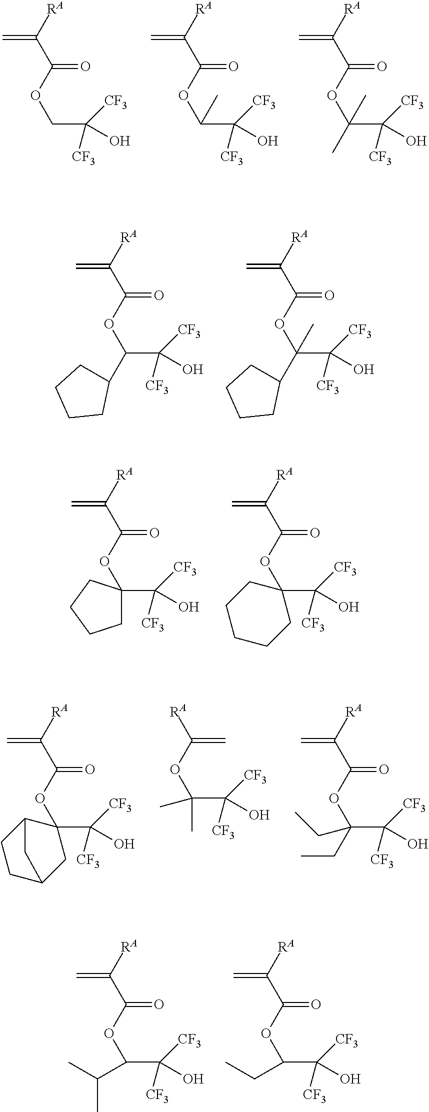

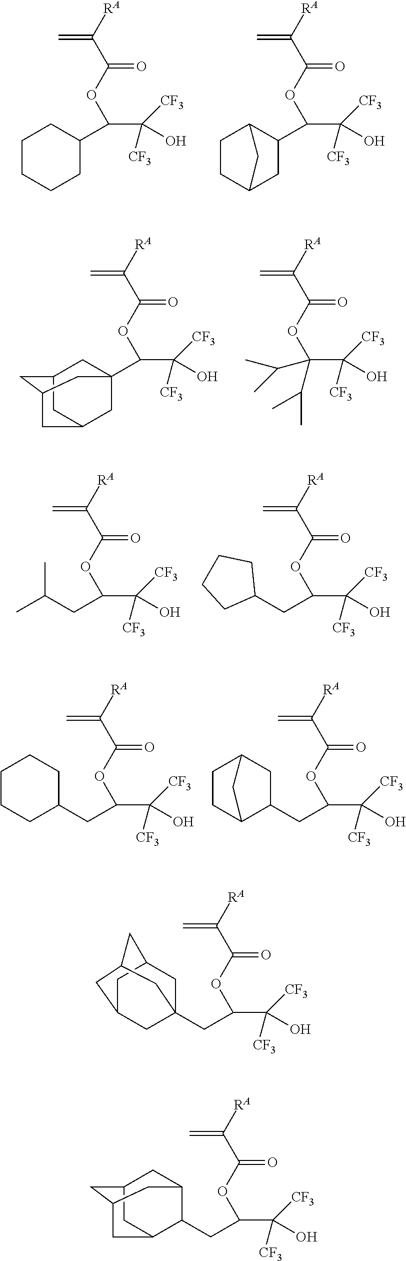

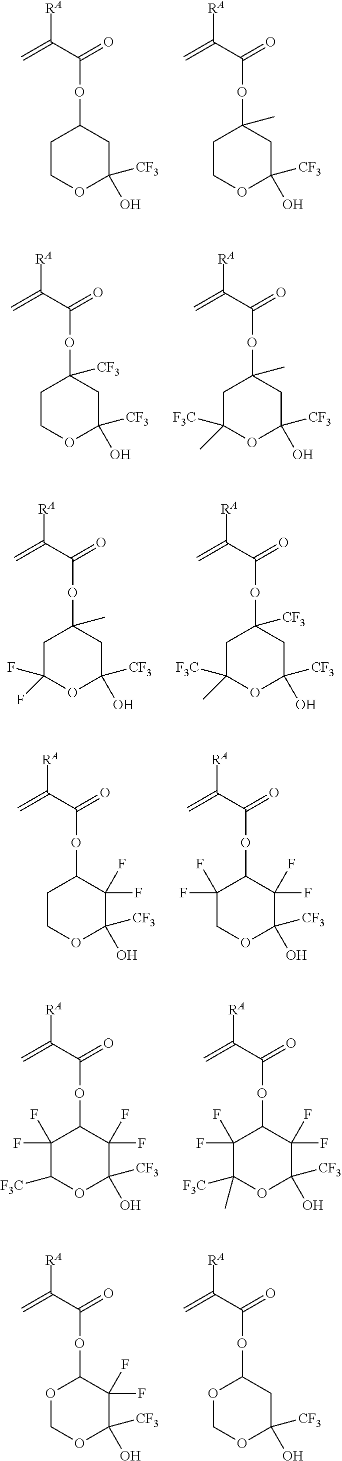

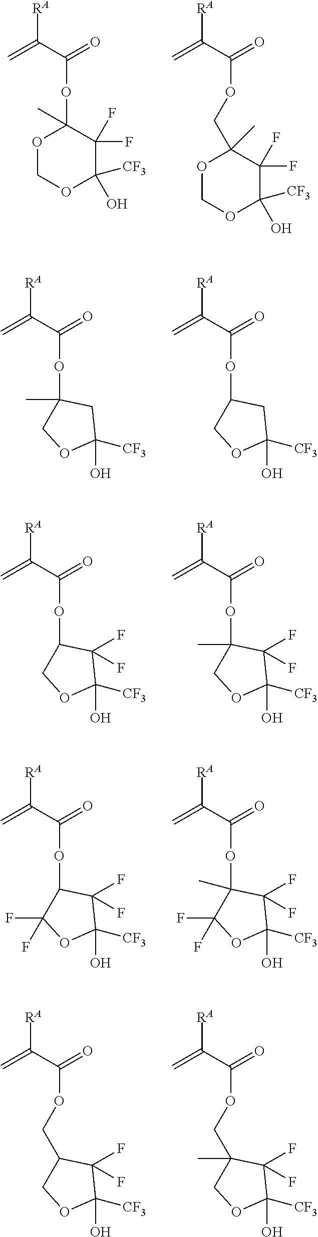

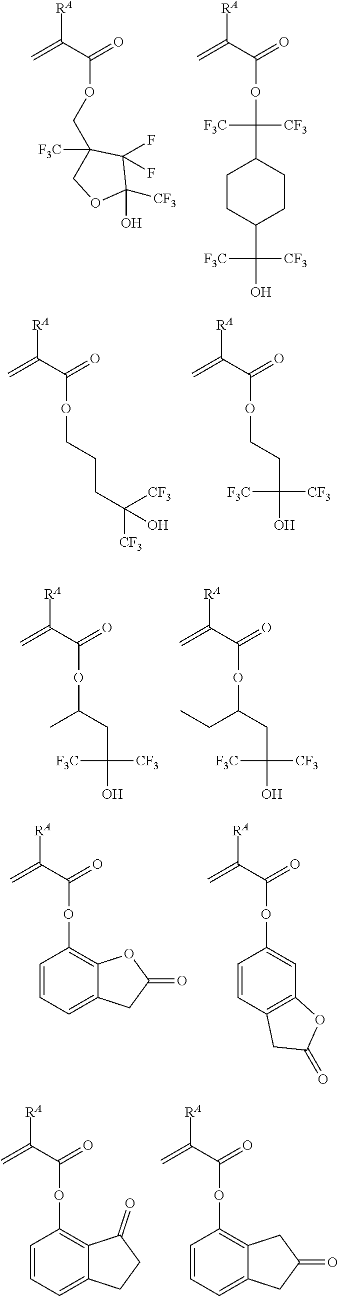

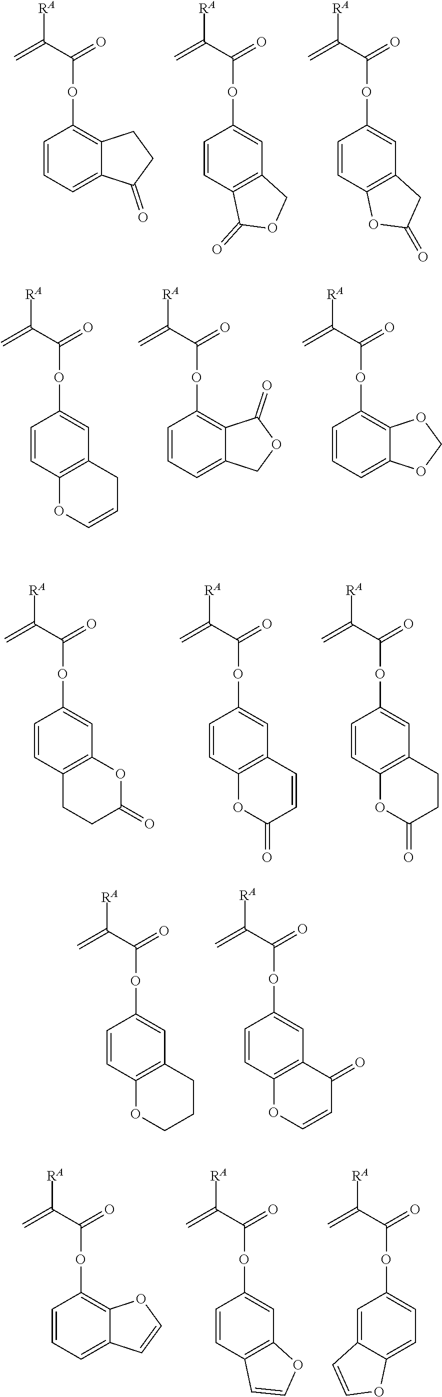

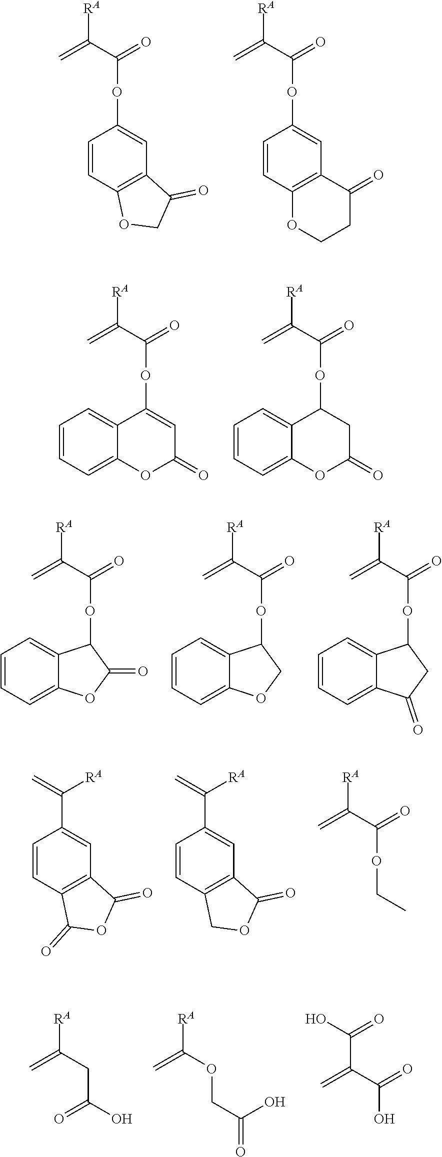

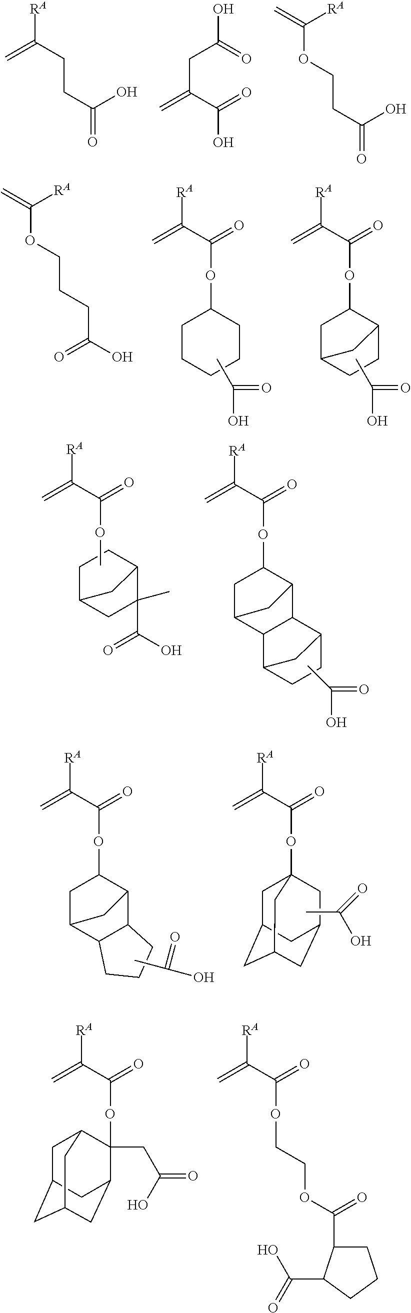

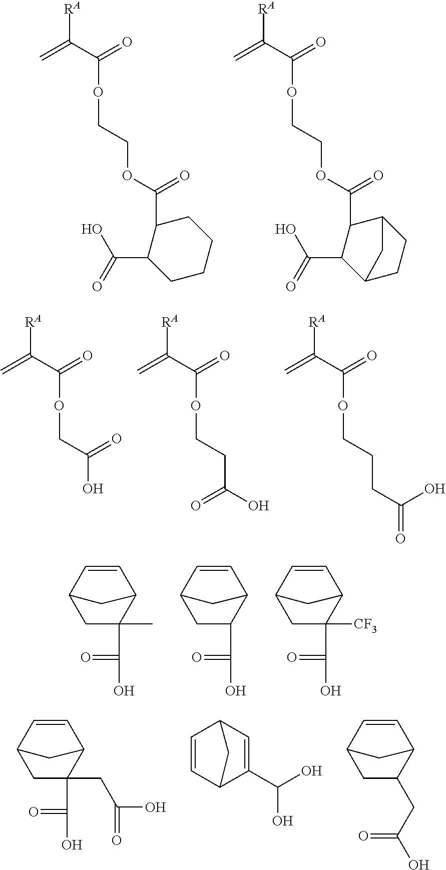

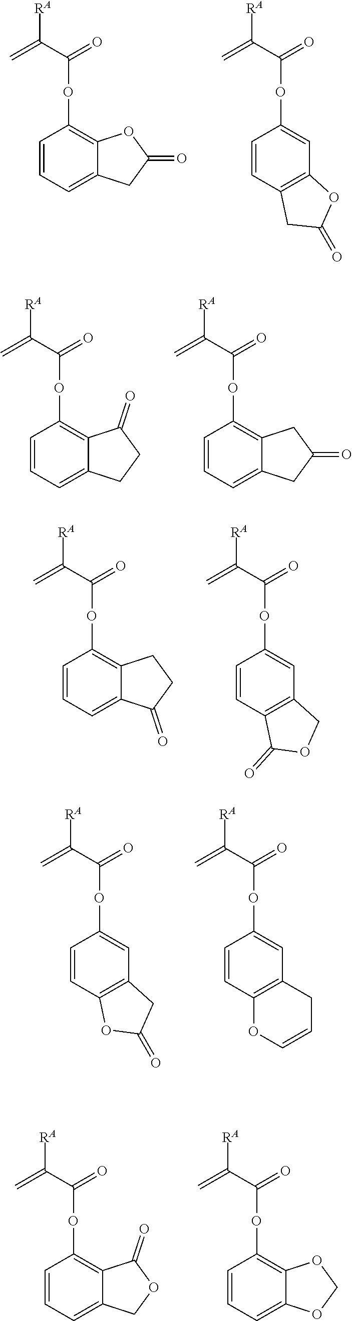

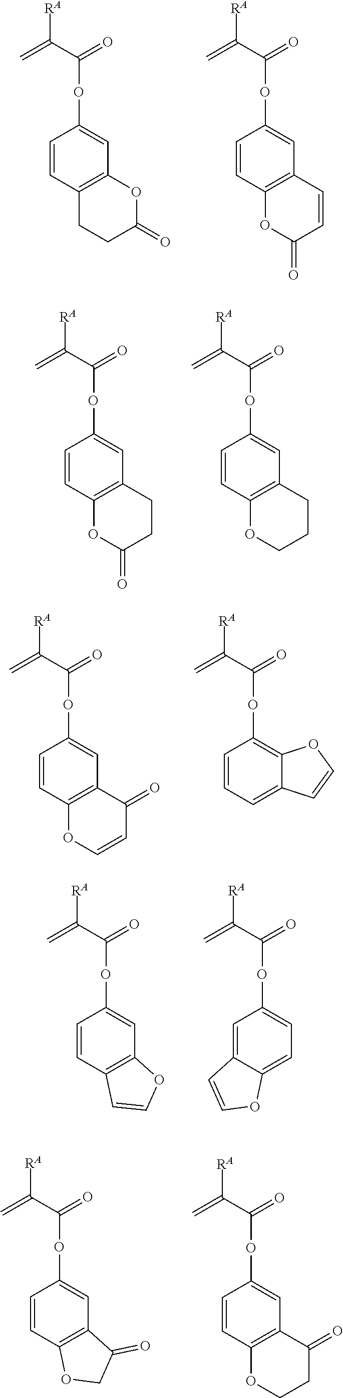

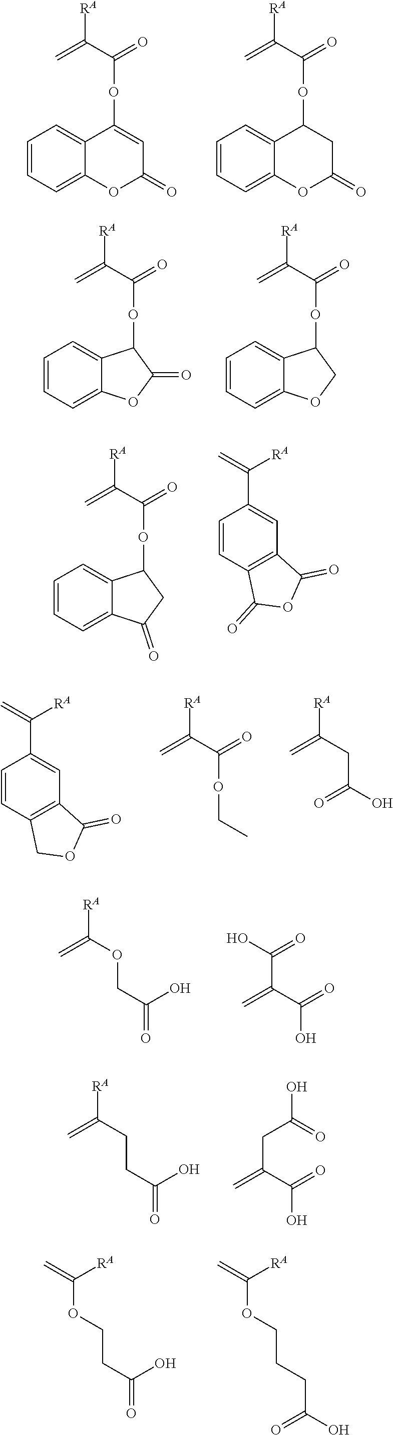

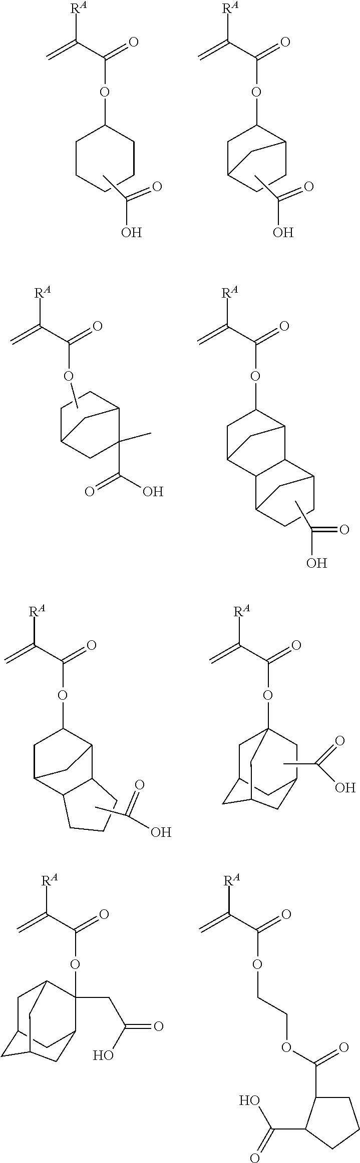

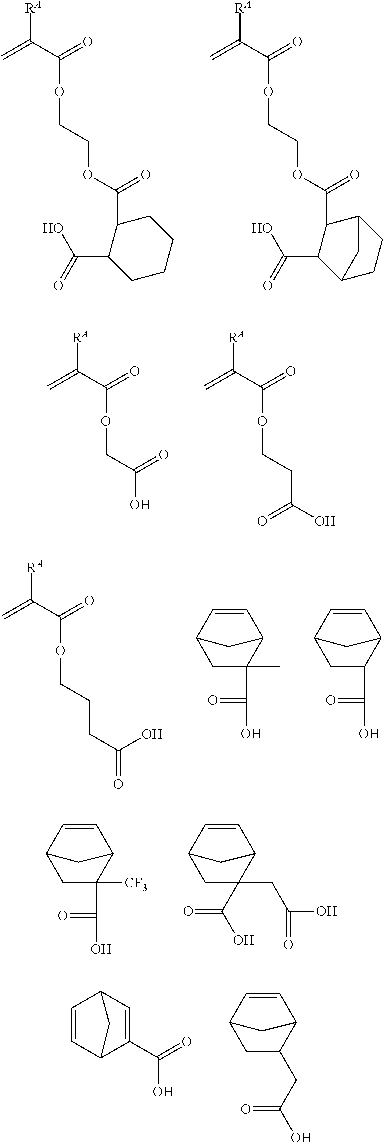

In another preferred embodiment, the base polymer may further comprise repeat units (d) derived from indene, benzofuran, benzothiophene, acenaphthylene, chromone, coumarin, and norbornadiene, or derivatives thereof. Suitable monomers are exemplified below.

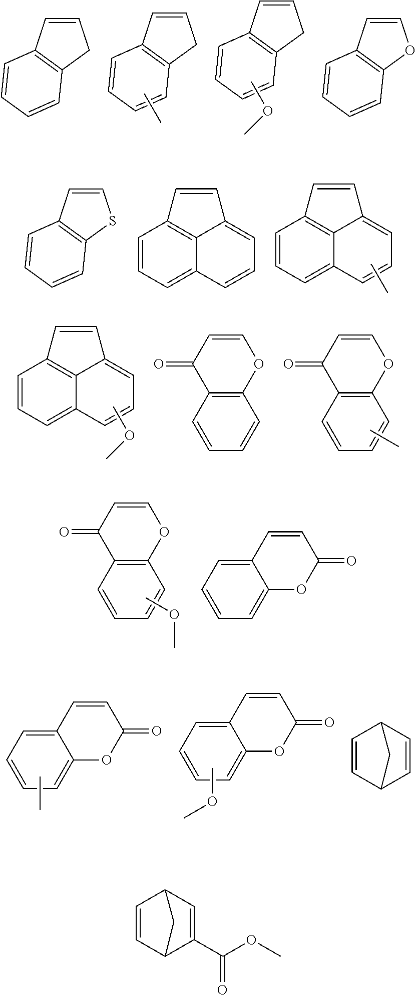

Furthermore, the base polymer may comprise repeat units (e) derived from styrene, vinylnaphthalene, vinylanthracene, vinylpyrene, methyleneindene, vinylpyridine, vinylcarbazole, or derivatives thereof.

In a further embodiment, the base polymer may comprise repeat units (f) derived from an onium salt having a polymerizable unsaturated bond. Specifically, the base polymer may comprise repeat units of at least one type selected from repeat units having formula (f1), repeat units having formula (f2), and repeat units having formula (f3). These units are simply referred to as repeat units (f1), (f2) and (f3), which may be used alone or in combination of two or more types.

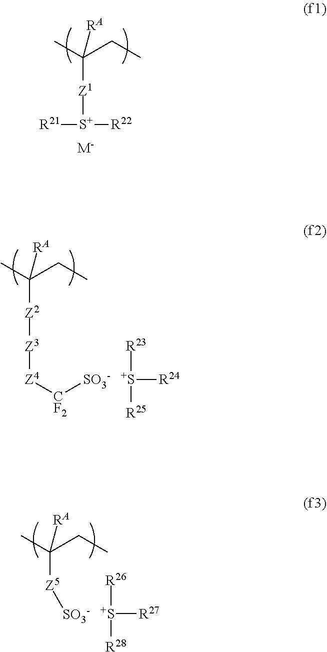

In formulae (f1) to (f3), R^{A} is each independently hydrogen or methyl. Z¹ is a single bond, C₁-C₆ aliphatic hydrocarbylene group, phenylene group, naphthylene group, or C₇-C₁₈ group obtained by combining the foregoing, —O—Z¹¹—, —C(═O)—O—Z¹¹—, or —C(═O)—NH—Z¹¹—. Z¹¹ is a C₁-C₆ aliphatic hydrocarbylene group, phenylene group, naphthylene group, or C₇-C₁₈ group obtained by combining the foregoing, which may contain a carbonyl moiety, ester bond, ether bond or hydroxy moiety. Z² is a single bond or ester bond. Z³ is a single bond, —Z³¹—C(═O)—O—, —Z³¹—O— or —Z³¹—O—C(═O)—. Z³¹ is a C₁-C₁₂ hydrocarbylene group, phenylene group, or C₇-C₁₈ group obtained by combining the foregoing, which may contain a carbonyl moiety, ester bond, ether bond, iodine or bromine. Z⁴ is methylene, 2,2,2-trifluoro-1,1-ethanediyl or carbonyl group. Z⁵ is a single bond, methylene, ethylene, phenylene, fluorinated phenylene, trifluoromethyl-substituted phenylene, —O—Z⁵¹—, —C(═O)—O—Z⁵¹—, or —C(═O)—NH—Z⁵¹—. Z⁵¹ is a C₁-C₆ aliphatic hydrocarbylene group, phenylene group, fluorinated phenylene group, or trifluoromethyl-substituted phenylene group, which may contain a carbonyl moiety, ester bond, ether bond, halogen or hydroxy moiety.

In formulae (f1) to (f3), R²¹ to R²⁸ are each independently halogen or a C₁-C₂₀ hydrocarbyl group which may contain a heteroatom. The hydrocarbyl group may be saturated or unsaturated and straight, branched or cyclic. Examples thereof are as exemplified above for R⁴ to R⁶ in formula (1). In these hydrocarbyl groups, some or all of the hydrogen atoms may be substituted by a moiety containing a heteroatom such as oxygen, sulfur, nitrogen or halogen and some constituent —CH₂— may be replaced by a moiety containing a heteroatom such as oxygen, sulfur or nitrogen, so that the group may contain a hydroxy moiety, fluorine, chlorine, bromine, iodine, cyano moiety, nitro moiety, carbonyl moiety, ether bond, ester bond, sulfonate ester bond, carbonate bond, lactone ring, sultone ring, carboxylic anhydride (—C(═O)—O—C(═O)—), or haloalkyl moiety. A pair of R²³ and R²⁴, or R²⁶ and R²⁷ may bond together to form a ring with the sulfur atom to which they are attached. Examples of the ring are as exemplified above for the ring that R⁴ and R⁵ in formula (1), taken together, form with the sulfur atom to which they are attached.

In formula (f1), M⁻ is a non-nucleophilic counter ion. Examples of the non-nucleophilic counter ion include halide ions such as chloride and bromide ions; fluoroalkylsulfonate ions such as triflate, 1,1,1-trifluoroethanesulfonate, and nonafluorobutanesulfonate; arylsulfonate ions such as tosylate, benzenesulfonate, 4-fluorobenzenesulfonate, and 1,2,3,4,5-pentafluorobenzenesulfonate; alkylsulfonate ions such as mesylate and butanesulfonate; imide ions such as bis(trifluoromethylsulfonyl)imide, bis(perfluoroethylsulfonyl)imide and bis(perfluorobutylsulfonyl)imide; methide ions such as tris(trifluoromethylsulfonyl)methide and tris(perfluoroethylsulfonyl)methide.

Also included are sulfonate ions having fluorine substituted at α-position as represented by the formula (f1-1) and sulfonate ions having fluorine substituted at α-position and trifluoromethyl at β-position as represented by the formula (f1-2).

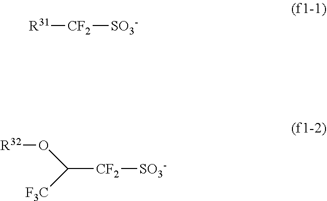

In formula (f1-1), R³¹ is hydrogen, or a C₁-C₂₀ hydrocarbyl group which may contain an ether bond, ester bond, carbonyl moiety, lactone ring, or fluorine atom. The hydrocarbyl group may be saturated or unsaturated and straight, branched or cyclic. Examples of the hydrocarbyl group are as will be exemplified later for R^{fa1} in formula (2A′).

In formula (f1-2), R³² is hydrogen, or a C₁-C₃₀ hydrocarbyl group or C₂-C₃₀ hydrocarbylcarbonyl group, which may contain an ether bond, ester bond, carbonyl moiety or lactone ring. The hydrocarbyl group and hydrocarbyl moiety in the hydrocarbylcarbonyl group may be saturated or unsaturated and straight, branched or cyclic. Examples of the hydrocarbyl group are as will be exemplified later for R^{fa1} in formula (2A′).

Examples of the cation in the monomer from which repeat unit (f1) is derived are shown below, but not limited thereto. R^{A} is as defined above.

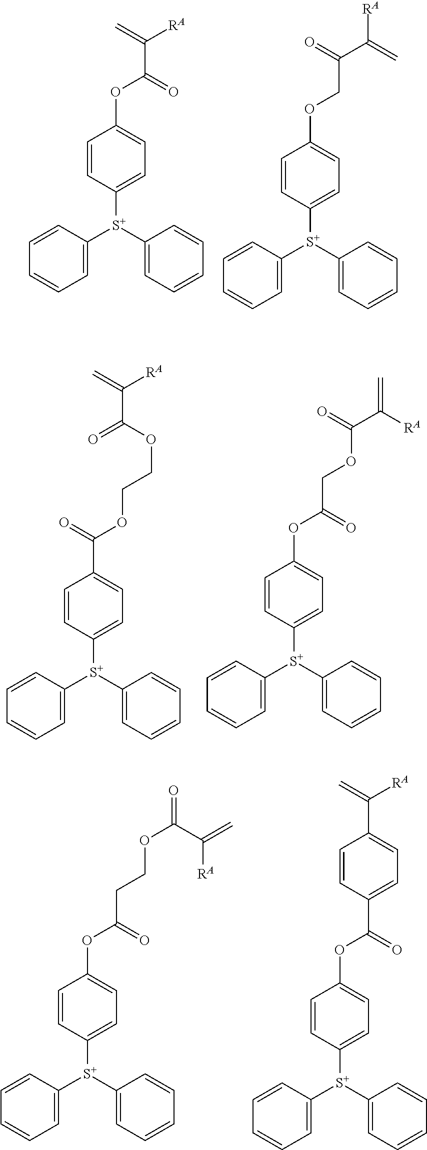

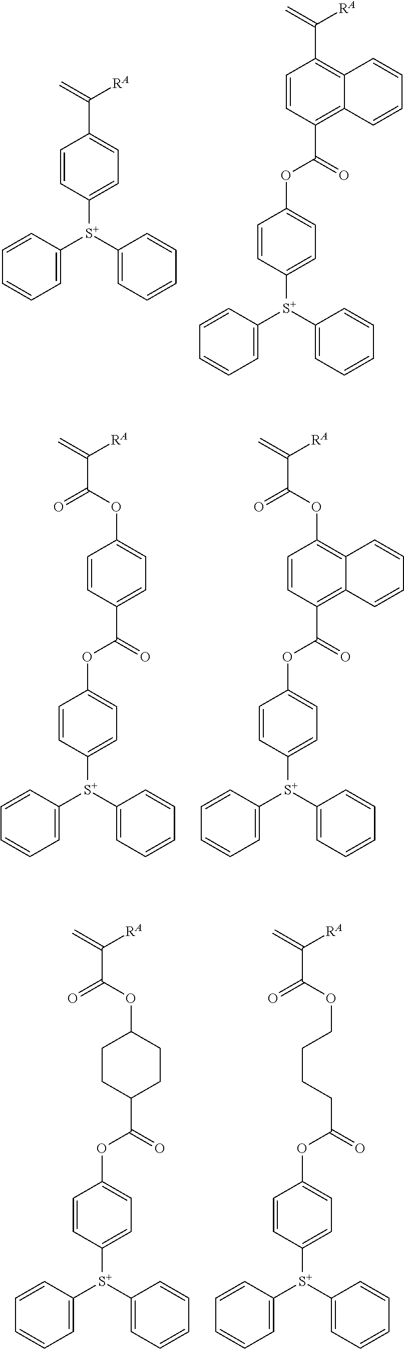

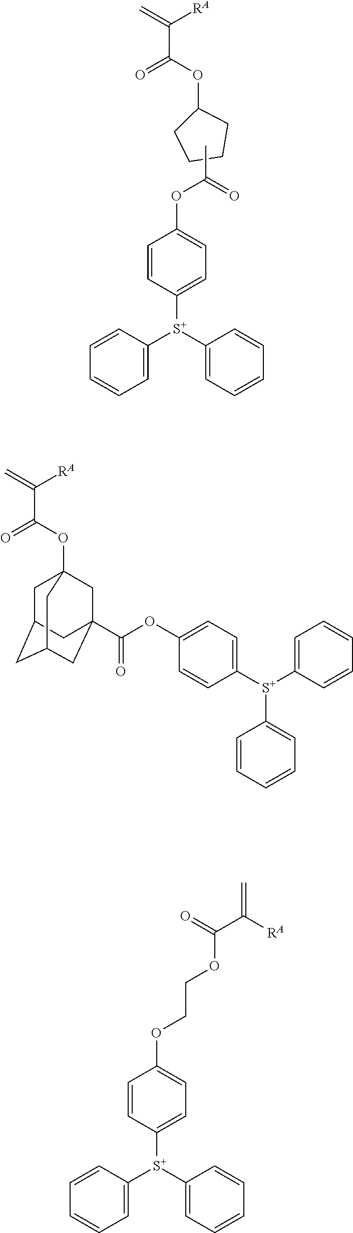

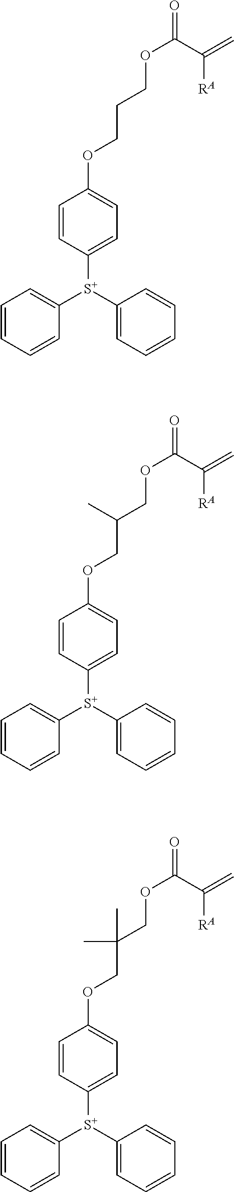

Examples of the cation in the monomer from which repeat unit (f2) or (f3) is derived are as exemplified above for the cation in Sulfonium Salt A.

Examples of the anion in the monomer from which repeat unit (f2) is derived are shown below, but not limited thereto. R^{A} is as defined above.

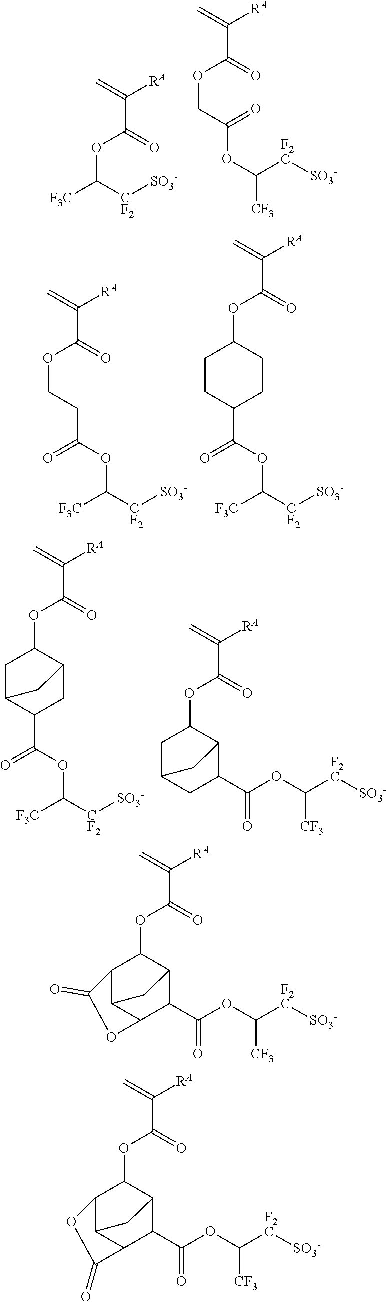

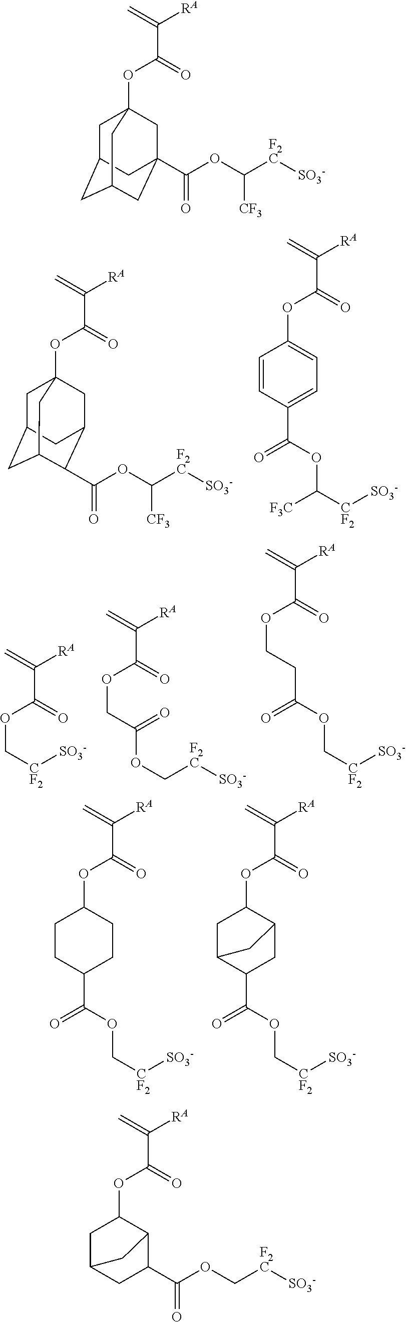

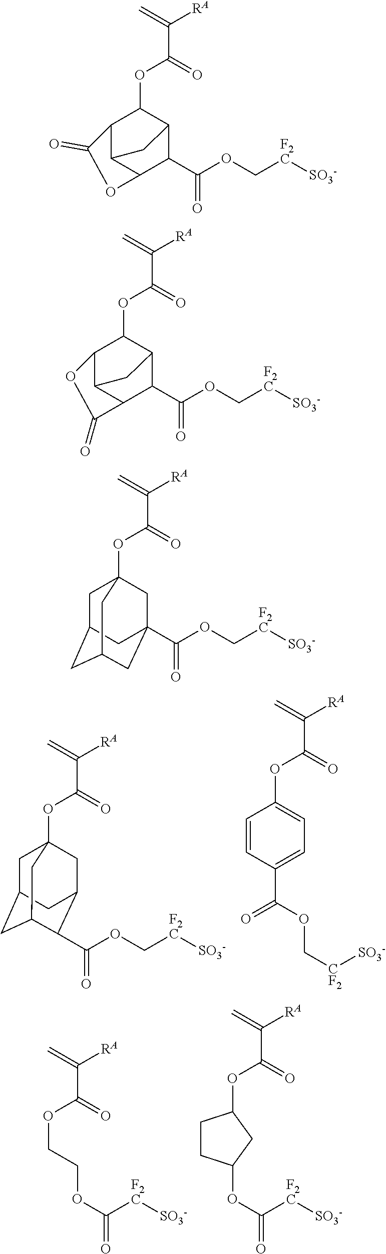

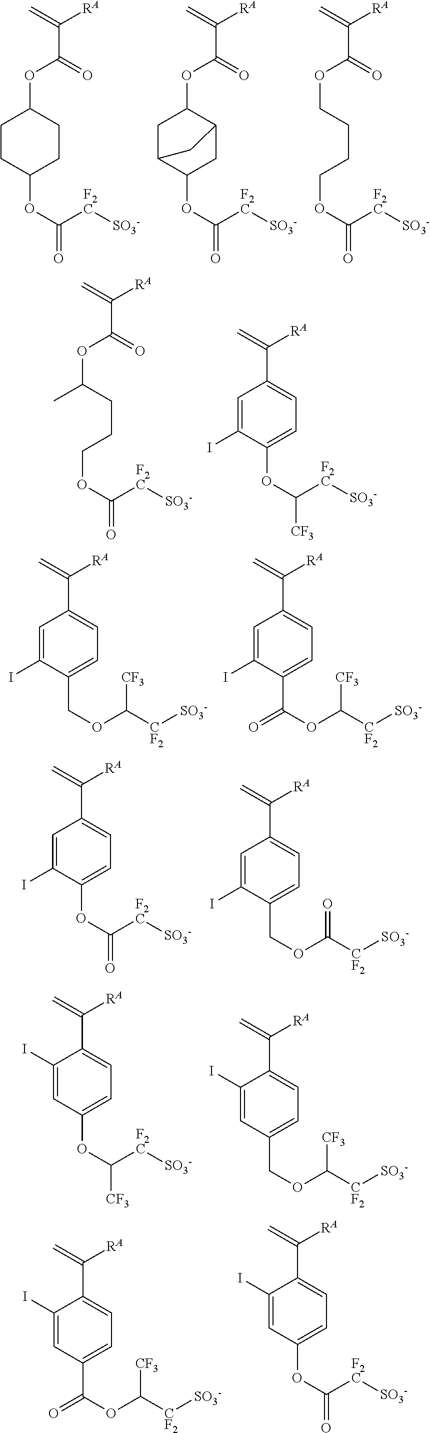

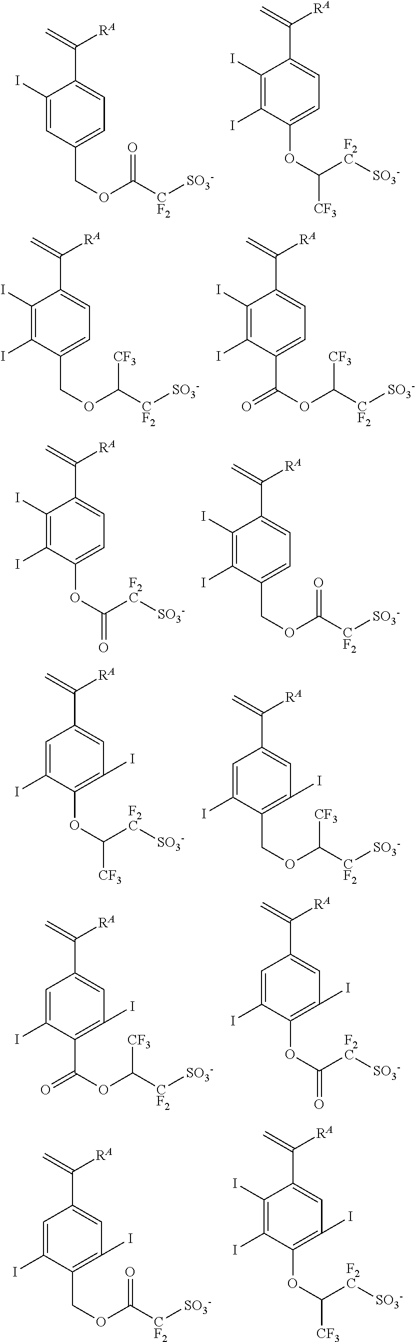

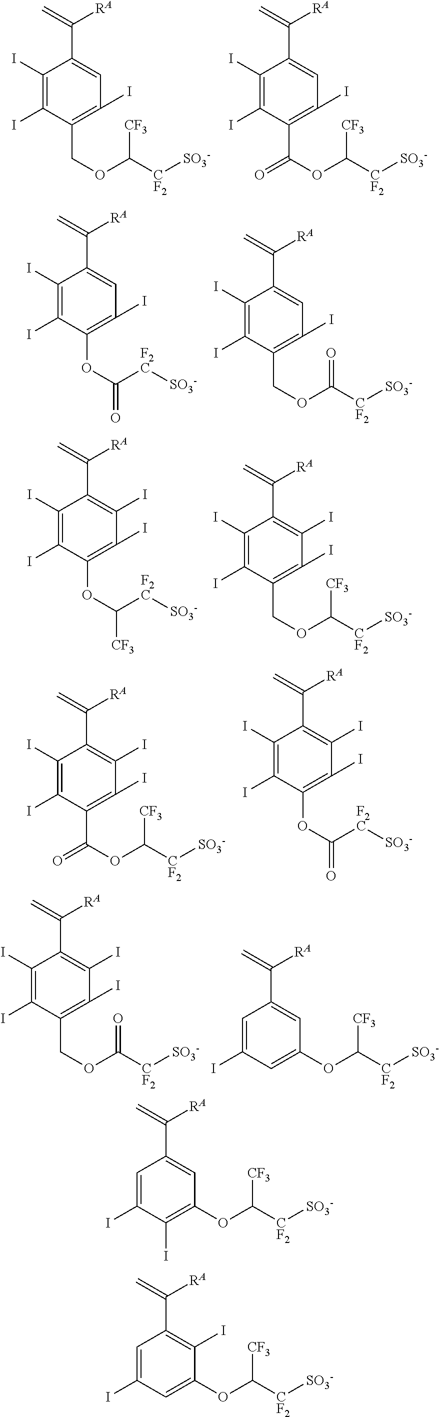

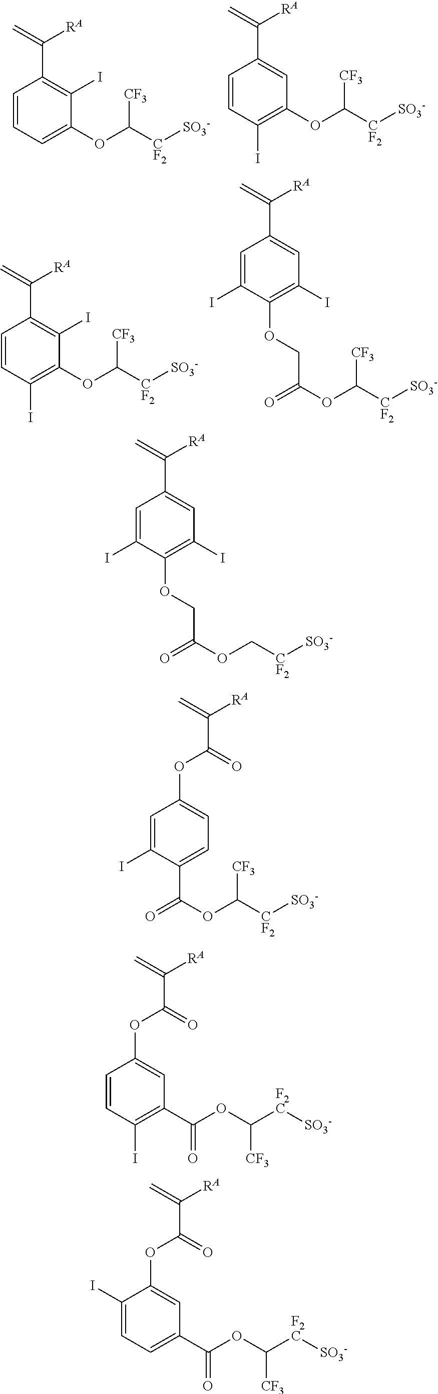

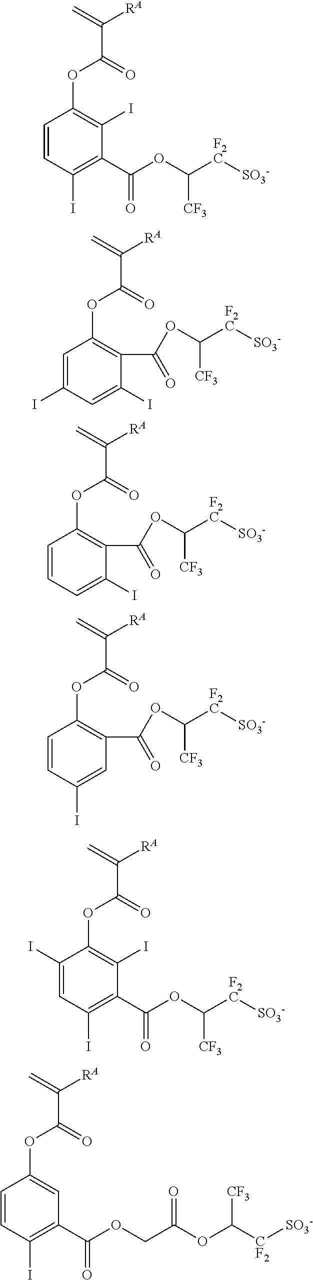

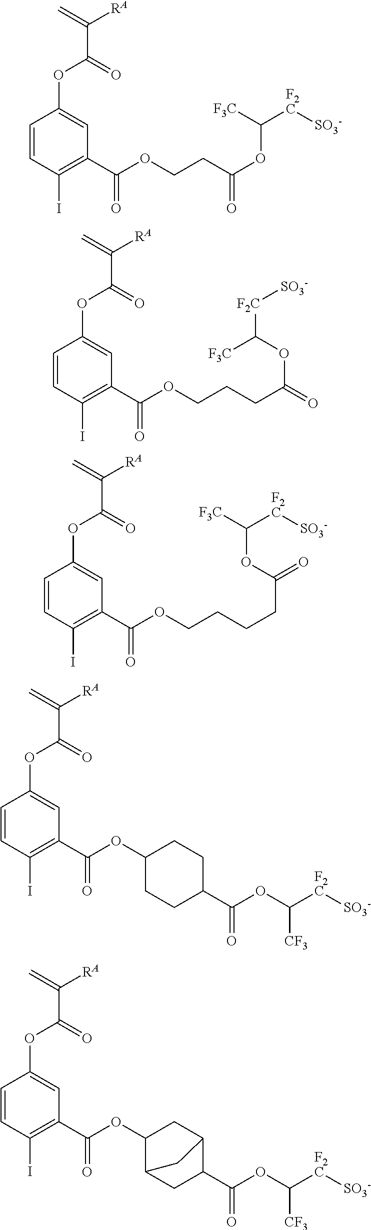

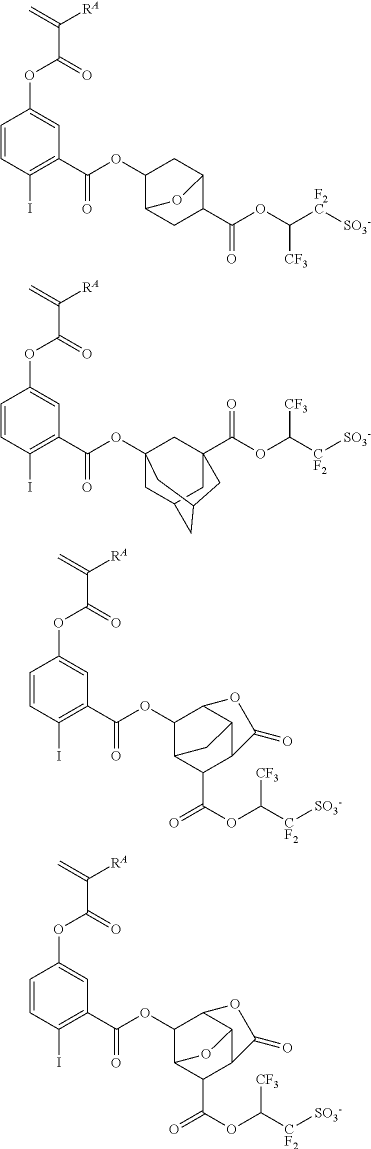

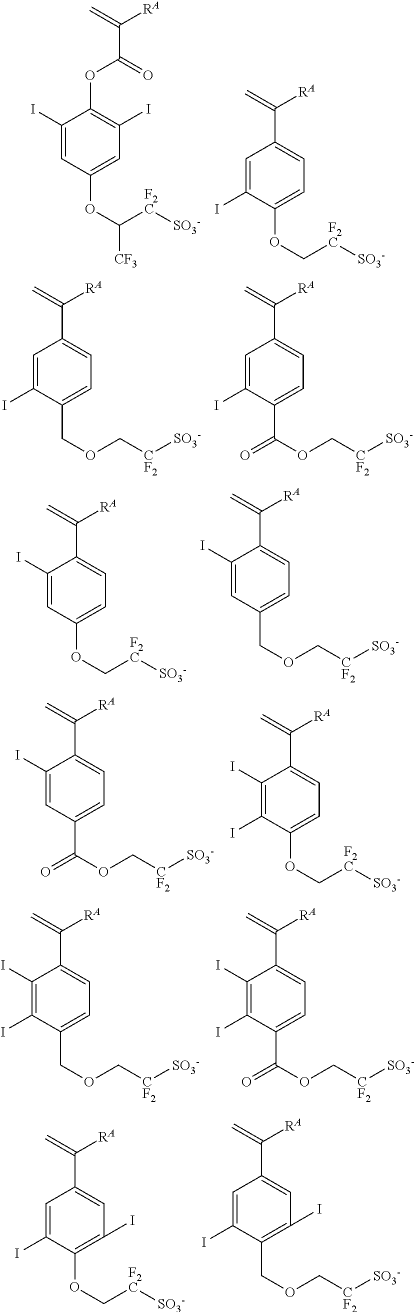

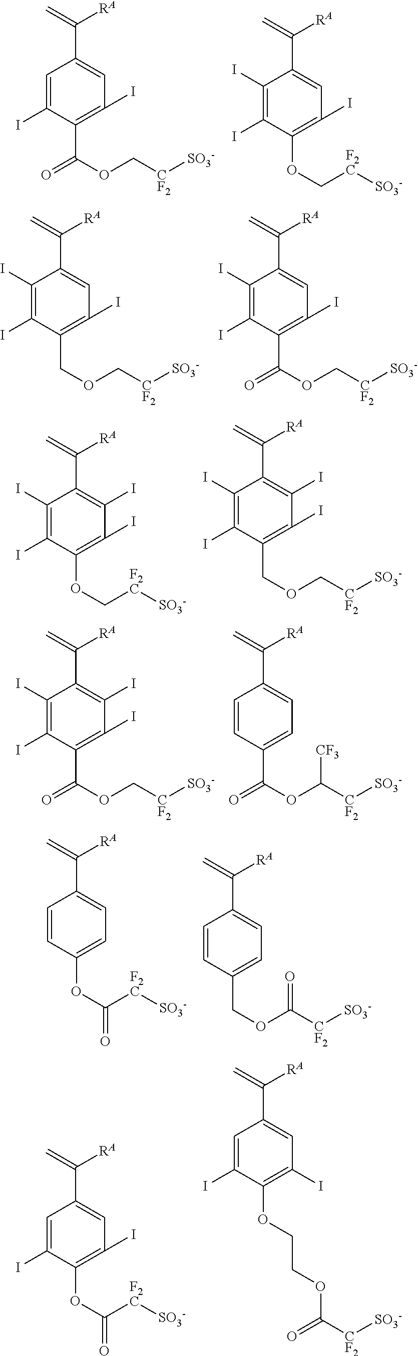

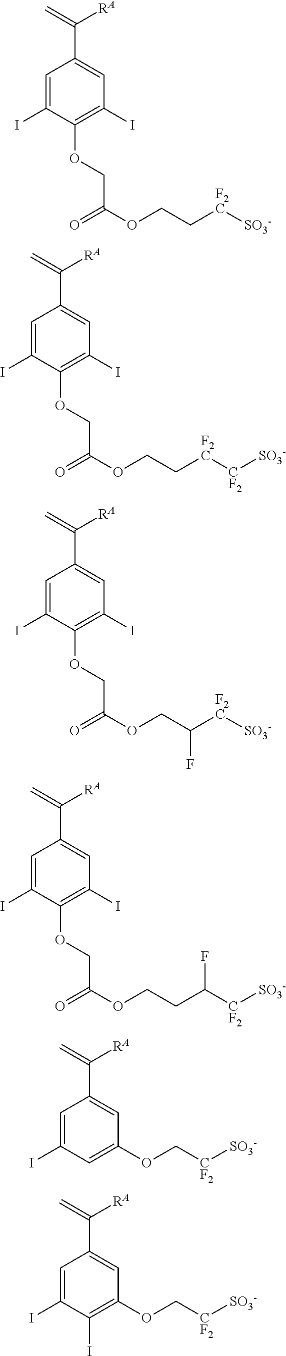

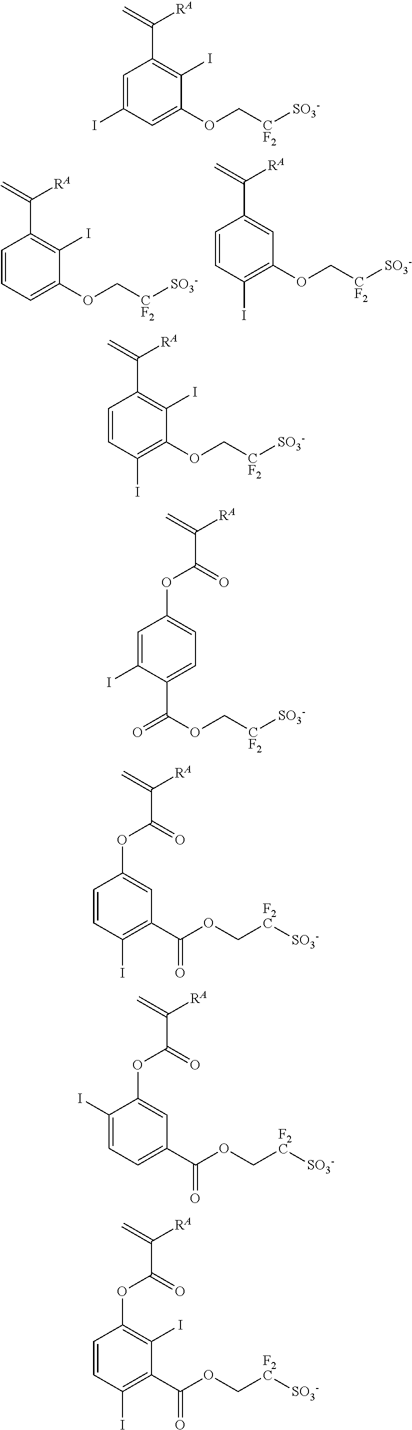

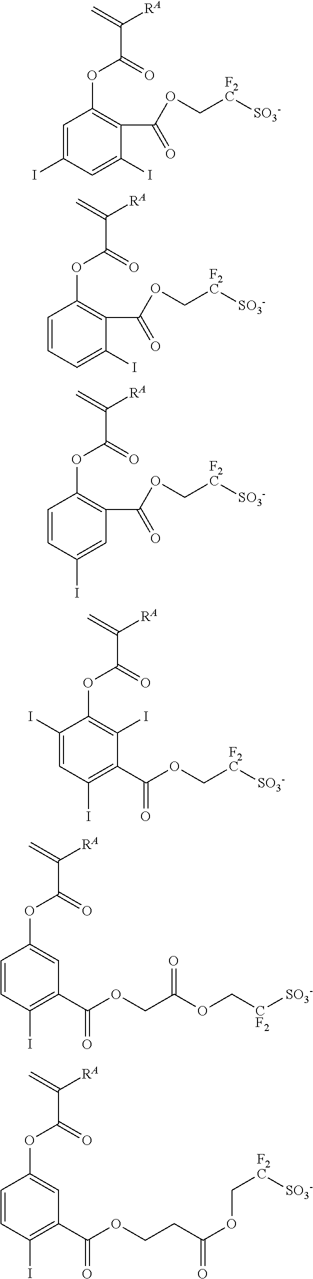

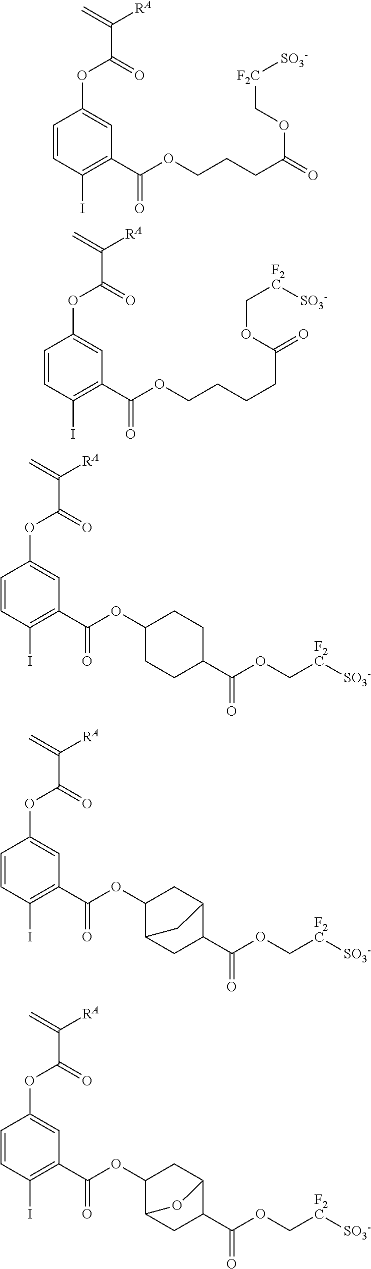

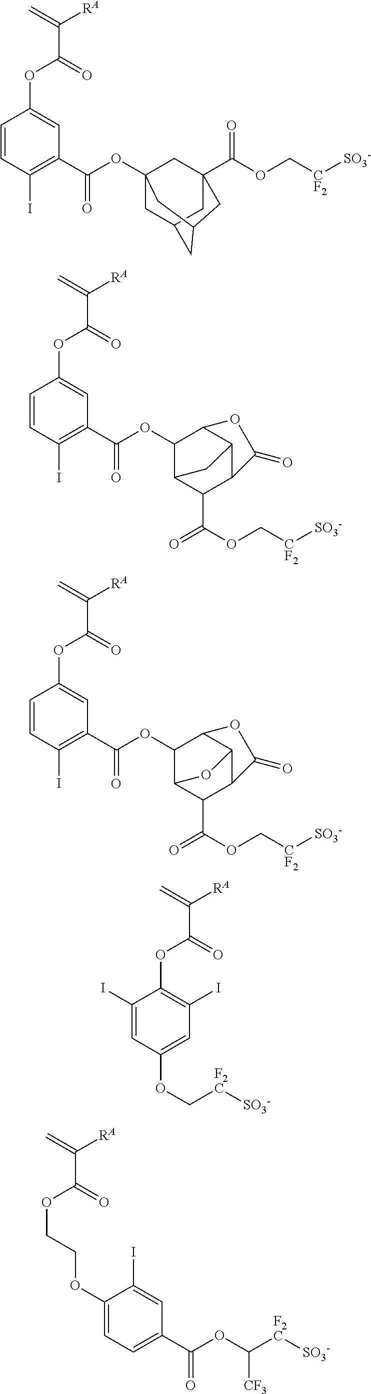

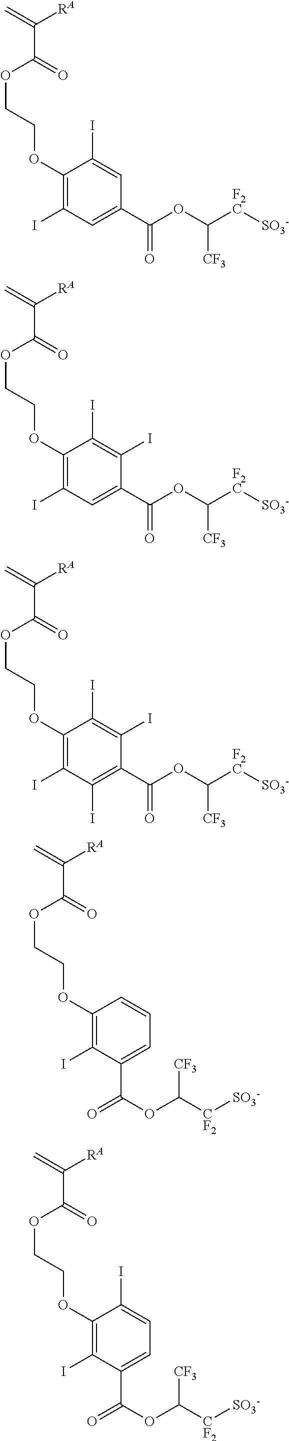

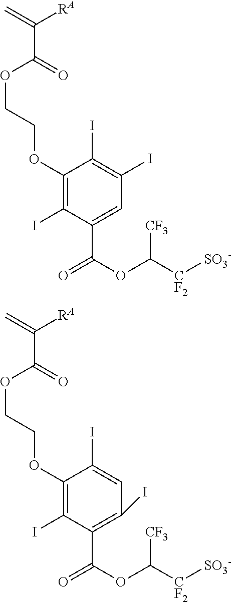

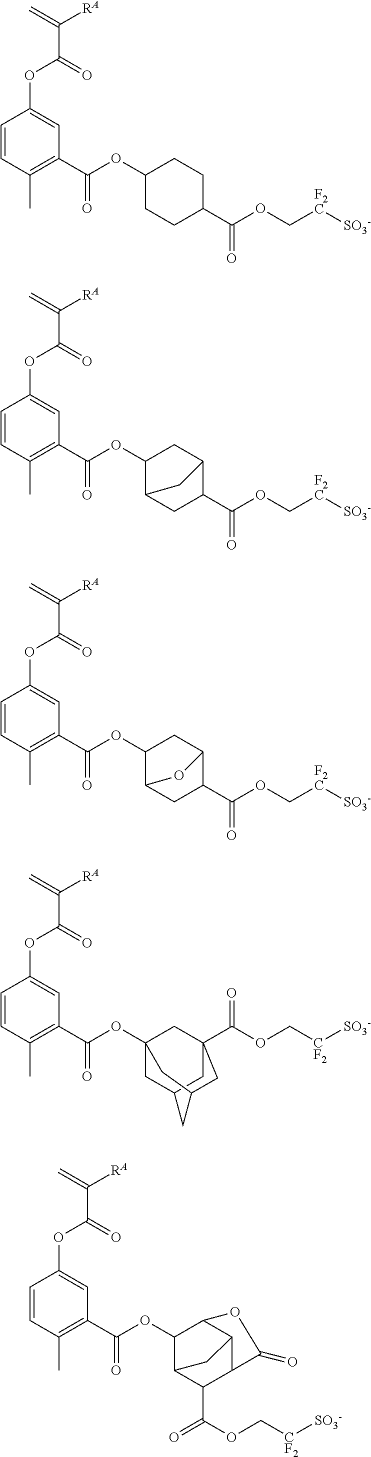

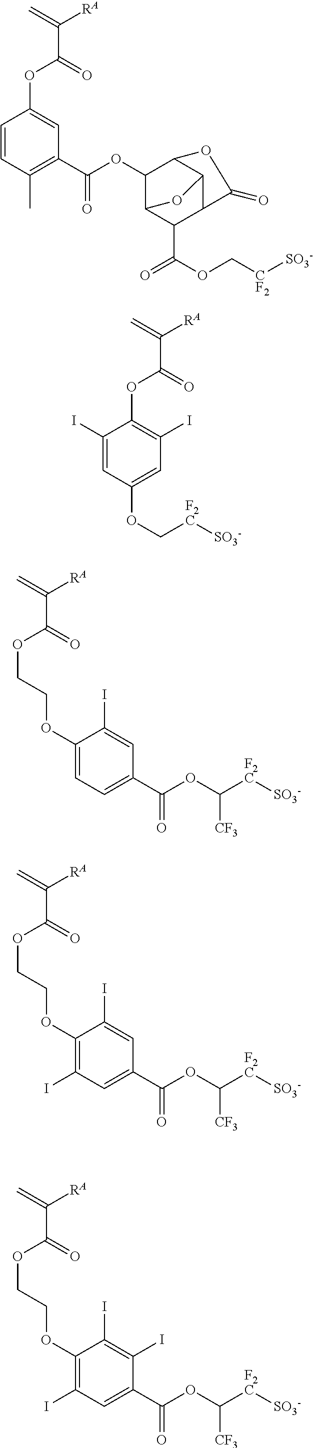

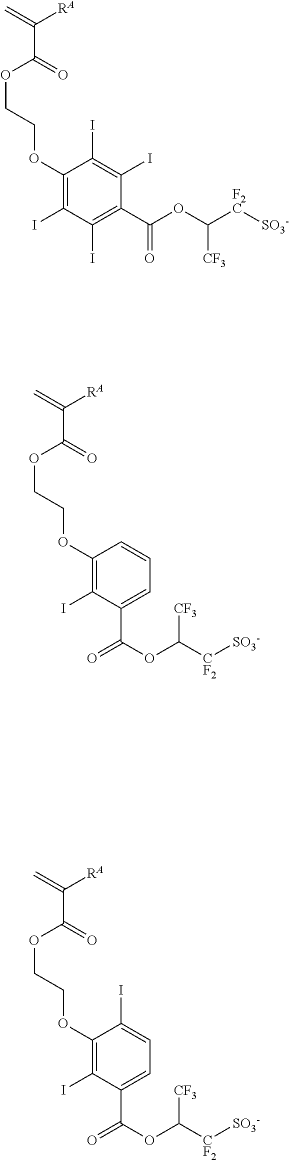

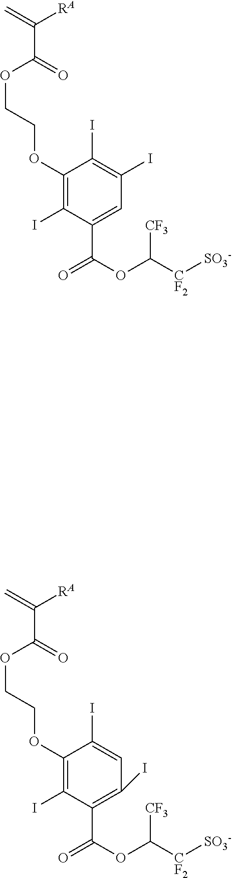

Examples of the anion in the monomer from which repeat unit (0) is derived are shown below, but not limited thereto. R^{A} is as defined above.

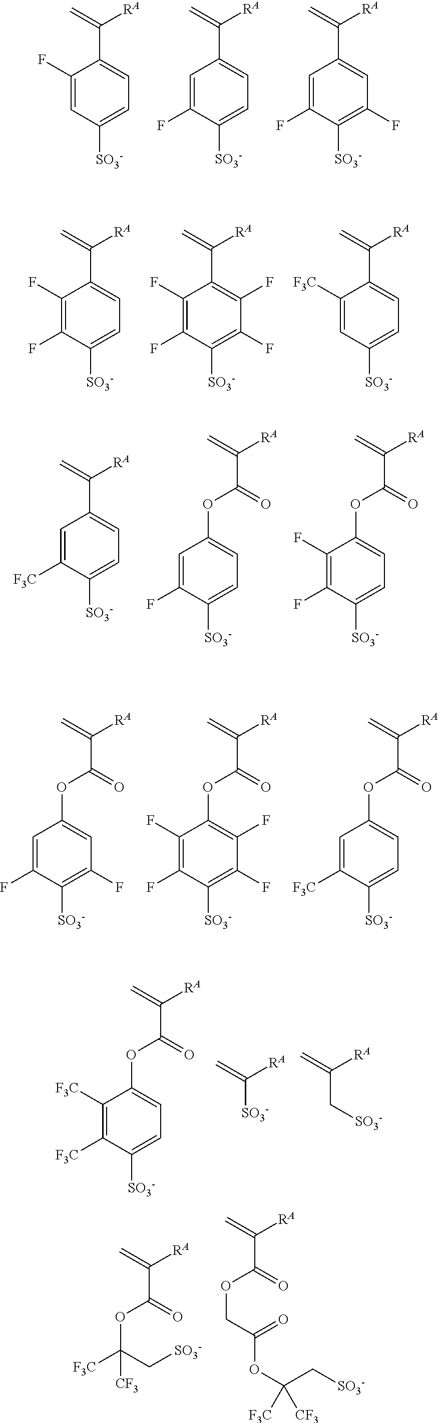

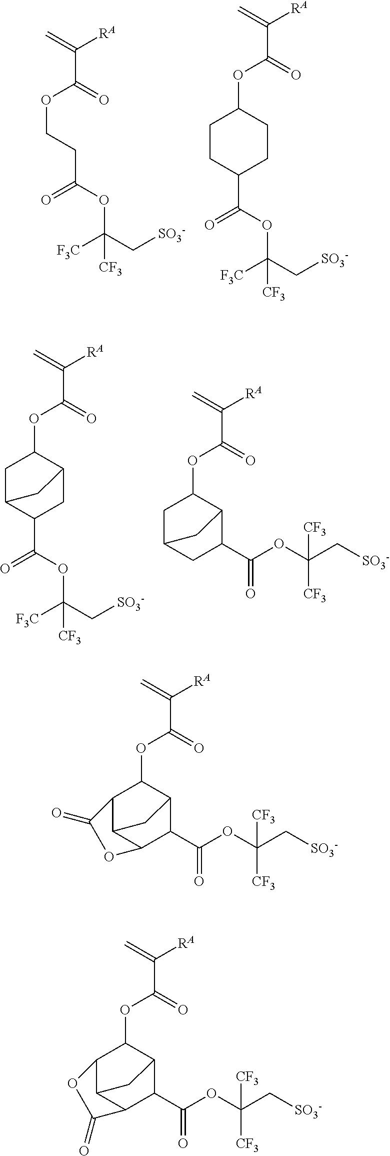

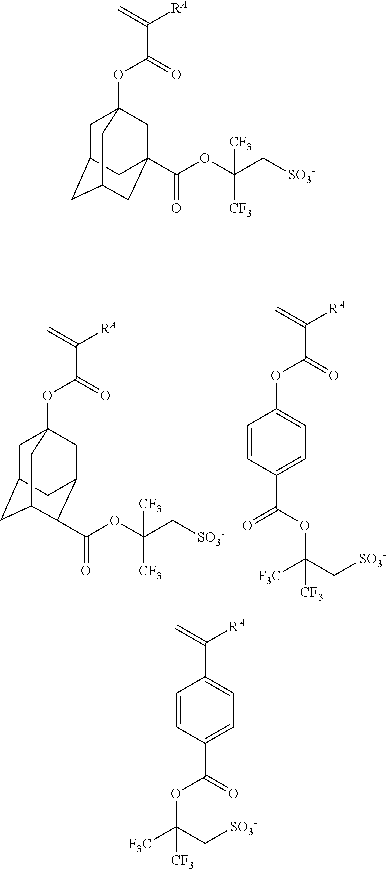

Repeat units (f1) to (3) have an acid generator function. The attachment of an acid generator to the polymer main chain is effective in restraining acid diffusion, thereby preventing a reduction of resolution due to blur by acid diffusion. Also, LWR or CDU is improved because the acid generator is uniformly distributed. Where a base polymer containing repeat units (f), i.e., polymer-bound acid generator is used, the blending of an acid generator of addition type (to be described later) may be omitted.

In the base polymer, a fraction of units (a1), (a2), (b), (c), (d), (e), (f1), (f2) and (f3) is: preferably 0≤a1≤0.9, 0≤a2≤0.9, 0≤a1+a2≤0.9, 0≤b≤0.9, 0≤c≤0.9, 0≤d≤0.5, 0≤e≤0.5, 0≤f1≤0.5, 0≤f2≤0.5, 0≤f3≤0.5, and 0≤f1+f2+f3≤0.5; more preferably 0≤a1≤0.8, 0≤a2≤0.8, 0≤a1+a2≤0.8, 0≤b≤0.8, 0≤c≤0.8, 0≤d≤0.4, 0≤e≤0.4, 0≤f1≤0.4, 0≤f2≤0.4, 0≤f3≤0.4, and 0≤f1+f2+f3≤0.4; and even more preferably 0≤a1≤0.7, 0≤a2≤0.7, 0≤a1+a2≤0.7, 0≤b≤0.7, 0≤c≤0.7, 0≤d≤0.3, 0≤e≤0.3, 0≤f1≤0.3, 0≤f2≤0.3, 0≤f3≤0.3, and 0≤f1+f2+f3≤0.3. Notably, a1+a2+b+c+d+e+f1+f2+f3=1.0.

The base polymer may be synthesized by any desired methods, for example, by dissolving one or more monomers selected from the monomers corresponding to the foregoing repeat units in an organic solvent, adding a radical polymerization initiator thereto, and heating for polymerization. Examples of the organic solvent which can be used for polymerization include toluene, benzene, tetrahydrofuran (THF), diethyl ether, and dioxane. Examples of the polymerization initiator used herein include 2,2′-azobisisobutyronitrile (AIBN), 2,2′-azobis(2,4-dimethylvaleronitrile), dimethyl 2,2-azobis(2-methylpropionate), benzoyl peroxide, and lauroyl peroxide. Preferably, the reaction temperature is 50 to 80° C. and the reaction time is 2 to 100 hours, more preferably 5 to 20 hours.

Where a monomer having a hydroxy group is copolymerized, the hydroxy group may be replaced by an acetal group susceptible to deprotection with acid, typically ethoxyethoxy, prior to polymerization, and the polymerization be followed by deprotection with weak acid and water. Alternatively, the hydroxy group may be replaced by an acetyl, formyl, pivaloyl or similar group prior to polymerization, and the polymerization be followed by alkaline hydrolysis.

When hydroxystyrene or hydroxyvinylnaphthalene is copolymerized, an alternative method is possible. Specifically, acetoxystyrene or acetoxyvinylnaphthalene is used instead of hydroxystyrene or hydroxyvinylnaphthalene, and after polymerization, the acetoxy group is deprotected by alkaline hydrolysis, for thereby converting the polymer product to hydroxystyrene or hydroxyvinylnaphthalene. For alkaline hydrolysis, a base such as aqueous ammonia or triethylamine may be used. Preferably the reaction temperature is −20° C. to 100° C., more preferably 0° C. to 60° C., and the reaction time is 0.2 to 100 hours, more preferably 0.5 to 20 hours.

The base polymer should preferably have a weight average molecular weight (Mw) in the range of 1,000 to 500,000, and more preferably 2,000 to 30,000, as measured by GPC versus polystyrene standards using tetrahydrofuran (THF) solvent. A Mw in the range ensures that the resist film is fully heat resistant and dissolvable in alkaline developer.

If a base polymer has a wide molecular weight distribution or dispersity (Mw/Mn), which indicates the presence of lower and higher molecular weight polymer fractions, there is a possibility that foreign matter is left on the pattern or the pattern profile is degraded. The influences of Mw and Mw/Mn become stronger as the pattern rule becomes finer. Therefore, the base polymer should preferably have a narrow dispersity (Mw/Mn) of 1.0 to 2.0, especially 1.0 to 1.5, in order to provide a resist composition suitable for micropatterning to a small feature size.

For forming a narrow dispersity polymer, not only ordinary radical polymerization, but living radical polymerization may also be employed. Suitable living radical polymerization processes include radical polymerization using nitroxide radicals, i.e., nitroxide-mediated radical polymerization (NMP), atom transfer radical polymerization (ATRP), and reversible addition-fragmentation chain transfer (RAFT) polymerization.

It is understood that a blend of two or more polymers which differ in compositional ratio, Mw or Mw/Mn is acceptable.

### [Acid Generator]

The resist composition may comprise an acid generator capable of generating a strong acid (referred to as acid generator of addition type, hereinafter). As used herein, the term “strong acid” refers to a compound having a sufficient acidity to induce deprotection reaction of an acid labile group on the base polymer in the case of a chemically amplified positive resist composition, or a compound having a sufficient acidity to induce acid-catalyzed polarity switch reaction or crosslinking reaction in the case of a chemically amplified negative resist composition. The inclusion of such an acid generator ensures that Sulfonium Salt A functions as a quencher and the inventive resist composition functions as a chemically amplified positive or negative resist composition.

The acid generator is typically a compound (PAG) capable of generating an acid upon exposure to actinic ray or radiation. Although the PAG used herein may be any compound capable of generating an acid upon exposure to high-energy radiation, those compounds capable of generating sulfonic acid, imide acid (imidic acid) or methide acid are preferred. Suitable PAGs include sulfonium salts, iodonium salts, sulfonyldiazomethane, N-sulfonyloxyimide, and oxime-O-sulfonate acid generators. Exemplary PAGs are described in JP-A 2008-111103, paragraphs [0122]-[0142] (U.S. Pat. No. 7,537,880).

As the PAG used herein, sulfonium salts having the formula (2-1) and iodonium salts having the formula (2-2) are also preferred.

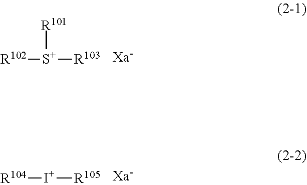

In formulae (2-1) and (2-2), R¹⁰¹ to R¹⁰⁵ are each independently halogen or a C₁-C₂₀ hydrocarbyl group which may contain a heteroatom. The hydrocarbyl group may be saturated or unsaturated and straight, branched or cyclic. Examples thereof are as exemplified above for the hydrocarbyl groups R⁴ to R⁶ in formula (1). A pair of R¹⁰¹ and R¹⁰² may bond together to form a ring with the sulfur atom to which they are attached. Examples of the ring are as exemplified above for the ring that R⁴ and R⁵ in formula (1), taken together, form with the sulfur atom to which they are attached.

Examples of the cation in the sulfonium salt having formula (2-1) are as exemplified above for the cation in Sulfonium Salt A.

Examples of the cation in the iodonium salt having formula (2-2) are shown below, but not limited thereto.

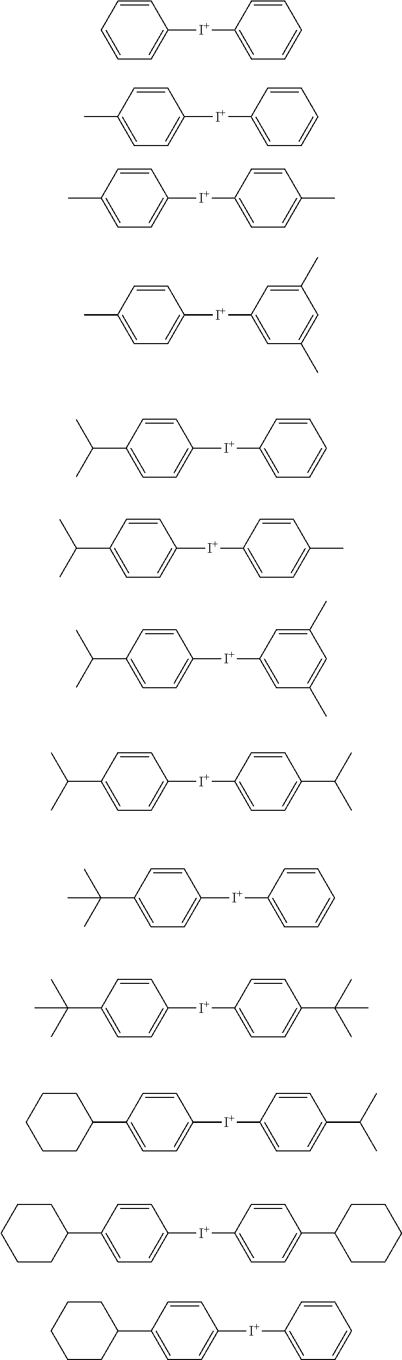

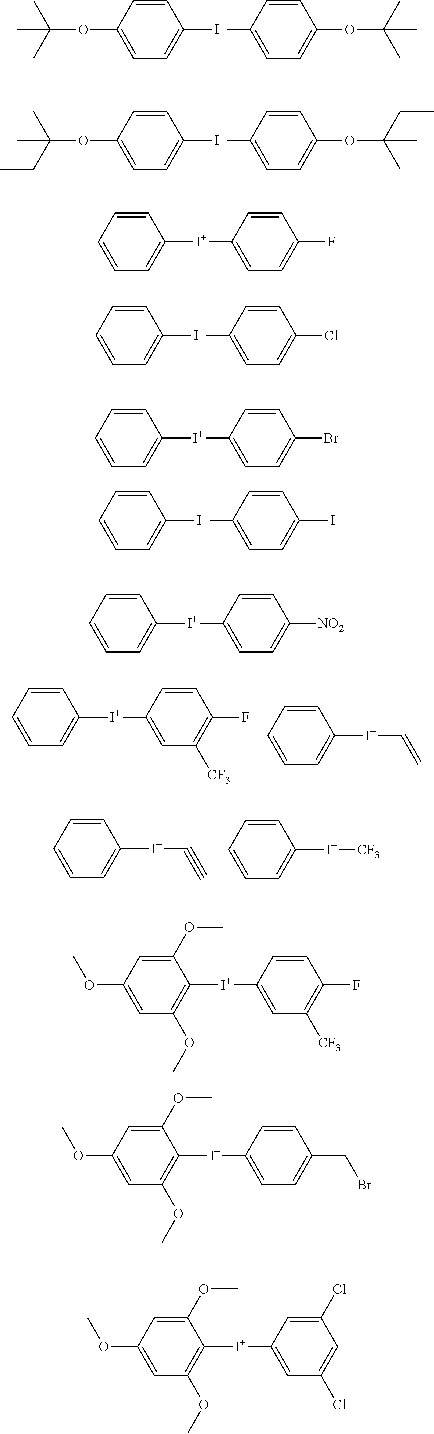

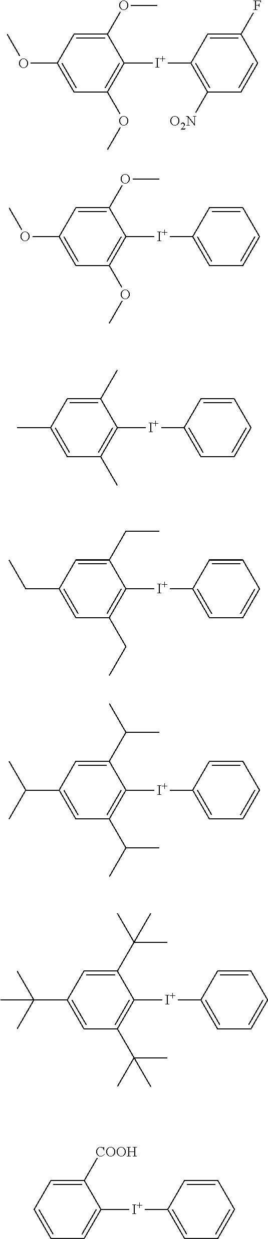

In formulae (2-1) and (2-2), Xa⁻ is an anion of the following formula (2A), (2B3), (2C) or (2D).

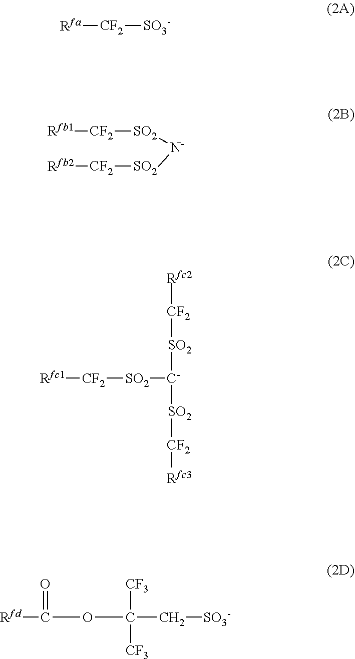

In formula (2A), R^{fa} is fluorine or a C₁-C₄₀ hydrocarbyl group which may contain a heteroatom. The hydrocarbyl group may be saturated or unsaturated and straight, branched or cyclic. Examples thereof are as will be exemplified later for hydrocarbyl group R^{fa1} in formula (2A′).

Of the anions of formula (2A), a structure having formula (2A′) is preferred.

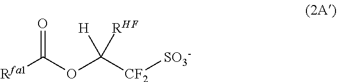

In formula (2A′), R^{HF} is hydrogen or trifluoromethyl, preferably trifluoromethyl.

R^{fa1} is a C₁-C₃₈ hydrocarbyl group which may contain a heteroatom. Suitable heteroatoms include oxygen, nitrogen, sulfur and halogen, with oxygen being preferred. Of the hydrocarbyl groups, those of 6 to 30 carbon atoms are preferred because a high resolution is available in fine pattern formation. The hydrocarbyl group R^{fa1} may be saturated or unsaturated and straight, branched or cyclic. Examples thereof include C₁-C₃₈ alkyl groups such as methyl, ethyl, n-propyl, isopropyl, n-butyl, isobutyl, sec-butyl, tert-butyl, pentyl, neopentyl, hexyl, heptyl, 2-ethylhexyl, nonyl, undecyl, tridecyl, pentadecyl, heptadecyl, and icosanyl; C₃-C₃₈ cyclic saturated hydrocarbyl groups such as cyclopentyl, cyclohexyl, 1-adamantyl, 2-adamantyl, 1-adamantylmethyl, norbornyl, norbornylmethyl, tricyclodecyl, tetracyclododecyl, tetracyclododecylmethyl, and dicyclohexylmethyl; C₂-C₃₈ unsaturated aliphatic hydrocarbyl groups such as allyl and 3-cyclohexenyl; C₆-C₃₈ aryl groups such as phenyl, 1-naphthyl, and 2-naphthyl; C₇-C₃₈ aralkyl groups such as benzyl and diphenylmethyl; and combinations thereof.

In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety. Examples of the heteroatom-containing hydrocarbyl group include tetrahydrofuryl, methoxymethyl, ethoxymethyl, methylthiomethyl, acetamidomethyl, trifluoroethyl, (2-methoxyethoxy)methyl, acetoxymethyl, 2-carboxy-1-cyclohexyl, 2-oxopropyl, 4-oxo-1-adamantyl, and 3-oxocyclohexyl.

With respect to the synthesis of the sulfonium salt having an anion of formula (2A′), reference is made to JP-A 2007-145797, JP-A 2008-106045, JP-A 2009-007327, and JP-A 2009-258695. Also useful are the sulfonium salts described in JP-A 2010-215608, JP-A 2012-041320, JP-A 2012-106986, and JP-A 2012-153644.

Examples of the anion having formula (2A) are as exemplified for the anion having formula (1A) in US 20180335696 (JP-A 2018-197853).

In formula (2B), R^{fb1} and R^{fb2} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (2A′). Preferably R^{fb1} and R^{fb2} each are fluorine or a straight C₁-C₄ fluorinated alkyl group. A pair of R^{fb1} and R^{fb2} may bond together to form a ring with the linkage (—CF₂—SO₂—N—SO₂—CF₂—) to which they are attached, and the ring-forming pair is preferably a fluorinated ethylene or fluorinated propylene group.

In formula (2C), R^{fc1}, R^{fc2} and R^{fc3} are each independently fluorine or a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1} in formula (2A′). Preferably R^{fc1}, R^{fc2} and R^{fc3} each are fluorine or a straight C₁-C₄ fluorinated alkyl group. A pair of R^{fc1} and R^{fc2} may bond together to form a ring with the linkage (—CF₂—SO₂—C—SO₂—CF₂—) to which they are attached, and the ring-forming pair is preferably a fluorinated ethylene or fluorinated propylene group.

In formula (2D), R^{fd} is a C₁-C₄₀ hydrocarbyl group which may contain a heteroatom. The hydrocarbyl group may be saturated or unsaturated and straight, branched or cyclic. Suitable hydrocarbyl groups are as exemplified above for R^{fa1}.

With respect to the synthesis of the sulfonium salt having an anion of formula (2D), reference is made to JP-A 2010-215608 and JP-A 2014-133723.

Examples of the anion having formula (2D) are as exemplified for the anion having formula (1D) in US 20180335696 (JP-A 2018-197853).

The compound having the anion of formula (2D) has a sufficient acid strength to cleave acid labile groups in the base polymer because it is free of fluorine at α-position of sulfo group, but has two trifluoromethyl groups at β-position. Thus the compound is a useful PAG.

Compounds having the formula (3) are also useful as the PAG.

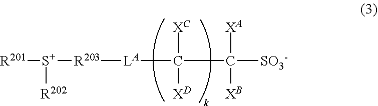

In formula (3), R²⁰¹ and R²⁰² are each independently halogen or a C₁-C₃₀ hydrocarbyl group which may contain a heteroatom. R²⁰³ is a C₁-C₃₀ hydrocarbylene group which may contain a heteroatom. Any two of R²⁰¹, R²⁰² and R²⁰³ may bond together to form a ring with the sulfur atom to which they are attached. Exemplary rings are as described above for the ring that R⁴ and R⁵ in formula (1), taken together, form with the sulfur atom to which they are attached.

The hydrocarbyl groups R²⁰¹ and R²⁰² may be saturated or unsaturated and straight, branched or cyclic. Examples thereof include C₁-C₃₀ alkyl groups such as methyl, ethyl, n-propyl, isopropyl, n-butyl, sec-butyl, tert-butyl, n-pentyl, tert-pentyl, n-hexyl, n-octyl, 2-ethylhexyl, n-nonyl, and n-decyl; C₃-C₃₀ cyclic saturated hydrocarbyl groups such as cyclopentyl, cyclohexyl, cyclopentylmethyl, cyclopentylethyl, cyclopentylbutyl, cyclohexylmethyl, cyclohexylethyl, cyclohexylbutyl, norbornyl, oxanorbornyl, tricyclo[5.2.1.0^{2,6}]decyl, and adamantyl; C₆-C₃₀ aryl groups such as phenyl, methylphenyl, ethylphenyl, n-propylphenyl, isopropylphenyl, n-butylphenyl, isobutylphenyl, sec-butylphenyl, tert-butylphenyl, naphthyl, methylnaphthyl, ethylnaphthyl, n-propylnaphthyl, isopropylnaphthyl, n-butylnaphthyl, isobutylnaphthyl, sec-butylnaphthyl, tert-butylnaphthyl, and anthracenyl; and combinations thereof. In the hydrocarbyl group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety.

The hydrocarbylene group R²⁰3 may be saturated or unsaturated and straight, branched or cyclic. Examples thereof include C₁-C₃₀ alkanediyl groups such as methanediyl, ethane-1,1-diyl, ethane-1,2-diyl, propane-1,3-diyl, butane-1,4-diyl, pentane-1,5-diyl, hexane-1,6-diyl, heptane-1,7-diyl, octane-1,8-diyl, nonane-1,9-diyl, decane-1,10-diyl, undecane-1,11-diyl, dodecane-1,12-diyl, tridecane-1,13-diyl, tetradecane-1,14-diyl, pentadecane-1,15-diyl, hexadecane-1,16-diyl, and heptadecane-1,17-diyl; C₃-C₃₀ cyclic saturated hydrocarbylene groups such as cyclopentanediyl, cyclohexanediyl, norbornanediyl and adamantanediyl; C₆-C₃₀ arylene groups such as phenylene, methylphenylene, ethylphenylene, n-propylphenylene, isopropylphenylene, n-butylphenylene, isobutylphenylene, sec-butylphenylene, tert-butylphenylene, naphthylene, methylnaphthylene, ethylnaphthylene, n-propylnaphthylene, isopropylnaphthylene, n-butylnaphthylene, isobutylnaphthylene, sec-butylnaphthylene and tert-butylnaphthylene; and combinations thereof. In the hydrocarbylene group, some or all of the hydrogen atoms may be substituted by a moiety containing a heteroatom such as oxygen, sulfur, nitrogen or halogen, or some constituent —CH₂— may be replaced by a moiety containing a heteroatom such as oxygen, sulfur or nitrogen, so that the group may contain a hydroxy, fluorine, chlorine, bromine, iodine, cyano, nitro, carbonyl, ether bond, ester bond, sulfonate ester bond, carbonate bond, lactone ring, sultone ring, carboxylic anhydride (—C(═O)—O—C(═O)—) or haloalkyl moiety. Of the heteroatoms, oxygen is preferred.

In formula (3), L^{A} is a single bond, ether bond or a C₁-C₂₀ hydrocarbylene group which may contain a heteroatom. The hydrocarbylene group may be saturated or unsaturated and straight, branched or cyclic. Examples thereof are as exemplified above for R²⁰³.

In formula (3), X^{A}, X^{B}, X^{C} and X^{D} are each independently hydrogen, fluorine or trifluoromethyl, with the proviso that at least one of X^{A}, X^{B}, X^{C} and X^{D} is fluorine or trifluoromethyl.

In formula (3), k is an integer of 0 to 3.

Of the PAGs having formula (3), those having the formula (3′) are preferred.

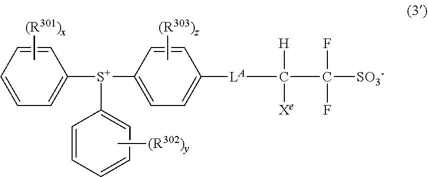

In formula (3′), L^{A} is as defined above. R^{HF} is hydrogen or trifluoromethyl, preferably trifluoromethyl. R³⁰¹, R³⁰² and R³⁰³ are each independently hydrogen or a C₁-C₂₀ hydrocarbyl group which may contain a heteroatom. The hydrocarbyl group may be saturated or unsaturated and straight, branched or cyclic. Examples thereof are as exemplified above for R^{fa1} in formula (2A′). The subscripts x and y are each independently an integer of 0 to 5, and z is an integer of 0 to 4.

Examples of the PAG having formula (3) are as exemplified for the PAG having formula (2) in JP-A 2017-026980.

Of the foregoing PAGs, those having an anion of formula (2A′) or (2D) are especially preferred because of reduced acid diffusion and high solubility in the solvent. Also those having formula (3′) are especially preferred because of extremely reduced acid diffusion.

Also a sulfonium or iodonium salt having an anion containing an iodized or brominated aromatic ring may be used as the PAG. Suitable are sulfonium and iodonium salts having the formulae (4-1) and (4-2).

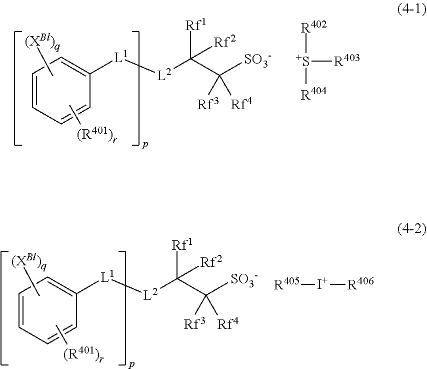

In formulae (4-1) and (4-2), p is an integer of 1 to 3, q is an integer of 1 to 5, and r is an integer of 0 to 3, and 1≤q+r≤5. Preferably, q is 1, 2 or 3, more preferably 2 or 3, and r is 0, 1 or 2.

In formulae (4-1) and (4-2), X^{BI} is iodine or bromine. A plurality of X^{BI} may be the same or different when p and/or q is 2 or more.

L¹ is a single bond, ether bond, ester bond, or a C₁-C₆ saturated hydrocarbylene group which may contain an ether bond or ester bond. The saturated hydrocarbylene group may be straight, branched or cyclic.

L² is a single bond or a C₁-C₂₀ divalent linking group when p is 1, and a C₁-C₂₀ (p+1)-valent linking group which may contain oxygen, sulfur or nitrogen when p is 2 or 3.

R⁴⁰¹ is a hydroxy group, carboxy group, fluorine, chlorine, bromine, amino group, or a C₁-C₂₀ hydrocarbyl, C₁-C₂₀ hydrocarbyloxy, C₂-C₂₀ hydrocarbylcarbonyl, C₂-C₂₀ hydrocarbyloxycarbonyl, C₂-C₂₀ hydrocarbylcarbonyloxy or C₁-C₂₀ hydrocarbylsulfonyloxy group, which may contain fluorine, chlorine, bromine, hydroxy, amino or ether bond, or —N(R^{401A})(R^{401B}), —N(R^{401C})—C(═O)—R^{401D} or —N(R^{401C})—C(═O)—O—R^{401D}. R^{401A} and R^{401B} are each independently hydrogen or a C₁-C₆ saturated hydrocarbyl group. R^{401C} is hydrogen or a C₁-C₆ saturated hydrocarbyl group which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. R^{401D} is a C₁-C₁₆ aliphatic hydrocarbyl group, C₆-C₁₄ aryl group or C₇-C₁₅ aralkyl group, which may contain halogen, hydroxy, C₁-C₆ saturated hydrocarbyloxy, C₂-C₆ saturated hydrocarbylcarbonyl or C₂-C₆ saturated hydrocarbylcarbonyloxy moiety. The aliphatic hydrocarbyl group may be saturated or unsaturated and straight, branched or cyclic. The hydrocarbyl, hydrocarbyloxy, hydrocarbylcarbonyl, hydrocarbyloxycarbonyl, hydrocarbylcarbonyloxy and hydrocarbylsulfonyloxy groups may be straight, branched or cyclic. A plurality of R⁴⁰¹ may be the same or different when p and/or r is 2 or more. Of these, R⁴⁰¹ is preferably hydroxy, —N(R^{401C})—C(═O)—R^{401D}, —N(R^{401C})—C(═O)—O—R^{401D}, fluorine, chlorine, bromine, methyl or methoxy.

In formulae (4-1) and (4-2), Rf¹ to Rf⁴ are each independently hydrogen, fluorine or trifluoromethyl, at least one of Rf¹ to Rf⁴ is fluorine or trifluoromethyl. Rf¹ and Rf², taken together, may form a carbonyl group. Preferably, both Rf³ and Rf⁴ are fluorine.

R⁴⁰² to R⁴⁰⁶ are each independently halogen or a C₁-C₂₀ hydrocarbyl group which may contain a heteroatom. The hydrocarbyl group may be saturated or unsaturated and straight, branched or cyclic. Examples thereof are as exemplified above for the hydrocarbyl groups R⁴ to R⁶ in formula (1). In the hydrocarbyl group, some or all of the hydrogen atoms may be substituted by hydroxy, carboxy, halogen, cyano, nitro, mercapto, sultone ring, sulfo, or sulfonium salt-containing moiety, and some constituent —CH₂— may be replaced by an ether bond, ester bond, carbonyl moiety, amide bond, carbonate bond or sulfonate ester bond. R⁴⁰² and R⁴⁰³ may bond together to form a ring with the sulfur atom to which they are attached. Exemplary rings are as described above for the ring that R⁴ and R⁵ in formula (1), taken together, form with the sulfur atom to which they are attached.

Examples of the cation in the sulfonium salt having formula (4-1) include those exemplified above as the cation in the sulfonium salt having formula (1). Examples of the cation in the iodonium salt having formula (4-2) include those exemplified above as the cation in the iodonium salt having formula (2-2).

Examples of the anion in the onium salts having formulae (4-1) and (4-2) are shown below, but not limited thereto. Herein X^{BI} is as defined above.

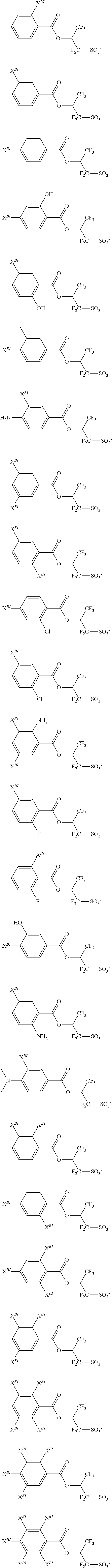

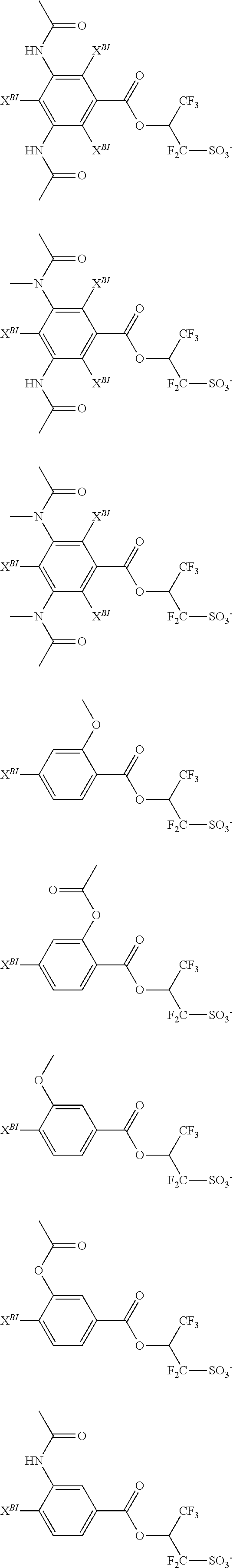

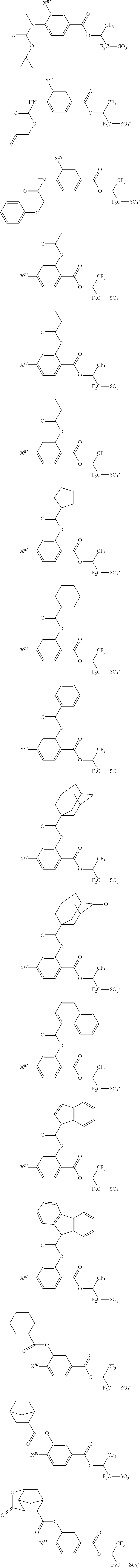

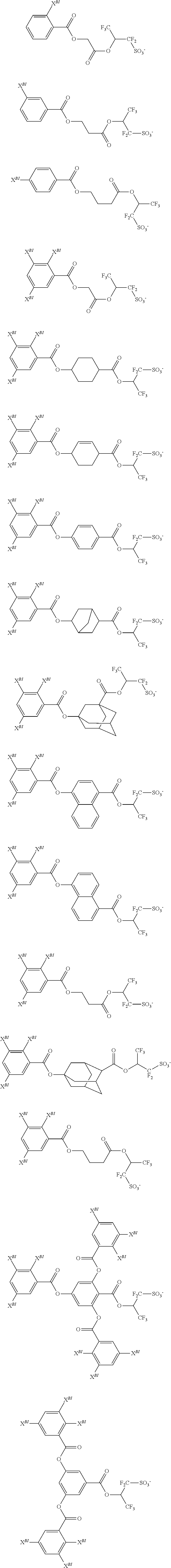

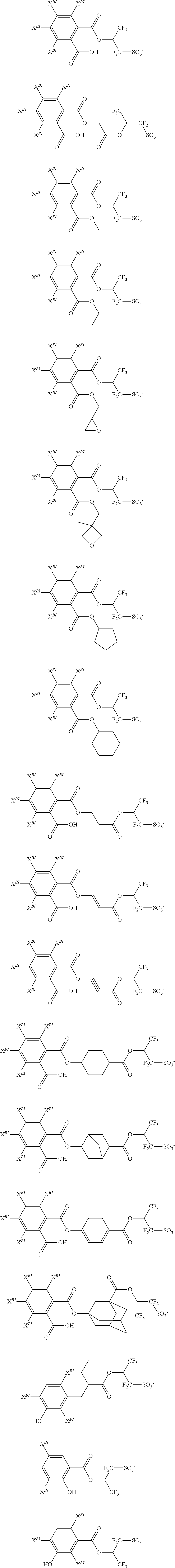

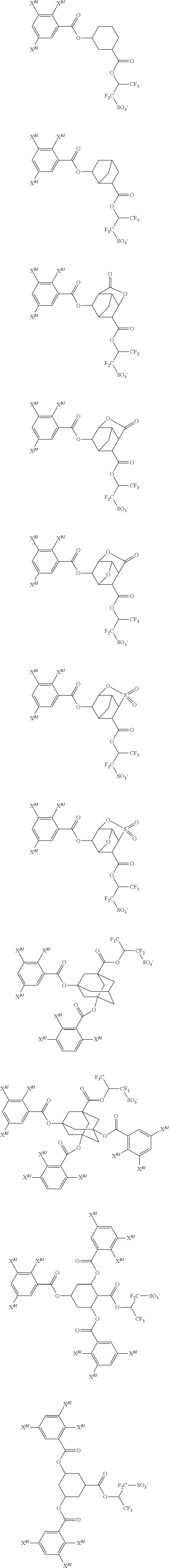

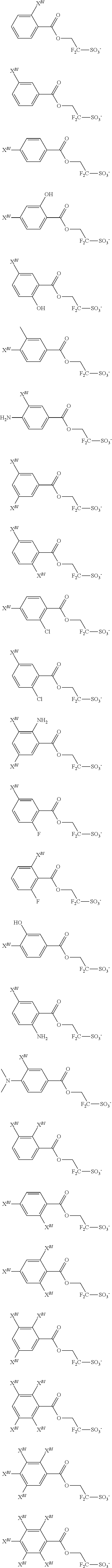

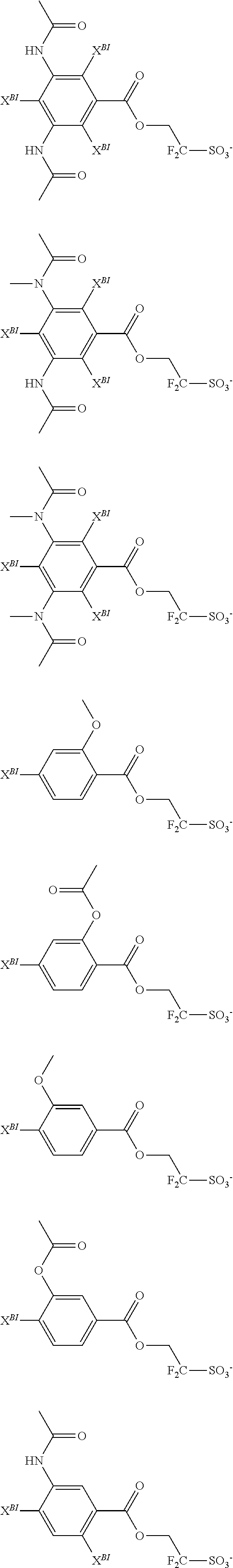

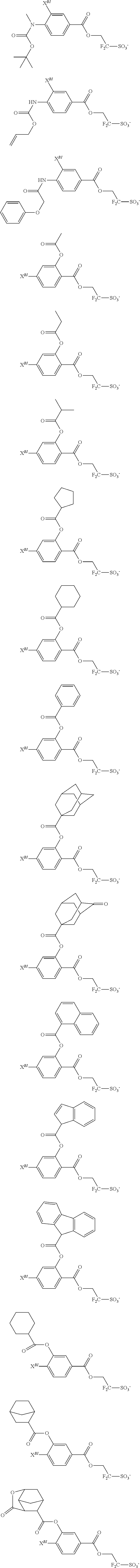

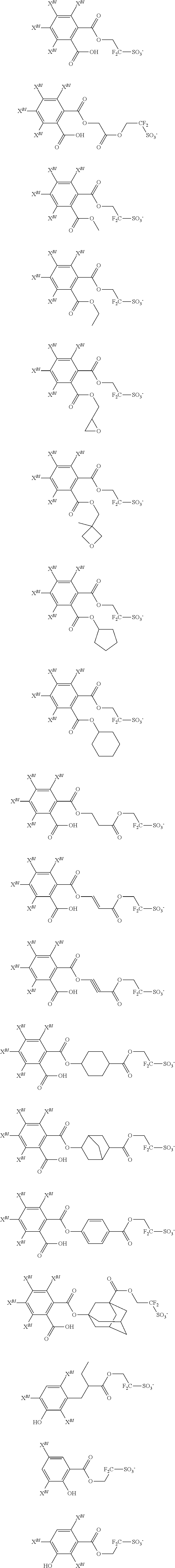

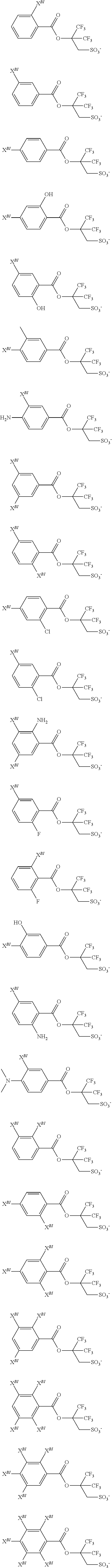

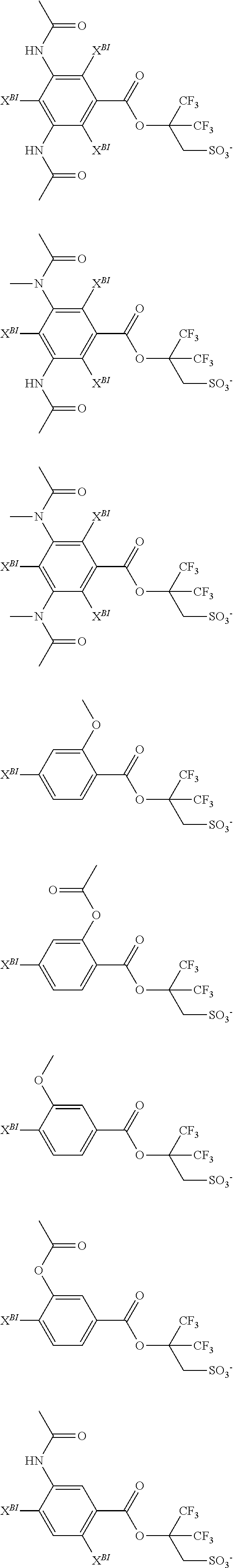

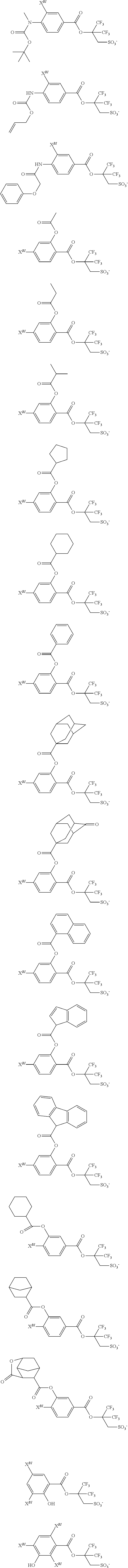

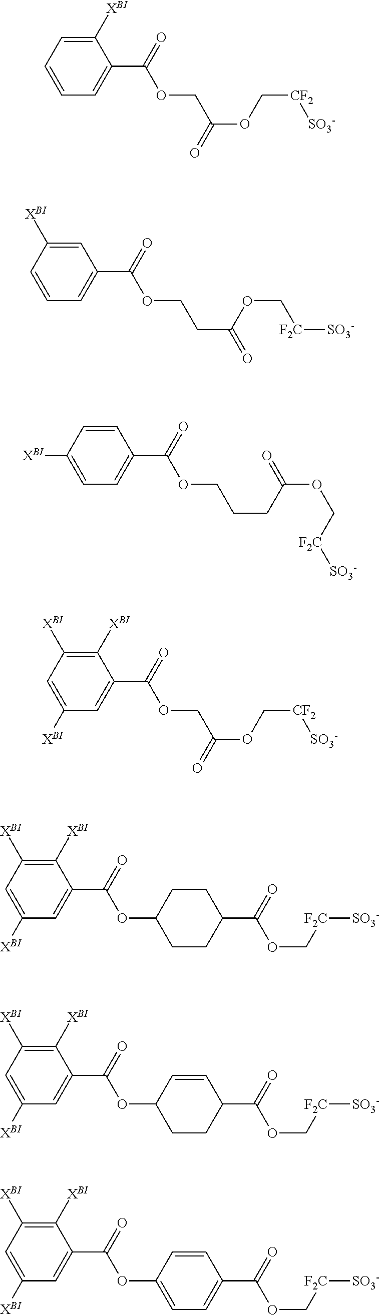

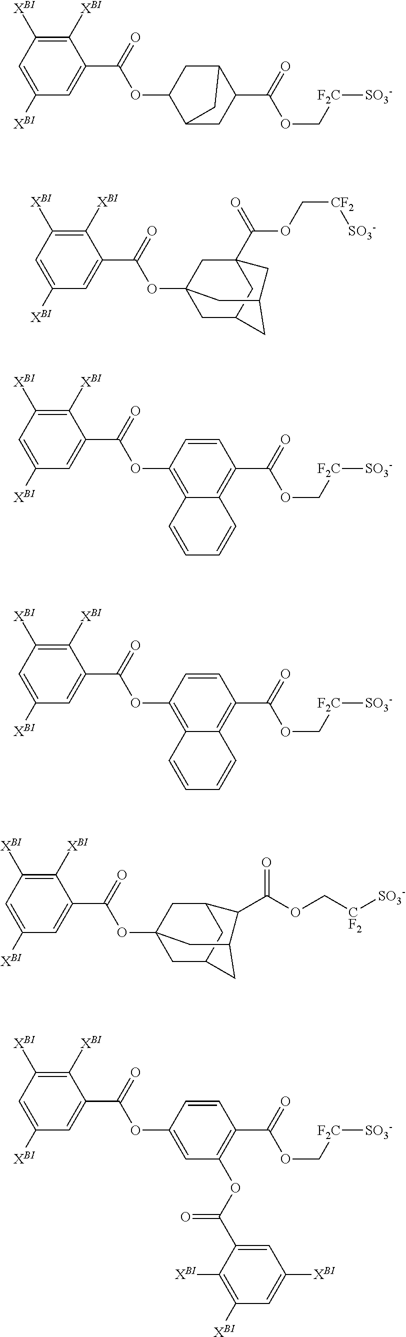

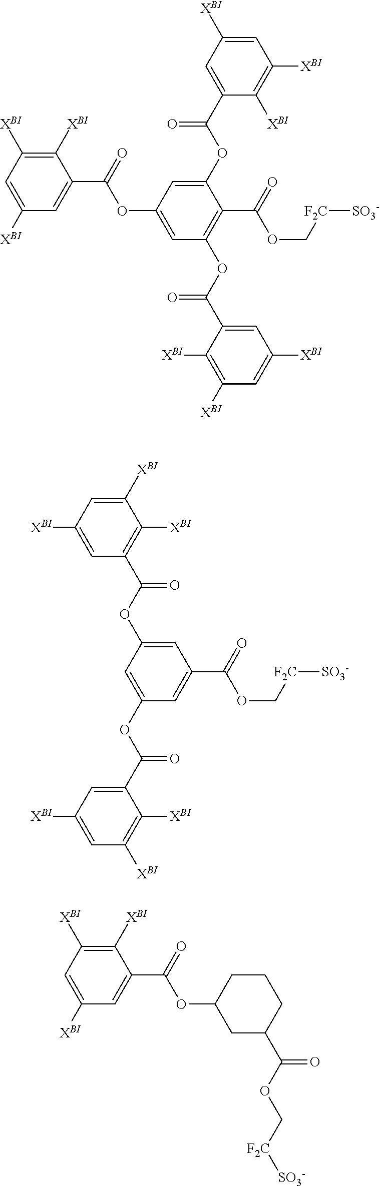

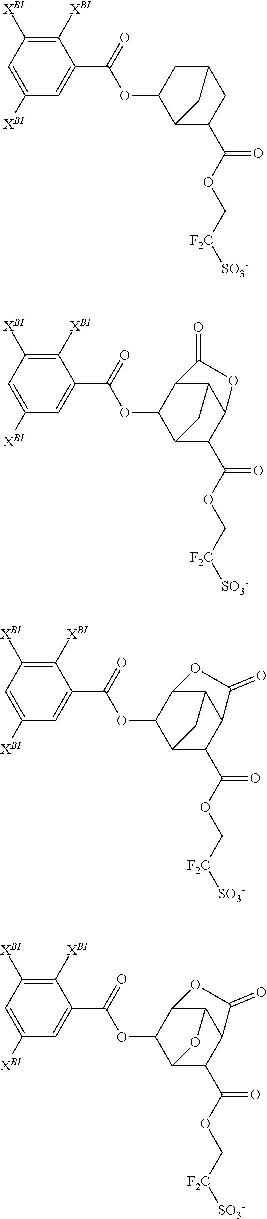

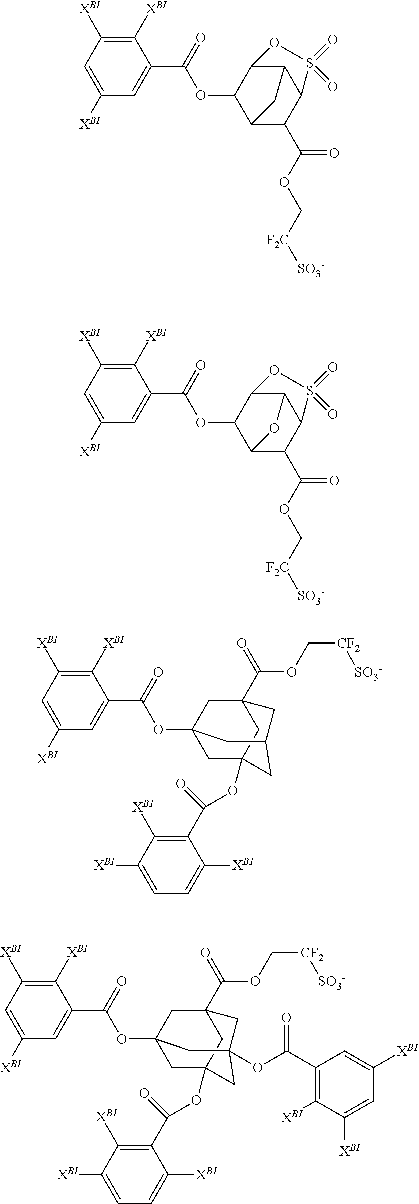

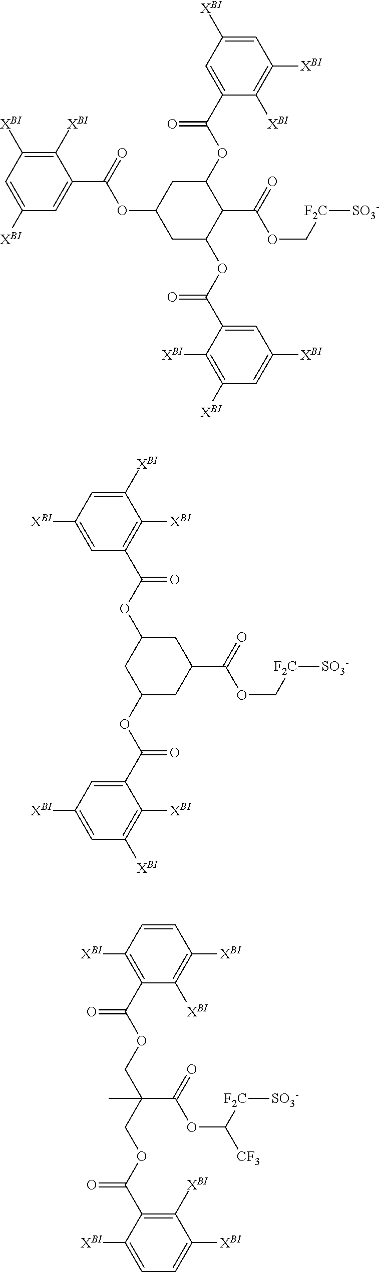

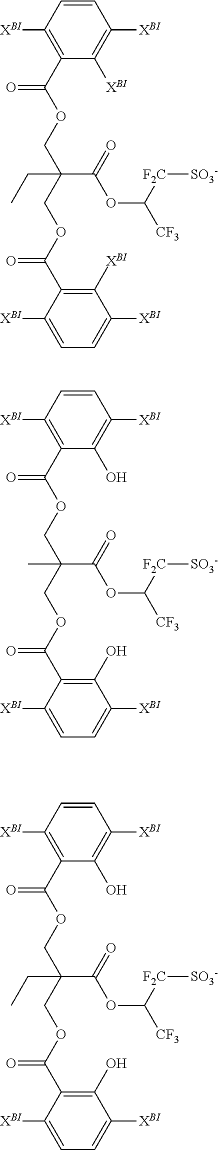

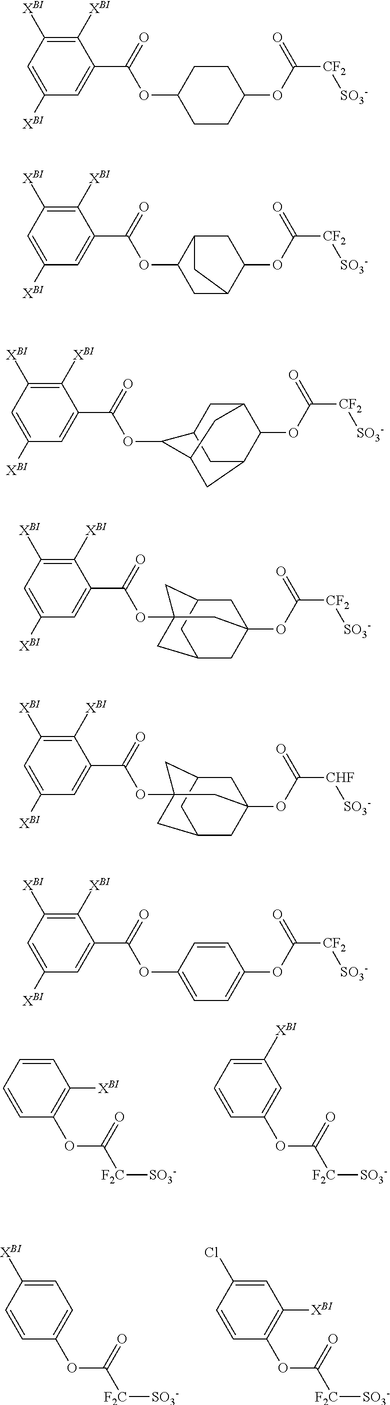

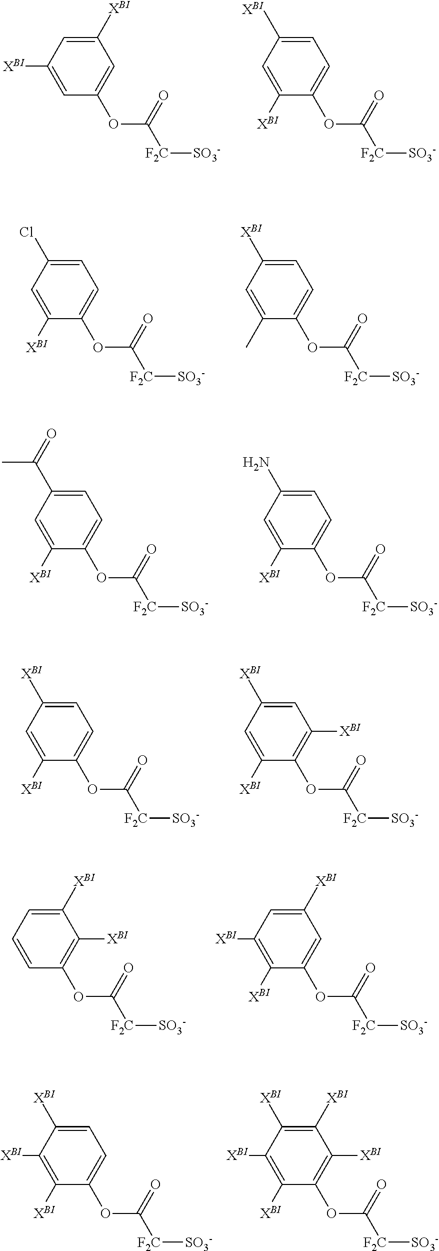

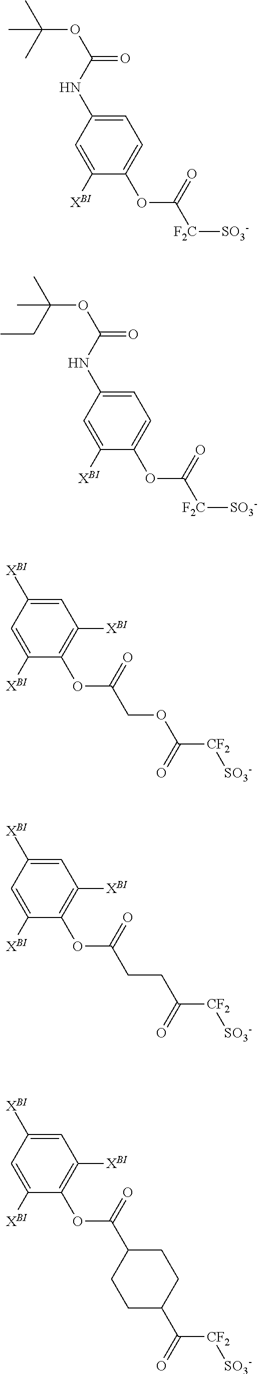

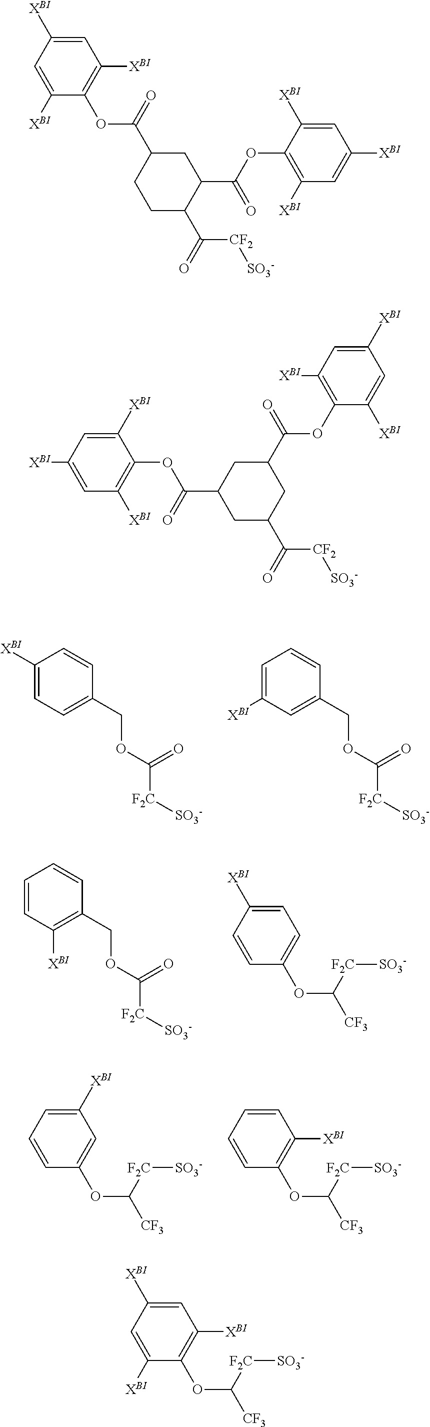

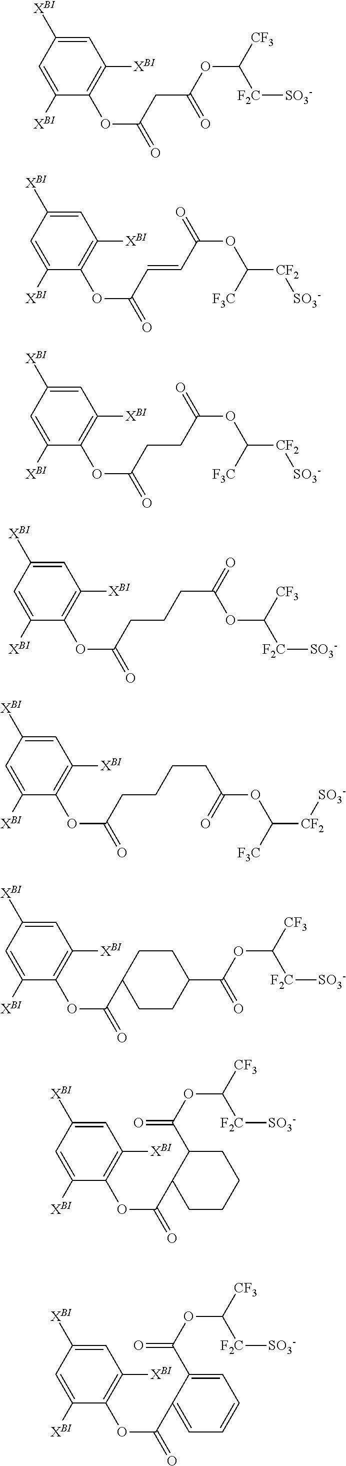

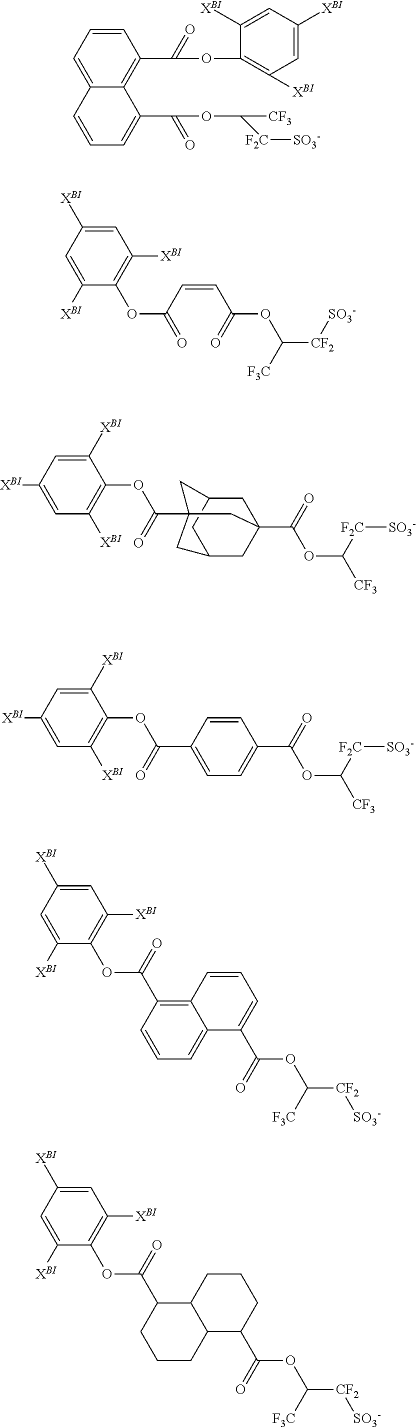

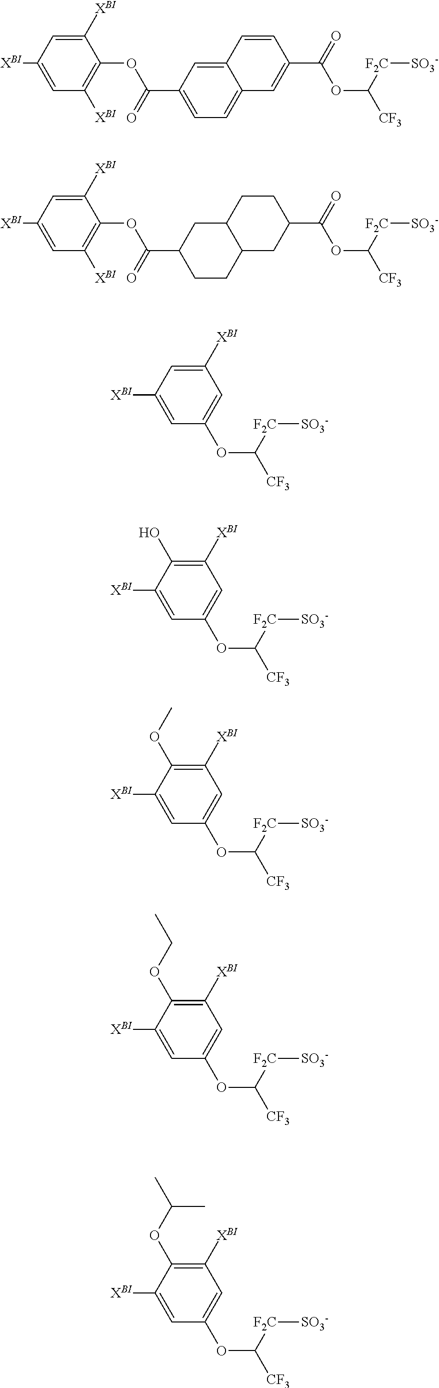

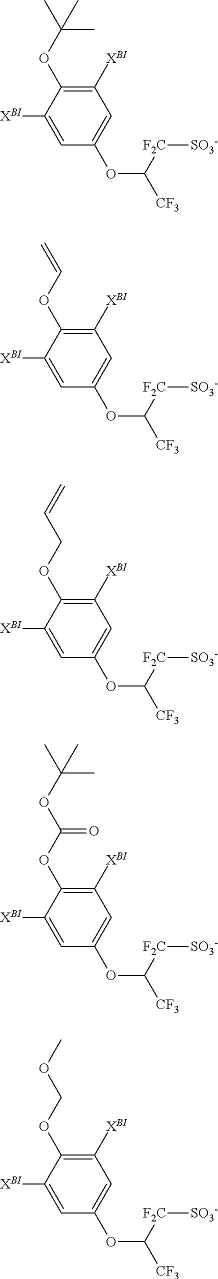

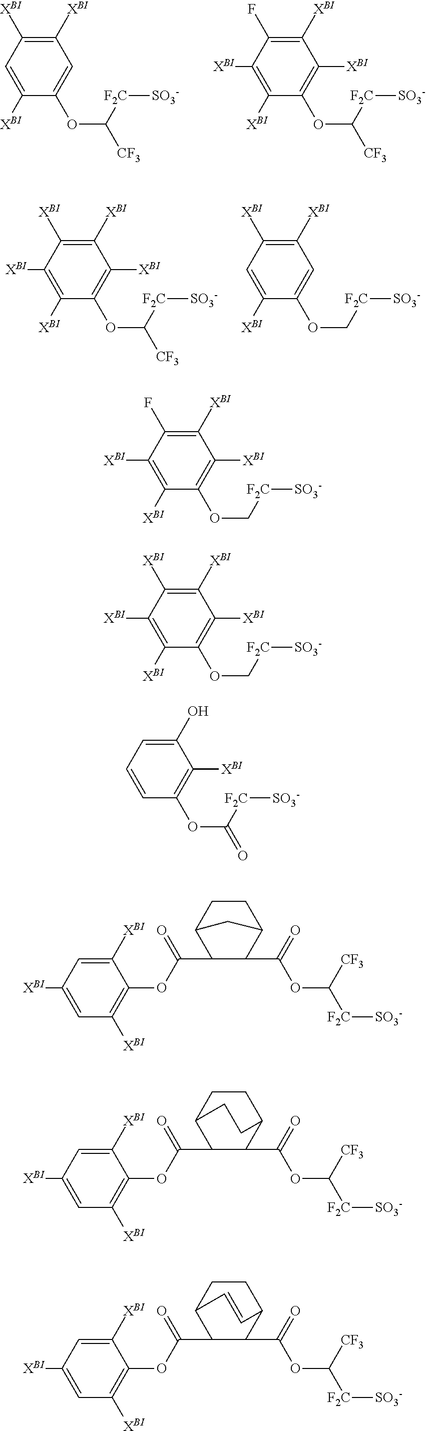

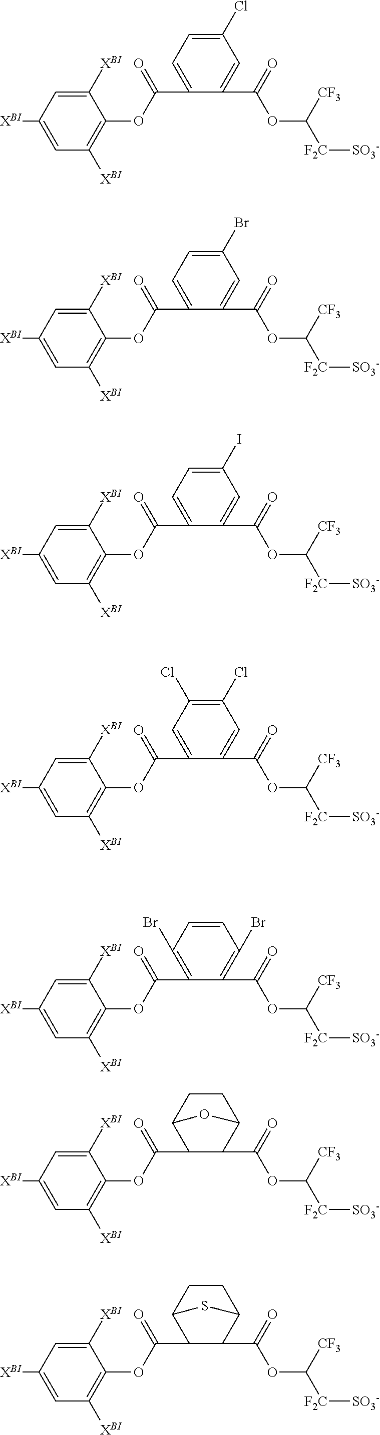

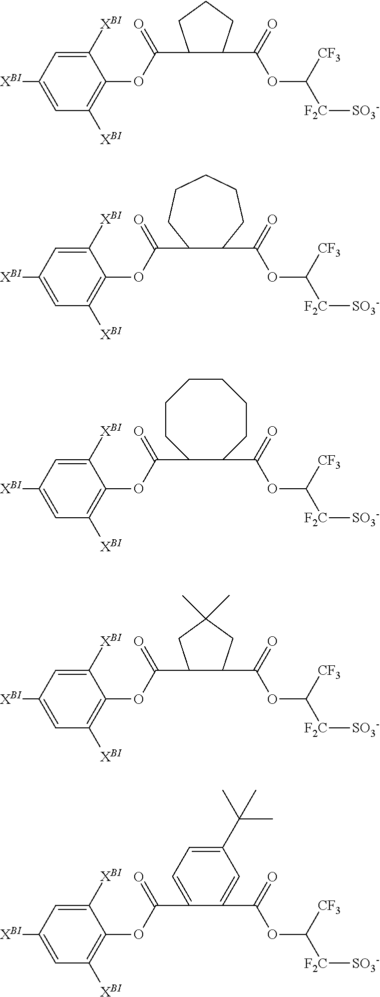

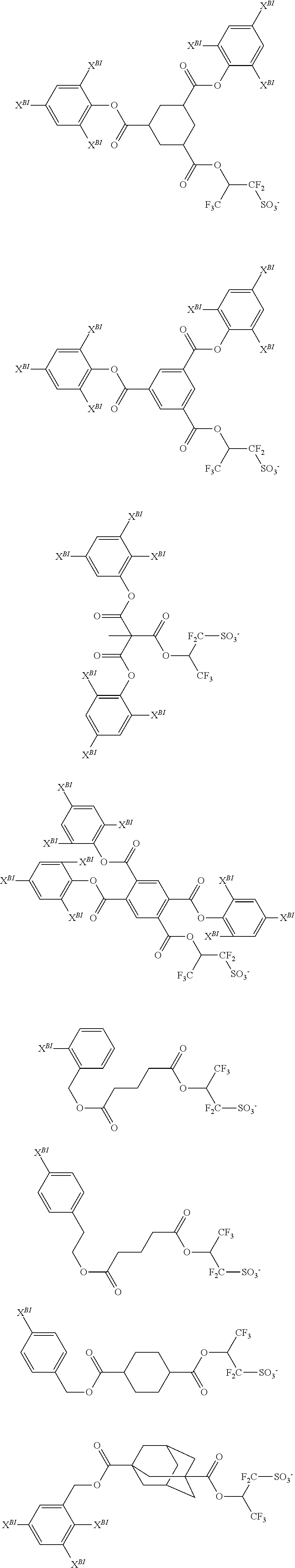

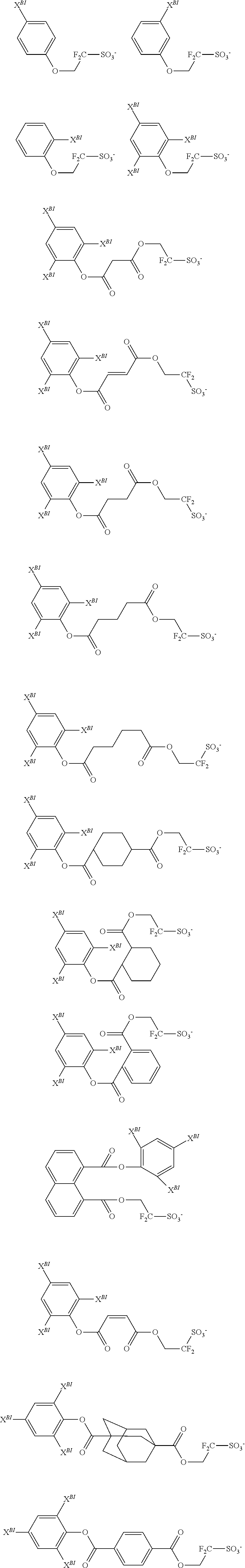

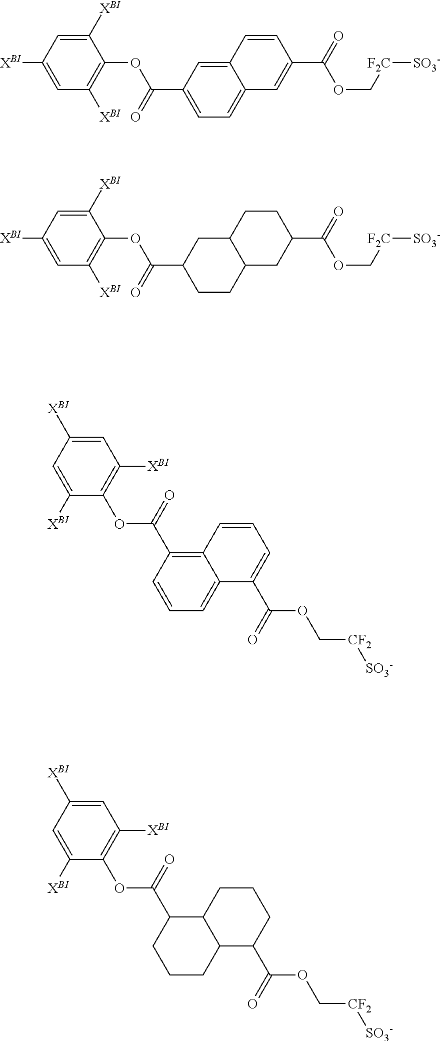

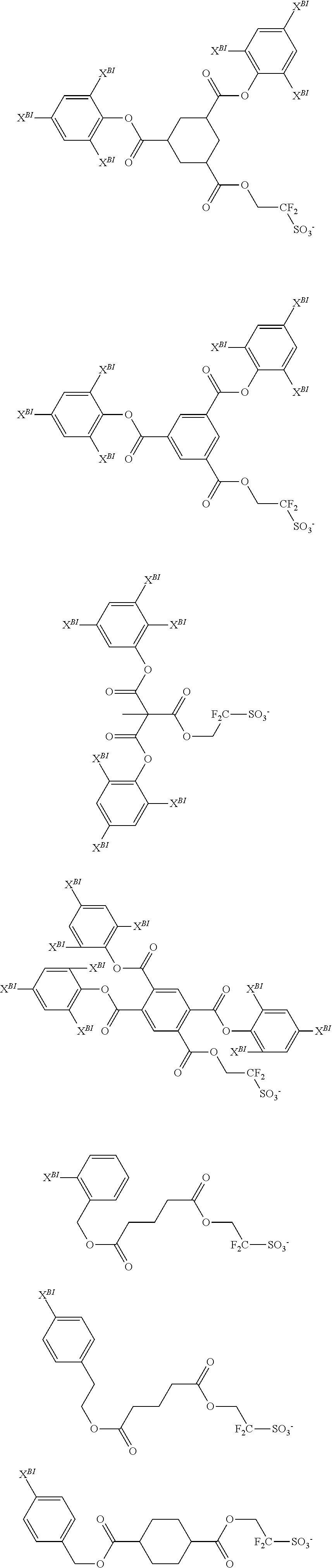

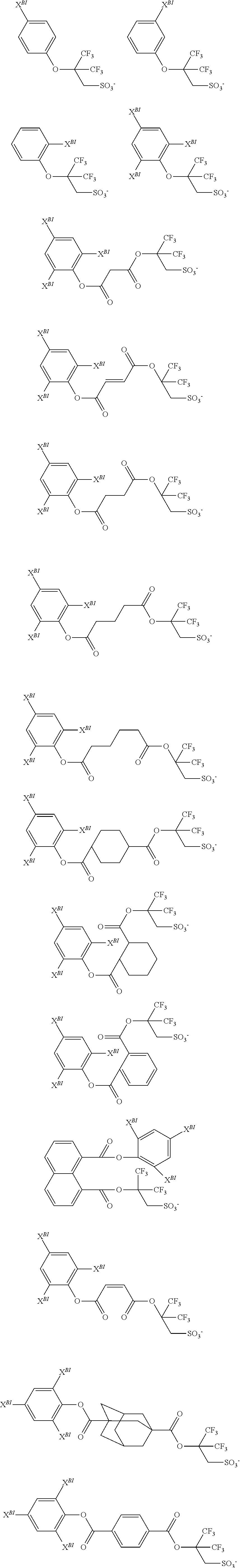

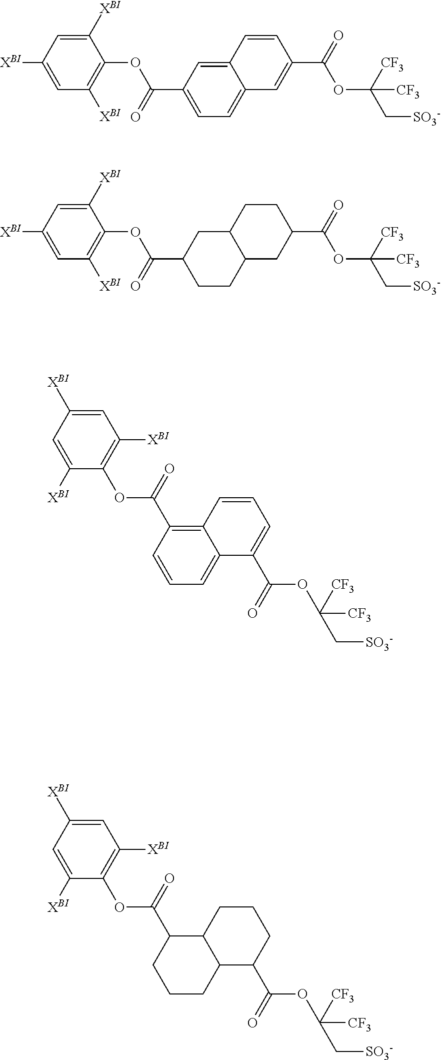

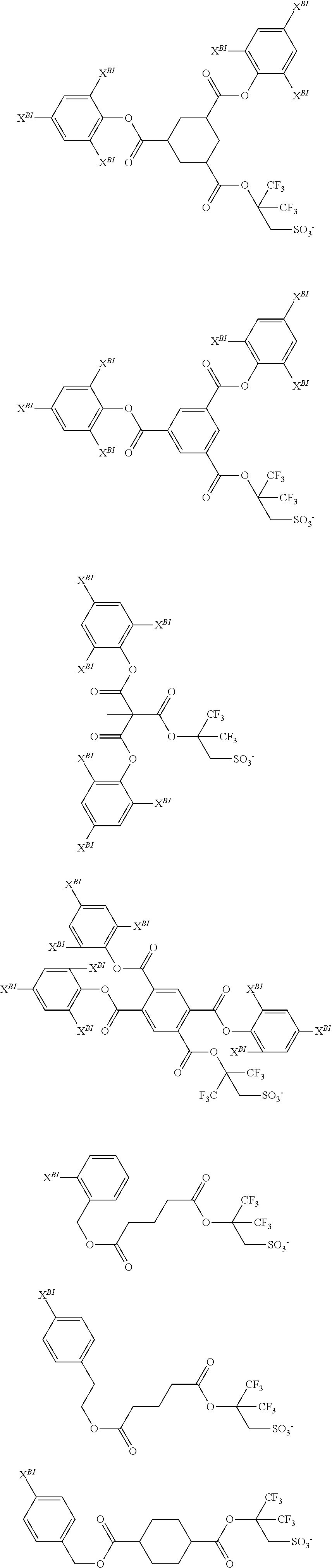

When used, the acid generator of addition type is preferably added in an amount of 0.1 to 50 parts, and more preferably 1 to 40 parts by weight per 100 parts by weight of the base polymer. The resist composition functions as a chemically amplified resist composition when the base polymer includes repeat units (f) and/or the acid generator of addition type is contained.

### [Organic Solvent]

An organic solvent may be added to the resist composition. The organic solvent used herein is not particularly limited as long as the foregoing and other components are soluble therein. Examples of the organic solvent are described in JP-A 2008-111103, paragraphs [0144]-[0145] (U.S. Pat. No. 7,537,880). Exemplary solvents include ketones such as cyclohexanone, cyclopentanone, methyl-2-n-pentyl ketone and 2-heptanone; alcohols such as 3-methoxybutanol, 3-methyl-3-methoxybutanol, 1-methoxy-2-propanol, 1-ethoxy-2-propanol, and diacetone alcohol (DAA); ethers such as propylene glycol monomethyl ether (PGME), ethylene glycol monomethyl ether, propylene glycol monoethyl ether, ethylene glycol monoethyl ether, propylene glycol dimethyl ether, and diethylene glycol dimethyl ether; esters such as propylene glycol monomethyl ether acetate (PGMEA), propylene glycol monoethyl ether acetate, ethyl lactate, ethyl pyruvate, butyl acetate, methyl 3-methoxypropionate, ethyl 3-ethoxypropionate, tert-butyl acetate, tert-butyl propionate, and propylene glycol mono-tert-butyl ether acetate; and lactones such as γ-butyrolactone, which may be used alone or in admixture.

The organic solvent is preferably added in an amount of 100 to 10,000 parts, and more preferably 200 to 8,000 parts by weight per 100 parts by weight of the base polymer.

### [Other Components]

In addition to the foregoing components, the resist composition may further comprise other components such as a surfactant, dissolution inhibitor, crosslinker, quencher other than Sulfonium Salt A, water repellency improver, and acetylene alcohol. Each of additional components may be used alone or in admixture of two or more.

Exemplary surfactants are described in JP-A 2008-111103, paragraphs [0165]-[0166]. Inclusion of a surfactant may improve or control the coating characteristics of the resist composition. When used, the surfactant is preferably added in an amount of 0.0001 to 10 parts by weight per 100 parts by weight of the base polymer.

When the resist composition is of positive tone, the inclusion of a dissolution inhibitor may lead to an increased difference in dissolution rate between exposed and unexposed areas and a further improvement in resolution. The dissolution inhibitor which can be used herein is a compound having at least two phenolic hydroxy groups on the molecule, in which an average of from 0 to 100 mol % of all the hydrogen atoms on the phenolic hydroxy groups are replaced by acid labile groups or a compound having at least one carboxy group on the molecule, in which an average of 50 to 100 mol % of all the hydrogen atoms on the carboxy groups are replaced by acid labile groups, both the compounds having a molecular weight of 100 to 1,000, and preferably 150 to 800. Typical are bisphenol A, trisphenol, phenolphthalein, cresol novolac, naphthalenecarboxylic acid, adamantanecarboxylic acid, and cholic acid derivatives in which the hydrogen atom on the hydroxy or carboxy group is replaced by an acid labile group, as described in U.S. Pat. No. 7,771,914 (JP-A 2008-122932, paragraphs [0155]-[0178]).

When the resist composition is of positive tone and contains a dissolution inhibitor, the dissolution inhibitor is preferably added in an amount of 0 to 50 parts, more preferably 5 to 40 parts by weight per 100 parts by weight of the base polymer.

When the resist composition is of negative tone, a negative pattern may be formed by adding a crosslinker to reduce the dissolution rate of a resist film in exposed area. Suitable crosslinkers include epoxy compounds, melamine compounds, guanamine compounds, glycoluril compounds and urea compounds having substituted thereon at least one group selected from among methylol, alkoxymethyl and acyloxymethyl groups, isocyanate compounds, azide compounds, and compounds having a double bond such as an alkenyloxy group. These compounds may be used as an additive or introduced into a polymer side chain as a pendant. Hydroxy-containing compounds may also be used as the crosslinker.

Examples of the epoxy compound include tris(2,3-epoxypropyl) isocyanurate, trimethylolmethane triglycidyl ether, trimethylolpropane triglycidyl ether, and triethylolethane triglycidyl ether. Examples of the melamine compound include hexamethylol melamine, hexamethoxymethyl melamine, hexamethylol melamine compounds having 1 to 6 methylol groups methoxymethylated and mixtures thereof, hexamethoxyethyl melamine, hexaacyloxymethyl melamine, hexamethylol melamine compounds having 1 to 6 methylol groups acyloxymethylated and mixtures thereof. Examples of the guanamine compound include tetramethylol guanamine, tetramethoxymethyl guanamine, tetramethylol guanamine compounds having 1 to 4 methylol groups methoxymethylated and mixtures thereof, tetramethoxyethyl guanamine, tetraacyloxyguanamine, tetramethylol guanamine compounds having 1 to 4 methylol groups acyloxymethylated and mixtures thereof. Examples of the glycoluril compound include tetramethylol glycoluril, tetramethoxyglycoluril, tetramethoxymethyl glycoluril, tetramethylol glycoluril compounds having 1 to 4 methylol groups methoxymethylated and mixtures thereof, tetramethylol glycoluril compounds having 1 to 4 methylol groups acyloxymethylated and mixtures thereof. Examples of the urea compound include tetramethylol urea, tetramethoxymethyl urea, tetramethylol urea compounds having 1 to 4 methylol groups methoxymethylated and mixtures thereof, and tetramethoxyethyl urea.

Suitable isocyanate compounds include tolylene diisocyanate, diphenylmethane diisocyanate, hexamethylene diisocyanate and cyclohexane diisocyanate. Suitable azide compounds include 1,1′-biphenyl-4,4′-bisazide, 4,4′-methylidenebisazide, and 4,4′-oxybisazide. Examples of the alkenyloxy group-containing compound include ethylene glycol divinyl ether, triethylene glycol divinyl ether, 1,2-propanediol divinyl ether, 1,4-butanediol divinyl ether, tetramethylene glycol divinyl ether, neopentyl glycol divinyl ether, trimethylol propane trivinyl ether, hexanediol divinyl ether, 1,4-cyclohexanediol divinyl ether, pentaerythritol trivinyl ether, pentaerythritol tetravinyl ether, sorbitol tetravinyl ether, sorbitol pentavinyl ether, and trimethylol propane trivinyl ether.

When the resist composition is of negative tone and contains a crosslinker, the crosslinker is preferably added in an amount of 0.1 to 50 parts, more preferably 1 to 40 parts by weight per 100 parts by weight of the base polymer.

The other quencher is typically selected from conventional basic compounds. Conventional basic compounds include primary, secondary, and tertiary aliphatic amines, mixed amines, aromatic amines, heterocyclic amines, nitrogen-containing compounds with carboxy group, nitrogen-containing compounds with sulfonyl group, nitrogen-containing compounds with hydroxy group, nitrogen-containing compounds with hydroxyphenyl group, alcoholic nitrogen-containing compounds, amide derivatives, imide derivatives, and carbamate derivatives. Also included are primary, secondary, and tertiary amine compounds, specifically amine compounds having a hydroxy group, ether bond, ester bond, lactone ring, cyano group, or sulfonic ester bond as described in JP-A 2008-111103, paragraphs [0146]-[0164], and compounds having a carbamate group as described in JP 3790649. Addition of a basic compound may be effective for further suppressing the diffusion rate of acid in the resist film or correcting the pattern profile.

Onium salts such as sulfonium, iodonium and ammonium salts of sulfonic acids which are not fluorinated at α-position as described in U.S. Pat. No. 8,795,942 (JP-A 2008-158339) and similar onium salts of carboxylic acid may also be used as the quencher. While an α-fluorinated sulfonic acid, imide acid, and methide acid are necessary to deprotect the acid labile group of carboxylic acid ester, an α-non-fluorinated sulfonic acid and a carboxylic acid are released by salt exchange with an α-non-fluorinated onium salt. An α-non-fluorinated sulfonic acid and a carboxylic acid function as a quencher because they do not induce deprotection reaction.

Also useful are quenchers of polymer type as described in U.S. Pat. No. 7,598,016 (JP-A 2008-239918). The polymeric quencher segregates at the resist surface and thus enhances the rectangularity of resist pattern. When a protective film is applied as is often the case in the immersion lithography, the polymeric quencher is also effective for preventing a film thickness loss of resist pattern or rounding of pattern top.

When used, the other quencher is preferably added in an amount of 0 to 5 parts, more preferably 0 to 4 parts by weight per 100 parts by weight of the base polymer.

To the resist composition, a water repellency improver may also be added for improving the water repellency on surface of a resist film. The water repellency improver may be used in the topcoatless immersion lithography. Suitable water repellency improvers include polymers having a fluoroalkyl group and polymers of specific structure having a 1,1,1,3,3,3-hexafluoro-2-propanol residue and are described in JP-A 2007-297590 and JP-A 2008-111103, for example. The water repellency improver should be soluble in the alkaline developer and organic solvent developer. The water repellency improver of specific structure having a 1,1,1,3,3,3-hexafluoro-2-propanol residue is well soluble in the developer. A polymer comprising repeat units having an amino group or amine salt may serve as the water repellent additive and is effective for preventing evaporation of acid during PEB, thus preventing any hole pattern opening failure after development. An appropriate amount of the water repellency improver is 0 to 20 parts, more preferably 0.5 to 10 parts by weight per 100 parts by weight of the base polymer.

Also, an acetylene alcohol may be blended in the resist composition. Suitable acetylene alcohols are described in JP-A 2008-122932, paragraphs [0179]-[0182]. An appropriate amount of the acetylene alcohol blended is 0 to 5 parts by weight per 100 parts by weight of the base polymer.

### [Process]

The resist composition is used in the fabrication of various integrated circuits. Pattern formation using the resist composition may be performed by well-known lithography processes. The process generally involves the steps of applying the resist composition onto a substrate to form a resist film thereon, exposing the resist film to high-energy radiation, and developing the exposed resist film in a developer. If necessary, any additional steps may be added.

Specifically, the resist composition is first applied onto a substrate on which an integrated circuit is to be formed (e.g., Si, SiO₂, SiN, SiON, TiN, WSi, BPSG, SOG, or organic antireflective coating) or a substrate on which a mask circuit is to be formed (e.g., Cr, CrO, CrON, MoSi₂, or SiO₂) by a suitable coating technique such as spin coating, roll coating, flow coating, dipping, spraying or doctor coating. The coating is prebaked on a hotplate preferably at a temperature of 60 to 150° C. for 10 seconds to 30 minutes, more preferably at 80 to 120° C. for 30 seconds to 20 minutes. The resulting resist film is generally 0.01 to 2 μm thick.

The resist film is then exposed to a desired pattern of high-energy radiation such as UV, deep-UV, EB, EUV of wavelength 3 to 15 nm, x-ray, soft x-ray, excimer laser light, γ-ray or synchrotron radiation. When UV, deep-UV, EUV, x-ray, soft x-ray, excimer laser light, γ-ray or synchrotron radiation is used as the high-energy radiation, the resist film is exposed thereto directly or through a mask having a desired pattern in a dose of preferably about 1 to 200 mJ/cm², more preferably about 10 to 100 mJ/cm². When EB is used as the high-energy radiation, the resist film is exposed thereto directly or through a mask having a desired pattern in a dose of preferably about 0.1 to 300 μC/cm², more preferably about 0.5 to 200 μC/cm². It is appreciated that the inventive resist composition is suited in micropatterning using KrF excimer laser, ArF excimer laser, EB, EUV, x-ray, soft x-ray, γ-ray or synchrotron radiation, especially in micropatterning using EB or EUV.

After the exposure, the resist film may be baked (PEB) on a hotplate or in an oven preferably at 30 to 150° C. for 10 seconds to 30 minutes, more preferably at 50 to 120° C. for 30 seconds to 20 minutes.

After the exposure or PEB, the resist film is developed in a developer in the form of an aqueous base solution for 3 seconds to 3 minutes, preferably 5 seconds to 2 minutes by conventional techniques such as dip, puddle and spray techniques. A typical developer is a 0.1 to 10 wt %, preferably 2 to 5 wt % aqueous solution of tetramethylammonium hydroxide (TMAH), tetraethylammonium hydroxide (TEAH), tetrapropylammonium hydroxide (TPAH), or tetrabutylammonium hydroxide (TBAH). In the case of positive tone, the resist film in the exposed area is dissolved in the developer whereas the resist film in the unexposed area is not dissolved. In this way, the desired positive pattern is formed on the substrate. In the case of negative tone, inversely the resist film in the exposed area is insolubilized whereas the resist film in the unexposed area is dissolved away.

In an alternative embodiment, a negative pattern can be obtained from the positive resist composition comprising a base polymer containing acid labile groups by effecting organic solvent development. The developer used herein is preferably selected from among 2-octanone, 2-nonanone, 2-heptanone, 3-heptanone, 4-heptanone, 2-hexanone, 3-hexanone, diisobutyl ketone, methylcyclohexanone, acetophenone, methylacetophenone, propyl acetate, butyl acetate, isobutyl acetate, pentyl acetate, butenyl acetate, isopentyl acetate, propyl formate, butyl formate, isobutyl formate, pentyl formate, isopentyl formate, methyl valerate, methyl pentenoate, methyl crotonate, ethyl crotonate, methyl propionate, ethyl propionate, ethyl 3-ethoxypropionate, methyl lactate, ethyl lactate, propyl lactate, butyl lactate, isobutyl lactate, pentyl lactate, isopentyl lactate, methyl 2-hydroxyisobutyrate, ethyl 2-hydroxyisobutyrate, methyl benzoate, ethyl benzoate, phenyl acetate, benzyl acetate, methyl phenylacetate, benzyl formate, phenylethyl formate, methyl 3-phenylpropionate, benzyl propionate, ethyl phenylacetate, and 2-phenylethyl acetate, and mixtures thereof.

At the end of development, the resist film is rinsed. As the rinsing liquid, a solvent which is miscible with the developer and does not dissolve the resist film is preferred. Suitable solvents include alcohols of 3 to 10 carbon atoms, ether compounds of 8 to 12 carbon atoms, alkanes, alkenes, and alkynes of 6 to 12 carbon atoms, and aromatic solvents. Specifically, suitable alcohols of 3 to 10 carbon atoms include n-propyl alcohol, isopropyl alcohol, 1-butyl alcohol, 2-butyl alcohol, isobutyl alcohol, t-butyl alcohol, 1-pentanol, 2-pentanol, 3-pentanol, t-pentyl alcohol, neopentyl alcohol, 2-methyl-1-butanol, 3-methyl-1-butanol, 3-methyl-3-pentanol, cyclopentanol, 1-hexanol, 2-hexanol, 3-hexanol, 2,3-dimethyl-2-butanol, 3,3-dimethyl-1-butanol, 3,3-dimethyl-2-butanol, 2-ethyl-1-butanol, 2-methyl-1-pentanol, 2-methyl-2-pentanol, 2-methyl-3-pentanol, 3-methyl-1-pentanol, 3-methyl-2-pentanol, 3-methyl-3-pentanol, 4-methyl-1-pentanol, 4-methyl-2-pentanol, 4-methyl-3-pentanol, cyclohexanol, and 1-octanol. Suitable ether compounds of 8 to 12 carbon atoms include di-n-butyl ether, diisobutyl ether, di-s-butyl ether, di-n-pentyl ether, diisopentyl ether, di-s-pentyl ether, di-t-pentyl ether, and di-n-hexyl ether. Suitable alkanes of 6 to 12 carbon atoms include hexane, heptane, octane, nonane, decane, undecane, dodecane, methylcyclopentane, dimethylcyclopentane, cyclohexane, methylcyclohexane, dimethylcyclohexane, cycloheptane, cyclooctane, and cyclononane. Suitable alkenes of 6 to 12 carbon atoms include hexene, heptene, octene, cyclohexene, methylcyclohexene, dimethylcyclohexene, cycloheptene, and cyclooctene. Suitable alkynes of 6 to 12 carbon atoms include hexyne, heptyne, and octyne. Suitable aromatic solvents include toluene, xylene, ethylbenzene, isopropylbenzene, t-butylbenzene and mesitylene.

Rinsing is effective for minimizing the risks of resist pattern collapse and defect formation. However, rinsing is not essential. If rinsing is omitted, the amount of solvent used may be reduced.

A hole or trench pattern after development may be shrunk by the thermal flow, RELACS® or DSA process. A hole pattern is shrunk by coating a shrink agent thereto, and baking such that the shrink agent may undergo crosslinking at the resist surface as a result of the acid catalyst diffusing from the resist layer during bake, and the shrink agent may attach to the sidewall of the hole pattern. The bake is preferably at a temperature of 70 to 180° C., more preferably 80 to 170° C., for a time of 10 to 300 seconds. The extra shrink agent is stripped and the hole pattern is shrunk.

### EXAMPLES

Examples of the invention are given below by way of illustration and not by way of limitation. All parts are by weight (pbw).

Quenchers Q-1 to Q-20 having the structure shown below were used in resist compositions.

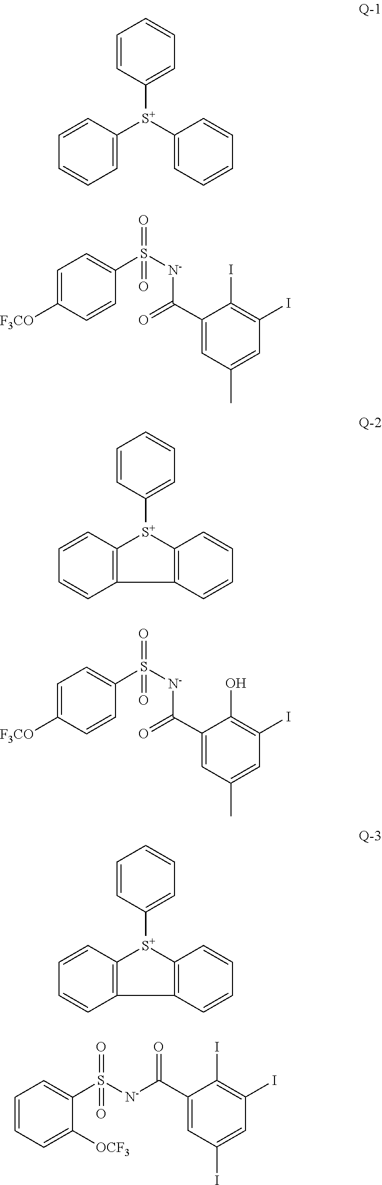 [SMILES: c1ccc([S+](c2ccccc2)c2ccccc2)cc1 ; Cc1cc(I)c(I)c(C(=O)[N-]S(=O)(=O)c2ccc(OC(F)(F)F)cc2)c1 ; c1ccc(-[s+]2c3ccccc3c3ccccc32)cc1 ; Cc1cc(I)c(O)c(C(=O)[N-]S(=O)(=O)c2ccc(OC(F)(F)F)cc2)c1 ; c1ccc(-[s+]2c3ccccc3c3ccccc32)cc1 ; O=C([N-]S(=O)(=O)c1ccccc1OC(F)(F)F)c1cc(I)cc(I)c1I]

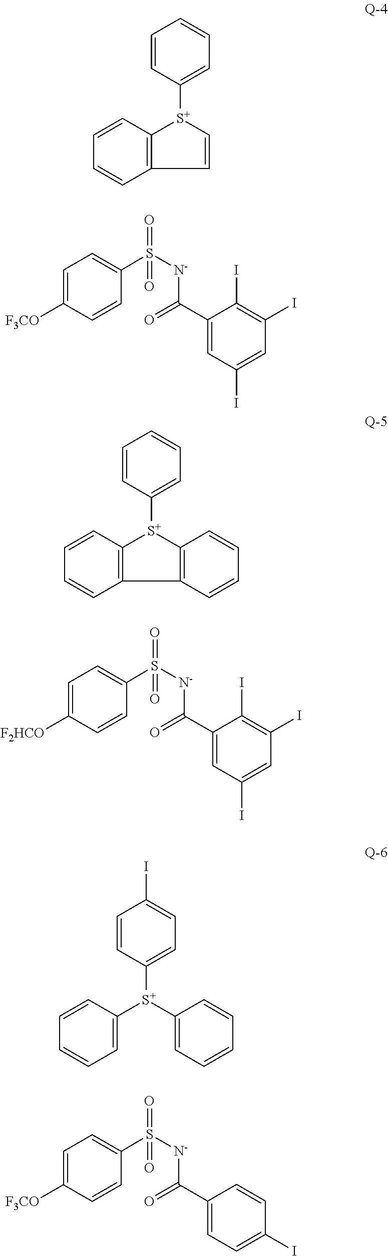 [SMILES: c1ccc(-[s+]2ccc3ccccc32)cc1 ; O=C([N-]S(=O)(=O)c1ccc(OC(F)(F)F)cc1)c1cc(I)cc(I)c1I ; c1ccc(-[s+]2c3ccccc3c3ccccc32)cc1 ; O=C([N-]S(=O)(=O)c1ccc(OC(F)F)cc1)c1cc(I)cc(I)c1I ; Ic1ccc([S+](c2ccccc2)c2ccccc2)cc1 ; O=C([N-]S(=O)(=O)c1ccc(OC(F)(F)F)cc1)c1ccc(I)cc1]

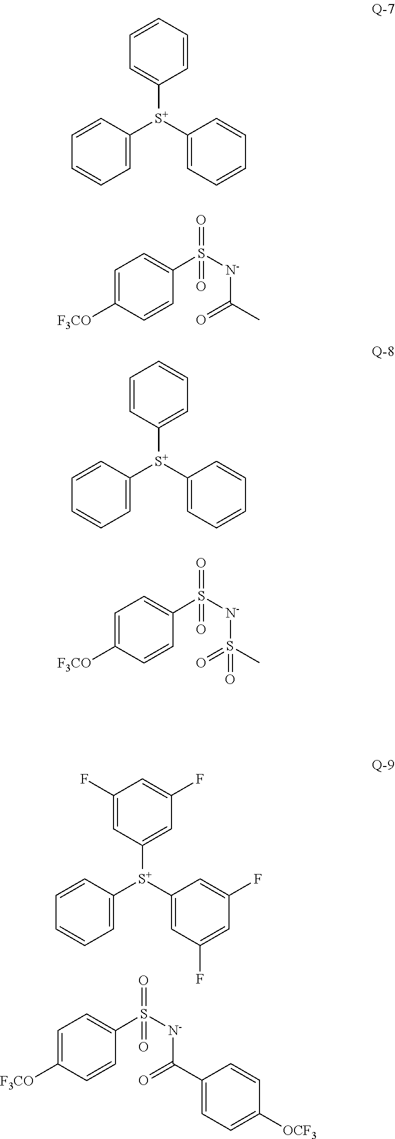 [SMILES: c1ccc([S+](c2ccccc2)c2ccccc2)cc1 ; CC(=O)[N-]S(=O)(=O)c1ccc(OC(F)(F)F)cc1 ; c1ccc([S+](c2ccccc2)c2ccccc2)cc1 ; CS(=O)(=O)[N-]S(=O)(=O)c1ccc(OC(F)(F)F)cc1 ; Fc1cc(F)cc([S+](c2ccccc2)c2cc(F)cc(F)c2)c1 ; O=C([N-]S(=O)(=O)c1ccc(OC(F)(F)F)cc1)c1ccc(OC(F)(F)F)cc1]

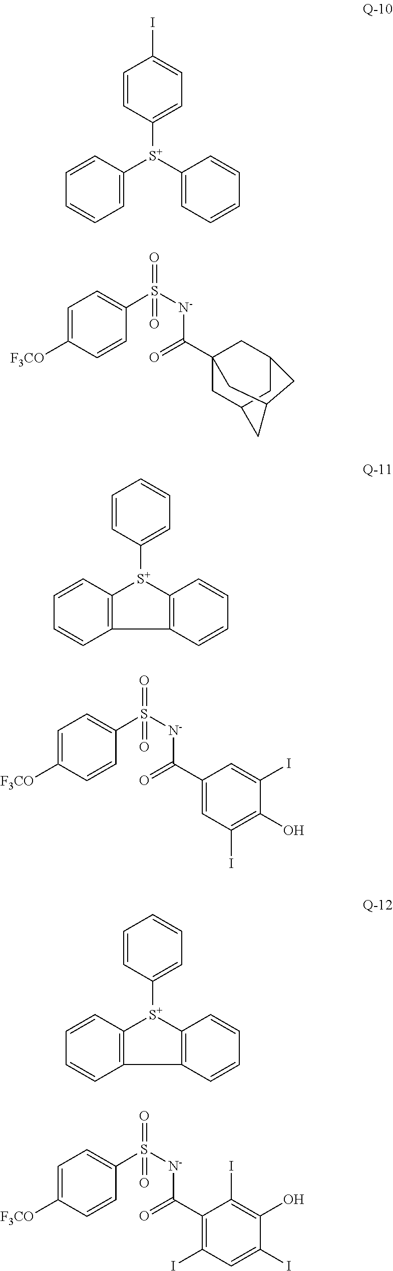 [SMILES: Ic1ccc([S+](c2ccccc2)c2ccccc2)cc1 ; O=C([N-]S(=O)(=O)c1ccc(OC(F)(F)F)cc1)C12CC3CC(CC(C3)C1)C2 ; c1ccc(-[s+]2c3ccccc3c3ccccc32)cc1 ; O=C([N-]S(=O)(=O)c1ccc(OC(F)(F)F)cc1)c1cc(I)c(O)c(I)c1 ; c1ccc(-[s+]2c3ccccc3c3ccccc32)cc1 ; O=C([N-]S(=O)(=O)c1ccc(OC(F)(F)F)cc1)c1c(I)cc(I)c(O)c1I]

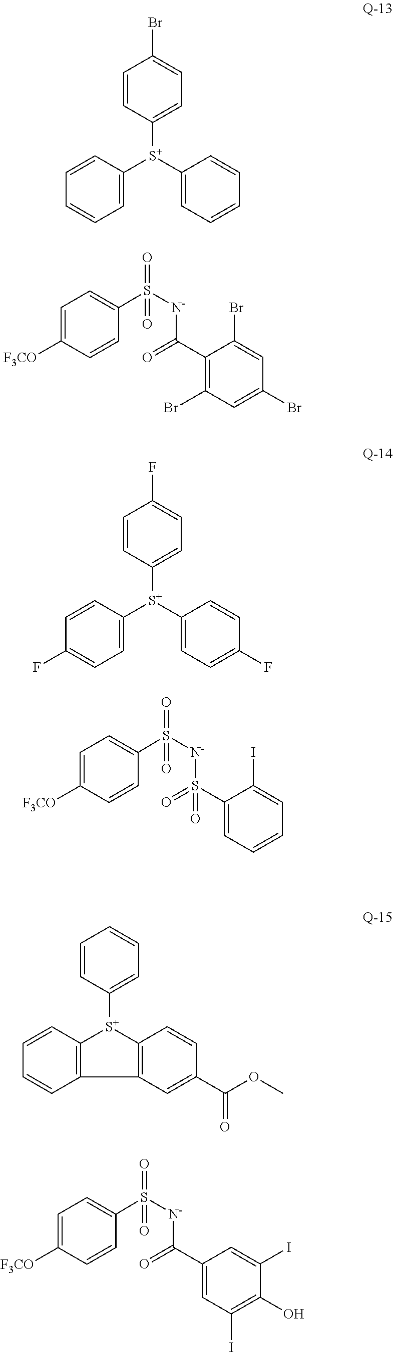 [SMILES: Brc1ccc([S+](c2ccccc2)c2ccccc2)cc1 ; O=C([N-]S(=O)(=O)c1ccc(OC(F)(F)F)cc1)c1c(Br)cc(Br)cc1Br ; Fc1ccc([S+](c2ccc(F)cc2)c2ccc(F)cc2)cc1 ; O=S(=O)([N-]S(=O)(=O)c1ccccc1I)c1ccc(OC(F)(F)F)cc1 ; COC(=O)c1ccc2c(c1)c1ccccc1[s+]2-c1ccccc1 ; O=C([N-]S(=O)(=O)c1ccc(OC(F)(F)F)cc1)c1cc(I)c(O)c(I)c1]

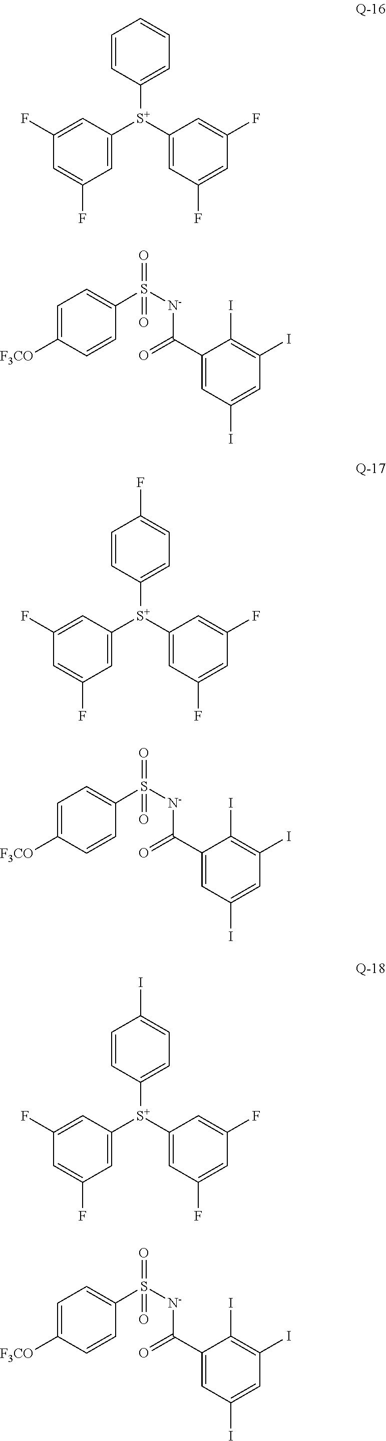 [SMILES: Fc1cc(F)cc([S+](c2ccccc2)c2cc(F)cc(F)c2)c1 ; O=C([N-]S(=O)(=O)c1ccc(OC(F)(F)F)cc1)c1cc(I)cc(I)c1I ; Fc1ccc([S+](c2cc(F)cc(F)c2)c2cc(F)cc(F)c2)cc1 ; O=C([N-]S(=O)(=O)c1ccc(OC(F)(F)F)cc1)c1cc(I)cc(I)c1I ; Fc1cc(F)cc([S+](c2ccc(I)cc2)c2cc(F)cc(F)c2)c1 ; O=C([N-]S(=O)(=O)c1ccc(OC(F)(F)F)cc1)c1cc(I)cc(I)c1I]

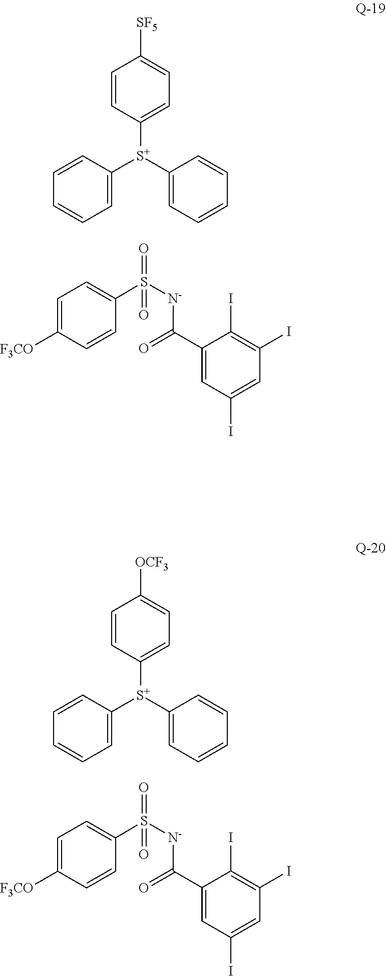

### [Synthesis Example] Synthesis of Base Polymers (Polymers P-1 to P-6)

Base polymers (Polymers P-1 to P-6) of the construction shown below were synthesized by combining selected monomers, and effecting copolymerization reaction in THF solvent, followed by crystallization from methanol, washing with hexane, isolation, and drying. The base polymers were analyzed for composition by ¹H-NMR spectroscopy and for Mw and Mw/Mn by GPC versus polystyrene standards using THF solvent.

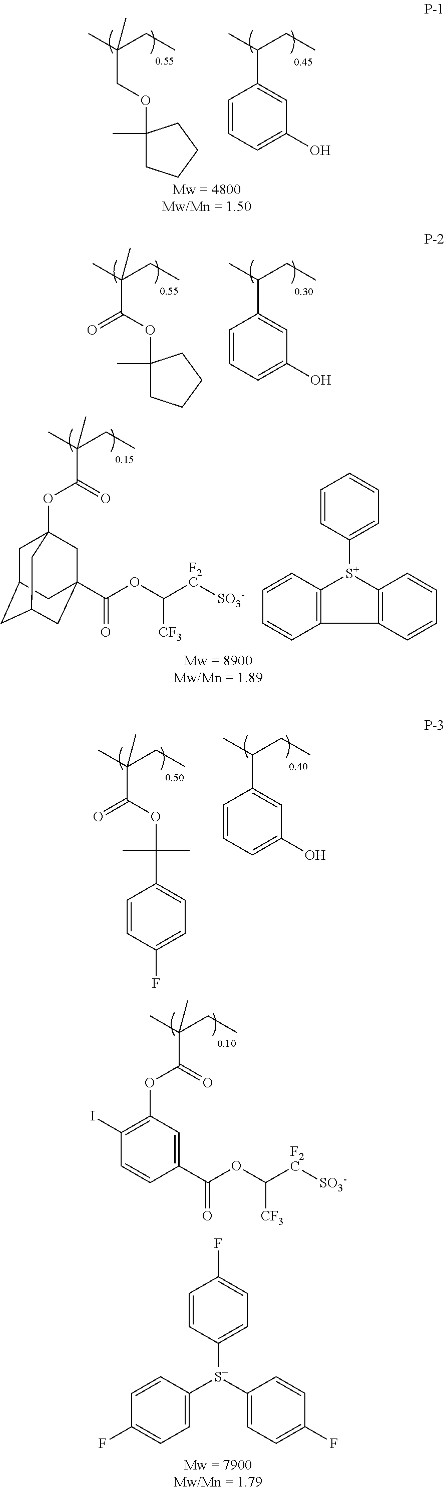

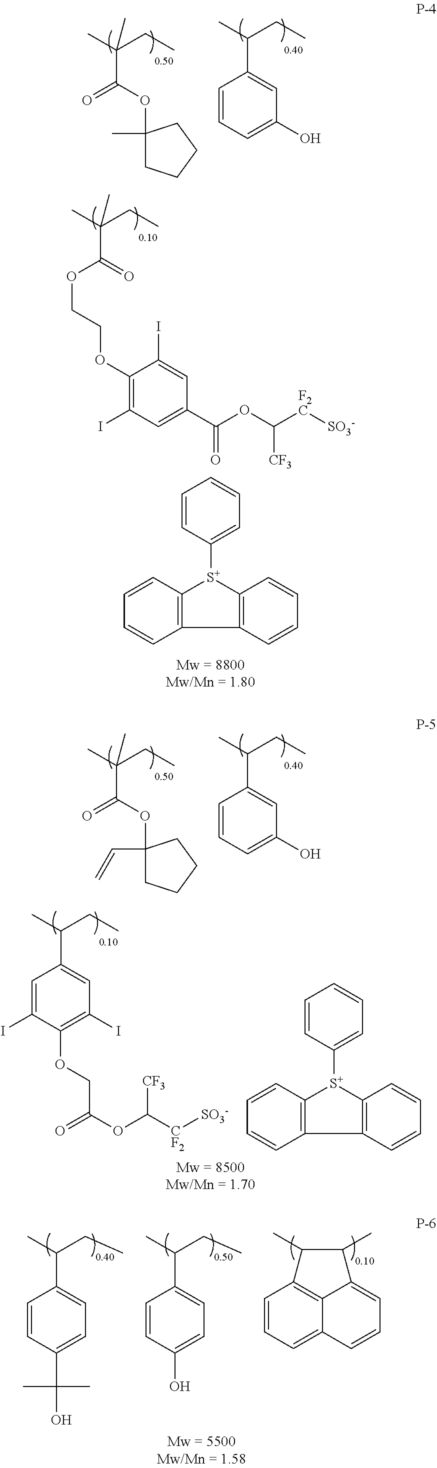

### [Examples 1 to 25 and Comparative Examples 1 to 4] Preparation and Evaluation of Resist Compositions

### (1) Preparation of Resist Compositions

Resist compositions were prepared by dissolving components in a solvent in accordance with the recipe shown in Tables 1 and 2 and filtering the solution through a filter having a pore size of 0.2 μm. The resist compositions of Examples 1 to 24 and Comparative Examples 1 to 3 were of positive tone whereas the resist compositions of Example 25 and Comparative Example 4 were of negative tone

The components in Tables 1 and 2 are identified below.

### Organic Solvents:

PGMEA (propylene glycol monomethyl ether acetate)
DAA (diacetone alcohol)
EL (ethyl lactate)

### Acid Generators: PAG-1 and PAG-2

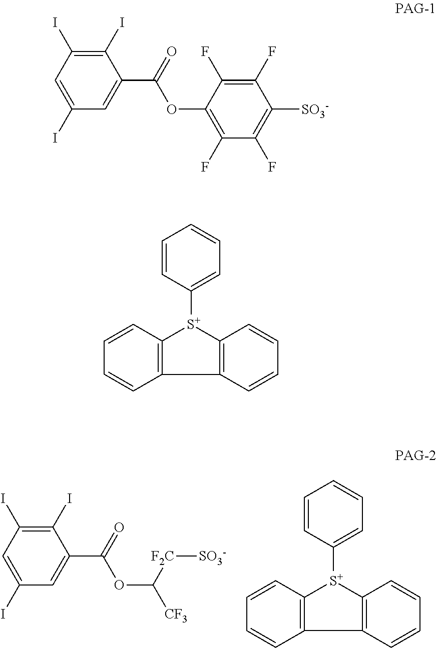

### Blend Quenchers: BQ-1 to BQ-4

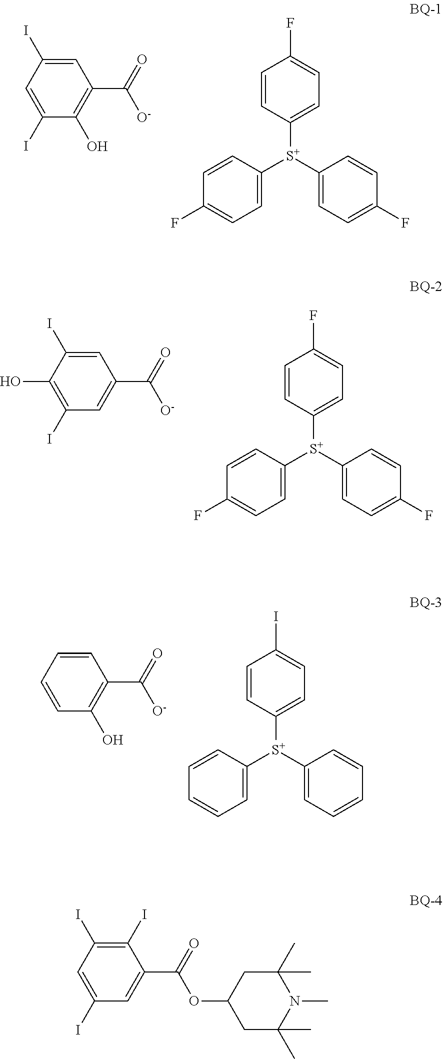

Comparative Quenchers: cQ-1 to cQ-3

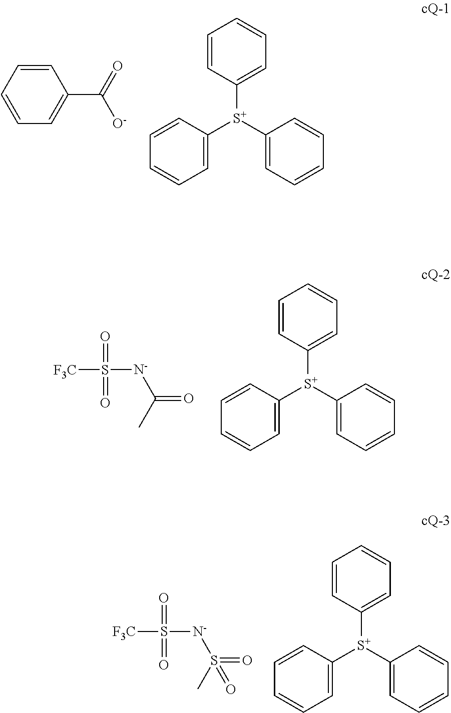 [SMILES: O=C([O-])c1ccccc1 ; c1ccc([S+](c2ccccc2)c2ccccc2)cc1 ; CC(=O)[N-]S(=O)(=O)C(F)(F)F ; c1ccc([S+](c2ccccc2)c2ccccc2)cc1 ; CS(=O)(=O)[N-]S(=O)(=O)C(F)(F)F ; c1ccc([S+](c2ccccc2)c2ccccc2)cc1]

### (2) EUV Lithography Test

Each of the resist compositions in Tables 1 and 2 was spin coated on a silicon substrate having a 20-nm coating of silicon-containing spin-on hard mask SHB-A940 (Shin-Etsu Chemical Co., Ltd., Si content 43 wt %) and prebaked on a hotplate at 100° C. for 60 seconds to form a resist film of 60 nm thick. Using an EUV scanner NXE3400 (ASML, NA 0.33, σ 0.9/0.6, quadrupole illumination), the resist film was exposed to EUV through a mask bearing a hole pattern at a pitch 44 nm (on-wafer size) and +20% bias. The resist film was baked (PEB) on a hotplate at the temperature shown in Tables 1 and 2 for 60 seconds and developed in a 2.38 wt % TMAH aqueous solution for 30 seconds to form a hole pattern having a size of 22 nm in Examples 1 to 24 and Comparative Examples 1 to 3 or a dot pattern having a size of 22 nm in Example 25 and Comparative Example 4.

The resist pattern was observed under CD-SEM (CG6300, Hitachi High-Technologies Corp.). The exposure dose that provides a hole or dot pattern having a size of 22 nm was determined and reported as sensitivity. The size of 50 holes or dots at that dose was measured, from which a 3-fold value (3σ) of the standard deviation (α) was computed and reported as CDU.

The resist compositions are shown in Tables 1 and 2 together with the sensitivity and CDU of EUV lithography.

TABLE 1

| | | | Acid | | | PEB | | |
|---|---|---|---|---|---|---|---|---|
| | | Polymer | generator | Quencher | Organic solvent | temp. | Sensitivity | CDU |
| | | (pbw) | (pbw) | (pbw) | (pbw) | (° C.) | (mJ/cm²) | (nm) |
| Example | 1 | P-1 | PAG-1 | Q-1 | PGMEA (3,000) | 80 | 34 | 3.1 |
| | | (100) | (24.7) | (9.85) | DAA (500) | | | |
| | 2 | P-1 | PAG-1 | Q-2 | PGMEA (3,000) | 80 | 35 | 3.2 |
| | | (100) | (24.7) | (8.73) | DAA (500) | | | |
| | 3 | P-1 | PAG-1 | Q-3 | PGMEA (3,000) | 80 | 32 | 3.0 |
| | | (100) | (24.7) | (9.83) | DAA (500) | | | |
| | 4 | P-1 | PAG-1 | Q-4 | PGMEA (3,000) | 80 | 33 | 3.2 |
| | | (100) | (24.7) | (9.33) | DAA (500) | | | |
| | 5 | P-1 | PAG-1 | Q-5 | PGMEA (3,000) | 80 | 32 | 3.1 |
| | | (100) | (24.7) | (9.65) | DAA (500) | | | |
| | 6 | P-1 | PAG-1 | Q-6 | PGMEA (3,000) | 80 | 33 | 3.2 |
| | | (100) | (24.7) | (8.59) | DAA (500) | | | |
| | 7 | P-1 | PAG-1 | Q-7 | PGMEA (3,000) | 80 | 31 | 3.3 |
| | | (100) | (24.7) | (5.45) | DAA (500) | | | |
| | 8 | P-1 | PAG-1 | Q-8 | PGMEA (3,000) | 80 | 30 | 3.4 |
| | | (100) | (24.7) | (5.81) | DAA (500) | | | |
| | 9 | P-1 | PAG-1 | Q-9 | PGMEA (3,000) | 80 | 30 | 3.2 |
| | | (100) | (24.7) | (7.63) | DAA (500) | | | |
| | 10 | P-1 | PAG-1 | Q-10 | PGMEA (3,000) | 80 | 33 | 3.2 |
| | | (100) | (24.7) | (7.91) | DAA (500) | | | |
| | 11 | P-1 | PAG-1 | Q-11 | PGMEA (3,000) | 80 | 30 | 3.1 |
| | | (100) | (24.7) | (8.73) | DAA (500) | | | |
| | 12 | P-1 | PAG-1 | Q-12 (5.00) | EL (3,000) | 80 | 29 | 3.0 |
| | | (100) | (24.7) | BQ-1 (3.53) | DAA (500) | | | |
| | 13 | P-1 | PAG-1 | Q-13 (4.61) | EL (3,500) | 80 | 31 | 3.2 |
| | | (100) | (24.7) | BQ-2 (3.53) | | | | |
| | 14 | P-1 | PAG-1 | Q-14 (4.11) | PGMEA (3,000) | 80 | 32 | 3.3 |
| | | (100) | (24.7) | BQ-3 (2.63) | DAA (500) | | | |
| | 15 | P-1 | PAG-2 | Q-15 (4.65) | PGMEA (3,000) | 80 | 32 | 3.2 |
| | | (100) | (24.3) | BQ-4 (3.26) | DAA (500) | | | |
| | 16 | P-2 | — | Q-2 | PGMEA (3,000) | 90 | 34 | 3.0 |
| | | (100) | | (8.73) | DAA (500) | | | |
| | 17 | P-3 | — | Q-2 | PGMEA (3,000) | 90 | 32 | 2.9 |
| | | (100) | | (8.73) | DAA (500) | | | |
| | 18 | P-4 | — | Q-2 | PGMEA (3,000) | 90 | 31 | 3.1 |
| | | (100) | | (8.73) | DAA (500) | | | |
| | 19 | P-5 | — | Q-2 | PGMEA (3,000) | 90 | 32 | 3.0 |
| | | (100) | | (8.73) | DAA (500) | | | |
| | 20 | P-1 | PAG-1 | Q-16 | PGMEA (3,000) | 80 | 29 | 3.1 |
| | | (100) | (24.7) | (10.57) | DAA (500) | | | |
| | 21 | P-1 | PAG-1 | Q-17 | PGMEA (3,000) | 80 | 28 | 3.2 |
| | | (100) | (24.7) | (10.75) | DAA (500) | | | |
| | 22 | P-1 | PAG-1 | Q-18 | PGMEA (3,000) | 80 | 30 | 3.3 |
| | | (100) | (24.7) | (11.83) | DAA (500) | | | |
| | 23 | P-1 | PAG-1 | Q-19 | PGMEA (3,000) | 80 | 31 | 3.1 |
| | | (100) | (24.7) | (11.11) | DAA (500) | | | |
| | 24 | P-1 | PAG-1 | Q-20 | PGMEA (3,000) | 80 | 32 | 3.1 |
| | | (100) | (24.7) | (10.69) | DAA (500) | | | |
| | 25 | P-6 | PAG-1 | Q-2 | PGMEA (3,000) | 110 | 36 | 3.7 |
| | | (100) | (24.7) | (8.73) | DAA (500) | | | |

TABLE 2

| | | | Acid | | | PEB | | |
|---|---|---|---|---|---|---|---|---|
| | | Polymer | generator | Quencher | Organic solvent | temp. | Sensitivity | CDU |
| | | (pbw) | (pbw) | (pbw) | (pbw) | (° C.) | (mJ/cm²) | (nm) |
| Comparative | 1 | P-1 | PAG-1 | cQ-1 | PGMEA (3,000) | 80 | 43 | 3.8 |
| Example | | (100) | (24.7) | (3.84) | DAA (500) | | | |
| | 2 | P-1 | PAG-1 | cQ-2 | PGMEA (3,000) | 80 | 31 | 4.1 |
| | | (100) | (24.7) | (4.53) | DAA (500) | | | |
| | 3 | P-1 | PAG-1 | cQ-3 | PGMEA (3,000) | 80 | 30 | 4.4 |
| | | (100) | (24.7) | (4.89) | DAA (500) | | | |
| | 4 | P-5 | PAG-1 | cQ-1 | PGMEA (3,000) | 110 | 45 | 5.0 |
| | | (100) | (24.7) | (3.84) | DAA (500) | | | |

It is demonstrated in Tables 1 and 2 that resist compositions comprising a sulfonium salt of N-carbonyltrifluoromethoxybenzenesulfonamide, N-carbonyldifluoromethoxybenzenesulfonamide, trifluoromethoxybenzenesulfonimide or difluoromethoxybenzenesulfonimide as the quencher offer a high sensitivity and improved CDU. Comparative resist compositions comprising a sulfonium salt of conventional sulfonamide or sulfonimide are insufficient to control acid diffusion and show inferior CDU despite a high sensitivity.

Japanese Patent Application No. 2023-123397 is incorporated herein by reference. Although some preferred embodiments have been described, many modifications and variations may be made thereto in light of the above teachings. It is therefore to be understood that the invention may be practiced otherwise than as specifically described without departing from the scope of the appended claims.

1. A resist composition comprising a quencher containing a sulfonium salt of at least one compound selected from N-carbonyltrifluoromethoxybenzenesulfonamide, N-carbonyldifluoromethoxybenzenesulfonamide, trifluoromethoxybenzenesulfonimide, and difluoromethoxybenzenesulfonimide.

2. The resist composition of claim 1 wherein the sulfonium salt has the formula (1): 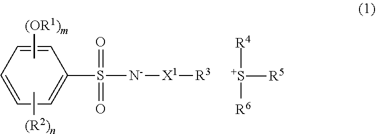
wherein m is an integer of 1 to 5, n is an integer of 0 to 4, m+n is from 1 to 5,
X¹ is —C(═O)— or —S(═O)₂—,
R¹ is trifluoromethyl or difluoromethyl,
R² is a C₁-C₆ saturated hydrocarbyl group, C₁-C₆ saturated hydrocarbyloxy group, hydroxy, carboxy, cyano, nitro or halogen,
R³ is a C₁-C₂₆ hydrocarbyl group which may contain at least one element selected from oxygen, sulfur, nitrogen, and halogen,
R⁴ to R⁶ are each independently halogen or a C₁-C₂₀ hydrocarbyl group which may contain a heteroatom, R⁴ and R⁵ may bond together to form a ring with the sulfur atom to which they are attached.

3. The resist composition of claim 1, further comprising a base polymer.

4. The resist composition of claim 3 wherein the base polymer comprises repeat units having the formula (a1) or repeat units having the formula (a2): 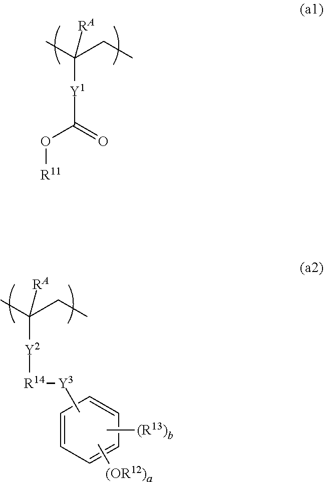
wherein R^{A} is each independently hydrogen or methyl,
Y¹ is a single bond, phenylene, naphthylene, or a C₁-C₁₂ linking group containing at least one moiety selected from ester bond, ether bond and lactone ring,
Y² is a single bond or ester bond,
Y³ is a single bond, ether bond or ester bond,
R¹¹ and R¹² are each independently an acid labile group,
R¹³ is fluorine, trifluoromethyl, cyano or a C₁-C₆ saturated hydrocarbyl group,
R¹⁴ is a single bond or a C₁-C₆ alkanediyl group in which some —CH₂— may be replaced by an ether bond or ester bond,
a is 1 or 2, b is an integer of 0 to 4, and a+b is from 1 to 5.

5. The resist composition of claim 4 which is a chemically amplified positive resist composition.

6. The resist composition of claim 3 wherein the base polymer is free of an acid labile group.

7. The resist composition of claim 6 which is a chemically amplified negative resist composition.

8. The resist composition of claim 3 wherein the base polymer comprises repeat units of at least one type selected from repeat units having the formula (f1), repeat units having the formula (f2), and repeat units having the formula (f3): 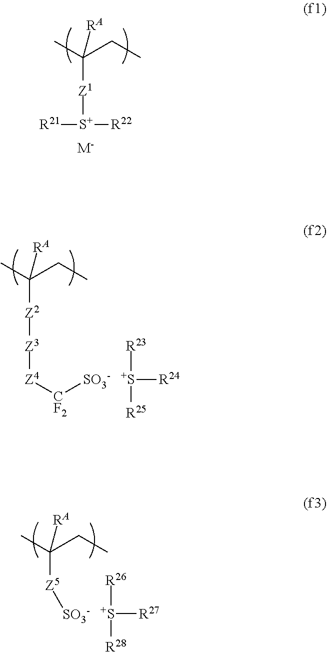
wherein R^{A} is each independently hydrogen or methyl,
Z¹ is a single bond, a C₁-C₆ aliphatic hydrocarbylene group, phenylene group, naphthylene group, or C₇-C₁₈ group obtained by combining the foregoing, or —O—Z¹¹—, —C(═O)—O—Z¹¹— or —C(═O)—NH—Z¹¹—, Z¹¹ is a C₁-C₆ aliphatic hydrocarbylene group, phenylene group, naphthylene group, or C₇-C₁₈ group obtained by combining the foregoing, which may contain a carbonyl moiety, ester bond, ether bond or hydroxy moiety,
Z² is a single bond or ester bond,
Z³ is a single bond, —Z³¹—C(═O)—O—, —Z³¹—O— or —Z³¹—O—C(═O)—, Z³¹ is a C₁-C₁₂ hydrocarbylene group, phenylene group, or C₇-C₁₈ group obtained by combining the foregoing, which may contain a carbonyl moiety, ester bond, ether bond, iodine or bromine,
Z⁴ is methylene, 2,2,2-trifluoro-1,1-ethanediyl or carbonyl,
Z⁵ is a single bond, methylene, ethylene, phenylene, fluorinated phenylene, trifluoromethyl-substituted phenylene group, —O—Z⁵¹—, —C(═O)—O—Z⁵¹—, or —C(═O)—NH—Z⁵¹—, Z⁵¹ is a C₁-C₆ aliphatic hydrocarbylene group, phenylene group, fluorinated phenylene group, or trifluoromethyl-substituted phenylene group, which may contain a carbonyl moiety, ester bond, ether bond, halogen or hydroxy moiety,
R²¹ to R²⁸ are each independently halogen or a C₁-C₂₀ hydrocarbyl group which may contain a heteroatom, a pair of R²³ and R²⁴ or R²⁶ and R²⁷ may bond together to form a ring with the sulfur atom to which they are attached, and
M⁻ is a non-nucleophilic counter ion.

9. The resist composition of claim 1, further comprising an acid generator capable of generating a strong acid.

10. The resist composition of claim 9 wherein the acid generator generates a sulfonic acid, imide acid or methide acid.

11. The resist composition of claim 1, further comprising an organic solvent.

12. The resist composition of claim 1, further comprising a surfactant.

13. A pattern forming process comprising the steps of applying the resist composition of claim 1 onto a substrate to form a resist film thereon, exposing the resist film to high-energy radiation, and developing the exposed resist film in a developer.

14. The process of claim 13 wherein the high-energy radiation is KrF excimer laser, ArF excimer laser, EB or EUV of wavelength 3 to 15 nm.
